(19) United States
(12) Patent Application Publication
(10) Pub. No.: US 2024/0336698 A1
(43) Pub. Date: Oct. 10, 2024

(54) CD38 Chimeric Co-Stimulating Receptor and Uses Thereof

(71) Applicants: Memorial Sloan Kettering Cancer Center, New York, NY (US); Sloan-Kettering Institute For Cancer Research, New York, NY (US); Memorial Hospital For Cancer and Allied Diseases, New York, NY (US); Stichting VUmc, Amsterdam (NL)

(72) Inventors: Maria Themeli, Utrecht (NL); Michel Sadelain, New York, NY (US); Maria Sjöstrand, New York, NY (US); Afroditi Katsarou, Utrecht (NL)

(21) Appl. No.: 18/283,547

(22) PCT Filed: Mar. 22, 2022
(86) PCT No.: PCT/US2022/021325

§ 371 Date: Sep. 22, 2023

Related U.S. Application Data
(60) Provisional application No. 63/275,327, filed on Nov. 3, 2021.
(60) Provisional application No. 63/274,250, filed on Nov. 1, 2021.
(60) Provisional application No. 63/164,355, filed on Mar. 22, 2021.

(51) Int. Cl. C07K 16/28; A61K 39/00; A61P 35/00; C07K 14/705; C07K 14/725

(52) U.S. Cl. CPC C07K 16/2896; A61K 39/4611; A61K 39/4631; A61K 39/464417; A61K 39/464426; A61P 35/00; C07K 14/7051; C07K 14/70521; C07K 16/2878; A61K 2239/13; A61K 2239/21; A61K 2239/28; C07K 2317/24; C07K 2317/622

(57) ABSTRACT

The presently disclosed subject matter provides uses of a chimeric costimulatory receptor (CCR) targeting CD38, and cells comprising a CD38 CCR and an antigen-recognizing receptor, and uses of such cells.

### CROSS-REFERENCE TO RELATED APPLICATIONS

This application claims priority to each of U.S. Provisional Application No. 63/275,327, filed Nov. 3, 2021, U.S. Provisional Application No. 63/274,250, filed Nov. 1, 2021, and U.S. Provisional Application No. 63/164,355, filed on Mar. 22, 2021, the content of each of which is incorporated by reference in its entirety.

### SEQUENCE LISTING

This application contains a Sequence Listing, which was submitted in ASCII format via EFS-Web, and is hereby incorporated by reference in its entirety. The ASCII copy, created on Mar. 22, 2022, is named 0727341333.txt and is 103,875 bytes in size.

### 1. INTRODUCTION

The presently disclosed subject matter provides uses of a chimeric co-stimulating receptor (CCR) targeting CD38 (CD38 CCR), e.g., for reducing and/or abolishing expression of CD38, and reducing and/or preventing Natural Killer (NK) cell depletion. Also provided are cells comprising a CD38 CCR and an antigen-recognizing receptor (e.g., a chimeric antigen receptor, a T-cell receptor (TCR), or a TCR like fusion molecule), compositions comprising the cells, and uses of the cells or compositions for treating and/or preventing tumors and/or pathogen infections.

### 2. BACKGROUND OF THE INVENTION

Cell-based immunotherapy is a therapy with curative potential for the treatment of cancer. T cells and other immune cells may be modified to target tumor antigens through the introduction of genetic material coding for an antigen recognizing receptor, e.g., a Chimeric antigen receptor (CAR) or a TCR like fusion molecule. Patient-engineered CAR T cells have demonstrated remarkable efficacy against a range of liquid and solid malignancies. However, treatment failure and relapses occur in a large fraction of patients. Therefore, there remain needs of improved immunotherapy.

### 3. SUMMARY OF THE INVENTION

The presently disclosed subject matter provides uses of a CD38 CCR in various methods, e.g., for reducing and/or abolishing expression of CD38, reducing and/or preventing Natural Killer (NK) cell depletion. For example, the presently disclosed subject matter provides methods of reducing and/or abolishing expression of CD38 in a cell. In certain embodiments, the method comprises transducing the cell to comprise a chimeric co-stimulating receptor (CCR) that binds to CD38. In certain embodiments, the method comprises introducing into the cell a nucleic acid molecule that encodes a chimeric co-stimulating receptor (CCR) that binds to CD38.

In certain embodiments, the expression of CD38 is an expression level of CD38 on the cell surface. In certain embodiments, the cell comprises an antigen-recognizing receptor that binds to an antigen.

Furthermore, the presently disclosed subject matter provides methods of reducing and/or preventing depletion of a Natural Killer (NK) cell. In certain embodiments, the method comprises transducing the cell to comprise a chimeric co-stimulating receptor (CCR) that binds to CD38. In certain embodiments, the method comprises introducing into the NK cell a nucleic acid molecule that encodes a chimeric co-stimulating receptor (CCR) that binds to CD38.

In certain embodiments, the NK cell comprises an antigen-recognizing receptor that binds to an antigen.

The presently disclosed subject matter further provides methods of treating and/or preventing a disease associated with and/or expressing CD38 in a subject. In certain embodiments, the method comprises administering to the subject (a) a CD38 drug and (b) a cell comprising (i) an antigen-recognizing receptor that binds to an antigen expressed on a tissue or cell of the disease, and (ii) a chimeric co-stimulating receptor (CCR) that binds to CD38.

In certain embodiments, the disease is a tumor. In certain embodiments, the tumor is selected from the group consisting of a hematological cancer, glioblastoma, and a solid tumor.

In certain embodiments, the tumor is a hematological cancer. In certain embodiments, the hematological cancer is selected from the group consisting of multiple myeloma (MM), Non-Hodgkin Lymphoma, Hodgkin Lymphoma, Chronic Lymphocytic Leukemia (CLL), Waldenstrom's Macroglobulinemia, acute lymphoblastic leukemia (ALL), lymphoma, and acute myeloid leukemia (AML). In certain embodiments, the tumor is multiple myeloma. In certain embodiments the antigen is a phosphoantigen In certain embodiments, the antigen is selected from the group consisting of BCMA, GPRC5D, FcRL5, CD41, CD48, P2RY10, CD44v6, CD70, CD123, Kappa light chain, NYESO-1, BOB1, ADGRE2, CLEC12A, CCR1, LILRB2, CD5, CD7, CD10, CD19, CD20, CD22, CD30, CD33, CD34, CD38, CD56, BCMA, CS-1, CD138, Lewis antigen (LeY), WT-1, and ITGB7.

In certain embodiments, the tumor is a solid tumor. In certain embodiments, the solid tumor is selected from the group consisting of breast cancer, colon cancer, non-small cell lung cancer, prostate cancer, gastric, esophageal and pancreatic cancers, hepatocellular carcinoma (HCC), and fibrolamellar carcinoma. In certain embodiments, the CD38 drug is an anti-CD38 antibody. In certain embodiments, the anti-CD38 antibody is isatuximab or daratumumab.

In certain embodiments, the cell is an immunoresponsive cell. In certain embodiments, the cell is a cell of the lymphoid lineage or a cell of the myeloid lineage. In certain embodiments, the cell is selected from the group consisting of T cells, B cells, Natural Killer (NK) cells, and dendritic cells. In certain embodiments, the cell is a T cell. In certain embodiments, the T cell is selected from the group consisting of a cytotoxic T lymphocyte (CTL), a γδ T cell, a tumor-infiltrating lymphocyte (TIL), a regulatory T cell, and a Natural Killer T (NKT) cell. In certain embodiments, the cell is a Natural Killer (NK) cell.

In certain embodiments, the antigen-recognizing receptor is selected from the group consisting of a T cell receptor (TCR), a TCR like fusion molecule, or a chimeric antigen receptor (CAR). In certain embodiments, the TCR like fusion molecule is a recombinant T cell receptor (TCR) comprising an antigen binding chain that comprises: (a) an antigen-binding fragment of an immunoglobulin variable region that binds to an antigen in an HLA-independent manner; and (b) a constant domain that is capable of associating with a CD3ζ polypeptide. In certain embodiments, the constant domain comprises a native or modified T Cell Receptor Alpha Constant (TRAC) polypeptide or a native or modified T Cell Receptor Beta Constant (TRBC) polypeptide. In certain embodiments, the CD3ζ polypeptide is fused to an intracellular domain of a co-stimulatory molecule or a portion thereof.

In certain embodiments, the antigen-recognizing receptor is a CAR. In certain embodiments, the CAR comprises an intracellular domain and an extracellular antigen-binding domain that binds to an antigen. In certain embodiments, the intracellular domain of the CAR comprises a CD3ζ polypeptide. In certain embodiments, the CD3ζ polypeptide is a native or modified CD3ζ polypeptide. In certain embodiments, the modified CD3ζ polypeptide comprises a native ITAM1, an ITAM2 variant consisting of two loss-of-function mutations, and an ITAM3 variant consisting of two loss-of-function mutations. In certain embodiments, the intracellular domain of the CAR further comprises an intracellular domain of a co-stimulatory molecule or a portion thereof.

In certain embodiments, the antigen is a tumor antigen or a pathogen antigen. In certain embodiments, the antigen is a tumor antigen. In certain embodiments, the antigen is a phosphoantigen. In certain embodiments, the tumor antigen is selected from the group consisting of CD19, carbonic anhydrase IX (CAIX), carcinoembryonic antigen (CEA), CD8, CD7, CD10, CD20, CD22, CD30, CD33, CLL1, CD34, CD41, CD44, CD49f, CD56, CD74, CD133, CD138, CD123, CD44V6, an antigen of a cytomegalovirus (CMV) infected cell, ENPP3, epithelial glycoprotein-2 (EGP-2), epithelial glycoprotein-40 (EGP-40), epithelial cell adhesion molecule (EpCAM), receptor tyrosine-protein kinase Erb-B2, Erb-B3, Erb-B4, folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, GPRC5D, FcRL5, ITGB7, NKCS1, EGF1R, EGFR-VIII, CD99, CD70, ADGRE2, CCR1, LILRB2, PRAME, CD48, P2RY10, CD70, BOB1, CLEC12A, CCR1, LILRB2, CD5, CD38, and CS-1.

In certain embodiments, the CCR comprises an extracellular antigen-binding domain that binds to CD38, and an intracellular domain that is capable of delivering a costimulatory signal to the cell but does not alone deliver an activation signal to the cell. In certain embodiments, the intracellular domain of the CCR comprises an intracellular domain of a co-stimulatory molecule or a portion thereof. In certain embodiments, the co-stimulatory molecule is selected from the group consisting of CD28, 4-1BB, OX40, CD17, CD40, CD154, CD97, CD11a/CD18, ICOS, DAP-10, CD2, CD244, CD27, and NKG2D. In certain embodiments, the co-stimulatory molecule is 4-1BB. In certain embodiments, the co-stimulatory molecule is CD28. In certain embodiments, the intracellular domain of the CCR comprises an intracellular domain of a CD28 co-stimulatory molecule and an intracellular domain of a 41-BB co-stimulatory molecule.

In addition, the presently disclosed subject matter provides cells comprising (a) an antigen-recognizing receptor that binds to an antigen; and (b) a chimeric co-stimulating receptor (CCR) that binds to CD38. In certain embodiments, the cell exhibits substantial cytolytic activity against cells that are single positive for the antigen.

In certain embodiments, the antigen-recognizing receptor binds to an antigen with a dissociation constant (K_{d}) of 1×10⁻⁸ M or less. In certain embodiments, the antigen is a tumor antigen or a pathogen antigen. In certain embodiments, the antigen is a phosphoantigen. In certain embodiments, the antigen is a tumor antigen. In certain embodiments, the tumor antigen is selected from the group consisting of CD19, carbonic anhydrase IX (CAIX), carcinoembryonic antigen (CEA), CD8, CD7, CD10, CD20, CD22, CD30, CD33, CLL1, CD34, CD41, CD44, CD49f, CD56, CD74, CD133, CD138, CD123, CD44V6, an antigen of a cytomegalovirus (CMV) infected cell, ENPP3, epithelial glycoprotein-2 (EGP-2), epithelial glycoprotein-40 (EGP-40), epithelial cell adhesion molecule (EpCAM), receptor tyrosine-protein kinase Erb-B2, Erb-B3, Erb-B4, folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, GPRC5D, FcRL5, ITGB7, NKCS1, EGF1R, EGFR-VIII, CD99, CD70, ADGRE2, CCR1, LILRB2, PRAME, CD48, P2RY10, CD70, BOB1, CLEC12A, CCR1, LILRB2, CD5, CD38, and CS-1.

In certain embodiments, the antigen-recognizing receptor is selected from the group consisting of a T cell receptor (TCR), a TCR like fusion molecule, or a chimeric antigen receptor (CAR). In certain embodiments, the TCR like fusion molecule is a recombinant T cell receptor (TCR) comprising an antigen binding chain that comprises: (a) an antigen-binding fragment of an immunoglobulin variable region that binds to an antigen in an HLA-independent manner; and (b) a constant domain that is capable of associating with a CD3ζ polypeptide. In certain embodiments, the constant domain comprises a native or modified TRAC polypeptide or a native or modified TRBC polypeptide. In certain embodiments, the CD3ζ polypeptide is fused to an intracellular domain of a co-stimulatory molecule or a portion thereof.

In certain embodiments, the antigen-recognizing receptor is a CAR. In certain embodiments, the CAR comprises an intracellular domain and an extracellular antigen-binding domain that binds to an antigen. In certain embodiments, the intracellular domain of the CAR comprises a CD3ζ polypeptide. In certain embodiments, the CD3ζ polypeptide is a native or modified CD3ζ polypeptide. In certain embodiments, the modified CD3ζ polypeptide comprises a native ITAM1, an ITAM2 variant consisting of two loss-of-function mutations, and an ITAM3 variant consisting of two loss-of-function mutations. In certain embodiments, the intracellular domain of the CAR further comprises an intracellular domain of a co-stimulatory molecule or a portion thereof.

In certain embodiments, the CCR comprises an extracellular antigen-binding domain that binds to CD38, and an intracellular domain that is capable of delivering a costimulatory signal to the cell but does not alone deliver an activation signal to the cell. In certain embodiments, the intracellular domain of the CCR comprises an intracellular domain of a co-stimulatory molecule or a portion thereof. In certain embodiments, the co-stimulatory molecule is selected from the group consisting of CD28, 4-1BB, OX40, CD17, CD40, CD154, CD97, CD11a/CD18, ICOS, DAP-10, CD2, NKG2D, CD244, and CD27. In certain embodiments, the co-stimulatory molecule is 4-1BB. In certain embodiments, the co-stimulatory molecule is CD28. In certain embodiments, the intracellular domain of the CCR comprises an intracellular domain of a CD28 co-stimulatory molecule and an intracellular domain of a 41-BB co-stimulatory molecule.

In certain embodiments, the cell is an immunoresponsive cell. In certain embodiments, the cell is selected from the group consisting of T cells, B cells, Natural Killer (NK) cells, and dendritic cells. In certain embodiments, the cell is a T cell. In certain embodiments, the T cell is selected from the group consisting of a cytotoxic T lymphocyte (CTL), a γδ T cell, a tumor-infiltrating lymphocyte (TIL), a regulatory T cell, and a Natural Killer T (NKT) cell. In certain embodiments, the cell is a Natural Killer (NK) cell.

The presently disclosed subject matter also provides compositions comprising the cells disclosed herein. In certain embodiments, the composition is a pharmaceutical composition that comprises a pharmaceutically acceptable carrier.

Furthermore, the presently disclosed subject matter provides nucleic acid compositions comprising (a) a first polynucleotide encoding an antigen-recognizing receptor that binds to an antigen; and (b) a second polynucleotide encoding a chimeric co-stimulating receptor (CCR) that binds to CD38. In certain embodiments, a cell comprising the composition exhibits substantial cytolytic activity against cells that are single positive for the antigen. The presently disclosed subject matter also provides nucleic acid compositions comprising (a) a first polynucleotide encoding an antigen-recognizing receptor that binds to CD38; and (b) a second polynucleotide encoding a chimeric co-stimulating receptor (CCR) that binds to an antigen.

The presently disclosed subject matter also provides vectors comprising the nucleic acid compositions disclosed herein.

Furthermore, the presently disclosed subject matter provides cells comprising the nucleic acid compositions disclosed herein. In certain embodiments, the cell is an immunoresponsive cell. In certain embodiments, the cell is selected from the group consisting of T cells, B cells, Natural Killer (NK) cells, and dendritic cells. In certain embodiments, the cell is a T cell. In certain embodiments, the T cell is selected from the group consisting of a cytotoxic T lymphocyte (CTL), a γδ T cell, a tumor-infiltrating lymphocyte (TIL), a regulatory T cell, and a Natural Killer T (NKT) cell. In certain embodiments, the cell is a Natural Killer (NK) cell.

The presently disclosed subject matter provides methods of treating and/or preventing a tumor and/or a pathogen infection in a subject. In certain embodiments, the method comprises administering to the subject the cells or compositions disclosed herein. In certain embodiments, the tumor or pathogen infection is associated with CD38, and/or expression of CD38. In certain embodiments, the method further comprises administering to the subject a CD38 drug. In certain embodiments, the CD38 drug is an anti-CD38 antibody.

The presently disclosed subject matter further provides an immune cell, such as a T cell or NK cell, expressing a chimeric antigen receptor (CAR) and a chimeric co-stimulatory receptor (CCR), wherein the CAR comprises an intracellular CD3ζ signaling domain; a co-stimulatory CD28 intracellular domain; a CD28 transmembrane domain; and an antigen specific binding domain binding to a first antigen; wherein the CCR comprises a co-stimulatory CD28 intracellular domain and a co-stimulatory 4-1BB intracellular domain; a CD28 transmembrane domain; and an antigen specific binding domain binding to a second antigen; wherein the antigen specific binding domains are present on the surface of the immune cell, and wherein the first antigen differs from the second antigen.

Said first and second antigen are, in certain embodiments, selected from CD41, CD48, P2RY10, CD44v6, CD70, CD123, Kappa light chain, NYESO-1, BOB1, ADGRE2, CLEC12A, CCR1, LILRB2, CD5, CD7, CD10, CD19, CD20, CD22, CD30, CD33, CD34, CD38, CD56, BCMA, CS-1, CD138, Lewis antigen (LeY), and WT-1. Said first and second antigen can be selected from the group consisting of CD41, CD48, P2RY10, CD44v6, CD70, CD123, Kappa light chain, NYESO-1, BOB1, ADGRE2, CLEC12A, CCR1, LILRB2, CD5, CD7, CD10, CD19, CD20, CD22, CD30, CD33, CD34, CD38, CD56, BCMA, CS-1, CD138, Lewis antigen (LeY), and WT-1.

Said first antigen can be CD19. Said second antigen can be CD38.

The intracellular signaling domain of the CAR can comprise the intracellular domain of human CD3ζ or a variant thereof. The intracellular domain of the CCR can comprises the human co-stimulatory CD28 intracellular domain and co-stimulatory domain of human 4-1BB, or a variant thereof.

The antigen specific binding domain of the CAR can be a single chain binding domain, e.g., a single chain Fv fragment. The antigen specific binding domain of the CCR is a single chain binding domain, such as a single chain Fv fragment. In certain embodiments, both the antigen specific binding domain of the CAR is a single chain binding domain, e.g., a single chain Fv fragment and the antigen specific binding domain of the CCR is a single chain binding domain, such as a single chain Fv fragment.

In certain embodiments, the co-stimulatory CD28 intracellular domains of the CAR and CCR are substantially identical.

The presently disclosed subject matter further provides a nucleic acid molecule that enables expression of a chimeric antigen receptor (CAR) and a chimeric co-stimulatory receptor (CCR) as defined herein above in a suitable immune cell, e.g., a human T-cell, or human NK cell. Said nucleic acid molecule can be present in a vector, e.g., in a viral vector.

The presently disclosed subject matter further provides a pharmaceutical composition, comprising the immune cell according to the invention, or the nucleic acid molecule according to the invention.

The presently disclosed subject matter further provides a method of producing an immune cell according to the invention, comprising providing immune cells, e.g., human immune cells, such as human T-cells, or human NK cells, and modifying the immune cells by enabling expression of a CAR and a CCR as defined herein above in the immune cells.

The presently disclosed subject matter further provides a method of treating a malignancy, such as a haematological malignancy, in a patient, the method comprising isolating immune cells, such as T cells or NK cells, whereby said immune cells can beisolated from the patient; modifying the immune cells by enabling expression of a CAR and a CCR as defined herein above in the immune cells; and administering the modified immune cells to the patient.

In certain methods of treating a malignancy, such as a haematological malignancy, in a patient, the method comprising the administration of a pharmaceutical composition comprising the immune cell according to the invention, or the nucleic acid molecule according to the invention.

In certain embodiments of treating a malignancy, such as a haematological malignancy, in a patient, the method further comprises the administration of a checkpoint inhibitor.

### 4. BRIEF DESCRIPTION OF THE FIGURES

The following Detailed Description, given by way of example, but not intended to limit the presently disclosed subject matter to specific embodiments described, may be understood in conjunction with the accompanying drawings.

FIG. 1 depicts the expression levels of CD38 in T cells expressing a CD38-targeted CAR.

FIG. 2 depicts T cells expressing a CD38 CCR are CD38 negative.

FIGS. 3A and 3B depict two representative examples of constructs disclosed herein in accordance with certain embodiments of the presently disclosed subject matter. FIG. 3A depicts insertion of a BCMA-targeted CAR and a CD38 CCR in the TRAC locus using homology-directed repair. CRISPR/Cas9 is used to induce a double stranded break. Both the BCMA-targeted CAR and the CD38 CCR are expressed under the TRAC promoter. FIG. 3B depicts expression of a BCMA-targeted CAR under the TRAC promoter and the expression of a CD38-CCR under another promoter of choice (e.g. the EF1a promoter) in the TRAC locus.

FIGS. 4A-4D represent various delivery methods of a CAR and a CD38 CCR to cells. FIG. 4A depicts the integration of a BCMA-targeted CAR and a CD38 CCR at the TRAC locus (knock-in (KI)), thereby knocking out the TRAC locus. In addition, a B2M knockout is introduced using a CRISPR approach comprising a gRNA molecule. FIG. 4B depicts the integration of a BCMA-targeted CAR at the TRAC locus (knock-in (KI)), thereby knocking out the TRAC locus. A CD38 CCR is integrated at exon 1 of the B2M locus, thereby introducing a B2M knockout. FIG. 4C depicts the integration of a BCMA-targeted CAR and a CD38 CCR at the TRAC locus (knock-in (KI)), thereby knocking out the TRAC locus. In addition, a B2M knockout is introduced using a CRISPR approach comprising a gRNA molecule. Furthermore, a CIITA knockout is introduced using a CRISPR approach comprising a gRNA molecule. FIG. 4D depicts the integration of a BCMA-targeted CAR at the TRAC locus (knock-in (KI)), thereby knocking out the TRAC locus. A CD38 CCR is integrated at exon 3 of the CIITA locus, thereby introducing a B2M CIITA knockout. In addition, a B2M knockout is introduced using a CRISPR approach comprising a gRNA molecule.

FIGS. 5A-5E represent combinatorial tumor targeting strategy with a CAR and a CCR resulting in enhanced cytotoxicity. FIG. 5A shows schematic representations of dual targeted CAR+CCR strategies. CD38 CCRs can be combined with any CAR targeting a malignancy-associated antigen target and enhance their function a) by increasing the binding avidity, b) by derivering full costimulation. The figure depicts different combination strategies including 1. a 1st generation CAR and a CD38-CD28 CCR, 2. a 1st generation CAR and a CD38-28BB CCR, 3. a 28z CAR and a CD38-BB CCR or 4. a 28z CAR and a CD38-28BB CCR. FIG. 5B shows schematic diagrams of the vectors used for CARs and CCRs. SP, signal peptide; P2A, “self-cleaving” 2A peptide; IC, intracellular domain; TM, transmembrane domain. FIG. 5C shows AKT1 phosphorylation (phosho-AKT1) in unstimulated CAR T cells assessed by flow cytometry. Representative result from one donor are shown. FIG. 5D shows lysis of luciferase expressing MM1.S cells (BCMA+CD38+). MM1.S were cocultured at different ratios with mock, CD38-28BB, CD38-BB, or CD38Δ T cells. Tumor cell killing was measured in a 16 hour BLI assay (n=4 per group). Data are presented as means±SEM. FIG. 5E shows tumor cell killing analysis. Calcein-loaded 3T3 cells transduced to express CD38 were co-cultured at indicated E:T ratios with either double-targeting BCMA-CAR+CD38-CCR T cells or single targeting CD38-28ζ CAR T cells. Tumor cell killing was measured after 4 hours (n=4 per group). Statistical analysis in (D) and (E) was performed by two-way ANOVA and subsequent multiple comparison, corrected by Turkey test. *p<0.05, ****p<0.0001.

FIGS. 6A-6E depict CD38-CCR engagement resulting in enhanced cytotoxicity of CAR T cells by increasing functional avidity. FIG. 6A shows lysis (determined in a 16 hour BLI assay) of a first generation BCMA-CAR combined with the CD38Δ construct lacking the intracellular costimulatory tail. BCMA-ζ+CD38Δ T cells and the BCMA-ζ+CD38-28BB T cells were incubated with MM1.S cells and at the indicated E:T ratios (n=4). Statistical analysis was performed with a two-way ANOVA and subsequent multiple comparison, corrected by Turkey test. ****p<0.0001. FIG. 6B shows MFI of AKT1 phosphorylation (pAKT1) in BCMA-CAR+CD38-CCR T cells activated by stimulation with UM9 cells for 15 minutes as assessed by flow cytometry (n=4 per group). Statistical analysis was performed by one-way ANOVA and subsequent multiple comparison, corrected by Turkey test. *p<0.05, **p<0.01. FIG. 6C shows the percentage of CAR T cells remaining bound to MM1.S target cells at 1000 pN is shown; data are presented as mean±SEM from 6 pooled measurements; p-values were calculated using a paired t-test. **p<0.01, ***p<0.001. FIG. 6D shows affinity characteristics for anti-CD38 antibodies used to generate scFvs. The surface plasmon resonance-determined dissociation constant (KD value, nmol/L) and half-effective concentration (EC50) when titrated on CHO-CD38 cells (μg/mL), described in (Drent et al., 2017. Mol Ther 25:1946-195829). FIG. 6E shows tumor cell lysis measured in a BLI assay. MM1.S cells were co-cultured at a 1:1 ratio with BCMA-CAR T cells co-expressing CD38-CCRs with gradually lower affinities for CD38 or CD38Δ (n=4 per group). Statistical analysis was performed with a paired t-test.

FIGS. 7A-7D depict CAR and CCR combinations restoring the cytotoxic capacity of T cells against low-antigen expressing tumor variants. FIG. 7A shows cell lysis (determined at 16 hours by BLI) of K562 cells with different BCMA expression and co-cultured with first, second, or third generation BCMA-CAR T cells and double BCMA-CAR+CD38-CCR T cells (BCMA-ζ+CD38-28BB or BCMA-28ζ+CD38-BB) at 1:1 effector to target ratio (n=3 per group). Statistical analysis performed with a two-way ANOVA and subsequent multiple comparison, corrected by Turkey test. **p<0.01, ****p<0.0001. FIG. 7B shows histograms of CD19 and CD38 expression for the NALM6 acute lymphoblastic leukemia cell line (WT) and on two NALM6 clones (clone 12.4 and clone 2). The histogram shows the unstained control. FIG. 7C shows specific lysis of NALM6 clone 2 when co-cultured with CD19-2, double CD19-ζ+CD38-28, or double CD19-2+CD38-28BB T cells (left) or with CD19-28ζ or double CD19-28ζ+CD38-BB (right) T cells. The open squares represent the lysis activity of the CD19 wild type NALM6 cell line when treated with CD19-28ζ CAR T cells. Statistical analysis was performed with using two-way ANOVAs and subsequent multiple comparison, corrected by Turkey test. *p<0.05. FIG. 7D shows specific cytolysis of NALM6 clone 12-4 (200 molecules per cell) and NALM6 clone 2 (20 molecules per cell) when cells were cocultured with CD19-28ζ or double CD19-28ζ+CD38-28BB T cells at indicated E:T ratios. The open squares represent the lysis activity of the CD19 WT NALM6 cell line when treated with CD19-282 CAR T cells. Statistical analysis was performed using two-way ANOVA and subsequent multiple comparison, corrected by Turkey test. ****p<0.0001.

FIG. 8 depicts co-expression of a CCR enhancing the expansion of CAR T cells. Cytokine secretion is shown in supernatants collected from co-cultures with MM1.S (E:T ratio 1:1) for 16 hours (n=4 per group). Statistical analysis was performed by paired t-tests. *p<0.05.

FIGS. 9A-9C 5. CD38-CCR can signal through in-trans binding of the antigen expressed on accessory cells without off-tumor toxicity. FIG. 9A shows cell lysis measured by BLI (after 4 hours) and a Calcein-AM release assay upon co-incubation of U266 cells and calcein-loaded 3T3-CD38 cells cocultured at a 3:1 E:T ratio with mock, BCMA-ζ, BCMA-ζ+CD38Δ, BCMA-ζ+CD38-28, BCMA-28ζ, BCMA-28ζ+CD38-BB or BCMA-ζ+CD38-28BB CAR T cells (n=4 per group). Statistical analysis was performed by a paired t-test. *p<0.05, **p<0.01. FIG. 9B shows cytokine secretion of cell supernatants from FIG. 9A harvested and measured with a flow cytometry-based assay (n=4 per group). Statistical analysis was performed with paired t-tests. *p<0.05, **p<0.01, ***p<0.001. FIG. 9C shows expansion of Mock, BCMA-ζ, BCMA-ζ+CD38Δ, BCMA-ζ+CD38-28, BCMA-28ζ, BCMA-28ζ+CD38-BB or BCMA-ζ+CD38-28BB CAR T cells added in a co-culture of irradiated BCMA+CD38-U266 cells and irradiated 3T3-38+ cells. Cell expansion was measured 7 days later. Statistical analysis was performed with one-way ANOVAs and subsequent multiple comparison, corrected by Turkey test. ****p<0.0001.

FIGS. 10A-10E depict BCMA-CAR+CD38-CCR T cells showing enhanced in vivo anti-tumor function, improved persistence, and reduced expression of exhaustion markers. FIG. 10A shows average tumor burden of mice quantified by BLI and depicted as units of photons per second per square centimeter per steradian (ph/sec/cm²/sr) (n=4 mice per group). Statistical analysis was performed using a two-way ANOVA and subsequent multiple comparison, corrected by Turkey test. *p<0.05. FIG. 10B shows absolute UM9 tumor cell (GFP+/CD38+) numbers in the scaffolds. Each dot represents one scaffold. FIG. 10C shows absolute CAR T cell numbers in the scaffolds. Each dot represents one scaffold. FIG. 10D shows MFI of BCMA on CAR T cells. Each plot represents one scaffold. FIG. 10E shows MFI of BCMA on CAR T cells correlated with the co-existence within the same scaffold. Each plot represents one scaffold. Statistical analysis performed with an unpaired t-test; **p<0.01.

FIGS. 11A-11F shows the CD19-CAR+CD38-CCR combination eliciting improved in vivo anti-tumor function against tumor variants with very low antigen density. FIG. 11A shows average tumor burden of mice injected with FFLuc-GFP NALM6 clone 12.4 cells over time. Tumor burden is depicted as units of photons per second per square centimeter per steradian (ph/sec/cm2/sr) (n=5 mice per group). FIG. 11B shows survival of mice injected with NALM6 clone 12-4. FIG. 11C shows average tumor burden of mice injected with FFLuc-GFP NALM6 clone 2 cells over time. Tumor burden is depicted as units of photons per second per square centimeter per steradian (ph/sec/cm2/sr); n=5 mice per group. FIG. 11D shows survival of mice injected with NALM6 clone 2. Statistical analysis of tumor burden (A and C) was performed with two-way ANOVAs and subsequent multiple comparison, corrected by Turkey test. *p<0.05. Statistical analysis of survival (B and D) was performed with log-rank tests. ***p<0.001, ****p<0.0001.

FIGS. 12A and 12B depict tumor burden in an in vivo acute lymphoblastic leukemia (ALL) model. FIG. 12A shows tumor burden for individual mice injected with NALM6 clone 12.4 and treated with mock, CD38-28BB, CD19-BBζ, CD19-28ζ, CD19-ζ+CD38-28BB, CD19-28ζ+CD38-BB or CD19-28ζ+CD38-28BB, as quantified by luminescence of the tumors (ph/sec/cm2/sr). FIG. 12B shows tumor burden for individual mice injected with Nalm6 clone 2 treated with mock, CD38-28BB, CD19-BBζ, CD19-28ζ, CD19-ζ+CD38-28BB, CD19-28ζ+CD38-BB or CD19-28ζ+CD38-28BB, as quantified by luminescence of the tumors (ph/sec/cm2/sr).

FIG. 13 depicts lysis of CD38-expressing mouse NIH 3T3 cells by T cells transduced with mock vector, CD38-28ζ alone, BCMA-28ζ alone, or by a combination of BCMA-28ζ+CD38-28BB. E:T means effector/target ratio.

### 5. DETAILED DESCRIPTION OF THE INVENTION

The presently disclosed subject matter provides uses of a chimeric co-stimulating receptor (CCR) targeting CD38 (referred to as “CD38 CCR”), e.g., for reducing and/or abolishing expression of CD38, reducing and/or preventing Natural Killer (NK) cell depletion. Also provided are cells comprising a CD38 CCR and an antigen-recognizing receptor (e.g., a chimeric antigen receptor, a T-cell receptor (TCR), or a TCR like fusion molecule), compositions comprising the cells, and uses of the cells or compositions for treating and/or preventing tumors and/or pathogen infections. The presently disclosed subject matter is based, at least in part, on the discovery that a CD38 CCR can abolish CD38 expression on T cells, e.g., engineered T cells, e.g., T cells expressing a CAR. Thus, a CD38 CCR can be used to knock out CD38. See Example 1. As shown in Example 1, T cells expressing a CAR that does not target CD38 has high expression of CD38, which can lead to fratricide killing of the CAR-T cells, when the CAR-T cells are used in combination with a CD38 drug, e.g., an anti-CD38 antibody. Thus, CAR-T cells that further express a CD38 CCR can be used in combination with an anti-CD38 antibody (e.g., daratumumab) for treating diseases associated with CD38.

The presently disclosed subject matter further aims to improve the efficacy of CAR immunotherapy for malignancies, especially haematological malignancies, by co-expression of two engineered receptors, namely a CAR and a CCR receptor, that are targeting different antigens. It was surprisingly found that if both receptors contain a transmembrane and a co-stimulatory intracellular domain of CD28, a superior effect on anti-tumor function was obtained. The co-expression of both receptors on immune cells is a successful strategy to improve cytotoxicity of said cells and eradication of antigen-low tumor variants and provide optimal co-stimulation to enhance immune cell persistence.

Non-limiting embodiments of the presently disclosed subject matter are described by the present specification and Examples.

For purposes of clarity of disclosure and not by way of limitation, the detailed description is divided into the following subsections:
5.1. Definitions;
5.2. CD38 CCR;
5.3. Cells;
5.4. Nucleic Acid Compositions and Vectors;
5.5. Formulations and Administration;
5.6. Methods;
5.7. Kits; and
5.8. Exemplary Embodiments.

### 5.1. Definitions

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

The singular forms “a”, “an” and “the”, as used herein, are intended to include the plural forms as well.

The term “or”, as used herein, includes any and all combinations of one or more of the associated listed items, unless the context clearly indicates otherwise (e.g. if an “either . . . or” construction is used).

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold, of a value.

As used herein, a “co-stimulatory molecule” refers to a cell surface molecule other than an antigen receptor or its ligand that can provide an efficient response of lymphocytes to an antigen. In certain embodiments, a co-stimulatory molecule can provide optimal lymphocyte activation. In certain embodiments, the co-stimulatory molecule comprises a co-stimulatory domain. The term “co-stimulatory domain”, as used herein, refers to a stimulation domain of a CAR and/or CCR that provides a secondary non-specific activation mechanism through the propagation of the primary specific signal.

As used herein, a “co-stimulatory ligand” refers to a molecule that upon binding to its receptor (e.g., a co-stimulatory molecule) produces a co-stimulatory response, e.g., an intracellular response that effects the stimulation provided when an antigen-recognizing receptor (e.g., a chimeric antigen receptor (CAR)) binds to its target antigen.

The term “adoptive immunotherapy”, also called “adoptive cell transfer” and “cellular adoptive immunotherapy”, as is used herein, refers to the administration of donor or autologous immune cells, e.g., T cells or NK cells, for the treatment of a disease or condition, wherein the disease or condition is characterized by an insufficient or inadequate immune response. Adoptive immunotherapy can be an appropriate treatment for any disease or condition where the elimination of infected or transformed cells has been demonstrated to be achieved by immune cells such as T cells and NK cells. For example, disease or conditions include, but are not limited to, malignancies such as, melanoma, prostate, breast, colorectal stomach, throat and neck, pancreatic, cervical, ovarian, bone, leukemia and lung cancer; viral infections such as hepatitis B, hepatitis C and immunodeficiency virus infections; bacterial infections such as tuberculosis, leprosy and listeriosis, and parasitic infections such as malaria. Types of adoptive cell therapy include chimeric antigen receptor T-cell (CAR T-cell) therapy.

By “immunoresponsive cell” or “immune cell” is meant a cell of hematopoictic origin that plays a role in the immune response and/or that functions in an immune response or a progenitor, or progeny thereof. In certain embodiments, the immunoresponsive cell is a cell of lymphoid lineage. In certain embodiments, the immunoresponsive cell is a lymphocyte. Non-limiting examples of cells of lymphoid lineage include T cells, Natural Killer (NK) cells, B cells, and stem cells from which lymphoid cells may be differentiated. In certain embodiments, the immunoresponsive cell is a cell of myeloid lineage. In certain embodiments, the immunoresponsive cell is a myeloid cell. Non-limiting examples of myeloid cells include monocytes, macrophages, cosinophils, mast cells, basophils, and granulocytes.

The term “T cell”, as used herein, refers to a CD4 T-cell or a CD8 T-cell. The term T-cell encompasses T helper (TH) cells such as TH1 cells, TH2 cells and TH17 cells.

The term “Natural Killer (NK) cell”, as used herein, refers to a subset of peripheral blood lymphocytes defined by the expression of CD56 or CD16 and the absence of the T cell receptor.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when an antigen-recognizing receptor (e.g, a TCR or a CAR) binds to an antigen, a formation of an immunological synapse occurs that includes clustering of many molecules near the bound receptor (e.g. CD4 or CD8, CD3γ/δ/ε/ζ, etc.). This clustering of membrane bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, CD40 and ICOS. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

The term “antigen-recognizing receptor” as used herein refers to a receptor that is capable of activating an immune or immunoresponsive cell (e.g., a T-cell) in response to its binding to an antigen.

The terms “chimeric co-stimulating receptor”, “chimeric co-stimulatory receptor (CCR)”, or “CCR” refer to a chimeric receptor that binds to an antigen, e.g., CD38, and provides a co-stimulatory signal, but does not provide an activation signal to a cell comprising the CCR. For example, but without any limitation, a CCR can be a type of chimeric antigen receptor (CAR) that mediates co-stimulation but does not by itself provide immune cell activation. Various CCRs are described in US20020018783 the contents of which are incorporated by reference in their entireties. CCRs mimic co-stimulatory signals, but unlike, CARs, do not provide an activation signal. In certain embodiments, the CCR lacks a CD3ζ polypeptide. In certain embodiments, when expressed on immune cells in combination with an antigen recognition receptor (e.g., a CAR or TCR that activates the cell), the CCR is targeted to a second antigen. In certain embodiments, a CCR comprises two or more co-stimulatory domains, such as a CD28 co-stimulatory domain and a co-stimulatory 4-1BB intracellular domain.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fe fragment of intact antibody, clear more rapidly from the circulation, and may have less non-specific tissue binding of an intact antibody (Wahl et al., J. Nucl. Med. 24:316-325 (1983). As used herein, antibodies include whole native antibodies, bispecific antibodies; chimeric antibodies; Fab, Fab′, single chain variable fragment (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat, E. A., et al. (1991) Sequences of Proteins of Immunological Interest, Fifth Edition, U.S. Department of Health and Human Services, NIH Publication No. 91-3242). In certain embodiments, the CDRs are identified according to the Kabat system. In certain embodiments, the CDRs are identified using the Kabat numbering system accessible at http://www.abysis.org/abysis/index.html. In certain embodiments, the CDRs are identified according to the IMGT system. In certain embodiments, the CDRs are identified using the IMGT numbering system accessible at http://www.abysis.org/abysis/index.html.

As used herein, the term “single-chain variable fragment”, “single-chain variable fragment (scFv)”, or “scFv” is a fusion protein of the variable regions of the heavy (V_{H}) and light chains (V_{L}) of an immunoglobulin covalently linked to form a V_{H}::V_{L} heterodimer. The V_{H} and V_{L} are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including V_{H}- and V_{L}-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27 (6): 455-51; Peter et al., J Cachexia Sarcopenia Muscle 2012 Aug. 12; Shich et al., J Imunol 2009 183 (4): 2277-85; Giomarelli et al., Thromb Hacmost 2007 97 (6): 955-63; Fife eta., J Clin Invst 2006 116 (8): 2252-61; Brocks et al., Immunotechnology 1997 3 (3): 173-84; Moosmayer et al., Ther Immunol 1995 2 (10:31-40). Agonistic scFvs having stimulatory activity have been described (see, e.g., Peter et al., J Bioi Chern 2003 25278 (38): 36740-7; Xie et al., Nat Biotech 1997 15 (8): 768-71; Ledbetter et al., Crit Rev Immunol 1997 17 (5-6): 427-55; Ho et al., BioChim Biophys Acta 2003 1638 (3): 257-66).

As used herein, the term “affinity” or “binding affinity” is meant a measure of binding strength. Affinity can depend on the closeness of stereochemical fit between antibody combining sites and antigen determinants, on the size of the area of contact between them, and/or on the distribution of charged and hydrophobic groups. For example, affinity refers to the apparent binding affinity, which is expressed as the equilibrium dissociation constant (Kd) between the binding domain and its epitope on a target molecule. Said binding affinity is the sum of the attractive and repulsive forces operating between the binding domain and its epitope. As used herein, the term “affinity” also includes “avidity”, which refers to the strength of the antigen-antibody bond after formation of reversible complexes. Methods for calculating the affinity of an antibody for an antigen are known in the art, including, but not limited to, various antigen-binding experiments, e.g., functional assays (e.g., flow cytometry assay).

The terms “chimeric antigen receptor (CAR)”, “chimeric antigen receptor” or “CAR”, as used herein, refer to a molecule comprising an extracellular antigen-binding domain that is fused to an intracellular signaling domain that is capable of activating or stimulating an immune or immunoresponsive cell, and a transmembrane domain. For example, but without any limitation, a CAR can be an artificial transmembrane protein receptor minimally comprising an antigen binding domain, a transmembrane domain and an intracellular signaling domain, capable of activating or stimulating immune cells. In certain embodiments, the extracellular antigen-binding domain of a CAR comprises an scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. In certain embodiments, the CAR is selected to have high binding affinity or avidity for the antigen.

The term “T-cell receptor (TCR)”, as used herein, refers to a molecule that is present on the surface of T cells that is responsible for recognizing fragments of antigen as peptides bound to major histocompatibility complex (MHC) molecules.

The term “intracellular signaling domain”, as used herein, refers to an intracellular domain which transduces an effector function signal and directs the cell to perform a specialized function. The term “effector function” refers to a specialized function of a cell. In certain embodiments, the effector function of a T-cell can be cytolytic activity or helper activity including the secretion of cytokines. In certain embodiments, an intracellular signaling domain is or comprises a CD3ζ intracellular signaling domain. In certain embodiments, an intracellular signaling domain is or comprises a human CD3ζ intracellular signaling domain, or a variant thereof.

The term “cluster of differentiation 3 (CD3)”, as is used herein, refers to a protein complex that is composed of a CD3γ chain, a CD3δ chain, and two CD3ε chains. These chains associate with a TCR and a CD3-zeta (ζ-chain) to generate an activation signal in T lymphocytes. The TCR, CD3-zeta, and the other CD3 chains together constitute a TCR complex.

The term “CD3ζ” or “CD3-zeta”, as used herein, refers to human leukocyte differentiation antigen 3 zeta. In certain embodiments, human CD3ζ is identified by UniProt entry P20963 or by NCBI Reference No: NP_932170. In certain embodiments, the intracellular domain of human CD3ζ corresponds to, comprises, or consists of amino acid residues 52-164 of UniProt entry P20963. In certain embodiments, the intracellular domain of human CD3ζ corresponds to, comprises, or consists of amino acid residues 52-164 of NCBI Reference No: NP_932170. CD3ζ comprise immunoreceptor tyrosine-based activation motifs (ITAMs). Phosphorylation of these motifs results in the activation of downstream signaling pathways.

The term “CD28”, as is used herein, refers to a T-cell-specific surface glycoprotein CD28 that provides co-stimulatory signals required for T cell activation and survival. In certain embodiments, the transmembrane domain corresponds to, comprises, or consists of amino acid residues 153-179 of Uniprot entry P10747. In certain embodiments, the transmembrane domain corresponds to, comprises, or consists of amino acid residues 153-179 of NCBI Reference No: NP_006130. In certain embodiments, the co-stimulatory domain corresponds to, comprises, or consists of the cytoplasmic part of CD28. In certain embodiments, the cytoplasmic part of CD28 corresponds to, comprises, or consists of amino acid residues 180-220 of Uniprot entry P10747. In certain embodiments, the cytoplasmic part of CD28 corresponds to, comprises, or consists of amino acid residues 180-220 of NCBI Reference No: NP_006130. This part comprises a so called YMNM motif, beginning at tyrosine 191, which is important for recruitment of SH2-domain containing proteins.

The term “OX40, or CD134”, as is used herein, refers to a member of the tumor necrosis factor (TNF) receptor family. In certain embodiments, the co-stimulatory signaling domain corresponds to, comprises, or consists of the cytoplasmic part of CD134. In certain embodiments, the cytoplasmic part of CD134 corresponds to, comprises, or consists of amino acid residues 236-277 of Uniprot entry P43489.

The term “ICOS, or CD278”, as is used herein, refers to CD28-related, co-stimulatory molecule that is expressed on activated T cells. In certain embodiments, the co-stimulatory signaling domain corresponds to, comprises, or consists of the cytoplasmic part of CD278. In certain embodiments, the cytoplasmic part of CD278 corresponds to, comprises, or consists of amino acid residues 162-199 of Uniprot entry Q9Y6W8.

The term “2B4, or CD244”, as is used herein, refers to a cell surface receptor expressed on natural killer cells and some T cells. In certain embodiments, the co-stimulatory signaling domain corresponds to, comprises, or consists of the cytoplasmic part of CD244. In certain embodiments, the cytoplasmic part of CD244 corresponds to, comprises, or consists of amino acid residues 251-370 of Uniprot entry Q9BZW8.

The term “CD27 polypeptide”, as is used herein, refers to a member of the TNF-receptor superfamily. This receptor plays a key role in regulating B-cell activation and immunoglobulin synthesis. In certain embodiments, the co-stimulatory signaling domain corresponds to, comprises, or consists of the cytoplasmic part of CD27. In certain embodiments, the cytoplasmic part of CD27 corresponds to, comprises, or consists of amino acid residues 213-260 of Uniprot entry P26842.

The term “CD40 polypeptide”, as is used herein, refers to a member of the TNF-receptor superfamily. This receptor plays a key in the induction of immunoglobulin secretion in B cells. In certain embodiments, the co-stimulatory signaling domain corresponds to, comprises, or consists of the cytoplasmic part of CD40. In certain embodiments, the cytoplasmic part of CD40 corresponds to, comprises, or consists of amino acid residues 216-277 of Uniprot entry P25942.

The term “NKG2D”, as is used herein, refers to is a transmembrane protein belonging to the CD94/NKG2 family of C-type lectin-like receptors. NKG2D is expressed by all NK cells, most NKT cells and subsets of γδ+ T cells (Wu et al., 1999. Science 285:730-732; Jamieson et al., 2002. Immunity 17:19-29). In addition, NKG2D is present on the cell surface of all human CD8+ T cells. NKG2D forms a complex with DNAX-activating protein of 10 kDa (DAP10) (Wu et al., 1999. Science 285:730-732). In certain embodiments, the co-stimulatory signaling domain corresponds to, comprises, or consists of the cytoplasmic part of NKG2D. In certain embodiments, the cytoplasmic part of NKG2D corresponds to, comprises, or consists of amino acid residues 1-51 of Uniprot entry P26718.

The term “4-1BB, or CD137”, as used herein, refers to a member of the tumor necrosis factor receptor (TNFR) superfamily with an amino acid sequence provided as UniProt entry Q07011 or a NCBI Ref. No.: NP_001552. In certain embodiments, the co-stimulatory intracellular signaling domain of human 4-1BB corresponds to, comprises, or consists of amino acid residues 214-255 of Uniprot entry Q07011. In certain embodiments, the co-stimulatory intracellular signaling domain of human 4-1BB corresponds to, comprises, or consists of amino acid residues 214-255 of NCBI Ref. No.: NP 001552.

As used herein, the term “substantially identical” or “substantially homologous” refers to a polypeptide or a nucleic acid molecule exhibiting at least about 50% identical or homologous to a reference amino acid sequence (for example, any of the amino acid sequences described herein) or a reference nucleic acid sequence (for example, any of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% identical or homologous over their entire length to the amino acid sequence or the nucleic acid sequence used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv703) disclosed herein. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., XBLAST and NBLAST) can be used.

As used herein, the term “a conservative sequence modification” refers to an amino acid modification that does not significantly affect or alter the binding characteristics of the reference antibody or antigen-binding fragment thereof comprising the amino acid sequence. Conservative modifications can include amino acid substitutions, additions and deletions. Modifications can be introduced into the extracellular antigen-binding domain of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function using the functional assays described herein. In certain embodiments, no more than one, no more than two, no more than three, no more than four, no more than five residues within a specified sequence or a CDR region are altered.

By “disease” is meant any condition, disease or disorder that damages or interferes with the normal function of a cell, tissue, or organ, e.g., neoplasm, and pathogen infection of cell.

By “effective amount” is meant an amount sufficient to have a therapeutic effect. In certain embodiments, an “effective amount” is an amount sufficient to arrest, ameliorate, or inhibit the continued proliferation, growth, or metastasis (e.g., invasion, or migration) of a neoplasm.

By “endogenous” is meant a nucleic acid molecule or polypeptide that is normally expressed in a cell or tissue.

By “exogenous” is meant a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated cell” is meant a cell that is separated from the molecular and/or cellular components that naturally accompany the cell.

The term “antigen-binding domain” or “antigen specific binding domain”, as used herein, refers to a domain capable of specifically binding a particular antigenic determinant or set of antigenic determinants present on a cell. For example, the antigen-binding domain can be a domain of a CAR and/or CRR which specifically binds to a particular antigen. In certain embodiments, said binding domain binds only to the target that is specified. While in certain embodiments binding to other targets cannot be excluded, such binding to another target would occur with a lower affinity, e.g., at least 10× lower than the affinity of binding to the specified target. For example, when a CD38-specific binding domain binds with a Kd of 5 μM to CD38, such CD38-specific binding domain can, in certain embodiments, bind to another target with a Kd of more than 10 μM, e.g., more than 50 μM. In certain embodiments, “antigen specific binding domain” comprises a single chain binding domain. Single chain binding domains can include a single chain variable domain (scFv), a camelid VHH molecule, a shark immunoglobulin-derived variable new antigen receptor, a tandem scFv, a scFab, an improved scFab (Koerber et al., 2015. J Mol Biol 427:576-86), or an antibody mimetic such as a designed ankyrin repeat protein, a binding protein that is based on a Z domain of protein A, a binding protein that is based on a fibronectin type III domain, engineered lipocalin, and a binding protein that is based on a human Fyn SH3 domain (Skerra, 2007. Current Opinion Biotechnol 18:295-304; Škrlec et al., 2015. Trends Biotechnol 33:408-418). In certain embodiments, an antigen specific binding domain is human or humanized. De-immunization and/or humanization is often used to reduce immunogenicity of non-human molecules. De-immunization involves the identification of linear T-cell epitopes in a binding domain of interest, using bioinformatics, and their subsequent replacement by site-directed mutagenesis to non-immunogenic sequences. Methods for de-immunization are known in the art, for example from WO098/52976. In certain approaches to circumvent immunogenicity of non-human binding domains that are or may be applied to humans involves humanization. Various recombinant DNA-based approaches have been established that are aimed at increasing the content of amino acid residues in binding domains that also occur at the same or a similar position in a human binding domain, while retaining specificity and affinity of the parental non-human binding domain. In certain embodiments, the amino acid residues are those residues that occur in binding domains, e.g., immunoglobulin-based binding domain, that are encoded by genomic germ line sequences. Methods for humanizing antibodies are known to a person skilled in the art and include grafting of CDRs (Queen et al., 1989. PNAS 86:10029; Carter et al., 1992. PNAS 89:4285; resurfacing (Padlan et. al., 1991. Mol Immunol 28:489; superhumanization (Tan et. al., 2002. J Immunol 169:1119), human string content optimization (Lazar et al., 2007. Mol Immunol 44:1986) and humaneering (Almagro et al., 2008. Frontiers Biosci 13:1619). Certain useful methods are described in the published international applications WO2011080350; WO2014033252 and WO2009004065; and in Qu et al., 1999. Clin. Cancer Res. 5:3095-3100; Ono et al., 1999. Mol. Immunol. 36:387-395. These methods rely on analyses of the antibody structure and sequence comparison of non-human and human antibodies in order to evaluate the impact of the humanization process into immunogenicity of the final product. Humanization can include the construction of chimeric binding bodies, e.g., antibodies, in which a non-human binding domain is attached, for example by amino acid bonding, to a human protein.

If required, an antigen specific binding domain can be fused to a transmembrane domain, co-stimulatory intracellular domain and an intracellular signaling domain through a linking group which provides conformational flexibility so that the antigen specific binding domain can associate and bind to its epitope. In certain embodiments, a linker group is a linker polypeptide comprising from 1 to about 60 amino acid residues, e.g., from 5 to about 40 amino acid residues, e.g., about 15 amino acid residues such as 10 amino acid residues, 11 amino acid residues, 12 amino acid residues, 13 amino acid residues, 14 amino acid residues, 15 amino acid residues, 16 amino acid residues, 17 amino acid residues, 18 amino acid residues, 19 amino acid residues or 20 amino acid residues. Non-limiting examples of such amino acid sequences include Gly-Ser linkers, for example of the type (Glyₓ Ser_{y})_{z} such as, for example, (Gly₄ Ser)₃, (Gly₄ Ser)₇ or (Gly₃ Ser₂)₃, as described in WO 99/42077, and the GS30, GS15, GS9 and GS7 linkers described in, for example, WO 06/040153 and WO 06/122825, as well as hinge-like regions, such as the hinge regions of naturally occurring heavy chain antibodies or similar sequences (such as described in WO 94/04678). An exemplary linker is a (Gly₄ Ser)₃ linker. In certain embodiments, a linker group can also be present between the transmembrane domain and an intracellular signaling domain, and/or between an intracellular signaling domain and a co-stimulatory domain and/or between two or more intracellular signaling domains.

In certain embodiments, the linker is a G4S linker. In certain embodiments, the linker comprises or consists of the amino acid sequence set forth in SEQ ID NO: 85, which is provided below:

[SEQ ID NO: 85]
GGGGSGGGGSGGGSGGGGS

In certain embodiments, the linker comprises or consists of the amino acid sequence set forth in SEQ ID NO: 86, which is provided below:

[SEQ ID NO: 86]
GGGGSGGGGSGGGGS

In certain embodiments, the linker comprises or consists of the amino acid sequence set forth in SEQ ID NO: 87, which is provided below:

[SEQ ID NO: 87]
GGGGSGGGGSGGGGSGGGSGGGGS

In certain embodiments, the linker comprises or consists of the amino acid sequence set forth in SEQ ID NO: 88, which is provided below:

[SEQ ID NO: 88]
GGGGSGGGGGGGGSGGGGSGGGSGGGGS

In certain embodiments, the linker comprises or consists of the amino acid sequence set forth in SEQ ID NO: 89, which is provided below:

[SEQ ID NO: 89]
GGGGS

In certain embodiments, the linker comprises or consists of the amino acid sequence set forth in SEQ ID NO: 90, which is provided below:

[SEQ ID NO: 90]
GGGGSGGGGS

In certain embodiments, the linker comprises or consists of the amino acid sequence set forth in SEQ ID NO: 91, which is provided below:

[SEQ ID NO: 91]
GGSGG

The terms “neoplasm”, “malignancy”, “tumor”, and “cancer”, as used herein, refer to a disease characterized by deregulated or uncontrolled cell growth or by pathological proliferation of a cell or tissue and its subsequent migration to or invasion of other tissues or organs. For example, neoplasm growth is typically uncontrolled and progressive, and occurs under conditions that would not elicit, or would cause cessation of, multiplication of normal cells. Neoplasm can affect a variety of cell types, tissues, or organs, including but not limited to an organ selected from bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasms include cancers, such as sarcomas, carcinomas, or plasmacytomas (malignant tumor of the plasma cells). In certain embodiments, the neoplasm is cancer.

The term “hematological malignancy”, as used herein, refers to a malignancy that affects blood cells and/or bone marrow cells, and includes lymphoma, leukemia, myeloma or other lymphoid malignancies such as plasmacytoma and Waldenstrom's macroglobulinemia, as well as cancers of the spleen and the lymph nodes.

By “specifically binds” is meant a polypeptide or a fragment thereof that recognizes and binds to a biological molecule of interest (e.g., a polypeptide), but which does not substantially recognize and bind other molecules in a sample, for example, a biological sample, which naturally includes a presently disclosed polypeptide.

The term “tumor antigen” as used herein refers to an antigen (e.g., a polypeptide) that is uniquely or differentially expressed on a tumor cell compared to a normal or non-neoplastic cell. In certain embodiments, a tumor antigen includes any polypeptide expressed by a tumor that is capable of activating or inducing an immune response via an antigen recognizing receptor or capable of suppressing an immune response via receptor-ligand binding.

The term “CD19”, as is used herein, refers to a transmembrane glycoprotein belonging to the immunoglobulin superfamily. CD19 is classified as a type I transmembrane protein, with a single transmembrane domain, a cytoplasmic C-terminus, and extracellular N-terminus. The amino acid sequence of human CD19 can be found as UniProt entry P15391. CD19 is a marker for B cell lymphomas and leukemias. CD19 is expressed on most B lineage malignancies, including, e.g., acute lymphoblastic leukaemia, chronic lymphocyte leukaemia and non-Hodgkin's lymphoma.

The terms “comprises”, “comprising”, and are intended to have the broad meaning ascribed to them in U.S. Patent Law and can mean “includes”, “including” and the like. The terms “comprise” and “comprising”, and conjugations thereof, are open language and specify the presence of stated features but do not preclude the presence or addition of one or more other features. It will be understood that when a particular step of a method is referred to as subsequent to another step, it can directly follow said other step or one or more intermediate steps may be carried out before carrying out the particular step, unless specified otherwise.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system, but can affect people with a poorly functioning or suppressed immune system.

As used herein, “a functional fragment” of a molecule or polypeptide includes a fragment of the molecule or polypeptide that retains at least about 80%, at least about 85%, at least about 90%, at least about 95%, or at least about 100% of the primary function of the molecule or polypeptide.

The term “nucleic acid molecule”, as is used herein, refers to a molecule consisting of nucleotides which are covalently linked to each other by phosphodiester-bonds of a sugar/phosphate-backbone. In certain embodiments, the nucleic acid molecules are DNA or RNA molecules. The term “nucleic acid molecule” also encompasses modified nucleic acid molecules, such as base-modified, sugar-modified or backbone-modified DNA or RNA molecules.

The term “vector”, as is used herein, refers to an isolated nucleic acid molecule which can be used to deliver a nucleic acid of the presently disclosed subject matter to the interior of a cell. Numerous vectors are known in the art including, but not limited to, linear polynucleotides, polynucleotides associated with ionic or amphiphilic compounds, plasmids, and viruses.

The term “viral vector”, as is used herein, is a vector that includes nucleic acid elements derived from viruses. Examples of viral vectors include, but are not limited to, adeno-associated viral vectors, alphavirus vectors, retroviral vectors, lentiviral vectors, and derivatives thereof such as vaccinia viral/retroviral chimeric vectors (Falkner and Holzer, 2004. Curr Gene Ther 4: 417-26). Said viral vector can be packaged into a viral particle comprising said viral nucleic acid elements and a nucleic acid of the presently disclosed subject matter, and is able to transduce said nucleic acid of the presently disclosed subject matter encoding a CAR and a CCR receptor.

The term “autologous”, as is used herein, refers to any material derived from the same individual to whom it is later to be re-introduced into.

The term “allogeneic”, as is used herein, refers to any material derived from a different member of the same species as the individual to whom the material is introduced. Two or more individuals are said to be allogeneic to one another when the genes at one or more loci are not identical.

Other aspects of the presently disclosed subject matter are described in the following disclosure and are within the ambit of the presently disclosed subject matter.

### 5.2. CD38 CCR

CD38 is a type II 45 kDa glycoprotein, which is usually present in the cellular surface membrane. In addition, CD38 is an intracellular signaling protein. NK cells predominantly express CD38 in a constitutive manner (Nagler et al., J. Immunol. (1989); 143:3183-3191), whereas CD3ζ precursors T and B lymphocytes show a pattern of expression whereby the CD38 molecule is upregulated upon activation or different functional status (Snoeck et al., Exp. Hematol (1993); 21:1480-1486). The contact of CD38 with certain molecules for lymphocyte function (e.g., TCR, BCR, CD19, and in NK-cells with FcγRIII/CD16) is required for signal transduction and generation of downstream processes, e.g., initiation of specific transcriptional programs, secretion of cytokines and activation of lymphocyte effector functions (Funaro et al., Eur. J. Immunol. (1993); 23:2407-2411; Kitanaka et al., J. Immunol. (1997); 159:184-192).

The term “CD38”, as is used herein, refers to a non-lineage-restricted, single pass type II transmembrane glycoprotein that synthesizes and hydrolyzes cyclic adenosine 5′-diphosphate-ribose, an intracellular calcium ion mobilizing messenger. CD38 is robustly expressed in almost all hematologic malignancies. Alternative names for this protein are ADP-ribosyl cyclase and 2-phospho-cyclic-ADP-ribose transferase. The gene encoding human CD38 is located on cytogenetic band 4p15. In certain embodiments, human CD38 is identified by UniProt accession number P28907.

The presently disclosed subject matter provides uses of a CD38 CCR for various methods, e.g., for reducing and/or abolishing expression of CD38 in a cell, and for reducing and/or preventing depletion of Natural Killer (NK) cells. In certain embodiments, the CD38 CCR reduces and/or abolishes an expression level of CD38 on the cell surface of the cell.

In certain embodiments, the CCR comprises an extracellular antigen-binding domain that binds to a CD38 and an intracellular domain that is capable of delivering a costimulatory signal to the cell but does not alone deliver an activation signal to a cell comprising the CCR. In certain embodiments, the CCR binds to a human CD38 polypeptide. In certain embodiments, human CD38 polypeptide comprises the amino acid sequence having a UniProt accession number P28907. In certain embodiments, the human CD38 polypeptide comprises the amino acid sequence having a NCBI Reference No. NP_001766 (SEQ ID NO: 1) or a fragment thereof. SEQ ID NO: 1 is provided below:

[SEQ ID NO: 1]
MANCEFSPVSGDKPCCRLSRRAQLCLGVSILVLILVVVLAVVVPRWRQQW
SGPGTTKRFPETVLARCVKYTEIHPEMRHVDCQSVWDAFKGAFISKHPCN
ITEEDYQPLMKLGTQTVPCNKILLWSRIKDLAHQFTQVQRDMFTLEDTLL
GYLADDLTWCGEFNTSKINYQSCPDWRKDCSNNPVSVFWKTVSRRFAEAA
CDVVHVMLNGSRSKIFDKNSTFGSVEVHNLQPEKVQTLEAWVIHGGREDS
RDLCQDPTIKELESIISKRNIQFSCKNIYRPDKFLQCVKNPEDSSCTSEI

In certain embodiments, the CCR binds to the extracellular portion of CD38 (e.g., human CD38). In certain embodiments, the CCR binds to the C-terminal extracellular portion of CD38 (e.g., human CD38).

In certain embodiments, the extracellular antigen-binding domain of the CCR comprises or is an scFv. The scFv can be a human scFv, a humanized scFv, or a murine scFv. In certain embodiments, the scFv is a murine scFv. In certain embodiments, the scFv is a human scFv. The scFv can be derived from fusing the variable heavy and light regions of an antibody. In certain embodiments, the scFv is derived from Fab's (instead of from an antibody, e.g., obtained from Fab libraries). In certain embodiments, the extracellular antigen-binding domain of the CCR comprises or is a Fab. In certain embodiments, the Fab is crosslinked. In certain embodiments, the extracellular antigen-binding domain of the CCR comprises or is a F (ab)₂.

In certain embodiments, the antigen specific binding domain of the CCR expressed on an immune cell according to the presently disclosed subject matter is a single chain binding domain. In certain embodiments, the single chain binding domain is a single chain Fv fragment. Suitable CD38 binding domains have been described in De Luca et al., 2018. Protein Engineering, Design and Selection 31:173-179 and in Drent et al., 2016. Haematologica 101:616-625.

Any of the foregoing molecules may be comprised in a fusion protein with a heterologous sequence to form the extracellular antigen-binding domain of the CCR.

In certain embodiments, the extracellular antigen-binding domain of the CCR comprises a heavy chain variable region (V_{H}) comprising a CDR1 comprising the amino acid sequence set forth in SEQ ID NO: 49 or a conservative modification thereof, a CDR2 comprising the amino acid sequence set forth in SEQ ID NO: 50 or a conservative modification thereof, and a CDR3 comprising the amino acid sequence set forth in SEQ ID NO: 51 or a conservative modification thereof. In certain embodiments, the extracellular antigen-binding domain of the CCR comprises a V_{H} comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51. In certain embodiments, the extracellular antigen-binding domain of the CCR comprises a light chain variable region (V_{L}) comprising a CDR1 comprising the amino acid sequence set forth in SEQ ID NO: 52 or a conservative modification thereof, a CDR2 comprising the amino acid sequence set forth in SEQ ID NO: 53 or a conservative modification thereof, and a CDR3 comprising the amino acid sequence set forth in SEQ ID NO: 54 or a conservative modification thereof. In certain embodiments, the extracellular antigen-binding domain of the CAR comprises a V_{L} comprising a CDR1 comprising the amino acid sequence set forth in SEQ ID NO: 52, a CDR2 comprising the amino acid sequence set forth in SEQ ID NO: 53, and a CDR3 comprising the amino acid sequence set forth in SEQ ID NO: 54.

In certain embodiments, the extracellular antigen-binding domain of the CCR comprises a V_{H} comprising a CDR1 comprising the amino acid sequence set forth in SEQ ID NO: 49 or a conservative modification thereof, a CDR2 comprising the amino acid sequence set forth in SEQ ID NO: 50 or a conservative modification thereof, and a CDR3 comprising the amino acid sequence set forth in SEQ ID NO: 51 or a conservative modification thereof; a V_{L} comprising a CDR1 comprising the amino acid sequence set forth in SEQ ID NO: 52 or a conservative modification thereof, a CDR2 comprising the amino acid sequence set forth in SEQ ID NO: 53 or a conservative modification thereof, and a CDR3 comprising the amino acid sequence set forth in SEQ ID NO: 54 or a conservative modification thereof. In certain embodiments, the extracellular antigen-binding domain of the CCR comprises a V_{H} comprising a CDR1 comprising amino acids having the sequence set forth in SEQ ID NO: 49, a CDR2 comprising the amino acid sequence set forth in SEQ ID NO: 50, and a CDR3 comprising the amino acid sequence set forth in SEQ ID NO: 51; and a V_{L} comprising a CDR1 comprising the amino acid sequence set forth in SEQ ID NO: 52, a CDR2 comprising the amino acid sequence set forth in SEQ ID NO: 53, and a CDR3 comprising the amino acid sequence set forth in SEQ ID NO: 54.

In certain embodiments, the extracellular antigen-binding domain of the CCR comprises a V_{H} comprising an amino acid sequence that is at least about 80% (e.g., at least about 85%, at least about 90%, or at least about 95%) homologous or identical to the amino acid sequence set forth in SEQ ID NO: 55 over its full length. For example, the extracellular antigen-binding domain of the first antigen-recognizing receptor comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 55. In certain embodiments, the extracellular antigen-binding domain of the CCR comprises a V_{H} comprising the amino acid sequence set forth in SEQ ID NO: 55.

In certain embodiments, the extracellular antigen-binding domain of the CCR comprises a V_{L} comprising an amino acid sequence that is at least about 80% (e.g., at least about 85%, at least about 90%, or at least about 95%) homologous or identical to the amino acid sequence set forth in SEQ ID NO: 56 over its full length. For example, the extracellular antigen-binding domain of the CCR comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 56. In certain embodiments, the extracellular antigen-binding domain of the CCR comprises a V_{L} comprising the amino acid sequence set forth in SEQ ID NO: 56.

In certain embodiments, the extracellular antigen-binding domain of the CCR comprises a V_{H} comprising an amino acid sequence that is at least about 80% (e.g., at least about 85%, at least about 90%, or at least about 95%) homologous or identical to the amino acid sequence set forth in SEQ ID NO: 55, and a V_{L} comprising an amino acid sequence that is at least about 80% (e.g., at least about 85%, at least about 90%, or at least about 95%) homologous or identical to the amino acid sequence set forth in SEQ ID NO: 56.

In certain embodiments, the extracellular antigen-binding domain of the CCR comprises a V_{H} comprising the amino acid sequence set forth in SEQ ID NO: 55. In certain embodiments, the extracellular antigen-binding domain of the CCR comprises a V_{L} comprising the amino acid sequence set forth in SEQ ID NO: 56. In certain embodiments, the extracellular antigen-binding domain of the CCR comprises V_{H} comprising the amino acid sequence set forth in SEQ ID NO: 55 and a V_{L} comprising the amino acid sequence set forth in SEQ ID NO: 56.

In certain embodiments, the extracellular antigen-binding domain of the CCR is an scFv that comprises or consist of the amino acid sequence set forth in SEQ ID NO: 57. SEQ ID NOs: 49-57 are provided in the following Table 1.

TABLE 1

| | anti-CD38 scFv | | |
|---|---|---|---|
| CDRs | 1 | 2 | 3 |
| V_{H} | SYAIS [SEQ ID | RIIRFLGIANYAQKFQG | EPGERDPDAVDI [SEQ |
| | NO: 49] | [SEQ ID NO: 50] | ID NO: 51] |
| V_{L} | RASQGIRSWIA [SEQ | AASSLQS [SEQ ID | QQYNSYPLT [SEQ ID |
| | ID NO: 52] | NO: 53] | NO: 54] |

| | |
|---|---|
| Full V_{H} | QVQLVQSGAEVKKPGSSVKVSCKAFGGTFSSYAISWVRQAPGQGLEWMGRIIRFLGIA |
| | NYAQKFQGRVTLIADKSTNTAYMELSSLRSEDTAVYYCAGEPGERDPDAVDIWGQGTM |
| | VTVSS [SEQ ID NO: 55] |
| Full V_{L} | DIQMTQSPSSISASVGDRVTITCRASQGIRSWIAWYQQKPEKAPKSLIYAASSLQSGV |
| | PSRFSGSGSGTDFTLTISSLOPEDFATYYCQQYNSYPLTFGGGTKVEIK [SEQ ID |
| | NO: 56] |
| scFv | QVQLVQSGAEVKKPGSSVKVSCKAFGGTFSSYAISWVRQAPGQGLEWMGRIIRFLGIA |
| | NYAQKFQGRVTLIADKSTNTAYMELSSLRSEDTAVYYCAGEPGERDPDAVDIWGQGTM |
| | VTVSSGGGGSGGGGSGGGGSDIQMTQSPSSISASVGDRVTITCRASQGIRSWIAWYQQ |
| | KPEKAPKSLIYAASSLQSGVPSRFSGSGSGTDFTLTISSLQPEDFATYYCQQYNSYPL |
| | TFGGGTKVEIK [SEQ ID NO: 57] |

In certain embodiments, the V_{H} and V_{L} are linked via a linker. In certain embodiments, the linker comprises a sequence set forth in SEQ ID NO: 85, SEQ ID NO: 86, SEQ ID NO: 87, SEQ ID NO: 88, SEQ ID NO: 89, SEQ ID NO: 90, or SEQ ID NO: 91. In certain embodiments, the linker comprises or consists of the amino acid sequence set forth in SEQ ID NO: 86. In certain embodiments, the extracellular antigen-binding domain of the CCR comprises a heavy chain variable region (V_{H}) comprising a CDR1 comprising the amino acid sequence set forth in SEQ ID NO: 58 or a conservative modification thereof, a CDR2 comprising the amino acid sequence set forth in SEQ ID NO: 59 or a conservative modification thereof, and a CDR3 comprising the amino acid sequence set forth in SEQ ID NO: 60 or a conservative modification thereof. In certain embodiments, the extracellular antigen-binding domain of the CCR comprises a V_{H} comprising a CDR1 comprising the amino acid sequence set forth in SEQ ID NO: 58, a CDR2 comprising the amino acid sequence set forth in SEQ ID NO: 59, and a CDR3 comprising the amino acid sequence set forth in SEQ ID NO: 60. In certain embodiments, the extracellular antigen-binding domain of the CCR comprises a light chain variable region (V_{L}) comprising a CDR1 comprising the amino acid sequence set forth in SEQ ID NO: 61 or a conservative modification thereof, a CDR2 comprising the amino acid sequence set forth in SEQ ID NO: 62 or a conservative modification thereof, and a CDR3 comprising the amino acid sequence set forth in SEQ ID NO: 63 or a conservative modification thereof. In certain embodiments, the extracellular antigen-binding domain of the CAR comprises a V_{L} comprising a CDR1 comprising the amino acid sequence set forth in SEQ ID NO: 61, a CDR2 comprising the amino acid sequence set forth in SEQ ID NO: 62, and a CDR3 comprising the amino acid sequence set forth in SEQ ID NO: 63.

In certain embodiments, the extracellular antigen-binding domain of the CCR comprises a V_{H} comprising a CDR1 comprising the amino acid sequence set forth in SEQ ID NO: 58 or a conservative modification thereof, a CDR2 comprising the amino acid sequence set forth in SEQ ID NO: 59 or a conservative modification thereof, and a CDR3 comprising the amino acid sequence set forth in SEQ ID NO: 60 or a conservative modification thereof; a V_{L} comprising a CDR1 comprising the amino acid sequence set forth in SEQ ID NO: 61 or a conservative modification thereof, a CDR2 comprising the amino acid sequence set forth in SEQ ID NO: 62 or a conservative modification thereof, and a CDR3 comprising the amino acid sequence set forth in SEQ ID NO: 63 or a conservative modification thereof. In certain embodiments, the extracellular antigen-binding domain of the CCR comprises a V_{H} comprising a CDR1 comprising amino acids having the sequence set forth in SEQ ID NO: 58, a CDR2 comprising the amino acid sequence set forth in SEQ ID NO: 59, and a CDR3 comprising the amino acid sequence set forth in SEQ ID NO: 60; and a V_{L} comprising a CDR1 comprising the amino acid sequence set forth in SEQ ID NO: 61, a CDR2 comprising the amino acid sequence set forth in SEQ ID NO: 62, and a CDR3 comprising the amino acid sequence set forth in SEQ ID NO: 63.

In certain embodiments, the extracellular antigen-binding domain of the CCR comprises a V_{H} comprising an amino acid sequence that is at least about 80% (e.g., at least about 85%, at least about 90%, or at least about 95%) homologous or identical to the amino acid sequence set forth in SEQ ID NO: 64 over its full length. For example, the extracellular antigen-binding domain of the first antigen-recognizing receptor comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 64. In certain embodiments, the extracellular antigen-binding domain of the CCR comprises a V_{H} comprising the amino acid sequence set forth in SEQ ID NO: 64.

In certain embodiments, the extracellular antigen-binding domain of the CCR comprises a V_{L} comprising an amino acid sequence that is at least about 80% (e.g., at least about 85%, at least about 90%, or at least about 95%) homologous or identical to the amino acid sequence set forth in SEQ ID NO: 65 over its full length. For example, the extracellular antigen-binding domain of the CCR comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 65. In certain embodiments, the extracellular antigen-binding domain of the CCR comprises a V_{L} comprising the amino acid sequence set forth in SEQ ID NO: 65.

In certain embodiments, the extracellular antigen-binding domain of the CCR comprises a V_{H} comprising an amino acid sequence that is at least about 80% (e.g., at least about 85%, at least about 90%, or at least about 95%) homologous or identical to the amino acid sequence set forth in SEQ ID NO: 64, and a V_{L} comprising an amino acid sequence that is at least about 80% (e.g., at least about 85%, at least about 90%, or at least about 95%) homologous or identical to the amino acid sequence set forth in SEQ ID NO: 65.

In certain embodiments, the extracellular antigen-binding domain of the CCR comprises a V_{H} comprising the amino acid sequence set forth in SEQ ID NO: 64. In certain embodiments, the extracellular antigen-binding domain of the CCR comprises a V_{L} comprising the amino acid sequence set forth in SEQ ID NO: 65. In certain embodiments, the extracellular antigen-binding domain of the CCR comprises V_{H} comprising the amino acid sequence set forth in SEQ ID NO: 64 and a V_{L} comprising the amino acid sequence set forth in SEQ ID NO: 65.

In certain embodiments, the extracellular antigen-binding domain of the CCR is an scFv that comprises or consist of the amino acid sequence set forth in SEQ ID NO: 66. SEQ ID NOs: 58-66 are provided in the following Table 2.

TABLE 2

| | anti-CD38 scFv | | |
|---|---|---|---|
| CDRs | 1 | 2 | 3 |
| V_{H} | SYAIS [SEQ ID | IIVFLGKVNYAQRFQ | EPGARDPDAFDI [SEQ |
| | NO: 58] | [SEQ ID NO: 59] | ID NO: 60] |
| V_{L} | RASQGIRSWLA [SEQ | AASSLQS [SEQ ID | QQYNNYPLT [SEQ ID |
| | ID NO: 61] | NO: 62] | NO: 63] |

| | |
|---|---|
| Full V_{H} | QVQLVQSGAEVKKPGSSVKVSCKPSGGTFRSYAISWVRQAPGQGLEWMRIIVFLGKVN |
| | YAQRFQGVTLTADKSTTTAYMELSSLRSEDTAVYYCTGEPGARDPDAFDIWGQGTMVT |
| | VSSVL [SEQ ID NO: 64] |
| Full V_{L} | DIQMTQSPSSLSASVGDRVTITCRASQGIRSWLAWYQQKPEKAPKSLIYAASSLQSGV |
| | PSRFSGSGSGTDFTLTISSLOPEDFATYYCQQYNNYPLTFGGGTKVEIK [ SEQ ID |
| | NO: 65] |
| scFv | QVQLVQSGAEVKKPGSSVKVSCKPSGGTFRSYAISWVRQAPGQGLEWMRIIVFLGKVN |
| | YAQRFQGVTLTADKSTTTAYMELSSLRSEDTAVYYCTGEPGARDPDAFDIWGQGTMVT |
| | VSSVLGGGGSGGGGSGGGGSDIQMTQSPSSLSASVGDRVTITCRASQGIRSWLAWYQQ |
| | KPEKAPKSLIYAASSLQSGVPSRFSGSGSGTDFTLTISSLQPEDFATYYCQQYNNYPL |
| | TFGGGTKVEIK [SEQ ID NO: 66] |

In certain embodiments, the V_{H} and V_{L} are linked via a linker. In certain embodiments, the linker comprises a sequence set forth in SEQ ID NO: 85, SEQ ID NO: 86, SEQ ID NO: 87, SEQ ID NO: 88, SEQ ID NO: 89, SEQ ID NO: 90, or SEQ ID NO: 91. In certain embodiments, the linker comprises or consists of the amino acid sequence set forth in SEQ ID NO: 86.

In certain embodiments, the extracellular antigen-binding domain of the CCR comprises a heavy chain variable region (V_{H}) comprising a CDR1 comprising the amino acid sequence set forth in SEQ ID NO: 67 or a conservative modification thereof, a CDR2 comprising the amino acid sequence set forth in SEQ ID NO: 68 or a conservative modification thereof, and a CDR3 comprising the amino acid sequence set forth in SEQ ID NO: 69 or a conservative modification thereof. In certain embodiments, the extracellular antigen-binding domain of the CCR comprises a V_{H} comprising a CDR1 comprising the amino acid sequence set forth in SEQ ID NO: 67, a CDR2 comprising the amino acid sequence set forth in SEQ ID NO: 68, and a CDR3 comprising the amino acid sequence set forth in SEQ ID NO: 69. In certain embodiments, the extracellular antigen-binding domain of the CCR comprises a light chain variable region (V_{L}) comprising a CDR1 comprising the amino acid sequence set forth in SEQ ID NO: 70 or a conservative modification thereof, a CDR2 comprising the amino acid sequence set forth in SEQ ID NO: 71 or a conservative modification thereof, and a CDR3 comprising the amino acid sequence set forth in SEQ ID NO: 72 or a conservative modification thereof. In certain embodiments, the extracellular antigen-binding domain of the CAR comprises a V_{L} comprising a CDR1 comprising the amino acid sequence set forth in SEQ ID NO: 70, a CDR2 comprising the amino acid sequence set forth in SEQ ID NO: 71, and a CDR3 comprising the amino acid sequence set forth in SEQ ID NO: 72.

In certain embodiments, the extracellular antigen-binding domain of the CCR comprises a V_{H} comprising a CDR1 comprising the amino acid sequence set forth in SEQ ID NO: 67 or a conservative modification thereof, a CDR2 comprising the amino acid sequence set forth in SEQ ID NO: 68 or a conservative modification thereof, and a CDR3 comprising the amino acid sequence set forth in SEQ ID NO: 69 or a conservative modification thereof; a V_{L} comprising a CDR1 comprising the amino acid sequence set forth in SEQ ID NO: 70 or a conservative modification thereof, a CDR2 comprising the amino acid sequence set forth in SEQ ID NO: 71 or a conservative modification thereof, and a CDR3 comprising the amino acid sequence set forth in SEQ ID NO: 72 or a conservative modification thereof. In certain embodiments, the extracellular antigen-binding domain of the CCR comprises a V_{H} comprising a CDR1 comprising amino acids having the sequence set forth in SEQ ID NO: 67, a CDR2 comprising the amino acid sequence set forth in SEQ ID NO: 68, and a CDR3 comprising the amino acid sequence set forth in SEQ ID NO: 69; and a V_{L} comprising a CDR1 comprising the amino acid sequence set forth in SEQ ID NO: 70, a CDR2 comprising the amino acid sequence set forth in SEQ ID NO: 71, and a CDR3 comprising the amino acid sequence set forth in SEQ ID NO: 72.

In certain embodiments, the extracellular antigen-binding domain of the CCR comprises a V_{H} comprising an amino acid sequence that is at least about 80% (e.g., at least about 85%, at least about 90%, or at least about 95%) homologous or identical to the amino acid sequence set forth in SEQ ID NO: 73 over its full length. For example, the extracellular antigen-binding domain of the first antigen-recognizing receptor comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 73. In certain embodiments, the extracellular antigen-binding domain of the CCR comprises a V_{H} comprising the amino acid sequence set forth in SEQ ID NO: 73.

In certain embodiments, the extracellular antigen-binding domain of the CCR comprises a V_{L} comprising an amino acid sequence that is at least about 80% (e.g., at least about 85%, at least about 90%, or at least about 95%) homologous or identical to the amino acid sequence set forth in SEQ ID NO: 74 over its full length. For example, the extracellular antigen-binding domain of the CCR comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 74. In certain embodiments, the extracellular antigen-binding domain of the CCR comprises a V_{L} comprising the amino acid sequence set forth in SEQ ID NO: 74.

In certain embodiments, the extracellular antigen-binding domain of the CCR comprises a V_{H} comprising an amino acid sequence that is at least about 80% (e.g., at least about 85%, at least about 90%, or at least about 95%) homologous or identical to the amino acid sequence set forth in SEQ ID NO: 73, and a V_{L} comprising an amino acid sequence that is at least about 80% (e.g., at least about 85%, at least about 90%, or at least about 95%) homologous or identical to the amino acid sequence set forth in SEQ ID NO: 74.

In certain embodiments, the extracellular antigen-binding domain of the CCR comprises a V_{H} comprising the amino acid sequence set forth in SEQ ID NO: 73. In certain embodiments, the extracellular antigen-binding domain of the CCR comprises a V_{L} comprising the amino acid sequence set forth in SEQ ID NO: 74. In certain embodiments, the extracellular antigen-binding domain of the CCR comprises V_{H} comprising the amino acid sequence set forth in SEQ ID NO: 73 and a V_{L} comprising the amino acid sequence set forth in SEQ ID NO: 74.

In certain embodiments, the extracellular antigen-binding domain of the CCR is an scFv that comprises or consist of the amino acid sequence set forth in SEQ ID NO: 75. In certain embodiments, the extracellular antigen-binding domain of the CCR is an scFv that comprises or consist of the amino acid sequence set forth in SEQ ID NO: 103. SEQ ID NOs: 67-75 and 103 are provided in the following Table 3.

TABLE 3

| | anti-CD38 scFv | | |
|---|---|---|---|
| CDRs | 1 | 2 | 3 |
| V_{H} | SYAIS [SEQ ID | RIIRFLGKTNHAQKFQG | EPGDRDPDAVDI [SEQ |
| | NO: 67] | [SEQ ID NO: 68] | ID NO: 69] |
| V_{L} | RASQGIRSWLA [SEQ | AASSLQS [SEQ ID | QQYNSYPLT [ SEQ ID |
| | ID NO: 70] | NO: 71] | NO: 72] |

| | |
|---|---|
| Full V_{H} | QVQLVQSGAEVKKPGSSVKVSCKAFGGTFSSYAISWVRQAPGQGLEWMGRIIRFLGKT |
| | NHAQKFQGRVTLTADKSTNTAYMELSSLRSEDTAVYYCAGEPGDRDPDAVDIWGQGTM |
| | VTVSS [SEQ ID NO: 73] |
| Full V_{L} | DIQMTQSPSSLSASVGDRVTITCRASQGIRSWLAWYQQKPEKAPKSLIYAASSLQSGV |
| | PSRFSGSGSGTDFTLTISSLQPEDFATYYCQQYNSYPLTFGGGTKVEIK [SEQ ID |
| | NO: 74] |
| scFv | QVQLVQSGAEVKKPGSSVKVSCKAFGGTFSSYAISWVRQAPGQGLEWMGRIIRFLGKT |
| (V_{H}-V_{L}) | NHAQKFQGRVTLTADKSTNTAYMELSSLRSEDTAVYYCAGEPGDRDPDAVDIWGQGTM |
| | VTVSSGGGGSGGGGSGGGGSDIQMTQSPSSLSASVGDRVTITCRASQGIRSWLAWYQQ |
| | KPEKAPKSLIYAASSLQSGVPSRFSGSGSGTDFTLTISSLQPEDFATYYCQQYNSYPL |
| | TFGGGTKVEIK [SEQ ID NO: 75] |
| scFv | DIQMTQSPSSLSASVGDRVTITCRASQGIRSWLAWYQQKPEKAPKSLIYAASSLQSGV |
| (V_{L}-V_{H}) | PSRFSGSGSGTDFTLTISSLQPEDFATYYCQQYNSYPLTFGGGTKVEIKGGGGSGGGG |
| | SGGGGSQVQLVQSGAEVKKPGSSVKVSCKAFGGTFSSYAISWVRQAPGQGLEWMGRII |
| | RFLGKTNHAQKFQGRVTLTADKSTNTAYMELSSLRSEDTAVYYCAGEPGDRDPDAVDI |
| | WGQGTMVTVSS [SEQ ID NO: 103] |

In certain embodiments, the V_{H} and V_{L} are linked via a linker. In certain embodiments, the linker comprises a sequence set forth in SEQ ID NO: 85, SEQ ID NO: 86, SEQ ID NO: 87, SEQ ID NO: 88, SEQ ID NO: 89, SEQ ID NO: 90, or SEQ ID NO: 91. In certain embodiments, the linker comprises or consists of the amino acid sequence set forth in SEQ ID NO: 86.

In certain embodiments, the extracellular antigen-binding domain of the CCR comprises a heavy chain variable region (V_{H}) comprising a CDR1 comprising the amino acid sequence set forth in SEQ ID NO: 76 or a conservative modification thereof, a CDR2 comprising the amino acid sequence set forth in SEQ ID NO: 77 or a conservative modification thereof, and a CDR3 comprising the amino acid sequence set forth in SEQ ID NO: 78 or a conservative modification thereof. In certain embodiments, the extracellular antigen-binding domain of the CCR comprises a V_{H} comprising a CDR1 comprising the amino acid sequence set forth in SEQ ID NO: 76, a CDR2 comprising the amino acid sequence set forth in SEQ ID NO: 77, and a CDR3 comprising the amino acid sequence set forth in SEQ ID NO: 78. In certain embodiments, the extracellular antigen-binding domain of the CCR comprises a light chain variable region (V_{L}) comprising a CDR1 comprising the amino acid sequence set forth in SEQ ID NO: 79 or a conservative modification thereof, a CDR2 comprising the amino acid sequence set forth in SEQ ID NO: 80 or a conservative modification thereof, and a CDR3 comprising the amino acid sequence set forth in SEQ ID NO: 81 or a conservative modification thereof. In certain embodiments, the extracellular antigen-binding domain of the CAR comprises a V_{L} comprising a CDR1 comprising the amino acid sequence set forth in SEQ ID NO: 79, a CDR2 comprising the amino acid sequence set forth in SEQ ID NO: 80, and a CDR3 comprising the amino acid sequence set forth in SEQ ID NO: 81.

In certain embodiments, the extracellular antigen-binding domain of the CCR comprises a V_{H} comprising a CDR1 comprising the amino acid sequence set forth in SEQ ID NO: 76 or a conservative modification thereof, a CDR2 comprising the amino acid sequence set forth in SEQ ID NO: 77 or a conservative modification thereof, and a CDR3 comprising the amino acid sequence set forth in SEQ ID NO: 78 or a conservative modification thereof; a V_{L} comprising a CDR1 comprising the amino acid sequence set forth in SEQ ID NO: 79 or a conservative modification thereof, a CDR2 comprising the amino acid sequence set forth in SEQ ID NO: 80 or a conservative modification thereof, and a CDR3 comprising the amino acid sequence set forth in SEQ ID NO: 81 or a conservative modification thereof. In certain embodiments, the extracellular antigen-binding domain of the CCR comprises a V_{H} comprising a CDR1 comprising amino acids having the sequence set forth in SEQ ID NO: 76, a CDR2 comprising the amino acid sequence set forth in SEQ ID NO: 77, and a CDR3 comprising the amino acid sequence set forth in SEQ ID NO: 78; and a V_{L} comprising a CDR1 comprising the amino acid sequence set forth in SEQ ID NO: 79, a CDR2 comprising the amino acid sequence set forth in SEQ ID NO: 80, and a CDR3 comprising the amino acid sequence set forth in SEQ ID NO: 81.

In certain embodiments, the extracellular antigen-binding domain of the CCR comprises a V_{H} comprising an amino acid sequence that is at least about 80% (e.g., at least about 85%, at least about 90%, or at least about 95%) homologous or identical to the amino acid sequence set forth in SEQ ID NO: 82 over its full length. For example, the extracellular antigen-binding domain of the first antigen-recognizing receptor comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 82. In certain embodiments, the extracellular antigen-binding domain of the CCR comprises a V_{H} comprising the amino acid sequence set forth in SEQ ID NO: 82.

In certain embodiments, the extracellular antigen-binding domain of the CCR comprises a V_{L} comprising an amino acid sequence that is at least about 80% (e.g., at least about 85%, at least about 90%, or at least about 95%) homologous or identical to the amino acid sequence set forth in SEQ ID NO: 83 over its full length. For example, the extracellular antigen-binding domain of the CCR comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 83. In certain embodiments, the extracellular antigen-binding domain of the CCR comprises a V_{L} comprising the amino acid sequence set forth in SEQ ID NO: 83.

In certain embodiments, the extracellular antigen-binding domain of the CCR comprises a V_{H} comprising an amino acid sequence that is at least about 80% (e.g., at least about 85%, at least about 90%, or at least about 95%) homologous or identical to the amino acid sequence set forth in SEQ ID NO: 82, and a V_{L} comprising an amino acid sequence that is at least about 80% (e.g., at least about 85%, at least about 90%, or at least about 95%) homologous or identical to the amino acid sequence set forth in SEQ ID NO: 83.

In certain embodiments, the extracellular antigen-binding domain of the CCR comprises a V_{H} comprising the amino acid sequence set forth in SEQ ID NO: 82. In certain embodiments, the extracellular antigen-binding domain of the CCR comprises a V_{L} comprising the amino acid sequence set forth in SEQ ID NO: 83. In certain embodiments, the extracellular antigen-binding domain of the CCR comprises V_{H} comprising the amino acid sequence set forth in SEQ ID NO: 82 and a V_{L} comprising the amino acid sequence set forth in SEQ ID NO: 83.

In certain embodiments, the extracellular antigen-binding domain of the CCR is an scFv that comprises or consist of the amino acid sequence set forth in SEQ ID NO: 84. SEQ ID NOs: 76-84 are provided in the following Table 4.

TABLE 4

| | anti-CD38 scFv | | |
|---|---|---|---|
| CDRs | 1 | 2 | 3 |
| V_{H} | SFAMS [SEQ ID | AISGSGGGTYYADSVKG | DKILWFGEPVEDY [SEQ |
| | NO: 76] | [SEQ ID NO: 77] | ID NO: 78] |
| V_{L} | RASQSVSSYLA [SEQ | DASNRAT [SEQ ID | QQRSNWPPT [SEQ ID |
| | ID NO: 79] | NO: 80] | NO: 81] |

| | |
|---|---|
| Full V_{H} | EVQLLESGGGLVQPGGSLRLSCAVSGFTFNSFAMSWVRQAPGKGLEWVSAISGSGGGT |
| | YYADSVKGRFTISRDNSKNTLYLQMNSLRAEDTAVYFCAKDKILWFGEPVFDYWGQGT |
| | LVTVSS [SEQ ID NO: 82] |
| Full V_{L} | EIVLTQSPATLSLSPGERATLSCRASQSVSSYLAWYQQKPGQAPRLLIYDASNRATGI |
| | PARFSGSGSGTDFTLTISSLEPEDFAVYYCQQRSNWPPTFGQGTKVEIK [SEQ ID |
| | NO: 83] |
| scFv | EVQLLESGGGLVQPGGSLRLSCAVSGFTFNSFAMSWVRQAPGKGLEWVSAISGSGGGT |
| | YYADSVKGRFTISRDNSKNTLYLQMNSLRAEDTAVYFCAKDKILWFGEPVFDYWGQGT |
| | LVTVSSGGSGGEIVLTQSPATLSLSPGERATLSCRASQSVSSYLAWYQQKPGQAPRLL |
| | IYDASNRATGIPARFSGSGSGTDFTLTISSLEPEDFAVYYCQQRSNWPPTFGQGTKVE |
| | IK [SEQ ID NO: 84] |

In certain embodiments, the V_{H} and V_{L} are linked via a linker. In certain embodiments, the linker comprises a sequence set forth in SEQ ID NO: 85, SEQ ID NO: 86, SEQ ID NO: 87, SEQ ID NO: 88, SEQ ID NO: 89, SEQ ID NO: 90, or SEQ ID NO: 91. In certain embodiments, the linker comprises or consists of the amino acid sequence set forth in SEQ ID NO: 91.

The V_{H} and/or V_{L} amino acid sequences having at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to a specific sequence (e.g., SEQ ID NOs: 55, 56, 64, 65, 73, 74, 82 and 83) may contain substitutions (e.g., conservative substitutions), insertions, or deletions relative to the specified sequence(s), but retain the ability to bind to a target antigen (e.g., CD38). In certain embodiments, a total of 1 to 10 amino acids are substituted, inserted and/or deleted in a specific sequence (e.g., SEQ ID NOs: 55, 56, 64, 65, 73, 74, 82 and 83). In certain embodiments, substitutions, insertions, or deletions occur in regions outside the CDRs (e.g., in the FRs) of the extracellular antigen-binding domain. In certain embodiments, the extracellular antigen-binding domain of the first antigen-recognizing receptor comprises V_{H} and/or V_{L} sequence selected from SEQ ID NOs: 55, 56, 64, 65, 73, 74, 82 and 83, including post-translational modifications of that sequence (SEQ ID NO: 55, 56, 64, 65, 73, 74, 82 and 83).

In certain embodiments, the intracellular domain of the CCR comprises at least an intracellular domain of a co-stimulatory molecule or a portion thereof. In certain embodiments, the co-stimulatory molecule is selected from the group consisting of CD28, 4-1BB, OX40, CD27, CD40, CD154, CD97, CD11a/CD18, ICOS, DAP-10, CD2, CD244, CD27, and NKG2D.

In certain embodiments, the intracellular domain of the CCR comprises a CD28 polypeptide, e.g., an intracellular domain of a CD28 or a portion thereof. In certain embodiments, the intracellular domain of the CCR comprises a human CD28 polypeptide (e.g., the intracellular domain of human CD28 or a portion thereof). In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% identical or homologous to the amino acid sequence set forth in SEQ ID NO: 2 or a fragment thereof, and/or may optionally comprise up to one or up to two or up to three conservative amino acid substitutions. In certain embodiments, the CD28 polypeptide comprises or consists of an amino acid sequence that is a consecutive portion of the amino acid sequence having an UniProt accession number P10747 or a NCBI Reference No: NP_006130 (SEQ ID NO: 2), which is at least about 20, or at least about 25, or at least about 30, and/or up to about 220 amino acids in length. In certain embodiments, the CD28 polypeptide comprises or consists of an amino acid sequence of amino acids 1 to 220, 1 to 50, 50 to 100, 100 to 150, 114 to 220, 150 to 200, 180 to 220, or 200 to 220 of SEQ ID NO: 2. In certain embodiments, the intracellular domain of the CCR comprises a CD28 polypeptide comprising or consisting of amino acids 180 to 220 of SEQ ID NO: 2. SEQ ID NO: 2 is provided below.

[SEQ ID NO: 2]
MLRLLLALNLFPSIQVTGNKILVKQSPMLVAYDNAVNLSCKYSYNLFSRE
FRASLHKGLDSAVEVCVVYGNYSQQLQVYSKTGFNCDGKLGNESVTFYLQ
NLYVNQTDIYFCKIEVMYPPPYLDNEKSNGTIIHVKGKHLCPSPLFPGPS
KPFWVLVVVGGVLACYSLLVTVAFIIFWVRSKRSRLLHSDYMNMTPRRPG
PTRKHYQPYAPPRDFAAYRS

In certain embodiments, the intracellular domain of the CCR comprises a CD28 polypeptide comprising or consisting of the amino acid sequence set forth in SEQ ID NO: 92. SEQ ID NO: 92 is provided below.

[SEQ ID NO: 92]
RSKRSRLLHSDYMNMTPRRPGPTRKHYQPYAPPRDFAAYRS

An exemplary nucleic acid sequence encoding the amino acid sequence of amino acids 180 to 220 of SEQ ID NO: 2 is set forth in SEQ ID NO: 3. SEQ ID NO: 3 is provided below.

[SEQ ID NO: 3]
AGGAGTAAGAGGAGCAGGCTCCTGCACAGTGACTACATGAACATGACTCC
CCGCCGCCCCGGGCCCACCCGCAAGCATTACCAGCCCTATGCCCCACCAC
GCGACTTCGCAGCCTATCGCTCC

In certain embodiments, the intracellular domain of the CCR comprises an intracellular domain of mouse CD28 or a portion thereof. In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% identical or homologous to the amino acid sequence having a NCBI Reference No: NP_031668.3 (or SEQ ID NO: 4) or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 4, which is at least about 20, or at least about 30, or at least about 40, or at least about 50, and up to 218 amino acids in length. In certain embodiments, the CD28 polypeptide comprised in the co-stimulatory signaling region comprises or consists of the amino acid sequence of amino acids 1 to 218, 1 to 50, 50 to 100, 100 to 150, 150 to 218, 178 to 218, or 200 to 218 of SEQ ID NO: 4. In certain embodiments, the intracellular domain of the CCR comprises a CD28 polypeptide comprising or consisting of the amino acid sequence of amino acids 178 to 218 of SEQ ID NO: 4. SEQ ID NO: 4 is provided below.

[SEQ ID NO: 4]
MTLRLLFLAL NFFSVQVTEN KILVKQSPLL VVDSNEVSLS
CRYSYNLLAK EFRASLYKGV NSDVEVCVGN GNFTYQPQFR
SNAEFNCDGD FDNETVTFRL WNLHVNHTDI YFCKIEFMYP
PPYLDNERSN GTIIHIKEKH LCHTQSSPKL FWALVVVAGV
LFCYGLLVTV ALCVIWTNSR RNRLLQSDYM NMTPRRPGLT
RKPYQPYAPA RDFAAYRP

In certain embodiments, the intracellular domain of the CCR comprises a 4-1BB polypeptide, e.g., an intracellular domain of a 4-1BB or a portion thereof. In certain embodiments, the CCR comprises a human 4-1BB polypeptide (e.g., an intracellular domain human 4-1BB or a portion thereof). In certain embodiments, the 4-1BB polypeptide comprises or consists of an amino acid sequence that is at least about 80%, at least about 85%, at least about 90%, at least about 95%, at least about 96%, at least about 97%, at least about 98%, or at least about 99%, at least about 100% identical or homologous to the sequence having an UniProt accession number Q07011 or a NCBI Ref. No.: NP_001552 (SEQ ID NO: 5) or a fragment thereof, and/or may optionally comprise up to one or up to two or up to three conservative amino acid substitutions. In certain embodiments, the 4-1BB polypeptide comprises or consists of an amino acid sequence that is a consecutive portion of SEQ ID NO: 5, which is at least about 20, or at least about 30, or at least about 40, or at least about 50, and/or up to about 50, up to about 60, up to about 70, up to about 80, up to about 90, up to about 100, up to about 200, or up to about 255 amino acids in length. In certain embodiments, the 4-1BB polypeptide comprises or consists of the amino acid sequence of amino acids 1 to 255, 1 to 50, 50 to 100, 100 to 150, 150 to 200, 200 to 255, or 214 to 255 of SEQ ID NO: 5. In certain embodiments, the intracellular domain of the CCR comprises a 4-1BB polypeptide comprising or consisting of the amino acid sequence of amino acids 214 to 255 of SEQ ID NO: 5. SEQ ID NO: 5 is provided below.

[SEQ ID NO: 5]
MGNSCYNIVA TLLLVLNFER TRSLQDPCSN CPAGTFCDNN
RNQICSPCPP NSFSSAGGQR TCDICRQCKG VFRTRKECSS
TSNAECDCTP GFHCLGAGCS MCEQDCKQGQ ELTKKGCKDC
CFGTFNDQKR GICRPWTNCS LDGKSVLVNG TKERDVVCGP
SPADLSPGAS SVTPPAPARE PGHSPQIISF FLALTSTALL
FLLFFLTLRF SVVKRGRKKL LYIFKQPFMR PVQTTQEEDG
CSCRFPEEEE GGCEL

In certain embodiments, the intracellular domain of the CCR comprises a 4-1BB polypeptide comprising or consisting of the amino acid sequence set forth in SEQ ID NO: 93. SEQ ID NO: 93 is provided below.

[SEQ ID NO: 93]
RGRKKLLYIFKQPFMRPVQTTQEEDGCSCRFPEEEEGGCEL

An exemplary nucleic acid sequence encoding the amino acid sequence of amino acids 214 to 255 of SEQ ID NO: 5 is set forth in SEQ ID NO: 95. SEQ ID NO: 95 is provided below.

[SEQ ID NO: 95]
CGGGGCAGAAAGAAACTCCTGTATATATTCAAACAACCATTTATGAGACC
AGTACAAACTACTCAAGAGGAAGATGGCTGTAGCTGCCGATTTCCAGAAG
AAGAAGAAGGAGGATGTGAACTG

In certain embodiments, the CCR comprises a mouse 4-1BB polypeptide (e.g., an intracellular domain mouse 4-1BB or a portion thereof). In certain embodiments, the 4-1BB polypeptide comprises or consists of an amino acid sequence that is at least about 80%, at least about 85%, at least about 90%, at least about 95%, at least about 96%, at least about 97%, at least about 98%, or at least about 99%, at least about 100% identical or homologous to the sequence having a NCBI Ref. No.: NP_035742 (SEQ ID NO: 48) or a fragment thereof, and/or may optionally comprise up to one or up to two or up to three conservative amino acid substitutions. In certain embodiments, the 4-1BB polypeptide comprises or consists of an amino acid sequence that is a consecutive portion of SEQ ID NO: 48, which is at least about 20, or at least about 30, or at least about 40, or at least about 50, and/or up to about 50, up to about 60, up to about 70, up to about 80, up to about 90, up to about 100, up to about 200, or up to about 256 amino acids in length. In certain embodiments, the 4-1BB polypeptide comprises or consists of the amino acid sequence of amino acids 1 to 255, 1 to 50, 50 to 100, 100 to 150, 150 to 200, 200 to 256, or 209 to 256 of SEQ ID NO: 48. In certain embodiments, the intracellular domain of the CCR comprises a 4-1BB polypeptide comprising or consisting of the amino acid sequence of amino acids 209 to 256 of SEQ ID NO: 48. SEQ ID NO: 48 is provided below.

[SEQ ID NO: 48]

| | |
|---|---|
| 1 | mgnncynvvv ivlllvgcek vgavqnscdn cqpgtfcrky |
| | npvckscpps tfssiggqpn |
| 61 | cnicrvcagy frfkkfcsst hnaececieg fhclgpqctr |
| | cekdcrpgqe ltkqgcktcs |
| 121 | lgtfndqngt gvcrpwtncs ldgrsvlktg ttekdvvcgp |
| | pvvsfspstt isvtpeggpg |
| 181 | ghslqvltlf laltsallla lifitllfsv lkwirkkfph |
| | ifkqpfkktt gaaqeedacs |
| 241 | crcpqeeegg gggyel |

In certain embodiments, the intracellular domain of the CCR comprises an intracellular domain of CD28 or a portion thereof. In certain embodiments, the intracellular domain of the CCR comprises an intracellular domain of 4-1BB or a portion thereof. In certain embodiments, the intracellular domain of the CCR comprises an intracellular domain of CD28 or a portion thereof, and an intracellular domain of 4-1BB or a portion thereof.

In certain embodiments, the CCR further comprises a transmembrane domain. In certain embodiments, the transmembrane domain of the CCR comprises a CD28 polypeptide (e.g., a transmembrane domain of CD28 or a portion thereof). In certain embodiments, the transmembrane domain of the CCR comprises a human CD28 polypeptide (e.g., a transmembrane domain of human CD28 or a portion thereof). In certain embodiments, the CD28 polypeptide comprises or consists of an amino acid sequence that is a consecutive portion of the amino acid sequence set forth in SEQ ID NO: 2 or a fragment thereof, which is at least about 20, or at least about 25, or at least about 30, and/or up to about 220 amino acids in length. In certain embodiments, the CD28 polypeptide comprises or consists of an amino acid sequence of amino acids 1 to 220, 1 to 50, 50 to 100, 100 to 150, 114 to 220, 150 to 200, 153 to 179, or 200 to 220 of SEQ ID NO: 2. In certain embodiments, the transmembrane domain of the CCR comprises a CD28 polypeptide comprising or consisting of amino acids 153 to 179 of SEQ ID NO: 2.

In certain embodiments, the transmembrane domain of the CCR comprises a CD28 polypeptide comprising or consists of the amino acid sequence set forth in SEQ ID NO: 94. SEQ ID NO: 94 is provided below.

[SEQ ID NO: 94]
FWVLVVVGGVLACYSLLVTVAFIIFWV

An exemplary nucleic acid sequence encoding the amino acid sequence of SEQ ID NO: 94 is set forth in SEQ ID NO: 96. SEQ ID NO: 96 is provided below.

[SEQ ID NO: 96]
ttttgggtgctggtggtggttggtggagtcctggcttgctatagcttgc
tagtaacagtggcctttattattttctgggtg

In certain embodiments, the transmembrane domain of the CCR comprises a murine CD28 polypeptide (e.g., a transmembrane domain of murine CD28 or a portion thereof). In certain embodiments, the CD28 polypeptide comprises or consists of an amino acid sequence that is a consecutive portion of the amino acid sequence set forth in SEQ ID NO: 4 or a fragment thereof, which is at least about 20, or at least about 25, or at least about 30, or at least about 40, or at least about 50, and/or up to about 218 amino acids in length. In certain embodiments, the CD28 polypeptide comprises or consists of an amino acid sequence of amino acids 1 to 218, 1 to 50, 50 to 100, 100 to 150, 150 to 218, 151 to 177, or 200 to 218 of SEQ ID NO: 4. In certain embodiments, the transmembrane domain of the CCR comprises a CD28 polypeptide comprising or consisting of amino acids 151 to 177 of SEQ ID NO: 4.

In certain embodiments, the transmembrane domain of the CCR comprises a 4-1BB polypeptide (e.g., a transmembrane domain of 4-1BB or a portion thereof). In certain embodiments, the transmembrane domain of the CCR comprises a human 4-1BB polypeptide (e.g., a transmembrane domain of human 4-1BB or a portion thereof). In certain embodiments, the 4-1BB polypeptide comprises or consists of an amino acid sequence that is a consecutive portion of the amino acid sequence having set forth in SEQ ID NO: 5, which is at least about 20, or at least about 30, or at least about 40, or at least about 50, and/or up to about 50, up to about 60, up to about 70, up to about 80, up to about 90, up to about 100, up to about 200, or up to about 255 amino acids in length. In certain embodiments, the 4-1BB polypeptide comprises or consists of the amino acid sequence of amino acids 1 to 255, 1 to 50, 50 to 100, 100 to 150, 150 to 200, 187 to 213, or 200 to 255 of SEQ ID NO: 5. In certain embodiments, the transmembrane domain of the CCR comprises a 4-1BB polypeptide comprising or consisting of the amino acid sequence of amino acids 187 to 213 of SEQ ID NO: 5.

In certain embodiments, the CCR comprises a) an extracellular antigen-binding domain that binds to CD38 (e.g., human CD38), and b) an intracellular domain comprising a 4-1BB polypeptide (e.g., a human 4-1BB polypeptide, e.g., an intracellular domain of 4-1BB (e.g., human 4-1BB) of a portion thereof). In certain embodiments, the CCR further comprises a transmembrane domain comprising a 4-1BB polypeptide (e.g., a human 4-1BB polypeptide, e.g., a transmembrane domain of 4-1BB (e.g., human 4-1BB) or a portion thereof).

In certain embodiments, the CCR comprises a) an extracellular antigen-binding domain that binds to CD38 (e.g., human CD38), and b) an intracellular domain comprising a CD28 polypeptide (e.g., a human CD28 polypeptide, e.g., an intracellular domain of CD28 (e.g., human CD28) of a portion thereof). In certain embodiments, the CCR further comprises a transmembrane domain comprising a CD28 polypeptide (e.g., a human CD28 polypeptide, e.g., a transmembrane domain of CD28 (e.g., human CD28) or a portion thereof).

In certain embodiments, the CCR comprises a) an extracellular antigen-binding domain that binds to CD38 (e.g., human CD38), and b) an intracellular domain comprising a CD28 polypeptide (e.g., a human CD28 polypeptide, e.g., an intracellular domain of CD28 (e.g., human CD28) of a portion thereof) and a 4-1BB polypeptide (e.g., a human 4-1BB polypeptide, e.g., an intracellular domain of 4-1BB (e.g., human 4-1BB) of a portion thereof). In certain embodiments, the CCR further comprises a transmembrane domain comprising a CD28 polypeptide (e.g., a human CD28 polypeptide, e.g., a transmembrane domain of CD28 (e.g., human CD28) or a portion thereof) and a 4-1BB polypeptide (e.g., a human 4-1BB polypeptide, e.g., a transmembrane domain of 4-1BB (e.g., human 4-1BB) or a portion thereof).

In certain embodiments, the CCR comprises a) an extracellular antigen-binding domain that binds to CD38 (e.g., human CD38), and b) an intracellular domain comprising a CD28 polypeptide (e.g., a human CD28 polypeptide, e.g., an intracellular domain of CD28 (e.g., human CD28) of a portion thereof) and a 4-1BB polypeptide (e.g., a human 4-1BB polypeptide, e.g., an intracellular domain of 4-1BB (e.g., human 4-1BB) of a portion thereof). In certain embodiments, the CCR further comprises a transmembrane domain comprising a CD28 polypeptide (e.g., a human CD28 polypeptide, e.g., a transmembrane domain of CD28 (e.g., human CD28) or a portion thereof).

In certain embodiments, the CCR comprises a) an extracellular antigen-binding domain that binds to CD38 (e.g., human CD38), and b) an intracellular domain comprising a CD28 polypeptide (e.g., a human CD28 polypeptide, e.g., an intracellular domain of CD28 (e.g., human CD28) of a portion thereof) and a 4-1BB polypeptide (e.g., a human 4-1BB polypeptide, e.g., an intracellular domain of 4-1BB (e.g., human 4-1BB) of a portion thereof). In certain embodiments, the CCR further comprises a transmembrane domain comprising a 4-1BB polypeptide (e.g., a human 4-1BB polypeptide, e.g., a transmembrane domain of 4-1BB (e.g., human 4-1BB) or a portion thereof).

In certain embodiments, the intracellular co-stimulatory signaling domain of a CCR stimulates or enhances an efficient response of the immune cell, comprising said CCR, upon binding to an antigen through the CD3ζ domain of a CAR. Said intracellular co-stimulatory signaling domain can include the intracellular portions of CD28, 4-1BB, OX40, ICOS, CD27, CD40, 2B4, NKG2D, or a relevant part thereof. Said intracellular co-stimulatory signaling domain can include the intracellular portions of human CD28, 4-1BB, OX40, ICOS, CD27, CD40, 2B4, NKG2D, or a relevant part thereof. As an alternative, or in addition, said intracellular co-stimulatory signaling domain can comprise the equivalent protein from a non-human species, e.g., mouse, rodent, monkey, ape and the like. As an alternative, or in addition, said intracellular co-stimulatory signaling domain can include a domain that is at least 80% identical, at least 90% identical, at least 95% identical, at least 99% identical, to the intracellular portions of human CD28, 4-1BB, OX40, ICOS, CD27, CD40, 2B4, NKG2D.

In certain embodiments, the CCR comprises the intracellular co-stimulatory domain of human 4-1BB and the co-stimulatory domain of CD28. In certain embodiments, the CCR comprises the intracellular co-stimulatory domain of human 4-1BB and the co-stimulatory domain of human CD28.

In certain embodiments, the methods disclosed herein provide cells and composition thereof comprising a CD38 CCR and a reduced and/or abolished expression of CD38. In certain embodiments, the cell comprising an antigen-recognizing receptor and the CD-38 CCR exhibits substantial cytolytic activity against cells that are single positive for the antigen. In certain embodiments, the cell comprising an antigen-recognizing receptor and the CD-38 CCR exhibits a greater degree of cytolytic activity against cells that are positive for both the first antigen and the second antigen as compared to against cells that are singly positive for the first antigen. In certain embodiments, the cell comprising the first antigen-recognizing receptor and CD38 CCR exhibits substantially no or negligible cytolytic activity against cells that are singly positive for the first antigen.

### 5.3. Cells

The presently disclosed subject matter provides cells comprising: a) a CD38 CCR (e.g., one disclosed in Section 5.2) and b) an antigen-recognizing receptor that binds to an antigen. In certain embodiments, the antigen-recognizing receptor binds to an antigen that is not CD38. In certain embodiments, the cell exhibits substantial cytolytic activity against cells that are single positive for the antigen.

In certain embodiments, the cell is selected from the group consisting of cells of lymphoid lineage and cells of myeloid lineage. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, Natural Killer (NK) cells, B cells, dendritic cells, and stem cells from which lymphoid cells may be differentiated. In certain embodiments, the stem cell is a pluripotent stem cell (e.g., embryonic stem cell).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., T_{EM} cells and T_{EMRA} cells, Regulatory T cells (also known as suppressor T cells), tumor-infiltrating lymphocyte (TIL), Natural Killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR or a TCR. The T cell can be a CD4⁺ T cell or a CD8⁺ T cell. In certain embodiments, the T cell is a CD4⁺ T cell. In certain embodiments, the T cell is a CD8⁺ T cell. In certain embodiments, the CD8⁺ T cell is CD4 independent. In certain embodiments, the T cell is derived from an induced pluripotent stem cell (iPSC). In certain embodiments, the T cell is a CD8⁺ T cell that is CD4 independent, and the CD8⁺ T cell is derived from an iPSC.

In certain embodiments, the cell is a NK cell. Natural Killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells).

In certain embodiments, the cell (e.g., T cell) is autologous. In certain embodiments, the cell (e.g., T cell) is non-autologous. In certain embodiments, the cell (e.g., T cell) is allogeneic. In certain embodiments, the cell (e.g., T cell) is derived in vitro from an engineered progenitor or stem cell.

In certain embodiments, the cell is a cell of the myeloid lineage. Non-limiting examples of cells of the myeloid lineage include monocytes, macrophages, neutrophils, basophils, cosinophils, erythrocytes, megakaryocytes, and stem cells from which myeloid cells may be differentiated. In certain embodiments, the stem cell is a pluripotent stem cell (e.g., an embryonic stem cell or an induced pluripotent stem cell).

The presently disclosed subject matter also provides a genetically modified immune cell expressing a CAR and a CCR disclosed herein. In certain embodiments, the immune cell can be any cell of hematopoictic origin that is able to express a CAR and CCR disclosed herein. In certain embodiments, the cell is a T cell or NK cell that, when activated by binding of the CAR and CCR to their respective targets, becomes a cytotoxic cell that kills the cell that expresses the targets of the CAR and CCR. In certain embodiments, the cell that expresses the targets of the CAR and CCR is a cancer cell, a cell that is infected, for example, with a virus, or a cell that is damaged in other ways.

In certain embodiments, the CAR expressed by an immune cell, for example a T cell or NK cell, comprises an antigen specific binding domain, a CD28 transmembrane domain, a co-stimulatory CD28 intracellular domain and an intracellular CD3ζ signaling domain. In certain embodiments, the CCR expressed by an immune cell, for example a T cell or NK cell, comprises an antigen specific binding domain, a CD28 transmembrane domain, a co-stimulatory CD28 intracellular domain, and a co-stimulatory 4-1BB intracellular domain.

### 5.3.1. Antigen-Recognizing Receptors

The antigen-recognizing receptor binds to an antigen. The antigen can be a tumor antigen or a pathogen antigen. In certain embodiments, the antigen-recognizing receptor is a chimeric antigen receptor (CAR). In certain embodiments, the antigen-recognizing receptor is a TCR like fusion molecule. In certain embodiments, the antigen-recognizing receptor is a T Cell Receptor (TCR).

In certain embodiments, the cell comprising the antigen-recognizing receptor that binds to the antigen and the CD38 CCR exhibits substantial cytolytic activity against cells that are single positive for the antigen.

In certain embodiments, the antigen-recognizing receptor binds to the antigen with a high binding affinity. In certain embodiments, the antigen-recognizing receptor binds to the antigen with a dissociation constant (K_{d}) of about 1×10⁻⁸ M or less, or about 1×10⁻⁹ M or less, or about 1×10⁻¹⁰ M or less

In certain embodiments, the antigen-recognizing receptor binds to the antigen with a low binding affinity. In certain embodiments, the antigen-recognizing receptor binds to the antigen with a dissociation constant (K_{d}) of about 1×10⁻⁸ M or more, about 1×10⁻⁷M or more, or about 1×10⁻⁶ M or more, or about 1×10⁻⁵ M or more.

### 5.3.1.1. Antigen

In certain embodiments, the antigen is a tumor antigen. Any tumor antigen (antigenic peptide) can be used in the tumor-related embodiments described herein. Sources of antigen include, but are not limited to, cancer proteins. The antigen can be expressed as a peptide or as an intact protein or portion thereof. The intact protein or a portion thereof can be native or mutagenized. In certain non-limiting embodiments, the antigen is a phosphoantigen. Non-limiting examples of tumor antigens include CD19, carbonic anhydrase IX (CAIX), carcinoembryonic antigen (CEA), CD8, CD7, CD10, CD20, CD22, CD30, CD33, CLL1, CD34, CD41, CD44, CD49f, CD56, CD74, CD133, CD138, CD123, CD44V6, an antigen of a cytomegalovirus (CMV) infected cell (e.g., a cell surface antigen), ENPP3, epithelial glycoprotein-2 (EGP-2), epithelial glycoprotein-40 (EGP-40), epithelial cell adhesion molecule (EpCAM), receptor tyrosine-protein kinase Erb-B2, Erb-B3, Erb-B4, folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, GPRC5D, FcRL5, ITGB7, NKCS1, EGF1R, EGFR-VIII, CD99, CD70, ADGRE2, CCR1, LILRB2, PRAME, CD48, P2RY10, CD70, BOB1, CLEC12A, CCR1, LILRB2, CD5, CD38, CS-1, Lewis antigen (LeY), and ERBB. In certain embodiments, the antigen specific binding domain of a CAR and/or CCR that is expressed by an immune cell disclosed herein can target any tumor antigen that is expressed on the surface of a tumor cell. In certain embodiment, the tumor is a hematological tumor.

In certain embodiments, the antigen is expressed on a tumor tissue or tumor cell. In certain embodiments, the tumor is hematological cancer. In certain embodiments, the hematological cancer is selected from the group consisting of multiple myeloma (MM), Non-Hodgkin Lymphoma, Hodgkin Lymphoma, Chronic Lymphocytic Leukemia (CLL), Waldenstrom's Macroglobulinemia, acute lymphoblastic leukemia (ALL), lymphoma, and acute myeloid leukemia (AML). In certain embodiments, the tumor is multiple myeloma. In certain embodiments, the antigen is a phosphoantigen. In certain embodiments, the antigen is selected from the group consisting of BCMA, GPRC5D, FcRL5, CD5, CD7, CD10, CD19, CD20, CD22, CD30, CD33, CD34, CD38, CD41, CD48, P2RY10, CD44v6, CD56, CD70, CD123, BCMA, CS-1, CD138, Kappa light chain, Lewis-Y (LeY), NY-ESO-1, BOB1, ADGRE2, CLEC12A, CCR1, LILRB2, CD5, CD7, CD10, CD19, CD20, CD22, CD30, CD33, CD34, CD38, CD56, BCMA, CS-1, CD138, Lewis antigen (LeY), WT-1, Wilms' tumor gene, and ITGB7. In certain embodiments, the antigen is BCMA. In certain embodiments, the antigen is expressed on a multiple myeloma (MM) tissue.

In certain embodiments, the antigen is expressed on a solid tumor tissue or a solid tumor cell. Solid tumors includes sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, cpendymoma, pincaloma, hemangioblastoma, acoustic neuroma, oligodenroglioma, schwannoma, meningioma, melanoma, neuroblastoma, and retinoblastoma).

In certain embodiments, the solid tumor is a CD38-expressing solid tumor. In certain embodiments, the solid tumor is associated with over-expression of CD38. In certain embodiments, the solid tumor is selected from the group consisting of breast cancer, colon cancer, non-small cell lung cancer, prostate cancer, gastric, esophageal and pancreatic cancers, hepatocellular carcinoma (HCC), and fibrolamellar carcinoma.

In certain embodiments, the antigen is not expressed or expressed at a non-detectable level in a non-tumor cell.

In certain embodiments, antigen specific binding domain targets one or more of CD5, CD7, CD10, CD19, CD20, CD22, CD30, CD33, CD34, CD38, CD41, CD48, P2RY10, CD44v6, CD56, CD70, CD123, BCMA, CS-1, CD138, Kappa light chain, Lewis-Y (LeY), NY-ESO-1, BOB1, ADGRE2, CLEC12A, CCR1, LILRB2, and Wilms' tumor gene.

CD5 encodes a member of the scavenger receptor cysteine-rich superfamily, which is a type-I transmembrane glycoprotein that is present on the surface of thymocytes, T lymphocytes and a subset of B lymphocytes. CD5 is a marker for B cell chronic lymphatic leukemia, B cell small lymphocytic lymphoma, mantle cell lymphoma, malignant T cells and thymic carcinoma. CD5 may also be expressed on lymphoma's, including atypical thymoma, Burkitt lymphoma, mucosa-associated lymphoid tissue (MALT) lymphoma and splenic lymphoma.

CD7 is a cell surface glycoprotein that is present on thymocytes and mature T cells. CD7 is a marker for T-cell acute lymphoblastic leukemia and other malignant immature T cells, stem cell lymphoma, chronic myelogenous leukemia, Down syndrome associated transient myeloproliferative disorder and acute myeloid leukemia.

CD10 is a cell surface enzyme with neutral metalloendopeptidase activity. CD10 is also known as CALLA (common acute lymphocytic leukemia antigen). CD10 is a marker for the common form of acute lymphocytic leukemia as well as for Burkitt lymphoma, angioimmunoblastic T cell lymphoma, and follicular germinal center lymphoma.

CD19 is a 95 kd transmembrane glycoprotein belonging to the immunoglobulin superfamily. CD19 is classified as a type I transmembrane protein, with a single transmembrane domain, a cytoplasmic C-terminus, and extracellular N-terminus. CD19 is a marker for B cell lymphomas and leukemias.

CD20 is a membrane-embedded surface molecule which plays a role in the development and differentiation of B-cells into plasma cells. CD20 is a marker for B cell lymphomas, pre B acute lymphocytic leukemia/lymphoblastic lymphoma, spindle cell thymoma and nodular lymphocyte predominant Hodgkin lymphoma.

CD22 is a transmembrane glycoprotein member of the immunoglobulin superfamily that may bind alpha2,6-linked sialic acid-bearing ligands. CD22 is a marker for hairy cell leukemia, pre B acute lymphocytic leukemia, blastic plasmacytoid dendritic cell neoplasm.

CD30 is a cell membrane protein of the tumor necrosis factor receptor family. CD30 is a marker for anaplastic large cell lymphoma, classic Hodgkin lymphoma and primary mediastinal large B cell lymphoma.

CD3ζ or sialic acid binding Ig-like lectin 3 (Siglec-3) is an immunoglobulin domain comprising transmembrane receptor. CD3ζ is expressed on cells of myeloid and on some lymphoid cells. CD3ζ is a marker for Acute Myeloid Leukemia, anaplastic large cell lymphoma, chronic myeloid leukemia, chronic myelomonocytic leukemia, myeloid/granulocytic sarcoma and Burkitt's lymphoma.

CD3ζ is a transmembrane phosphoglycoprotein protein which may act as a cell-cell adhesion factor. CD3ζ is a marker for Acute Myeloid Leukemia.

CD41 is an integrin alpha chain IIb protein that in humans is encoded by the ITGA2B gene. Alpha chain 2b, together with the integrin beta 3, form a fibrinogen receptor that is expressed in platelets. CD41 is a marker for acute megakaryoblastic leukemia (AML-M7).

CD48 is a glycosylphosphatidylinositol-anchored protein (GPI-AP) found on the surface of immune cells such as NK cells, T cells, monocytes, and basophils, and participates in adhesion and activation pathways in these cells. CD48 is known to be expressed on multiple myeloma cells and other cancers of B cell origin, e.g. non-Hodgkins lymphoma, chronic lymphocytic leukemia, monoclonal gammopathy of unknown significance (MGUS), Waldenstrom's macroglobulinemia, primary/systemic amyloidosis and follicular lymphoma.

P2RY10 or P2Y Receptor Family Member 10, is a purinergic G-protein coupled receptor that is preferentially activated by adenosine and uridine nucleotides. P2RY10 has been reported to be a tumor microenvironment-associated gene and a biomarker of metastatic melanoma.

CD38 is a 46-kDa type II transmembrane glycoprotein. CD38 is uniformly highly expressed in almost all hematological malignancies, including MM, CLL, ALL, AML and lymphoma, and can thus be used as a broad hematologic malignancy-associated target.

CD44, also termed homing cell adhesion molecule (HCAM) or lymphocyte homing receptor, is a cell-surface glycoprotein involved in cell-cell interactions, cell adhesion and migration. A splice variant of CD44, termed CD44v6, is a marker for non-Hodgkin's lymphoma.

CD56, also termed neural cell adhesion molecule (NCAM), is a homophilic binding glycoprotein expressed on the surface of neurons, glia and skeletal muscle cells. CD56 is a marker for NK lymphomas.

CD70 is a cytokine that belongs to the tumor necrosis factor (TNF) ligand family and is a ligand for TNFRSF27/CD27. CD70 is expressed on the surface of activated T and B lymphocytes. CD70 is a marker for cutaneous T-cell lymphoma.

CD123, or Interleukin (IL)-3 receptor, is a glycoprotein that, together with a Beta Common subunit, forms the heterodimeric IL3 receptor. CD123 is expressed on plasmacytoid monocytes. CD123 is a marker for leukemic stem cells.

B-cell maturation antigen (BCMA) is a cell surface receptor of the TNF receptor superfamily which recognizes B-cell activating factor (BAFF). BCMA is a marker for B cell leukemia, B cell lymphomas and multiple myeloma.

CS-1 a member of the CD2 family of cell surface receptors that is expressed on NK cells and on activated B cells. CS-1, also termed CD319 or SLAMF7, is a marker for malignant plasma cells, especially malignant myeloma plasma cells.

Kappa light chain is encoded by the immunoglobulin kappa locus on chromosome 2 and is expressed in B-cells. Kappa light chain is a marker for B-cell lymphoma and neoplastic plasma cells, such as multiple myeloma. CD138, also termed syndecan, is a transmembrane heparan sulfate proteoglycan. CD138 is a marker for keratoacanthoma, myeloma, plasmablastic lymphoma, primary effusion lymphoma and pyothorax associated lymphoma.

LeY is an oligosaccharide that is overexpressed on many epithelial cancers and hematological malignancies (including AML) but has limited expression on normal healthy tissues.

NY-ESO-1, or cancer/testis antigen 1B, is a protein having no homology with any known protein. NY-ESO-1 belongs to an expanding family of immunogenic testicular antigens that are aberrantly expressed in human cancers in a lineage-nonspecific fashion. NY-ESO-1 is a marker for multiple myeloma.

BOB1 is a transcription factor that is localized intracellularly, but HLA-presenting Bob1-derived polypeptides are accessible to the cell surface of the T cell receptor (TCR) and can therefore be recognized by T cells. BOB1 is a marker for multiple hematological malignancies such as ALL, CLL, MCL and MM

ADGRE2, also termed Adhesion G Protein-Coupled Receptor E2 or Egf-Like Module Containing, Mucin-Like, Hormone Receptor-Like 2 (EMR2) is a human myeloid-restricted adhesion G protein-coupled receptor. ADGRE2 is a marker for acute myeloid leukemia.

CLEC12A, or C-type lectin domain family 12 member, is a member of the C-type lectin/C-type lectin-like domain (CTL/CTLD) superfamily. Members of this family share a common protein fold and have diverse functions, such as cell adhesion, cell-cell signaling, glycoprotein turnover, and roles in inflammation and immune response. CLEC12A is a marker for acute myeloid leukemia.

CCR1, or CD191, is a member of the beta chemokine receptor family, which belongs to family of G protein-coupled, 7 transmembrane receptors. CCR1 is a marker for multiple myeloma.

LILRB2, also termed Leukocyte immunoglobulin like receptor B2 or CD85d, is a member of the leukocyte immunoglobulin-like receptor (LIR) family. The receptor is expressed on immune cells. LILRB2 is expressed on NK cells, T cells, monocytes/macrophages, dendritic cells and cosinophils. LILRB2 is a marker for acute myeloid leukemia.

Wilms' tumor gene (WT1) encodes a zinc finger domain comprising DNA binding protein. WT1 is expressed at high levels in most of acute myelocytic, acute lymphocytic, and chronic myelocytic leukemia.

Phosphoantigens include, but are not limited to, isopentenyl pyrophosphate (IPP). IPP is overproduced in cancer cells as a result of a dysregulated mevalonate pathway (Gober et al., 2003. J Exp Med 197:163-168).

In certain embodiments, targeting two antigens instead of one can overcome antigen escape and/or antigen loss relapses. Therefore, the presently disclosed subject matter provides an immune cell expressing a CAR and a CCR, wherein the CAR comprises an antigen specific binding domain binding to a first antigen and the CCR comprises an antigen specific binding domain binding to a second antigen, wherein the first antigen differs from the second antigen. It is understood that the first and second antigen are different proteins and not different epitopes from the same protein. In certain embodiments, the first antigen is CD19 and the second antigen is CD38.

In certain embodiments, the antigen is a pathogen antigen. In certain embodiments, the antigen is a pathogen antigen expressed on a virus. Non-limiting examples of viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2-parenterally transmitted (i.e. Hepatitis C); Norwalk and related viruses, and astroviruses).

In certain embodiments, the antigen is a pathogen antigen expressed on a bacterium. Non-limiting examples of bacteria include Pasteurella, Staphylococci, Streptococcus, Escherichia coli, Pseudomonas species, and Salmonella species. Specific examples of infectious bacteria include but are not limited to, Helicobacter pyloris, Borelia burgdorferi, Legionella, Legionella pneumophilia, Mycobacteria sps (e.g. M. tuberculosis, M. avium, M. intracellulare, M. kansaii, M. gordonae, M. leprae), Staphylococcus aureus, Staphylococcus epidermidis, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (Anaerobic sps.), Streptococcus pneumoniae, pathogenic Campylobacter sp., Campylobacter jejuni, Enterococcus sp., Haemophilus influenzae, Bacillus antracis, Corynebacterium diphtheriae, corynebacterium sp., Erysipelothrix rhusiopathiae, Clostridium spp., Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israelli. Mycoplasma, Pseudomonas aeruginosa, Pseudomonas fluorescens, Corynobacteria diphtheriae, Bartonella henselae, Bartonella quintana, Coxiella burnetii, chlamydia, shigella, Yersinia enterocolitica, Yersinia pseudotuberculosis, Listeria monocytogenes, Mycoplasma spp., Vibrio cholerae, Borrelia, Francisella, Brucella melitensis, Proteus mirabilis, and Proteus.

In certain embodiments, the antigen is a pathogen antigen expressed on a fungi. Non-limiting examples of fungi include Absidia corymbifera, Acremoniumfalciforme, A. kiliense, A. recifei, Ajellomyces dermatitidis, A. capsulata, Aspergillus spp., (e.g., A. flavus, A. fumigatus’, A. nidulans, A. niger, A. terreus), Candida spp. (e.g., C. albicans, C. glabrata, C. guillermondii, C. krusei, C. parapsilosis, C. ke yr, C. tropicalis), C. neoformans, Cunninghamella elegans, Emmonsia parva, Epidermophyton floccosum, Exophialia dermitidis, E. werneckii, E. jeanselmei, E. spinifera, E. richardsiae, Filobasidiella neoformans, Fonsecaea compacta, F. pedrosoi, Histoplasma capsulatum, Leptoshaeria senegarlensis, Madurella mycetomatis, M. grisea, Malassezia furfur, Microsporum spp, Neotestudina rosatii, Paracoccidioides brasiliensis, Penicillium marneffei, Phialophora verrucosa, Piedraia hortae, Pneumocystis carinii, Pseudallescheria boydii, Pyrenochaeta romeroi, Rhizomucor pusillus, Sporothrix schenckii, Trichophyton spp, Trichosporon beigelii, and Xylohypha bantiana.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), a viral antigen present in influenza virus, or a viral antigen present in SARS coronavirus.

### 5.3.1.2. Chimeric Antigen Receptors (CARs)

In certain embodiments, the antigen-recognizing receptor is a chimeric antigen receptor (CAR). CARs are engineered receptors, which graft or confer a specificity of interest onto an immune effector cell. CARs can be used to graft the specificity of a monoclonal antibody onto a T cell; with transfer of their coding sequence facilitated by, for example, retroviral vectors.

There are three generations of CARS. “First generation” CARs are typically composed of an extracellular antigen-binding domain (e.g., an scFv) that binds to a target antigen, and an intracellular signaling domain. In certain embodiments, the CAR further comprises a transmembrane domain. “First generation” CARs can provide de novo antigen recognition and cause activation of both CD4⁺ and CD8⁺ T cells through their CD3ζ chain signaling domain in a single fusion molecule, independent of HLA-mediated antigen presentation. In certain embodiments, a “first generation CAR” refers to a CAR containing a single intracellular signaling domain. “Second generation” CARs include a signaling domain of a co-stimulatory molecule (e.g., CD28, 4-1BB, ICOS, OX40, CD27, CD40 and NKG2D) to the intracellular signaling domain of the CAR to provide co-stimulation signals to the T cell. “Second generation” CARs comprise those that provide both co-stimulation (e.g., CD28 or 4-1BB) and activation (CD3). In certain embodiments, “second generation CAR” refers to a CAR comprising an intracellular signaling domain and a co-stimulatory signaling domain. “Third generation” CARs comprise those that provide multiple co-stimulation (e.g., CD28 and 4-1BB) and activation (CD3ζ). In certain embodiments, “third generation CAR” refers to a CAR comprising an intracellular signaling domain and two co-stimulatory signaling domains. In certain embodiments, a CAR comprises a CD3ζ intracellular signaling domain. In certain embodiments, a CAR comprises a human CD3ζ intracellular signaling domain, or a variant thereof.

In certain embodiments, the antigen-recognizing receptor is a CAR comprising an extracellular antigen-binding domain that binds to the antigen (e.g., one disclosed in Section 5.3.1.1 or one disclosed in Table 5), and an intracellular signaling domain. In certain embodiments, the CAR further comprises a transmembrane domain. In certain embodiments, the CAR further comprises a hinger/spacer region.

In certain embodiments, the extracellular antigen-binding domain of the CAR binds to the antigen with a high binding affinity. In certain embodiments, the extracellular antigen-binding domain of the CAR binds to the antigen with a dissociation constant (K_{d}) of about 1×10⁻⁸ M or less, or about 1×10⁻⁹ M or less, or about 1×10⁻¹⁰ M or less

In certain embodiments, the extracellular antigen-binding domain of the CAR binds to the antigen with a low binding affinity. In certain embodiments, the extracellular antigen-binding domain of the CAR binds to the antigen with a dissociation constant (Kd) of about 1×10⁻⁸ M or more, about 1×10⁻⁷ M or more, or about 1×10⁻⁶ M or more, or about 1×10⁻⁵ M or more.

Binding of the extracellular antigen-binding domain (for example, in an scFv) can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March 1986, which is incorporated by reference herein). The radioactive isotope can be detected by such means as the use of a γ counter or a scintillation counter or by autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and YPet).

The extracellular antigen-binding domain can comprise or be an scFv, a Fab (which is optionally crosslinked), or a F (ab)₂. In certain embodiments, any of the foregoing molecules may be comprised in a fusion protein with a heterologous sequence to form the extracellular antigen-binding domain. In certain embodiments, the extracellular antigen-binding domain comprises or is an scFv. In certain embodiments, the scFv is a human scFv. In certain embodiments, the scFv is a humanized scFv. In certain embodiments, the scFv is a murine scFv.

In certain embodiments, the antigen specific binding domain of the CAR expressed on an immune cell is a single chain binding domain, e.g., a single chain Fv fragment. In certain embodiments, both the antigen specific binding domain of the CAR expressed on an immune cell is a single chain binding domain (e.g., a single chain Fv fragment) and the antigen specific binding domain of the CCR expressed on an immune cell is a single chain binding domain (e.g., a single chain Fv fragment). Non-limiting examples of single chain binding domains are provided in Table 5.

TABLE 5
Targeting sequences

| Target | Amino Acid sequence | Reference |
|---|---|---|
| CD19 | MLLLVTSLLLCELPHPAFLLIPDIQMTQTTSSLSASLGDRVTISCRASQDISKYLNWYQQKPDGTVKLLIYHTSRLHSGVPSRFSGS | Kochenderfer et al., 2010. |
| | GSGTDYSLTISNLEQEDIATYFCQQGNTLPYTFGGGTKLEITGSTSGSGKPGSGEGSTKGEVKLQESGPGLVAPSQSLSVTCTVSGV | Blood 116: 4099-4102 |
| | SLPDYGVSWIRQPPRKGLEWLGVIWGSETTYYNSALKSRLTIIKDNSKSQVFLKMNSLQTDDTAIYYCAKHYYYGGSYAMDYWGQGT | |
| | SVTVSS (SEQ ID NO: 97) | |
| CD19 | QVQLLESGAELVRPGSSVKISCKASGYAFSSYWMNWVKQRPGQGLEWIGQIWPGDGDTNYNGKFKG | Bejcek et al., 1995. Cancer |
| | KATLTADESSSTAYMQLSSLRSEDSAVYSCARRETTTVGRYYYAMDYWGQGttVTGGGGSGGGGSGGGGSKLVLTQSPASLAVSLGQ | Res 55: 2346-2351 |
| | RATISCKASQSVDYDGDSYLNWYQQIPGQPPKLLIYDASNLVSGIPPRFSGSGSGTDFTLNIHPVEKVDAATYHCQQSTEDPWTFGG | |
| | GTKLEIKRRS (SEQ ID NO: 98) | |
| CD38 | EVQLLESGGGLVQPGGSLRLSCAVSGFTFNSFAMSWVRQAPGKGLEWVSAISGSGGGTYYADSVKGRFTISRDNSKNTLYLQMNSLR | De Luca et al., 2018. Protein |
| | AEDTAVYFCAKDKILWFGEPVFDYWGQGTLVTVSSGGSGGEIVLTQSPATLSLSPGERATLSCRASQSVSSYLAWYQQKPGQAPRLL | Engineering, Design and |
| | IYDASNRATGIPARFSGSGSGTDFTLTISSLEPEDFAVYYCQQRSNWPPTFGQGTKVEIK [SEQ ID NO: 84] | Selection 31: 173-179 |
| CD38 | QVQLVQSGAEVKKPGSSVKVSCKAFGGTFSSYAISWVRQAPGQGLEWMGRIIRFLGIANYAQKFQGRVTLIADKSTNTAYMELSSLR | Drent et al., 2016. |
| | SEDTAVYYCAGEPGERDPDAVDIWGQGTMVTVSSGGGGSGGGGSGGGGSDIQMTQSPSSISASVGDRVTITCRASQGIRSWIAWYQQ | Haematologica 101: 616-625 |
| | KPEKAPKSLIYAASSLQSGVPSRFSGSGSGTDFTLTISSLQPEDFATYYCQQYNSYPLTFGGGTKVEIK (SEQ ID NO: 57) | |
| CD38 | QVQLVQSGAEVKKPGSSVKVSCKPSGGTFRSYAISWVRQAPGQGLEWMRIIVFLGKVNYAQRFQGVTLTADKSTTTAYMELSSLRSE | Drent et al., 2016. |
| | DTAVYYCTGEPGARDPDAFDIWGQGTMVTVSSVLGGGGSGGGGSGGGGSDIQMTQSPSSLSASVGDRVTITCRASQGIRSWLAWYQQ | Haematologica 101: 616-625 |
| | KPEKAPKSLIYAASSLQSGVPSRFSGSGSGTDFTLTISSLQPEDFATYYCQQYNNYPLTFGGGTKVEIK [SEQ ID NO: 66] | |
| CD38 | QVQLVQSGAEVKKPGSSVKVSCKAFGGTFSSYAISWVRQAPGQGLEWMGRIIRFLGKTNHAQKFQGRVTLTADKSTNTAYMELSSLR | Drent et al., 2016. |
| | SEDTAVYYCAGEPGDRDPDAVDIWGQGTMVTVSSGGGGSGGGGSGGGGSDIQMTQSPSSLSASVGDRVTITCRASQGIRSWLAWYQQ | Haematologica 101: 616-625 |
| | KPEKAPKSLIYAASSLQSGVPSRFSGSGSGTDFTLTISSLQPEDFATYYCQQYNSYPLTFGGGTKVEIK [SEQ ID NO: 75] | |
| CD38 | DIQMTQSPSSLSASVGDRVTITCRASQGIRSWLAWYQQKPEKAPKSLIYAASSLQSGVPSRESGSGSGTDFTLTISSLQPEDFATYY | Drent et al., 2016. |
| | CQQYNSYPLTFGGGTKVEIKGGGGSGGGGSGGGGSQVQLVQSGAEVKKPGSSVKVSCKAFGGTFSSYAISWVRQAPGQGLEWMGRII | Haematologica 101: 616-625 |
| | RFLGKTNHAQKFQGRVTLTADKSTNTAYMELSSLRSEDTAVYYCAGEPGDRDPDAVDIWGQGTMVTVSS [SEQ ID NO: 103] | |

Transmembrane domain

| Origin | Amino Acid sequence | Reference |
|---|---|---|
| CD28 | FWVLVVVGGVLACYSLLVTVAFIIFWV (SEQ ID NO: 94) | UniProtKB-P10747 |

Intracellular signaling domain

| Origin | Amino Acid sequence | Reference |
|---|---|---|
| CD3 | LRVKFSRSADAPAYQQGQNQLYNELNLGRREEYDVLDKRRGRDPEMGGKPQRRKNPQEGLYNELQKDKMAEAYSEIGMKGERRRGKG | UniProtKB-P20963-1 |
| zeta | HDGLYQGLSTATKDTYDALHMQALPPR (SEQ ID NO. 99) | |

Intracellular co-stimulatory domain

| Origin | Amino Acid sequence | Reference |
|---|---|---|
| CD28 | RSKRSRLLHSDYMNMTPRRPGPTRKHYQPYAPPRDFAAYRS (SEQ ID NO: 92) | UniProtKB-P10747 |
| 4-1BB | RGRKKLLYIFKQPFMRPVQTTQEEDGCSCRFPEEEEGGCEL (SEQ ID NO: 93) | UniProtKB-Q07011 |
| CD134 | LAILLALYLLRRDQRLPPDAHKPPGGGSFRTPIQEEQADAHSTLAKI (SEQ ID NO: 100) | UniProtKB-P43489 |
| ICOS | CWLTKKKYSSSVHDPNGEYMFMRAVNTAKKSRLTDVTL (SEQ ID NO: 101) | UniProtKB-Q9Y6W8 |
| 2B4 | VWRRKRKEKQSETSPKEFLTIYEDVKDLKTRRNHEQEQTFPGGGSTIYSMIQSQSSAPTSQEPAYTLYSLIQPSRKSGSRKRNHSPS | UniProtKB-Q9BZW8 |
| | FNSTIYEVIGKSQPKAQNPARLSRKELENEDVYS (SEQ ID NO: 102) | |
| CD27 | QRRKYRSNKGESPVEPAEPCHYSCPREEEGSTIPIQEDYRKPEPACSP (SEQ ID NO: 45) | UniProtKB-P26842 |
| CD40 | VLVFIKKVAKKPTNKAPHPKQEPQEINFPDDLPGSNTAAPVQETLHGCQPVTQEDGKESRISVQERQ (SEQ ID NO: 46) | UniProtKB-P25942 |
| NKG2D | MGWIRGRRSRHSWEMSEFHNYNLDLKKSDFSTRWQKQRCPVVKSKCRENAS (SEQ ID NO: 47) | UniProtKB-P26718 |

In certain embodiments, the single chain binding domain is a CD19-targeting binding domain. An example of a CD19-targeting binding domain is derived from the mouse hybridoma FMC-63 for CD19 (Kochenderfer et al., 2010. Blood 116:4099-4102). Further suitable CD19 binding domains have been described in Alabanza et al., 2017. Mol Therapy 25:2452-2465 and in Bejcek et al., 1995. Cancer Res 55:2346-2351.

In certain embodiments, the antigen-recognizing receptor is a CAR comprising an extracellular antigen-binding domain that binds to BCMA. In certain embodiments, the CAR is a BCMA-targeted CAR disclosed in International Patent Publication No. WO16/090320, which is incorporated by reference hereby in its entirety.

In certain embodiments, the antigen-recognizing receptor is a CAR comprising an extracellular antigen-binding domain that binds to CD38.

In certain embodiments, the CAR comprises a transmembrane domain. Different transmembrane domains result in different receptor stability. After antigen recognition, receptors cluster and a signal are transmitted to the cell. In accordance with the presently disclosed subject matter, the transmembrane domain of the first antigen-recognizing receptor can comprise a native or modified transmembrane domain of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof.

In certain embodiments, the transmembrane domain of the CAR comprises a CD28 polypeptide. In certain embodiments, the transmembrane domain of the CAR comprises a CD8 polypeptide. In certain embodiments, the transmembrane domain of the CAR comprises a 4-1BB polypeptide.

In certain embodiments, the CAR further comprises a hinge/spacer region that links the extracellular antigen-binding domain to the transmembrane domain. The hinge/spacer region can be flexible enough to allow the antigen binding domain to orient in different directions to facilitate antigen recognition. In certain embodiments, the hinge/spacer region of the CAR comprises a native or modified hinge region of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof. The hinge/spacer region can be the hinge region from IgG1, or the CH2CH3 region of immunoglobulin and portions of CD3, a portion of a CD28 polypeptide (e.g., a portion of SEQ ID NO: 2), a portion of a CD8 polypeptide, a variation of any of the foregoing which is at least about 80%, at least about 85%, at least about 90%, at least about 95%, or at least about 100% homologous or identical thereto, or a synthetic spacer sequence.

In certain embodiments, the CAR comprises a hinge/spacer region comprising a native or modified hinge region of a CD28 polypeptide.

In certain embodiments, the hinge/spacer region is positioned between the extracellular antigen-binding domain and the transmembrane domain. In certain embodiments, the hinge/spacer region comprises a CD8 polypeptide, a CD28 polypeptide, a CD3ζ polypeptide, a CD4 polypeptide, a 4-1BB polypeptide, an OX40 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof. In certain embodiments, the transmembrane domain comprises a CD8 polypeptide, a CD28 polypeptide, a CD3ζ polypeptide, a CD4 polypeptide, a 4-1BB polypeptide, an OX40 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof.

In certain embodiments, the transmembrane domain and the hinge/spacer region are derived from the same molecule. In certain embodiments, the transmembrane domain and the hinge/spacer region are derived from different molecules. In certain embodiments, the hinge/spacer region comprises a CD28 polypeptide and the transmembrane domain comprises a CD28 polypeptide. In certain embodiments, the hinge/spacer region comprises a CD28 polypeptide and the transmembrane domain comprises a CD28 polypeptide. In certain embodiments, the hinge/spacer region comprises a CD84 polypeptide and the transmembrane domain comprises a CD84 polypeptide. In certain embodiments, the hinge/spacer region comprises a CD166 polypeptide and the transmembrane domain comprises a CD166 polypeptide. In certain embodiments, the hinge/spacer region comprises a CD8a polypeptide and the transmembrane domain comprises a CD8a polypeptide. In certain embodiments, the hinge/spacer region comprises a CD8b polypeptide and the transmembrane domain comprises a CD8b polypeptide. In certain embodiments, the hinge/spacer region comprises a CD28 polypeptide and the transmembrane domain comprises an ICOS polypeptide.

In certain embodiments, the CAR comprises an intracellular signaling domain. In certain embodiments, the intracellular signaling domain of the CAR comprises a CD3ζ polypeptide. CD3ζ can activate or stimulate a cell (e.g., a cell of the lymphoid lineage, e.g., a T-cell). Wild type (“native”) CD3ζ comprises three functional immunoreceptor tyrosine-based activation motifs (ITAMs), three functional basic-rich stretch (BRS) regions (BRS1, BRS2 and BRS3). CD3ζ transmits an activation signal to the cell (e.g., a cell of the lymphoid lineage, e.g., a T-cell) after antigen is bound. The intracellular signaling domain of the CD3ζ-chain is the primary transmitter of signals from endogenous TCRs.

In certain embodiments, the intracellular signaling domain of the CAR comprises a native CD3. In certain embodiments, the native CD3ζ comprises or consists of an amino acid sequence that is at least about 85%, about 90%, about 95%, about 96%, about 97%, about 98%, about 99% or about 100% identical or homologous to the amino acid sequence having a NCBI Reference No: NP_932170 (SEQ ID NO: 6) or a fragment thereof, and/or may optionally comprise up to one or up to two or up to three conservative amino acid substitutions. In certain embodiments, the CD3ζ polypeptide comprises or consists of an amino acid sequence that is a consecutive portion of SEQ ID NO: 6, which is at least about 20, or at least about 30, or at least about 40, or at least about 50, and up to about 164 amino acids in length. In certain embodiments, the native CD3ζ comprises or consists of amino acids 1 to 164, 1 to 50, 50 to 100, 52 to 164, 100 to 150, or 150 to 164 of SEQ ID NO: 6. In certain embodiments, the intracellular signaling domain of the CAR comprises a native CD3ζ comprising or consisting of amino acids 52 to 164 of SEQ ID NO: 6. SEQ ID NO: 6 is provided below:

[SEQ ID NO: 6]
MKWKALFTAA ILQAQLPITE AQSFGLLDPK LCYLLDGILF
IYGVILTALF LRVKFSRSAD APAYQQGQNQ LYNELNLGRR
EEYDVLDKRR GRDPEMGGKP QRRKNPQEGL YNELQKDKMA
EAYSEIGMKG ERRRGKGHDG LYQGLSTATK DTYDALHMQA LPPR

In certain embodiments, the native CD3ζ comprises or consists of an amino acid sequence that is at least about 85%, about 90%, about 95%, about 96%, about 97%, about 98%, about 99% or about 100% identical or homologous to the amino acid sequence set forth in SEQ ID NO: 7 or SEQ ID NO: 99. SEQ ID NO: 7 is provided below:

[SEQ ID NO: 7]
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

In certain embodiments, the intracellular signaling domain of the CAR comprises a modified CD3ζ polypeptide. In certain embodiments, the modified CD3ζ polypeptide comprises one, two or three ITAMs. In certain embodiments, the modified CD3ζ polypeptide comprises a native ITAM1. In certain embodiments, the native ITAM1 comprises or consists of the amino acid sequence set forth in SEQ ID NO: 8.

[SEQ ID NO: 8]
QNQLYNELNLGRREEYDVLDKR

An exemplary nucleic acid sequence encoding the amino acid sequence of SEQ ID NO: 8 is set forth in SEQ ID NO: 9, which is provided below.

[SEQ ID NO: 9]
CAGAACCAGCTCTATAACGAGCTCAATCTAGGACGAAGAGAGGAGTACG
ATGTTTTGGACAAGAGA

In certain embodiments, the modified CD3ζ polypeptide comprises an ITAM1 variant comprising one or more loss-of-function mutations. In certain embodiments, the ITAM1 variant comprises or consists of two loss-of-function mutations. In certain embodiments, each of the one or more (e.g., two) loss of function mutations comprises a mutation of a tyrosine residue in ITAM1. In certain embodiments, the ITAM1 variant consists of two loss-of-function mutations. In certain embodiments, the ITAM1 variant comprises or consists of the amino acid sequence set forth in SEQ ID NO: 10, which is provided below.

[SEQ ID NO: 10]
QNQLFNELNLGRREEFDVLDKR

An exemplary nucleic acid sequence encoding the amino acid sequence of SEQ ID NO: 10 is set forth in SEQ ID NO: 11, which is provided below.

[SEQ ID NO: 11]
CAGAACCAGCTCTTTAACGAGCTCAATCTAGGACGAAGAGAGGAGTTCG
ATGTTTTGGACAAGAGA

In certain embodiments, the modified CD3ζ polypeptide comprises a native ITAM2. In certain embodiments, the native ITAM2 comprises or consists of the amino acid sequence set forth in SEQ ID NO: 12, which is provided below.

[SEQ ID NO: 12]
QEGLYNELQKDKMAEAYSEIGMK

An exemplary nucleic acid sequence encoding the amino acid sequence of SEQ ID NO: 12 is set forth in SEQ ID NO: 13, which is provided below.

[SEQ ID NO: 13]
CAGGAAGGCCTGTACAATGAACTGCAGAAAGATAAGATGGCGGAGGCCT
ACAGTGAGATTGGGATGAAA

In certain embodiments, the modified CD3ζ polypeptide comprises an ITAM2 variant. In certain embodiments, the ITAM2 variant comprises or consists of one or more loss-of-function mutations. In certain embodiments, the ITAM2 variant comprises or consists of two loss-of-function mutations. In certain embodiments, each of the one or more (e.g., two) the loss of function mutations comprises a mutation of a tyrosine residue in ITAM2. In certain embodiments, the ITAM1 variant consists of two loss-of-function mutations. In certain embodiments, the ITAM2 variant comprises or consists of the amino acid sequence set forth in SEQ ID NO: 14, which is provided below.

[SEQ ID NO: 14]
QEGLFNELQKDKMAEAFSEIGMK

An exemplary nucleic acid sequence encoding the amino acid sequence of SEQ ID NO: 14 is set forth in SEQ ID NO: 15, which is provided below.

[SEQ ID NO: 15]
CAGGAAGGCCTGTTCAATGAACTGCAGAAAGATAAGATGGCGGAGGCCT
TCAGTGAGATTGGGATGAAA

In certain embodiments, the modified CD3ζ polypeptide comprises a native ITAM3. In certain embodiments, the native ITAM3 comprises or consists of the amino acid sequence set forth in SEQ ID NO: 16, which is provided below.

[SEQ ID NO: 16]
HDGLYQGLSTATKDTYDALHMQ

An exemplary nucleic acid sequence encoding the amino acid sequence of SEQ ID NO: 16 is set forth in SEQ ID NO: 17, which is provided below.

[SEQ ID NO: 17]
CACGATGGCCTTTACCAGGGTCTCAGTACAGCCACCAAGGACACCTACG
ACGCCCTTCACATGCAG

In certain embodiments, the modified CD3ζ polypeptide comprises an ITAM3 variant. In certain embodiments, the ITAM3 variant comprises or consists of two loss-of-function mutations. In certain embodiments, each of the one or more (e.g., two) the loss of function mutations comprises a mutation of a tyrosine residue in ITAM3. In certain embodiments, the ITAM3 variant comprises or consists of two loss-of-function mutations. In certain embodiments, the ITAM3 variant comprises or consists of the amino acid sequence set forth in SEQ ID NO: 18, which is provided below.

[SEQ ID NO: 18]
HDGLFQGLSTATKDTFDALHMQ

An exemplary nucleic acid sequence encoding the amino acid sequence of SEQ ID NO: 18 is set forth in SEQ ID NO: 19, which is provided below.

[SEQ ID NO: 19]
CACGATGGCCTTTTCCAGGGGCTCAGTACAGCCACCAAGGACACCTTCGA
CGCCCTTCACATGCAG

Various modified CD3ζ polypeptides and CARs comprising modified CD3ζ polypeptides are disclosed in International Patent Application Publication No. WO2019/133969, which is incorporated by reference hereby in its entirety.

In certain embodiments, the intracellular signaling domain of the CAR comprises a modified CD3ζ polypeptide comprising a native ITAM1, an ITAM2 variant comprising or consisting of one or more (e.g., two) loss-of-function mutations, and an ITAM3 variant comprising or consisting of one or more (e.g., two) loss-of-function mutations. In certain embodiments, the intracellular signaling domain of the CAR comprises a modified CD3ζ polypeptide comprising a native ITAM1, an ITAM2 variant consisting of two loss-of-function mutations, and an ITAM3 variant consisting of two loss-of-function mutations. In certain embodiments, the intracellular signaling domain of the CAR comprises a modified CD3ζ polypeptide comprising a native ITAM1 consisting of the amino acid sequence set forth in SEQ ID NO: 8, an ITAM2 variant consisting of the amino acid sequence set forth in SEQ ID NO: 14, and an ITAM3 variant consisting of the amino acid sequence set forth in SEQ ID NO: 18. In certain embodiments, the CAR is designated as “1XX”. In certain embodiments, the modified CD3ζ polypeptide comprises or consists of the amino acid sequence set forth in SEQ ID NO: 20. SEQ ID NO: 20 is provided below:

[SEQ ID NO: 20]
RVKFSRSADA PAYQQGQNQL YNELNLGRRE EYDVLDKRRG
RDPEMGGKPR RKNPQEGLFN ELQKDKMAEA FSEIGMKGER
RRGKGHDGLF QGLSTATKDT FDALHMQALP PR

In certain embodiments, the intracellular signaling domain of the CAR comprises a modified CD3ζ polypeptide comprising or consisting of an amino acid sequence that is at least about 80%, at least about 85%, at least about 90%, at least about 95%, at least about 96%, at least about 97%, at least about 98%, or at least about 99%, at least about 100% identical to SEQ ID NO: 20 or a fragment thereof, and/or may optionally comprise up to one or up to two or up to three conservative amino acid substitutions.

An exemplary nucleic acid sequence encoding the amino acid sequence of SEQ ID NO: 20 is set forth in SEQ ID NO: 21, which is provided below.

[SEQ ID NO: 21]
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTTCAATGAACTGCAGAAAGATAAGAT
GGCGGAGGCCTTCAGTGAGATTGGGATGAAAGGCGAGCGCCGGAGGGGCA
AGGGGCACGATGGCCTTTTCCAGGGGCTCAGTACAGCCACCAAGGACACC
TTCGACGCCCTTCACATGCAGGCCCTGCCCCCTCGC

In certain embodiments, the intracellular signaling domain of the CAR further comprises at least one co-stimulatory signaling region. In certain embodiments, the at least one co-stimulatory region comprises a co-stimulatory molecule or a portion thereof. In certain embodiments, the at least one co-stimulatory region comprises at least an intracellular domain of at least one co-stimulatory molecule or a portion thereof. Non-limiting examples of co-stimulatory molecules include CD28, 4-1BB, OX40, CD27, CD40, CD154, CD97, CD11a/CD18, ICOS, DAP-10, CD2, and NKG2D.

In certain embodiments, the intracellular signaling domain of the CAR comprises a co-stimulatory signaling region comprising a CD28 polypeptide, e.g., an intracellular domain of CD28 or a portion thereof. In certain embodiments, the intracellular signaling domain of the CAR comprises a co-stimulatory signaling region comprising an intracellular domain of human CD28 or a portion thereof. In certain embodiments, the intracellular signaling domain of the CAR comprises a co-stimulatory signaling region comprising a CD28 polypeptide comprising or consisting of amino acids 180 to 220 of SEQ ID NO: 2. In certain embodiments, the intracellular signaling domain of the CAR comprises a co-stimulatory signaling region comprising an intracellular domain of mouse CD28 or a portion thereof. In certain embodiments, the intracellular signaling domain of the CAR comprises a co-stimulatory signaling region comprising a CD28 polypeptide comprising or consisting of the amino acid sequence of amino acids 178 to 218 of SEQ ID NO: 4.

In certain embodiments, the intracellular signaling domain of the CAR comprises a co-stimulatory signaling region that comprises a 4-1BB polypeptide, e.g., an intracellular domain of 4-1BB or a portion thereof. In certain embodiments, the intracellular signaling domain of the CAR comprises a co-stimulatory signaling region comprising an intracellular domain of human 4-1BB or a portion thereof. In certain embodiments, the intracellular signaling domain of the CAR comprises a co-stimulatory signaling region comprising a 4-1BB polypeptide comprising or consisting of amino acids 214 to 255 of SEQ ID NO: 5. In certain embodiments, the intracellular signaling domain of the CAR comprises a co-stimulatory signaling region comprising an intracellular domain of mouse 4-1BB or a portion thereof. In certain embodiments, the intracellular signaling domain of the CAR comprises a co-stimulatory signaling region comprising a 4-1BB polypeptide comprising or consisting of the amino acid sequence of amino acids 209 to 256 of SEQ ID NO: 48.

In certain embodiments, the intracellular signaling domain of the CAR comprises two co-stimulatory signaling regions, wherein the first co-stimulatory signaling region comprises an intracellular domain of a first co-stimulatory molecule or a portion thereof, and the second co-stimulatory signaling region comprises an intracellular domain of a second co-stimulatory molecule or a portion thereof. The first and second co-stimulatory molecules are independently selected from the group consisting of CD28, 4-1BB, OX40, CD27, CD40, CD154, CD97, CD11a/CD18, ICOS, DAP-10, CD2, and NKG2D. In certain embodiments, the intracellular signaling domain of the antigen-recognizing receptor comprises two co-stimulatory signaling regions, wherein the first co-stimulatory signaling region comprises an intracellular domain of CD28 or a portion thereof and the second co-stimulatory signaling region comprises an intracellular domain of 4-1BB or a portion thereof.

In certain embodiments, the intracellular co-stimulatory signaling domain of a CAR stimulates or enhances an efficient response of the immune cell, comprising said CAR, upon binding to an antigen through the CD3ζ domain of the CAR. Said intracellular co-stimulatory signaling domain can include the intracellular portions of CD28, 4-1BB, OX40, ICOS, CD27, CD40, 2B4, NKG2D, or a relevant part thereof. Said intracellular co-stimulatory signaling domain can include the intracellular portions of human CD28, 4-1BB, OX40, ICOS, CD27, CD40, 2B4, NKG2D, or a relevant part thereof. As an alternative, or in addition, said intracellular co-stimulatory signaling domain can comprise the equivalent protein from a non-human species, e.g., mouse, rodent, monkey, ape and the like. As an alternative, or in addition, said intracellular co-stimulatory signaling domain can include a domain that is at least 80% identical, at least 90% identical, at least 95% identical, at least 99% identical, to the intracellular portions of human CD28, 4-1BB, OX40, ICOS, CD27, CD40, 2B4, NKG2D.

In certain embodiments, the intracellular signaling domain of the CAR comprises the intracellular domain of human CD3ζ. In certain embodiments, the CAR comprises an intracellular signaling domain and a co-stimulatory intracellular domain. In certain embodiments, the CAR comprises a CD3ζ signaling domain and a CD28 co-stimulatory domain, e.g., a human CD3ζ signaling domain and a human CD28 co-stimulatory domain.

In certain embodiments, the CAR and CCR that are expressed by an immune cell disclosed herein, each comprise a CD28 transmembrane domain and a co-stimulatory CD28 intracellular domain. Said CD28 transmembrane domain is fused to an antigen specific binding domain. Said intracellular domain can correspond to the cytoplasmic part of human CD28, corresponding to amino acid residues 180-220 of UniProt entry P10747. The transmembrane domain can correspond to the transmembrane region of human CD28, corresponding to amino acid residues 153-179 of UniProt entry P10747.

In certain embodiments, the co-stimulatory CD28 intracellular domains of the CAR and the CCR expressed by an immune cell according to the presently disclosed subject matter are substantially identical. Said co-stimulatory CD28 intracellular domains are at least 80% identical, at least 90% identical, at least 95% identical, at least 99% identical, or 100% identical.

In certain embodiments, the CAR comprises an extracellular antigen-binding domain that binds to BCMA (e.g., human BCMA), a transmembrane domain comprising a CD28 polypeptide (e.g., human CD28 polypeptide, e.g., a transmembrane domain of CD28 (e.g., human CD28) or a portion thereof), a hinge/spacer region derived from a CD28 polypeptide (e.g., human CD28), an intracellular signaling domain comprising a modified CD3ζ polypeptide (e.g., a modified human CD3ζ polypeptide) comprising a native ITAM1, an ITAM2 variant consisting of two loss-of-function mutations, and an ITAM3 variant consisting of two loss-of-function mutations, and a co-stimulatory signaling region comprising a CD28 polypeptide (e.g., a human CD28 polypeptide, e.g., an intracellular domain of CD28 (e.g., human CD28) of a portion thereof). In certain embodiments, the CAR is designated as “BCMA-28z1XX”.

In certain embodiments, the CAR comprises an extracellular antigen-binding domain that binds to BCMA (e.g., human BCMA), a hinge/spacer region, a transmembrane domain comprising a CD28 polypeptide (e.g., human CD28 polypeptide, e.g., a transmembrane domain of CD28 (e.g., human CD28) or a portion thereof), a co-stimulatory signaling region comprising a CD28 polypeptide (e.g., a human CD28, e.g., an intracellular domain of CD28 (e.g., human CD28) of a portion thereof), and an intracellular signaling domain comprising a modified CD3ζ polypeptide (e.g., a modified human CD3ζ polypeptide) comprising a native ITAM1, an ITAM2 variant consisting of two loss-of-function mutations, and an ITAM3 variant consisting of two loss-of-function mutations.

In addition, the extracellular antigen-binding domain can comprise a leader or a signal peptide that directs the nascent protein into the endoplasmic reticulum. Signal peptide or leader can be essential if the CAR is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. In certain embodiments, the signal peptide is covalently joined to the 5′ terminus (N-terminus) of the extracellular antigen-binding domain of the CAR. Exemplary leader sequences include, but is not limited to, a human IL-2 signal sequence (e.g., MYRMQLLSCIALSLALVTNS [SEQ ID NO: 22]), a mouse IL-2 signal sequence (e.g., MYSMQLASCVTLTLVLLVNS [SEQ ID NO: 23]); a human kappa leader sequence (e.g., METPAQLLFLLLLWLPDTTG [SEQ ID NO: 24]), a mouse kappa leader sequence (e.g., METDTLLLWVLLLWVPGSTG [SEQ ID NO: 25]); a human CD8 leader sequence (e.g., MALPVTALLLPLALLLHAARP [SEQ ID NO: 26]); a truncated human CD8 signal peptide (e.g., MALPVTALLLPLALLLHA [SEQ ID NO: 27]); a human albumin signal sequence (e.g., MKWVTFISLLFSSAYS [SEQ ID NO: 28]); and a human prolactin signal sequence (e.g., MDSKGSSQKGSRLLLLLVVSNLLLCQGVVS [SEQ ID NO: 29]).

In certain embodiments, the signal peptide comprises a CD8 polypeptide, e.g., the CAR comprises a truncated CD8 signal peptide. In certain embodiments, the signal peptide comprises or consists of the amino acid sequence set forth in SEQ ID NO: 27.

### 5.3.1.3. T Cell Receptors (TCRs)

In certain embodiments, the antigen-recognizing receptor is a TCR. A TCR is a disulfide-linked heterodimeric protein consisting of two variable chains expressed as part of a complex with the invariant CD3 chain molecules. A TCR is found on the surface of T cells, and is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains: Variable (V) region and a Constant (C) region. The Constant region is proximal to the cell membrane, followed by a transmembrane region and a short cytoplasmic tail. The variable region binds to the peptide/MHC complex. The variable domain of both chains each has three complementarity determining regions (CDRs).

In certain embodiments, a TCR can form a receptor complex with three dimeric signaling modules CD3δ/ε, CD3γ/ε and CD247 ζ/ζ or ζ/η. When a TCR complex engages with its antigen and MHC (peptide/MHC), the T cell expressing the TCR complex is activated.

In certain embodiments, the antigen-recognizing receptor is an endogenous TCR. In certain embodiments, the antigen-recognizing receptor is naturally occurring TCR.

In certain embodiments, the antigen-recognizing receptor is an exogenous TCR. In certain embodiments, the antigen-recognizing receptor is a recombinant TCR. In certain embodiments, the antigen-recognizing receptor is a non-naturally occurring TCR. In certain embodiments, the non-naturally occurring TCR differs from any naturally occurring TCR by at least one amino acid residue. In certain embodiments, the non-naturally occurring TCR differs from any naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues. In certain embodiments, the non-naturally occurring TCR is modified from a naturally occurring TCR by at least one amino acid residue. In certain embodiments, the non-naturally occurring TCR is modified from a naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues.

### 5.3.1.4. TCR Like Fusion Molecules

In certain embodiments, the antigen-recognizing receptor is a TCR like fusion molecule. Non-limiting examples of TCR fusion molecules include HLA-Independent TCR-based Chimeric Antigen Receptor (also known as “HIT-CAR”, e.g., those disclosed in International Patent Application No. PCT/US19/017525, which is incorporated by reference in its entirety), and T cell receptor fusion constructs (TRuCs) (e.g., those disclosed in Bacuerle et al., “Synthetic TRUC receptors engaging the complete T cell receptor for potent anti-tumor response,” Nature Communications volume 10, Article number: 2087 (2019), which is incorporated by reference in its entirety).

In certain embodiments, the TCR like fusion molecule is a recombinant T cell receptor (TCR). In certain embodiments, the TCR like fusion molecule comprises an antigen binding chain that comprises an extracellular antigen-binding domain and a constant domain, wherein the TCR like fusion molecule binds to the first antigen in an HLA-independent manner. Thus, in certain embodiments, the TCR like fusion molecule is an HLA-independent (or non-HLA restricted) TCR (referred to as “HIT-CAR” or “HIT”). In certain embodiments, the constant domain comprises a TCR constant region selected from the group consisting of a native or modified TRAC polypeptide, a native or modified TRBC polypeptide, a native or modified TRDC polypeptide, a native or modified TRGC polypeptide and any variants or functional fragments thereof. In certain embodiments, the constant domain comprises a native or modified TRAC polypeptide. In certain embodiments, the constant domain comprises a native or modified TRBC polypeptide. In certain embodiments, the constant domain is capable of forming a homodimer or a heterodimer with another constant domain. In certain embodiments, the antigen binding chain is capable of associating with a CD3ζ polypeptide. In certain embodiments, the antigen binding chain, upon binding to an antigen, is capable of activating the CD3ζ polypeptide associated to the antigen binding chain. In certain embodiments, the activation of the CD3ζ polypeptide is capable of activating an immunoresponsive cell. In certain embodiments, the TCR like fusion molecule is capable of integrating with a CD3 complex and providing HLA-independent antigen recognition. In certain embodiments, the TCR like fusion molecule replaces an endogenous TCR in a CD3/TCR complex. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a ligand for a cell-surface receptor, a receptor for a cell surface ligand, an antigen binding portion of an antibody or a fragment thereof or an antigen binding portion of a TCR. In certain embodiments, the extracellular antigen-binding domain of the TCR like fusion molecule comprises an antigen binding portion of an antibody. In certain embodiments, the extracellular antigen-binding domain of the TCR like fusion molecule comprises an scFv. In certain embodiments, the extracellular antigen-binding domain of the TCR like fusion molecule comprises one or two immunoglobulin variable region(s). In certain embodiments, the extracellular antigen-binding domain of the TCR like fusion molecule comprises a heavy chain variable region (V_{H}) of an antibody. In certain embodiments, the extracellular antigen-binding domain of the TCR like fusion molecule comprises a light chain variable region (V_{L}) of an antibody. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a V_{H} of an antibody, wherein the V_{H} is capable of dimerizing with another extracellular antigen-binding domain comprising a V_{L} of the antibody and form a fragment variable (Fv), e.g., an scFv. In certain embodiments, the extracellular antigen-binding domain of the TCR like fusion molecule comprises a V_{L} of an antibody, wherein the V_{L} is capable of dimerizing with another extracellular antigen-binding domain comprising a V_{H} of the antibody and form a fragment variable (Fv), e.g., an scFv.

In certain embodiments, the extracellular antigen-binding domain of the TCR like fusion molecule comprises an antigen binding portion of a TCR. In certain embodiments, the extracellular antigen-binding domain of the TCR like fusion molecule comprises an antigen binding portion of an antibody or a fragment thereof. In certain embodiments, the extracellular antigen-binding domain of the TCR like fusion molecule comprises a heavy chain variable region (V_{H}) and/or a light chain variable region (V_{L}) of an antibody. In certain embodiments, the extracellular antigen-binding domain comprises a single-chain variable fragment (scFv). In certain embodiments, the extracellular antigen-binding domain comprises a heavy chain-only antibodies (VHH). In certain embodiments, the extracellular antigen-binding domain comprises a Fab, which is optionally crosslinked. In certain embodiments, the extracellular antigen-binding domain comprises a F (ab)₂. In certain embodiments, any of the foregoing molecules can be comprised in a fusion protein with a heterologous sequence to form the extracellular antigen-binding domain.

In certain embodiments, the extracellular antigen-binding domain of the TCR like fusion molecule comprises a heavy chain variable region (V_{H}) and/or a light chain variable region (V_{L}) of an antibody, wherein the V_{H} or the V_{L} is capable of dimerizing with another extracellular antigen-binding domain comprising a V_{L} or a V_{H} (e.g., forming a fragment variable (Fv)). In certain embodiments, the Fv is a human Fv. In certain embodiments, the Fv is a humanized Fv. In certain embodiments, the Fv is a murine Fv. In certain embodiments, the Fv is identified by screening a Fv phage library with an antigen-Fc fusion protein. Additional extracellular antigen-binding domains targeting an interested antigen can be obtained by sequencing an existing scFv or a Fab region of an existing antibody targeting the same antigen.

In certain embodiments, the V_{H} and V_{L} are linked via a linker.

In certain embodiments, the antigen binding chain of the TCR like fusion molecule further comprises a constant domain. In certain embodiments, the constant domain comprises a hinge/spacer region and a transmembrane domain. In certain embodiments, the constant domain is capable of forming a homodimer or a heterodimer with another constant domain. In certain embodiments, the constant domain dimerizes through one or more disulfide-links. In certain embodiments, the antigen binding chain of the TCR like fusion molecule is capable of forming a trimer or oligomer with one or more identical or different constant domains.

In certain embodiments, the constant domain comprises a TCR constant region, e.g., T cell receptor alpha constant region (TRAC), T cell receptor beta constant region (TRBC, e.g., TRBC1 or TRBC2), T cell receptor gamma constant region (TRGC, e.g., TRGC1 or TRGC2), T cell receptor delta constant region (TRDC) or any variants or functional fragments thereof.

In certain embodiments, the constant domain of the TCR like fusion molecule comprises a native or modified TRAC polypeptide. In certain embodiments, the TRAC polypeptide comprises an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 30 or a fragment thereof, and/or may optionally comprise up to one or up to two or up to three conservative amino acid substitutions. SEQ ID NO: 30 is provided below.

[SEQ ID NO: 30]
IQNPDPAVYQLRDSKSSDKSVCLFTDFDSQTNVSQSKDSDVYITDKTVLD
MRSMDFKSNSAVAWSNKSDFACANAFNNSIIPEDTFFPSPESSCDVKLVE
KSFETDTNLNFQNLSVIGFRILLLKVAGFNLLMTLRLWSS

In certain embodiments, the TRAC polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence encoded by a transcript expressed by the gene of NCBI Genbank ID: 28755, NG_001332.3, range 925603 to 930229 (SEQ ID NO: 31) or a fragment thereof, and/or may optionally comprise up to one or up to two or up to three conservative amino acid substitutions. SEQ ID NO: 31 is provided below.

| [SEQ ID NO: 31] | |
|---|---|
| 1 | atatccagaa ccctgaccct gccgtgtacc agctgagaga ctctaaatcc agtgacaagt |
| 61 | ctgtctgcct attcaccgat tttgattctc aaacaaatgt gtcacaaagt aaggattctg |
| 121 | atgtgtatat cacagacaaa actgtgctag acatgaggtc tatggacttc aagagcaaca |
| 181 | gtgctgtggc ctggagcaac aaatctgact ttgcatgtgc aaacgccttc aacaacagca |
| 241 | ttattccaga agacaccttc ttccccagcc caggtaaggg cagctttggt gccttcgcag |
| 301 | gctgtttcct tgcttcagga atggccaggt tctgcccaga gctctggtca atgatgtcta |
| 361 | aaactcctct gattggtggt ctcggcctta tccattgcca ccaaaaccct ctttttacta |
| 421 | agaaacagtg agccttgttc tggcagtcca gagaatgaca cgggaaaaaa gcagatgaag |
| 481 | agaaggtggc aggagagggc acgtggccca gcctcagtct ctccaactga gttcctgcct |
| 541 | gcctgccttt gctcagactg tttgcccctt actgctcttc taggcctcat tctaagcccc |
| 601 | ttctccaagt tgcctctcct tatttctccc tgtctgccaa aaaatctttc ccagctcact |
| 661 | aagtcagtct cacgcagtca ctcattaacc caccaatcac tgattgtgcc ggcacatgaa |
| 721 | tgcaccaggt gttgaagtgg aggaattaaa aagtcagatg aggggtgtgc ccagaggaag |
| 781 | caccattcta gttgggggag cccatctgtc agctgggaaa agtccaaata acttcagatt |
| 841 | ggaatgtgtt ttaactcagg gttgagaaaa cagctacctt caggacaaaa gtcagggaag |
| 901 | ggctctctga agaaatgcta cttgaagata ccagccctac caagggcagg gagaggaccc |
| 961 | tatagaggcc tgggacagga gctcaatgag aaaggagaag agcagcaggc atgagttgaa |
| 1021 | tgaaggaggc agggccgggt cacagggcct tctaggccat gagagggtag acagtattct |
| 1081 | aaggacgcca gaaagctgtt gatcggcttc aagcagggga gggacaccta atttgctttt |
| 1141 | cttttttttt tttttttttt tttttttttt tgagatggag ttttgctctt gttgcccagg |
| 1201 | ctggagtgca atggtgcatc ttggctcact gcaacctccg cctcccaggt tcaagtgatt |
| 1261 | ctcctgcctc agcctcccga gtagctgaga ttacaggcac ccgccaccat gcctggctaa |
| 1321 | ttttttgtat ttttagtaga gacagggttt cactatgttg gccaggctgg tctcgaactc |
| 1381 | ctgacctcag gtgatccacc cgcttcagcc tcccaaagtg ctgggattac aggcgtgagc |
| 1441 | caccacaccc ggcctgcttt tcttaaagat caatctgagt gctgtacgga gagtgggttg |
| 1501 | taagccaaga gtagaagcag aaagggagca gttgcagcag agagatgatg gaggcctggg |
| 1561 | cagggtggtg gcagggaggt aaccaacacc attcaggttt caaaggtaga accatgcagg |
| 1621 | gatgagaaag caaagagggg atcaaggaag gcagctggat tttggcctga gcagctgagt |
| 1681 | caatgatagt gccgtttact aagaagaaac caaggaaaaa atttggggtg cagggatcaa |
| 1741 | aactttttgg aacatatgaa agtacgtgtt tatactcttt atggcccttg tcactatgta |
| 1801 | tgcctcgctg cctccattgg actctagaat gaagccaggc aagagcaggg tctatgtgtg |
| 1861 | atggcacatg tggccagggt catgcaacat gtactttgta caaacagtgt atattgagta |
| 1921 | aatagaaatg gtgtccagga gccgaggtat cggtcctgcc agggccaggg gctctcccta |
| 1981 | gcaggtgctc atatgctgta agttccctcc agatctctcc acaaggaggc atggaaaggc |
| 2041 | tgtagttgtt cacctgccca agaactagga ggtctggggt gggagagtca gcctgctctg |
| 2101 | gatgctgaaa gaatgtctgt ttttcctttt agaaagttcc tgtgatgtca agctggtcga |
| 2161 | gaaaagcttt gaaacaggta agacaggggt ctagcctggg tttgcacagg attgcggaag |
| 2221 | tgatgaaccc gcaataaccc tgcctggatg agggagtggg aagaaattag tagatgtggg |
| 2281 | aatgaatgat gaggaatgga aacagcggtt caagacctgc ccagagctgg gtggggtctc |
| 2341 | tcctgaatcc ctctcaccat ctctgacttt ccattctaag cactttgagg atgagtttct |
| 2401 | agcttcaata gaccaaggac tctctcctag gcctctgtat tcctttcaac agctccactg |
| 2461 | tcaagagagc cagagagagc ttctgggtgg cccagctgtg aaatttctga gtcccttagg |
| 2521 | gatagcccta aacgaaccag atcatcctga ggacagccaa gaggttttgc cttctttcaa |
| 2581 | gacaagcaac agtactcaca taggctgtgg gcaatggtcc tgtctctcaa gaatcccctg |
| 2641 | ccactcctca cacccaccct gggcccatat tcatttccat ttgagttgtt cttattgagt |
| 2701 | catccttcct gtggtagcgg aactcactaa ggggcccatc tggacccgag gtattgtgat |
| 2761 | gataaattct gagcacctac cccatcccca gaagggctca gaaataaaat aagagccaag |
| 2821 | tctagtcggt gtttcctgtc ttgaaacaca atactgttgg ccctggaaga atgcacagaa |
| 2881 | tctgtttgta aggggatatg cacagaagct gcaagggaca ggaggtgcag gagctgcagg |
| 2941 | cctcccccac ccagcctgct ctgccttggg gaaaaccgtg ggtgtgtcct gcaggccatg |
| 3001 | caggcctggg acatgcaagc ccataaccgc tgtggcctct tggttttaca gatacgaacc |
| 3061 | taaactttca aaacctgtca gtgattgggt tccgaatcct cctcctgaaa gtggccgggt |
| 3121 | ttaatctgct catgacgctg cggctgtggt ccagctgagg tgaggggcct tgaagctggg |
| 3181 | agtggggttt agggacgcgg gtctctgggt gcatcctaag ctctgagagc aaacctccct |
| 3241 | gcagggtctt gcttttaagt ccaaagcctg agcccaccaa actctictac ttcttcctgt |
| 3301 | tacaaattcc tcttgtgcaa taataatggc ctgaaacgct gtaaaatatc ctcatttcag |
| 3361 | ccgcctcagt tgcacttctc ccctatgagg taggaagaac agttgtttag aaacgaagaa |
| 3421 | actgaggccc cacagctaat gagtggagga agagagacac ttgtgtacac cacatgcctt |
| 3481 | gtgttgtact tctctcaccg tgtaacctcc tcatgtcctc tctccccagt acggctctct |
| 3541 | tagctcagta gaaagaagac attacactca tattacaccc caatcctggc tagagtctcc |
| 3601 | gcaccctcct cccccagggt ccccagtcgt cttgctgaca actgcatcct gttccatcac |
| 3661 | catcaaaaaa aaactccagg ctgggtgcgg gggctcacac ctgtaatccc agcactttgg |
| 3721 | gaggcagagg caggaggagc acaggagctg gagaccagcc tgggcaacac agggagaccc |
| 3781 | cgcctctaca aaaagtgaaa aaattaacca ggtgtggtgc tgcacacctg tagtcccagc |
| 3841 | tacttaagag gctgagatgg gaggatcgct tgagccctgg aatgttgagg ctacaatgag |
| 3901 | ctgtgattgc gtcactgcac tccagcctgg aagacaaagc aagatcctgt ctcaaataat |
| 3961 | aaaaaaaata agaactccag ggtacatttg ctcctagaac tctaccacat agccccaaac |
| 4021 | agagccatca ccatcacatc cctaacagtc ctgggtcttc ctcagtgtcc agcctgactt |
| 4081 | ctgttcttcc tcattccaga tctgcaagat tgtaagacag cctgtgctcc ctcgctcctt |
| 4141 | cctctgcatt gcccctcttc tccctctcca aacagaggga actctcctac ccccaaggag |
| 4201 | gtgaaagctg ctaccacctc tgtgcccccc cggcaatgcc accaactgga tcctacccga |
| 4261 | atttatgatt aagattgctg aagagctgcc aaacactgct gccaccccct ctgttccctt |
| 4321 | attgctgctt gtcactgcct gacattcacg gcagaggcaa ggctgctgca gcctcccctg |
| 4381 | gctgtgcaca ttccctcctg ctccccagag actgcctccg ccatcccaca gatgatggat |
| 4441 | cttcagtggg ttctcttggg ctctaggtcc tgcagaatgt tgtgaggggt ttattttttt |
| 4501 | ttaatagtgt tcataaagaa atacatagta ttcttcttct caagacgtgg ggggaaatta |
| 4561 | tctcattatc gaggccctgc tatgctgtgt atctgggcgt gttgtatgtc ctgctgccga |
| 4621 | tgccttc |

In certain embodiments, the constant domain of the TCR like fusion molecule comprises a native or modified TRBC polypeptide. In certain embodiments, the constant domain of the TCR like fusion molecule comprises a native or modified TRBC2 polypeptide. In certain embodiments, the TRBC2 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 32 or a fragment thereof, and/or may optionally comprise up to one or up to two or up to three conservative amino acid substitutions. SEQ ID NO: 32 is provided below.

[SEQ ID NO: 32]
DLKNVFPPEVAVFEPSEAEISHTQKATLVCLATGF3YPDHVELSWWVNGK
EVHSGVSTDPQPLKEQPALNDSRYCLSSRLRVSATFWQNPRNHFRCQVQF
YGLSENDEWTQDRAKPVTQIVSAEAWGRADCGFTSESYQQGVLSATILYE
ILLGKATLYAVLVSALVLMAMVKRKDSRG

In certain embodiments, the constant domain of the TCR like fusion molecule comprises a native or modified TRBC1 polypeptide. In certain embodiments, the TRBC1 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 33 or a fragment thereof, and/or may optionally comprise up to one or up to two or up to three conservative amino acid substitutions. SEQ ID NO: 33 is provided below.

[SEQ ID NO: 33]
LNKVFPPEV AVFEPSEAEI SHTQKATLVC LATGFFPDHV
ELSWWVNGKE VHSGVSTDPQ PLKEQPALND SRYCLSSRLR
VSATFWQNPR NHFRCQVQFY GLSENDEWTQ DRAKPVTQIV
SAEAWGRADC GFTSVSYQQG VLSATILYEI LLGKATLYAV
LVSALVLMAM VKRKDF

In certain embodiments, the TRBC polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence encoded by a transcript expressed by a gene of NCBI Genbank ID: 28639, NG_001333.2, range 645749 to 647196 (TRBC1, SEQ ID NO: 34), NCBI Genbank ID: 28638, NG_001333.2 range 655095 to 656583 (TRBC2, SEQ ID NO: 35) or a fragment thereof, and/or may optionally comprise up to one or up to two or up to three conservative amino acid substitutions. SEQ ID NOS: 34 and 35 are provided below.

| [SEQ ID NO: 34] | |
|---|---|
| 1 | aggacctgaa caaggtgttc ccacccgagg tcgctgtgtt tgagccatca gaagcagaga |
| 61 | tctcccacac ccaaaaggcc acactggtgt gcctggccac aggcttcttc cccgaccacg |
| 121 | tggagctgag ctggtgggtg aatgggaagg aggtgcacag tggggtcagc acagacccgc |
| 181 | agcccctcaa ggagcagccc gccctcaatg actccagata ctgcctgagc agccgcctga |
| 241 | gggtctcggc caccttctgg cagaaccccc gcaaccactt ccgctgtcaa gtccagttct |
| 301 | acgggctctc ggagaatgac gagtggaccc aggatagggc caaacccgtc acccagatcg |
| 361 | tcagcgccga ggcctggggt agagcaggtg agtggggcct ggggagatgc ctggaggaga |
| 421 | ttaggtgaga ccagctacca gggaaaatgg aaagatccag gtagcagaca agactagatc |
| 481 | caaaaagaaa ggaaccagcg cacaccatga aggagaattg ggcacctgtg gttcattctt |
| 541 | ctcccagatt ctcagcccaa cagagccaag cagctgggtc ccctttctat gtggcctgtg |
| 601 | taactctcat ctgggtggtg ccccccatcc ccctcagtgc tgccacatgc catggattgc |
| 661 | aaggacaatg tggctgacat ctgcatggca gaagaaagga ggtgctgggc tgtcagagga |
| 721 | agctggtctg ggcctgggag tctgtgccaa ctgcaaatct gactttactt ttaattgcct |
| 781 | atgaaaataa ggtctctcat ttattttcct ctccctgctt tctttcagac tgtggcttta |
| 841 | cctcgggtaa gtaagccctt ccttttcctc tccctctctc atggttcttg acctagaacc |
| 901 | aaggcatgaa gaactcacag acactggagg gtggagggtg ggagagacca gagctacctg |
| 961 | tgcacaggta cccacctgtc cttcctccgt gccaacagtg tcctaccagc aaggggtcct |
| 1021 | gtctgccacc atcctctatg agatcctgct agggaaggcc accctgtatg ctgtgctggt |
| 1081 | cagcgccctt gtgttgatgg ccatggtaag caggagggca ggatggggcc agcaggctgg |
| 1141 | aggtgacaca ctgacaccaa gcacccagaa gtatagagtc cctgccagga ttggagctgg |
| 1201 | gcagtaggga gggaagagat ttcattcagg tgcctcagaa gataacttgc acctctgtag |
| 1261 | gatcacagtg gaagggtcat gctgggaagg agaagctgga gtcaccagaa aacccaatgg |
| 1321 | atgttgtgat gagccttact atttgtgtgg tcaatgggcc ctactacttt ctctcaatcc |
| 1381 | tcacaactcc tggctcttaa taacccccaa aactttctct tctgcaggtc aagagaaagg |
| 1441 | atttctga |
| [SEQ ID NO: 35] | |
| 1 | aggacctgaa aaacgtgttc ccacccgagg tcgctgtgtt tgagccatca gaagcagaga |
| 61 | tctcccacac ccaaaaggcc acactggtat gcctggccac aggcttctac cccgaccacg |
| 121 | tggagctgag ctggtgggtg aatgggaagg aggtgcacag tggggtcagc acagacccgc |
| 181 | agcccctcaa ggagcagccc gccctcaatg actccagata ctgcctgagc agccgcctga |
| 241 | gggtctcggc caccttctgg cagaaccccc gcaaccactt ccgctgtcaa gtccagttct |
| 301 | acgggctctc ggagaatgac gagtggaccc aggatagggc caaacccgtc acccagatcg |
| 361 | tcagcgccga ggcctggggt agagcaggtg agtggggcct ggggagatgc ctggaggaga |
| 421 | ttaggtgaga ccagctacca gggaaaatgg aaagatccag gtagcggaca agactagatc |
| 481 | cagaagaaag ccagagtgga caaggtggga tgatcaaggt tcacagggtc agcaaagcac |
| 541 | ggtgtgcact tcccccacca agaagcatag aggctgaatg gagcacctca agctcattct |
| 601 | tccttcagat cctgacacct tagagctaag ctttcaagtc tccctgagga ccagccatac |
| 661 | agctcagcat ctgagtggtg tgcatcccat tctcttctgg ggtcctggtt tcctaagatc |
| 721 | atagtgacca cttcgctggc actggagcag catgagggag acagaaccag ggctatcaaa |
| 781 | ggaggctgac tttgtactat ctgatatgca tgtgtttgtg gcctgtgagt ctgtgatgta |
| 841 | aggctcaatg tccttacaaa gcagcattct ctcatccatt tttcttcccc tgttttcttt |
| 901 | cagactgtgg cttcacctcc ggtaagtgag tctctccttt ttctctctat ctttcgccgt |
| 961 | ctctgctctc gaaccagggc atggagaatc cacggacaca ggggcgtgag ggaggccaga |
| 1021 | gccacctgtg cacaggtgcc tacatgctct gttcttgtca acagagtctt accagcaagg |
| 1081 | ggtcctgtct gccaccatcc tctatgagat cttgctaggg aaggccacct tgtatgccgt |
| 1141 | gctggtcagt gccctcgtgc tgatggccat ggtaaggagg agggtgggat agggcagatg |
| 1201 | gcagtaggga gggaagagat ttcattcagg tgcctcagaa gataacttgc acctctgtag |
| 1261 | gcctaatata tcctatcacc tcaatgaaac cataatgaag ccagactggg gagaaaatgc |
| 1321 | agggaatatc acagaatgca tcatgggagg atggagacaa ccagcgagcc ctactcaaat |
| 1381 | taggcctcag agcccgcctc ccctgcccta ctcctgctgt gccatagccc ctgaaaccct |
| 1441 | gaaaatgttc tctcttccac aggtcaagag aaaggattcc agaggctag |

In certain embodiments, the constant domain of the TCR like fusion molecule comprises a native or modified TRGC polypeptide. In certain embodiments, the constant domain of the TCR like fusion molecule comprises a native or modified TRGC1 polypeptide. In certain embodiments, the TRGC1 polypeptide comprises or consists of an amino acid sequence that is at least about 85%, about 90%, about 95%, about 96%, about 97%, about 98%, about 99% or 100% homologous or identical to the amino acid sequence set forth in SEQ ID NO: 36, which is provided below.

[SEQ ID NO: 36]
DKQLDADVSP KPTIFLPSIA ETKLQKAGTY LCLLEKFFPD
VIKIHWQEKK SNTILGSQEG NTMKINDTYM KFSWLTVPEK
SLDKEHRCIV RHENNKNGVD QEIIFPPIKT DVITMDPKDN
CSKDANDTLL LQLTNTSAYY MYLLLLLKSV VYFAIITCCL
LRRTAFCCNG EKS

In certain embodiments, the constant domain of the TCR like fusion molecule comprises a native or modified TRGC2 polypeptide. In certain embodiments, the TRGC2 polypeptide comprises or consists of an amino acid sequence that is at least about 85%, about 90%, about 95%, about 96%, about 97%, about 98%, about 99% or 100% homologous or identical to the amino acid sequence set forth in SEQ ID NO: 37, which is provided below.

[SEQ ID NO: 37]
DKQLDADVSP KPTIFLPSIA ETKLQKAGTY LCLLEKFFPD
IIKIHWQEKK SNTILGSQEG NTMKTNDTYM KFSWLTVPEE
SLDKEHRCIV RHENNKNGID QEIIFPPIKT DVTTVDPKYN
YSKDANDVIT MDPKDNWSKD ANDTLLLQLT NTSAYYTYLL
LLLKSVVYFA IITCCLLRRT AFCCNGEKS

In certain embodiments, the TRGC polypeptide comprises or consists of an amino acid sequence that is at least about 85%, about 90%, about 95%, about 96%, about 97%, about 98%, about 99% or 100% homologous or identical to the amino acid sequence encoded by a transcript expressed by a gene of NCBI Genbank ID: 6966, NG_001336.2, range 108270 to 113860 (TRGC1, SEQ ID NO: 38), NCBI Genbank ID: 6967, NG_001336.2, range 124376 to 133924 (TRGC2, SEQ ID NO: 39) or a fragment thereof, and/or may optionally comprise up to one or up to two or up to three conservative amino acid substitutions. SEQ ID NOs: 38 and 39 are provided below.

| [SEQ ID NO: 38] | |
|---|---|
| 1 | ataaacaact tgatgcagat gtttccccca agcccactat ttttcttcct tcaattgctg |
| 61 | aaacaaagct ccagaaggct ggaacatacc tttgtcttct tgagaaattt ttccctgatg |
| 121 | ttattaagat acattggcaa gaaaagaaga gcaacacgat tctgggatcc caggagggga |
| 181 | acaccatgaa gactaacgac acatacatga aatttagctg gttaacggtg ccagaaaagt |
| 241 | cactggacaa agaacacaga tgtatcgtca gacatgagaa taataaaaac ggagttgatc |
| 301 | aagaaattat ctttcctcca ataaagacag gtatgtgttt acgcatatca tctgtcagaa |
| 361 | cacttctttg aaagtgaatg ctgcattttt tcctttcagt attaatgaaa aacaaacata |
| 421 | aatctttctt aaatattgtt acatttaatg gtagcataaa tgccctgcta cttttctata |
| 481 | gaattaaaat ggtataggtt ttggagaaaa caaaattgaa aaagttactg aaggtttgtc |
| 541 | agcctcagct ccattatcca aaataagaaa gtcacgtgct ggtttttagg gttgttagat |
| 601 | ggattaaaga aacaacatac acagaagcat ctagcaacgt gacacgtggt aaacgctcaa |
| 661 | aaagtgttct cccttctttt gatgacttta cttgatcagg aaataacata tatatgtctt |
| 721 | tcaggaatgt tctgcccaag caggagagtc actcacctca atcttgctac ccacaaagtt |
| 781 | taacctaaaa acaacgggtt cattgttgac aaaatgatgt ttatctgttg ttgacagaat |
| 841 | gatgtttatc taaaaacagt tccaattttc tatttccttt gctgagacac aaaggggagg |
| 901 | caaatgtgca aagcttgagg gtagtcttac cactgtgctt aagtgttctg atttttctag |
| 961 | tgatcagggc aaaataaaaa gtatagtaag ttccaaggca gtgaatatta tacaggagag |
| 1021 | aagttacagt tttataatgt gttttccttt acactaaatt ctaaaagtaa aaagtctttt |
| 1081 | tttttttttg acagagtttc actcttgttg cccaagcagg tgtgctatgg tatgatctca |
| 1141 | gctcactgca acctccacct cccgggttca agtgattctc ttacttcagc ctcccgacag |
| 1201 | gctgggattg caggcgcctg ccaccacacc tggctaattt ttgtgttttt agtagagatg |
| 1261 | gggtttcacc atgttggcca ggctggtctc aaattcctga cctcaagtga tccatccacc |
| 1321 | tcggcctcca agtgctggga ttatgggcgt cagccactgt gcccagccta aaagtaaaat |
| 1381 | gtctttcatg agcttcccaa ggcagctacg ttaaggagga cacttctctt aatgtcattc |
| 1441 | tacagtagat ttctaatgct ctttcttgga agtttgtttt tctgagaaaa gctaaaaata |
| 1501 | taacatggaa gtgatcatat tatataatca atgaagtgct tttcaaggag ataaaactaa |
| 1561 | tctggtccac acttgcaacc aaccttgatt gagagagaga gagaactcag gatacacttg |
| 1621 | aagattttat tatggggaac agttacttta ttctttttac ctcaatcaat gcatggaaat |
| 1681 | aagtgatagt cattttcatt tatcttttaa taaatgaagt caccatgagg aaaataaaaa |
| 1741 | gacattgaaa acccattaaa gtcagccctt aaagatattt ggacatgcag acttgataac |
| 1801 | taacgtttgc attcttgaga cttacccaaa acccatacct caagtccaag tttttagaat |
| 1861 | tcatgaaata aagatctcag tgagtgcata aaattgcgca ccagaatcat atccgtatag |
| 1921 | acaagaacac atctactaga aaaataataa accaacacac caatgcaact gtgttttctt |
| 1981 | ctgttttaaa gtatgttgtc tttgtatgca tgtttgcttc ttcctttttt tttttaacat |
| 2041 | cacagataaa ttcaactctc acctcaggtt ttattgagag aactgtcaat gtgacttggc |
| 2101 | ctctgtcttt ctagtcccag aaagaattgc actgaaatct gagctcctgt aataaaaaca |
| 2161 | accatttgct gagagtaatt aacatactga aagagatttt cttagagtac acaatggtga |
| 2221 | cattatattg cctctttata aataactttc tatctatttc tgtggattat tcctacaaag |
| 2281 | tacttttcat atgtccaatt tcttttcttc ccctacaact actgtctgaa tactggctct |
| 2341 | gctatttgct gatatgattc tcggcaagtt gcctgcactt tttaaacttt atttcctcat |
| 2401 | tcagaacatg gggccataca taatacaact cacttcagtg ttattgggga attaaacaaa |
| 2461 | aaatgcatgg gaagcattta acatagtgcc tgacacaata atgagtactc agtagatgtt |
| 2521 | agcttttatt aatattgttg ttgttatgtc cagaaacact atacctccag aaaatcatgg |
| 2581 | gtacttgctg gggacattgg ggatatgcat gatttggaaa agaatgactg ctttttttgc |
| 2641 | ttagatgaga aatttttcta agccagactc cttcaaatat gtaagattct gttgtggatt |
| 2701 | caaggactga aagaattctt ggccgagtgt ggtggcttat ccctgtaatc ccagcatttt |
| 2761 | gtgaggacaa ggcaggaaga ttgcttgagt ccaggagttt gaaaccagcc tgcgcaacat |
| 2821 | ggcgaaaccc tgtctctaca aaaaatacaa acattagctc ggagtgagtg ctgacatgtg |
| 2881 | cctgtactcc cagctactca gaaggctgag atgggaggat ctcatgagcc tggggagttt |
| 2941 | gaggcttcag tgagccgtga tgacaccgta ctatactcca ctccagcctg ggtgacagtg |
| 3001 | agaccctgcc tcaaaaaaca aacaaacaaa caaacaaaac aaaattaatc tttttgctga |
| 3061 | tgtcatgtca gcagtgtgtg ttgaaggctg taaagcagcc atttgttcag tttatttttc |
| 3121 | cattgaacaa gtatttatca aaaacatact ttgtggcagt cactatgcta ggagctatga |
| 3181 | atacagaagg aaaagtaaat gctcttggat actacactcc agttgtgata aaaaagaaaa |
| 3241 | aatgtattct tcaccaactt caacatcttg atgtgcaaaa acataataca tgaattagat |
| 3301 | ctacctaatt acacagaatt agaccaattg tttctggaat tgtgggctca tatttttaat |
| 3361 | aactgtcctc ctgcctctct gtcgacaggt tttataaata ttcatttaat tacacacaca |
| 3421 | cacacgaaca attgactagt acttgctctc attcttctag atgtcatcac aatggatccc |
| 3481 | aaagacaatt gttcaaaaga tgcaaatggt aagcttttgt gtttttccct tcctcctgat |
| 3541 | cattttgttt tgaacttctc tggcttgaaa aatcagggaa tggattttgc taggttggat |
| 3601 | gctgcagaat ggacctagtg atattttaaa ttagtccctc attttctagg agttgtatta |
| 3661 | acaaacctaa ctactgcttt ggggtatgag atgactgtaa attagagagg gtacagtggt |
| 3721 | atagtgatat gcttttaatt atttcaaaaa aaagatttta ttcattcatg tgtctttttt |
| 3781 | ctttttcttt tctttttttt ttttttttgg acagagtctt gctctgtcac ccaggctgga |
| 3841 | gtgcggtggc agtatctcag ctcaccacaa cctccgcctc ccggcttcaa gtgattctcc |
| 3901 | tgcctcagct tctcgagtag ctgggactac aggcgcgtgc caccatgccc ggctaatttt |
| 3961 | tgtattttta gtagagttgg ggtttcacca tgttggccag gatggcctcg aatttgtgac |
| 4021 | ctcgtgatct gccccctcgc cctcccgaac tgttgggatt acaggcgtga gtcactgtgc |
| 4081 | ccggcctcct gtcctgtctt ttgtttaatg actgggaaaa acatgatacc atgttgcttc |
| 4141 | tcgagttgtt ttgttttagt ctttggtctt tgctagtagc taataacacg aactagtgtt |
| 4201 | tatcaagtgc tttttacaca gaagggcttg ggctgtgttc tgcattttct tgtttaaccc |
| 4261 | tcttaaaact cctataaaat ggtacatatt tttctcccaa tttacagtcc ctttaaagca |
| 4321 | aataattata aaaatcccta tacatgtcac acagctagat ctgggatttc aaatcaggcc |
| 4381 | atcaaacaaa gagtttatgt acttagtaag ttttctgttc tttttctaca atagagtcag |
| 4441 | atagcaagaa attaccaagc caggaacctg aaacaaaacg gacatcatgt ggggctgggt |
| 4501 | gggtgcatgg gctttgcaga ctggactttc actccagctc ttttaatgat taggtgtaag |
| 4561 | tgacctacat tttgtgagca acagttttct catcagccaa caaagaataa ttacaccaga |
| 4621 | ttcacagtta ttgaagagat aaaggcatga atgtgagatg tctggcatag ggcatctcat |
| 4681 | ttagcagaca cagaatgagt acttgtttct ggctttttct ctctacatat gcacaaagaa |
| 4741 | tgcgactaga agcatgggct ctagccctgc tcaactttcc tctatttcca ataccaaggg |
| 4801 | gctctgactt aggctgccac accaggcaag gagggcagta ccacctcact tgaccaaggg |
| 4861 | cagggagtca cggacacatc acttcttgag atccttttcc acaccaagga ctgatgtttc |
| 4921 | tggaattctc actttatgaa gacaaaacat ataaatggaa attttctcag gtagagactc |
| 4981 | actcttgtag ctcattgagt aggcactagt ggtccacccc cactgtcttt acttattcct |
| 5041 | tgacatcaca tatctcttgc aaaacctcaa ataatattaa atgcaatcac ccaataatag |
| 5101 | catagccata attagaggca tttaggaaag acaggtgagt gtgccacaac tacctaacac |
| 5161 | atcagcaaat ctggattaac cactttcttt gattttccac aatgcaacct tactttttaa |
| 5221 | tagttgggaa tgttctaagt gaatttagca gaggttgtta atcaacttga aagctgaatt |
| 5281 | ctgacttgtc tgactcttgg tggtgctggt agcagtagat gtttactttt aggttttggt |
| 5341 | ggtggtggaa tatcacttca acgtaaatca tcagaaataa gtatttgtga acccctctcg |
| 5401 | cattaatgta tcttattctg taaaaagaac atgtgcaatt tctcttagat acactactgc |
| 5461 | tgcagctcac aaacacctct gcatattaca tgtacctcct cctgctcctc aagagtgtgg |
| 5521 | tctattttgc catcatcacc tgctgtctgc ttagaagaac ggctttctgc tgcaatggag |
| 5581 | agaaatcata a |
| [SEQ ID NO: 39] | |
| 1 | ataaacaact tgatgcagat gtttccccca agcccactat ttttcttcct tcgattgctg |
| 61 | aaacaaaact ccagaaggct ggaacatacc tttgtcttct tgagaaattt ttcccagata |
| 121 | ttattaagat acattggcaa gaaaagaaga gcaacacgat tctgggatcc caggagggga |
| 181 | acaccatgaa gactaacgac acatacatga aatttagctg gttaacggtg ccagaagagt |
| 241 | cactggacaa agaacacaga tgtatcgtca gacatgagaa taataaaaac ggaattgatc |
| 301 | aagaaattat ctttcctcca ataaagacag gtatgtgttt acacatatca tctgtcagaa |
| 361 | cacttctttg aaagtgaatg ctgcattttt tcctttcagt attaatgaaa aacataaatc |
| 421 | tttcttaaaa attgttacat ttaatggtag cgtaaatgcc ctgctacttt tctatagaat |
| 481 | taaaatggta taggttttgg agaaaacaaa attgaaaaag ttgctgaagg tttgtcagcc |
| 541 | tcagctccat tatccaaaat aagaaagtca cgtgctggtt tttagggttg ttagatggat |
| 601 | taaagaaaca acatacacag aagcatctag caacgtgaca cgtggtaaac gctcaaaaag |
| 661 | tgttctccct tcttttgatg actttacttg atcaggaaat aacatatata tgtctttcag |
| 721 | gaatgttctg cccaagcagg agagtcactc acctcaatct tgctacccac aaagtttaac |
| 781 | ctaaaaacaa cgggttcatt gttgacaaaa taatgtttat ctgaagataa ctgtagatca |
| 841 | tatttatctg tagataatgt ttatctgtgg agtgtggctc tacaaaacat agaatagtct |
| 901 | tggtcactgc agttttatag aggccttggg tttttcagag tttcatttta tatatcacca |
| 961 | taaagtaaca tttcataatt acaggttggt aaggcttaca tgtacaaaca ttcttccatt |
| 1021 | ttccataata aatgcatttc ctgccattgg tgaatgcagc tcaataaaca tttattgtac |
| 1081 | aattatgaca cgccaggctt agtggaaatg tggatgaaca gacaaggatg agttactgtc |
| 1141 | ctaaggatga tgcatgacag tgcagagaat atactctctt cctgatcact cagggtcact |
| 1201 | catgattcat gcgcgaggtc ccaaaacagt gcctttgatg cagattctgt acatctctag |
| 1261 | acgattggtc caagggctga atgtgctctg gcccagtggt ccagtctgtc actatatgtc |
| 1321 | aacatcctga atatgaacat aacagtccaa catctcaaga gtgggcatga aaaggactca |
| 1381 | ttttgtgctt tttcctgtgg ttaacaagtc ctttttagcc tgggggaaca agcattaaca |
| 1441 | aaatgtttga agatctttgc cacgtaccat tccaaatttc tagggtaagt ctttagcttt |
| 1501 | tcagatcctg agtttctgca atgatcaaat gtgatttgga cagttgcgtt gactttctcc |
| 1561 | tggggctata atggagtgca aaggaaacaa tggcagggaa aatgcttgct ttcaaaatgg |
| 1621 | tagcatggat gtgttcattc gtgtagttac tgtattaggt atagcctttc ctgaaactaa |
| 1681 | ctgaagtggg gttataaaaa cagtcccaat tttctatttc ctttgctgag acacaaagag |
| 1741 | gagacaaaag agcaaagctt gagggtagtt ttaccactgt gcttaagtgt tctgattttt |
| 1801 | ccagtgatca gggtgaaata aaaagcatag taagttccag ggcagtgaat accatacagg |
| 1861 | agacaagtta cagttttata atgtgtttta ctttacacta aattctaaaa gtaaaatgtc |
| 1921 | tttttttttt tccgagacag agtttcactc ttgtagccca ggcaggagtg ctatggtgtg |
| 1981 | atctcggctc acagcaacct ccacctccca gtttcaagcg attcttctgc ctcagcctcc |
| 2041 | cgagaagttg aaattacagg tgcctggcac catatctcgc taattattct atttttagta |
| 2101 | gagatcgggt tttaccatgt tggccaggct ggtctcgaac tcctgacttc aagtgatcca |
| 2161 | cccgcctcag cctcccaaag tgctgggatt acaggtgtga gtcactgtgc cggacctaac |
| 2221 | agtaaaatgt ctttcatgtg cttctcaagg caactacatt aaggaggaca catctcttaa |
| 2281 | tgtcattcta cagtagattt ctaatgctct ttcttggaag tttgtttttc tgagaagagc |
| 2341 | taaaaatata ataacatgga agtgatcata ttatataatc aatgaagtgc tttcaaagga |
| 2401 | gataaaacta acctggtctg catttgcaac cagccttgat tgagagagag agaactcagg |
| 2461 | atacacttag agattttatt atggggaata gttactttat tcattttacc tcaatcaatg |
| 2521 | catggaaata agtgacagtc attttcattt atcttttaat aaataaagtc accatgagga |
| 2581 | aaatgaaaac ccattaaagt cagtccttaa agatatttgg acatgcagac atgataacta |
| 2641 | acatttccat tcgtgagact tacccaaaac ctatacctca agtccatttc ttagaataca |
| 2701 | tgaaataaag atctcagtga gtgtataaaa ctgcacacca gaatcatatc cgtatagaca |
| 2761 | agaatacatc tactagaaaa atataaacca aaacaccaag gtgactctgt ttttttctgt |
| 2821 | tttaaaatat gttgtctttg tatgcatgtt tgcttcttcc tttttttttt taaacatcgc |
| 2881 | agataaattc aactctcacc tcagttgaga gagaactgtc aatgtgactt ggcctctctc |
| 2941 | tttctagtcc cagaaagaat tgcactgaaa tgctgagctc ctgtaataaa aatgaccatt |
| 3001 | tgctgagagt aattaacata ctgaaagaga ttttcttaga atagtgcaca atggcccaat |
| 3061 | ggtgacatta tattgtctct ttataaatta ttttctatct atttctgtgg attatttcta |
| 3121 | caaagcactt ttcatatgtc caattccttt tattccccta caagtactga ctgactactg |
| 3181 | gctctgctgt tcactgatat gactttcggc aagttgcctg cactttttaa acgttatttc |
| 3241 | ctcattcaga acatggggcc atacaaaata caactcactt cagtgttatt ggggaattaa |
| 3301 | acaaataaat gcatgggaag catttaacat agtgcctgac acaataatga gcactcagta |
| 3361 | gatgttagct tttattaata ttgttgttgc tatgtccaga aacactatac ctccagaaaa |
| 3421 | tcatgggtac ttgctgggga cgttggggat atgcatgatt ttgaaaggag tgactgctct |
| 3481 | ttactgctca gatgagaaat ttttctaagc cagactcctt caaacatgta agattctgtt |
| 3541 | gtggattcta ggactgaaag aattcttggc cgagtgtggt ggcttatcct ggtaatctca |
| 3601 | tcatttggga ggacaaggca ggaagattgc ttgagcccag gagttggaaa caagcctgga |
| 3661 | caacatggcg aaaccctgtc tctacaaaaa atacaaacat tagctggtca tgggagtgag |
| 3721 | tgcctgtact cccagctact caggaggcta agataggagg atcacctgag cctgggcagt |
| 3781 | ttgaggtttc agtgagccgt gatgacacca tactatactc cactccagcc tgggtgacag |
| 3841 | tgacatcctg cctcaaaaaa acccccaaaa ttattctttt tgctgatttc atgtcagcag |
| 3901 | tgtgtgctga aggctgtaaa gtagccactt gttctgttta tttttccatt gaacaagtat |
| 3961 | ttatcaaaaa cgtactttgt ggaaggcact gtgctaggaa ctatgcatac agaaggaaaa |
| 4021 | ccaaatgttc ttggatacta cactccagtt gtgataaaaa agaaaaaagt attcttcaca |
| 4081 | aacttcaaca ttttgatgtg caaaaacata atatatgaat tagatctacc taactacaca |
| 4141 | gaattagacc aattatttct gggattatgg gctcatattt ttaataactg tcctcctacc |
| 4201 | tctctgttga caggttttat aaatattcat ttaattacac acagtcacag acacactcag |
| 4261 | acacacacac atacacacac acacacacct tgacaaataa tgggcatgaa caattgactg |
| 4321 | gtacttgctc tcattcttct agatgtcacc acagtggatc ccaaatacaa ttattcaaag |
| 4381 | gatgcaaatg gtaagttttt gtgtttttta tttcctcctg atcattttaa gttttgaact |
| 4441 | tctctggctt gaaaaatcag ggaatggatt ttgctaggtt ggatgctgca gaatggacct |
| 4501 | aatcatattt taaattagtc cctctttttc taggagttgt attaacaaac ctaactactg |
| 4561 | cttcatgtaa gagatgactg taaattgaag ggtacagtga tatgctttca gttatttcaa |
| 4621 | aaaacagact ttactcatcc atgtgtcttt tttcttttct tttttttctt ttttgagacg |
| 4681 | gagtctcgct ctgttgaaca ggctggattg cagtgacgcg atctcacctc actacaacct |
| 4741 | ccgcctctgg agttcaagcg attctccagc ctcagcttct caagtagctg ggactacagg |
| 4801 | cacatgccac catgtccggg tcatctttgt atttttagca gagaccgggt ttcactatgt |
| 4861 | tggccaggct ggtctagaat tcctgacttc gtgatctgcc ccctcagccc tccgaagtgc |
| 4921 | tgggattaca gacgtgagtc actgtgcccg gcctaacagt aaaatgtctt tcatgcgctt |
| 4981 | ctcaaggcaa ctacgttaag gaggacactt ctcttaatgt cattctacag tagatttcta |
| 5041 | atgctctttc ttggaagttt gtttttctga gaaaagctaa aaatataaca tggaagtgat |
| 5101 | catattgtat aatcaatgaa gtgcttttca aggagataaa actaatctgg tccacgtttg |
| 5161 | caaccaacct tgattgagag agagagagaa ctcaggatac acttggagat tttattatgg |
| 5221 | ggaatagtta ctttattctt ttttcctcaa tcaattcatg gaaataagtg atagtcatat |
| 5281 | tcatttatct tttaataaat gaagtcacca tgaggaaaat aaaaagacat tgaaaaccca |
| 5341 | ttaaagttag cccttaaaga tatttggaca tgcagacttg ataactaacg tttgcattct |
| 5401 | tgagacttac ccaaaaccca tacctcaagt ccatgttttt agaattcatg aaataaagat |
| 5461 | ctcagtgagt gcataaaatt gcgcaccaga atcatatccg tatagacaag aacacatcta |
| 5521 | ctagaaaaat aataaaccaa cacaccaatg caactgtgtt ttcttctgtt ttaaaatatg |
| 5581 | ttgtctttgt atgcatgttt gcttcttcct tttttttttt taacatcaca gataaattca |
| 5641 | actctcacct caggttttat tgagagaact gtcaatgtga cttggcctct gtctttctag |
| 5701 | tcccagaaag aatcgcactg aaatgctgag ctcctgtaat aaaaatgacc atttgctgag |
| 5761 | agtaattaac atactgaaag agattttctt agagtacaca atggtgacat tatattgtct |
| 5821 | ctttataaat aactttctat ctatttctgt ggattattcc tacaaagtac ttttcatatg |
| 5881 | tccagtttct tttcttcccc tacaactacc gtctgaatac tggctctgct atttgctgat |
| 5941 | atgattctcg gcaagttgcc tgcacttttt aaactttatt tcctcattca gaacatgggg |
| 6001 | ccatgtaata ctcatgtacg tgagtattac gtaataatgc tcacttaagt gttactgggg |
| 6061 | aattaaacaa aaaaatgcat ggcaagcatt taacatagtg cctgacacaa taatgagcac |
| 6121 | tcagtagatg ttagatttta ttaatattgt tgttgttatg tccggaaaca ctatacctcc |
| 6181 | agaaaatcat gggtacttgc ttgggatgtt ggggatatgc atgatttgga aaggtatgac |
| 6241 | tgcttttttc tgcttagatg agaaattttt ctaagccaga ctccttcaaa tatgtaagat |
| 6301 | tctgttgtgg attctaggac ggaaagaatt cttggtcagg tgtggtttct tatccctgta |
| 6361 | atcccagaat tttgggagga caaggcagga agattgcttg agcccaggag tttgaaacca |
| 6421 | gcctgggcaa caagacgaaa ccctgtctct acaaaagtac ataaattagc ttggcttggt |
| 6481 | ggtgtgtgcc tgtattacca gctattcggg agactgagat gggaggatct cctgaacctg |
| 6541 | tgaagtttga ggcttcagtg agccgtgatg acaccatact atactcgact ccagcctgtg |
| 6601 | cgacagtgag actctgcgtc aaaaaaaaaa ccccaaaatt attgtttttg ctgatttcag |
| 6661 | gtcagcagtg tgtgctgaag ggtgtaaagt agccacttga tcagtttatt tttccactga |
| 6721 | acaagtattt atcaaaaaca tactttgtgg tctgtttttg ataaataaaa aggcactgtg |
| 6781 | ctaggagcca tgaatacaga aggaaaacca aatgttcttg gatactacac tccagttgtg |
| 6841 | ataaaaaaga aaaatgtatt cttcacgaac ttcaacattt tgatatgcaa aaacatagta |
| 6901 | tataaattag atctacctga ttacgtagaa tcagaccaat tatttctgga attgagggct |
| 6961 | catattttta ataactgtcc tcctgcctct ctgttgacag gttttataaa tattcattta |
| 7021 | attacacaca cacacacaca caccttgaca aataatggac atgaacaatt gactagtact |
| 7081 | tgctctcatt cttctagatg tcatcacaat ggatcccaaa gacaattggt caaaagatgc |
| 7141 | aaatggtaag cttttgtgtt tttcctttcc tcctgatcat tttaagtttt gaacttctct |
| 7201 | ggcttgaaaa atcagggaat gggccgggtg cggtggctca cgcctgtaat cccagcactt |
| 7261 | tgggaggccg aggcgggcgg atcacgaggt caggagatcg agaccatccc ggctaaaacg |
| 7321 | gtgaaacccc gtctctacta aaaatacaaa aaattagccg ggcttagtgg cgggcgcctg |
| 7381 | tagtcccagc tacttgggag gctgaggcag gagaatggcg tgaacccggg aggcggagct |
| 7441 | tgcagtgagc cgagattgcg ccactgcact ccactccagc ctgggcgaca gagcgagact |
| 7501 | ccgtctcaaa aaaaaaaaaa aaaaaaaaaa aagaaaaatc agggaatgga ttttgctagg |
| 7561 | ttggatgctg cagaatggac ctagtgatat tttaaattag tccctctttt tctaggagtt |
| 7621 | gtattaacaa acctaactac tgcttcgggt atgagatgac tgtaaattag agggtacagt |
| 7681 | gatatgcttt cagttatttc aaaaaacaga ctttattcat ccgtctgtct tttttttttt |
| 7741 | tttttttttt tttttttgag acggaggagt ctcactctat cacccaggct ggagtgcagt |
| 7801 | ggcgcgatct cggctcacca taacctccgc cttactggtt caagcgattc tccagcctca |
| 7861 | gcttctcaag tagctgggac tacaggtgca caccaccata cctggctaat ttttgtattt |
| 7921 | ttaatagaga tggggtttca ccacgctggc caggatggtc ttgaattctt gacctcgtga |
| 7981 | tctgccccct cgggctccca aacttctggg attataggcg tgagccactg tgcccggcct |
| 8041 | tctgtctttt gttataatga ctggggaaaa catgatacca tgttgcttct tgagttgttt |
| 8101 | tgttttagtc tttggtcttt gctagtagct aataacacga actagtgttt atcaagtgct |
| 8161 | ttttacacag aagggcttgt tctgcatttt ctagtttaat catcttaata ctcctataaa |
| 8221 | gtagtacaat atattttctc ccattttaca gtccctttaa agtaaataac tataaaaatc |
| 8281 | ccttatacat gtcacacagc taggtctggc atttcaaatc aggacatcaa acaaagaatt |
| 8341 | cgtgcagtta ctaagtcctc tattttttct acaatagaaa aaatagcaag aattacagat |
| 8401 | agcaagacat tacaaggcag gaatctgaaa cgaaagggac ataatgtggg gctgggtggg |
| 8461 | tgcatgagct ttgcagacta gactttcatt ccagctcttt taatgattag gtgtaagtga |
| 8521 | cctacatttt gtgagtaaca gttttctcat cagccaacta agaataatta caccagattc |
| 8581 | acagttattg aagagataag ggcatgaatg tgagatgtct ggcgtagggt atctcattta |
| 8641 | gcagacacag aatgaatact tgtttctggc tttttctctc tacatatgca caaagaatgt |
| 8701 | gactagaagc attggctcta gccctgctca actttcctct atttccaata ccaaggggct |
| 8761 | ctgacttagg ctgccacacc aggcaaggag gggcagtacc acctcacttg accaagggca |
| 8821 | gggagtcacg gacacatcac ttcctgagat ccttttccac accaaggact gatgtttctg |
| 8881 | gaattctcac tttatgaaga caaaacatat aaatggaaat ttctgcagga agagactcac |
| 8941 | tcttgtagct cattgagtag gcactagtgg tccaccccca ctgtctttac ttattccttg |
| 9001 | acatcacata tctcttgtaa aacctcaaat aatgttaaat gcaatcaccc aataatagca |
| 9061 | tagccataat tagaggcatt taggaaagac aggtgagtgt gccacaacta cctaacacat |
| 9121 | cagcaaatct ggattaacca ctttctttga ttttccacaa tgcaacctta ctttttaata |
| 9181 | gttgggaatg ttctaagtga atttagcaga ggttgttaat caacttgaaa gctgaattct |
| 9241 | gacttgtctg actcttggtg gtgctggtag cagtagatgt ttacttttag gttttggtgg |
| 9301 | tggtggaata tcacttcaac gtaaatcatc agaaataagt atttgtgaac ccctctcgca |
| 9361 | ttaatatatc ttattctgta aaaagaacat gtgcaatttc tcttagatac actactgctg |
| 9421 | cagctcacaa acacctctgc atattacacg tacctcctcc tgctcctcaa gagtgtggtc |
| 9481 | tcatcacctg ctgtctgctt agaagaacgg ctttctgctg caatggagag |
| 9541 | aaatcataa |

In certain embodiments, the constant domain of the TCR like fusion molecule comprises a native or modified TRDC polypeptide. In certain embodiments, the TRDC polypeptide comprises or consists of an amino acid sequence that is at least about 85%, about 90%, about 95%, about 96%, about 97%, about 98%, about 99% or 100% homologous or identical to the amino acid sequence set forth in SEQ ID NO: 40, which is provided below.

[SEQ ID NO: 40]
SQPHTKPSVF VMKNGTNVAC LVKEFYPKDI RINLVSSKKI
TEFDPAIVIS PSGKYNAVKL GKYEDSNSVT CSVQHDNKTV
HSTDFEVKTD STDHVKPKET ENTKQPSKSC HKPKAIVHTE
KVNMMSLTVL GLRMLFAKTV AVNFLLTAKL FFL

In certain embodiments, the TCR like fusion molecule comprises a hinge/spacer region that links the extracellular antigen-binding domain to the constant domain. The hinge/spacer region can be flexible enough to allow the antigen binding domain to orient in different directions to facilitate antigen recognition. In certain embodiments, the hinge/spacer region can be the hinge region from IgG1, or the CH₂CH₃ region of immunoglobulin and portions of CD3, a portion of a CD28 polypeptide, a portion of a CD8 polypeptide, a variation of any of the foregoing which is at least about 80%, at least about 85%, at least about 90%, or at least about 95% homologous or identical thereto, or a synthetic spacer sequence. In certain non-limiting embodiments, the hinge/spacer region of the CAR can comprise a native or modified hinge region of a CD3ζ polypeptide, a CD40 polypeptide, a 4-1BB polypeptide, an OX40 polypeptide, a CD166 polypeptide, a CD166 polypeptide, a CD8a polypeptide, a CD8b polypeptide, an ICOS polypeptide, an ICAM-1 polypeptide, a CTLA-4 polypeptide, a synthetic polypeptide (not based on a protein associated with the immune response), or a combination thereof.

In certain embodiments, the TCR like fusion molecule comprises an antigen binding chain, which does not comprise an intracellular domain. In certain embodiments, the antigen binding chain is capable of associating with a CD3ζ polypeptide. In certain embodiments, the antigen binding chain comprises a constant domain, which is capable of associating with a CD3ζ polypeptide. In certain embodiments, the CD3ζ polypeptide is endogenous. In certain embodiments, the CD3ζ polypeptide is exogenous. In certain embodiments, binding of the antigen binding chain to an antigen is capable of activating the CD3ζ polypeptide associated to the antigen binding chain. In certain embodiments, the exogenous CD3ζ polypeptide is fused to or integrated with a costimulatory molecule disclosed herein.

In certain embodiments, the TCR like fusion molecule comprises an antigen binding chain that comprises an intracellular domain. In certain embodiments, the intracellular domain comprises a CD3ζ polypeptide. In certain embodiments, binding of the antigen binding chain to an antigen is capable of activating the CD3ζ polypeptide of the antigen binding chain.

In certain embodiments, the CD3ζ polypeptide comprises or consists of an amino acid sequence that is at least about 85%, about 90%, about 95%, about 96%, about 97%, about 98%, about 99% or about 100% homologous to the amino acid sequence set forth in SEQ ID NO: 6 or a fragment thereof, and/or may optionally comprise up to one or up to two or up to three conservative amino acid substitutions. In certain embodiments, the CD3ζ polypeptide comprises or consists of an amino acid sequence that is a consecutive portion of SEQ ID NO: 6, which is at least about 20, or at least about 30, or at least about 40, or at least about 50, and up to about 164 amino acids in length. In certain embodiments, the CD3ζ comprises or consists of the amino acid sequence of amino acids 1 to 164, 1 to 50, 50 to 100, 52 to 164, 100 to 150, or 150 to 164 of SEQ ID NO: 6. In certain embodiments, the CD3ζ polypeptide comprises or consists of the amino acid sequence of amino acids 52 to 164 of SEQ ID NO: 6.

In certain embodiments, the CD3ζ polypeptide comprises or consists of an amino acid sequence that is at least about 85%, about 90%, about 95%, about 96%, about 97%, about 98%, about 99% or about 100% homologous or identical to SEQ ID NO: 7 or a fragment thereof, and/or may optionally comprise up to one or up to two or up to three conservative amino acid substitutions. In certain embodiments, the CD3ζ polypeptide comprises or consists of the amino acid sequence set forth in SEQ ID NO: 7.

In certain embodiments, the TCR like fusion molecule comprises an antigen binding chain that comprises an intracellular domain, wherein the intracellular domain comprises a co-stimulatory signaling region. In certain embodiments, the intracellular domain comprises a co-stimulatory signaling region and a CD3ζ polypeptide. In certain embodiments, the intracellular domain comprises a co-stimulatory signaling region and does not comprise a CD3ζ polypeptide. In certain embodiments, the co-stimulatory signaling region comprises at least an intracellular domain of a co-stimulatory molecule disclosed herein.

In certain embodiments, the TCR like fusion molecule is capable of associating with a CD3 complex (also known as “T-cell co-receptor”). In certain embodiments, the TCR like fusion molecule and the CD3 complex form an antigen recognizing receptor complex similar to a native TCR/CD3 complex. In certain embodiments, the CD3 complex is endogenous. In certain embodiments, the CD3 complex is exogenous. In certain embodiments, the TCR like fusion molecule replaces a native and/or an endogenous TCR in the CD3/TCR complex. In certain embodiments, the CD3 complex comprises a CD3γ chain, a CD3δ chain, and two CD3ε chains.

In certain embodiments, the CD3γ chain comprises or consists of an amino acid sequence that is at least about 85%, about 90%, about 95%, about 96%, about 97%, about 98%, about 99% or about 100% homologous or identical to the amino acid sequence having a NCBI reference number: NP_000064.1 (SEQ ID NO: 41) or a fragment thereof, and/or may optionally comprise up to one or up to two or up to three conservative amino acid substitutions. SEQ ID NO: 41 is provided below.

| [SEQ ID NO: 41] | |
|---|---|
| 1 | MEQGKGLAVL ILAIILLQGT LAQSIKGNHL VKVYDYQEDG SVLLTCDAEA KNITWFKDGK |
| 61 | MIGFLTEDKK KWNLGSNAKD PRGMYQCKGS QNKSKPLQVY YRMCQNCIEL NAATISGFLF |
| 121 | AEIVSIFVLA VGVYFIAGQD GVRQSRASDK QTLLPNDQLY QPLKDREDDQ YSHLQGNQLR |
| 181 | RN |

In certain embodiments, the CD3δ chain comprises or consists of an amino acid sequence that is at least about 85%, about 90%, about 95%, about 96%, about 97%, about 98%, about 99% or about 100% homologous or identical to the amino acid sequence having a NCBI reference numbers: NP_000723.1 (SEQ ID NO: 42) or a fragment thereof, or the amino acid sequence having a NCBI reference numbers: NP_001035741.1 (SEQ ID NO: 43) or a fragment thereof, and/or may optionally comprise up to one or up to two or up to three conservative amino acid substitutions. SEQ ID NOs: 42 and 43 are provided below.

| [SEQ ID NO: 42] | |
|---|---|
| 1 | MEHSTFLSGL VLATLLSQVS PFKIPIEELE DRVFVNCNTS ITWVEGTVGT LLSDITRLDL |
| 61 | GKRILDPRGI YRCNGTDIYK DKESTVQVHY RMCQSCVELD PATVAGIIVT DVIATLLLAL |
| 121 | GVFCFAGHET GRLSGAADTQ ALLRNDQVYQ PLRDRDDAQY SHLGGNWARN K |
| [SEQ ID NO: 43] | |
| 1 | MEHSTFLSGL VLATLLSQVS PFKIPIEELE DRVFVNCNTS ITWVEGTVGT LLSDITRLDL |
| 61 | GKRILDPRGI YRCNGTDIYK DKESTVQVHY RTADTQALLR NDQVYQPLRD RDDAQYSHLG |
| 121 | GNWARNK |

In certain embodiments, the CD3ε chain comprises or consists of an amino acid sequence that is at least about 85%, about 90%, about 95%, about 96%, about 97%, about 98%, about 99% or about 100% homologous or identical to the amino acid sequence having a NCBI reference number: NP_000724.1 (SEQ ID NO: 44) or a fragment thereof, and/or may optionally comprise up to one or up to two or up to three conservative amino acid substitutions. SEQ ID NO: 44 is provided below.

| [SEQ ID NO: 44] | |
|---|---|
| 1 | mqsgthwrvl glcllsvgvw gqdgneemgg itqtpykvsi sgttviltcp qypgseilwq |
| 61 | hndkniggde ddknigsded hlslkefsel eqsgyyvcyp rgskpedanf ylylrarvce |
| 121 | ncmemdvmsv ativivdici tggllllvyy wsknrkakak pvtrgagagg rqrgqnkerp |
| 181 | ppvpnpdyep irkgqrdlys glnqrri |

In certain embodiments, the TCR like fusion molecule exhibits a greater antigen sensitivity than a CAR targeting the same antigen. In certain embodiments, the TCR like fusion molecule is capable of inducing an immune response when binding to an antigen that has a low density on the surface of a tumor cell. In certain embodiments, cells comprising the TCR like fusion molecule can be used to treat a subject having tumor cells with a low expression level of a surface antigen, e.g., from a relapse of a disease, wherein the subject received treatment which leads to residual tumor cells. In certain embodiments, the tumor cells have a low density of a target molecule on the surface of the tumor cells. In certain embodiments, a target molecule having a low density on the cell surface has a density of less than about 5,000 molecules per cell, less than about 4,000 molecules per cell, less than about 3,000 molecules per cell, less than about 2,000 molecules per cell, less than about 1,500 molecules per cell, less than about 1,000 molecules per cell, less than about 500 molecules per cell, less than about 200 molecules per cell, or less than about 100 molecules per cell. In certain embodiments, a target molecule having a low density on the cell surface has a density of less than about 2,000 molecules per cell. In certain embodiments, a target molecule having a low density on the cell surface has a density of less than about 1,500 molecules per cell. In certain embodiments, a target molecule having a low density on the cell surface has a density of less than about 1,000 molecules per cell. In certain embodiments, a target molecule having a low density on the cell surface has a density of between about 4,000 molecules per cell and about 2,000 molecules per cell, between about 2,000 molecules per cell and about 1,000 molecules per cell, between about 1,500 molecules per cell and about 1,000 molecules per cell, between about 2,000 molecules per cell and about 500 molecules per cell, between about 1,000 molecules per cell and about 200 molecules per cell, or between about 1,000 molecules per cell and about 100 molecules per cell.

Various TCR like fusion molecules are disclosed in International Patent Application Publication No. WO2019/133969, which is incorporated by reference hereby in its entirety.

### 5.3.2. Gene Disruption of B2M Locus

In certain embodiments, a presently disclosed cell further comprises a gene disruption of a B2M locus. In certain embodiments, the gene disruption of the B2M locus results in a non-functional beta 2-microglobulin. In certain embodiments, the gene disruption of the B2M locus results in knockout of the B2M gene expression.

In certain embodiments, the gene disruption of the B2M locus (e.g., knockout of the B2M locus) is generated using a non-viral method. Non-viral approaches can also be employed for genetic modification of a cell. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Any targeted genome editing methods can also be used to generate the gene disruption of the B2M locus. In certain embodiments, the gene disruption of the B2M locus is generated by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, a CRISPR system is used to generate the gene disruption of the B2M locus.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

In certain embodiments, the B2M locus is disrupted using a gRNA to knockout expression of B2M.

In certain embodiments, zinc-finger nucleases are used to generate the gene disruption of the B2M locus. A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

In certain embodiments, a TALEN system is used to generate the gene disruption of the B2M locus. Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

In certain embodiments, the gene disruption of the B2M locus can be a disruption of the coding region of the B2M locus and/or a disruption of the non-coding region of the CD70 locus. In certain embodiments, the gene disruption of the B2M locus comprises a disruption of the coding region of the B2M locus. In certain embodiments, the gene disruption of the B2M locus comprises an insertion at the coding region of the B2M locus. Human B2M protein comprises two exons: exon 1 and exon 2. In certain embodiments, the gene disruption of the B2M locus comprises a disruption at one or more of exon 1 and exon 2 of the B2M locus. In certain embodiments, the gene disruption of the B2M locus comprises a disruption at exon 1 of the B2M locus. In certain embodiments, the gene disruption of the B2M locus comprises an insertion at exon 1 of the B2M locus.

In certain embodiments, the gene disruption of the B2M locus is produced prior to the expression of the first antigen-recognizing receptor in the cell.

Immune rejection of stem cells is due to the expression of human leucocyte antigen class I molecules (HLA-I) on the surface of these cells (Zhang et al., J Cell Mol Med. (2020): 24:695-710). HLA-I presents “non-self” antigens to CD8⁺ T cells that eliminate the transplanted cells through direct cytotoxic effect (Zhang 2020). Due to the polymorphic nature of the HLA-I genes, it is often difficult to identify a perfect match between donor and recipient prior to transplantation (Zhang 2020). HLA-I comprises a heavy chain and a light chain, which is also called B2-microglobulin (B2M). HLA-I structure is disrupted and non-functional when the B2M gene is deleted (Zhang 2020). In certain embodiments, the gene disruption of the B2M locus can reduce stem cell-induced immune rejection, thereby making the cells more suitable for an allogeneic setting.

### 5.3.3. Gene Disruption of CIITA Locus

In certain embodiments, a presently disclosed cell further comprises a gene disruption of a Class II transactivator (CIITA) locus. In certain embodiments, the gene disruption of the CIITA locus results in a non-functional MHC class II transactivator. In certain embodiments, the gene disruption of the CIITA locus results in knockout of the CIITA gene expression.

Any methods to generate the gene disruption of the B2M locus as disclosed in Section 5.3.6. can be used to generate the gene disruption of the CIITA locus. In certain embodiments, the gene disruption of the CIITA locus is generated by a method comprising a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the gene disruption of the CIITA locus can be a disruption of the coding region of the CIITA locus and/or a disruption of the non-coding region of the CIITA locus. In certain embodiments, the gene disruption of the CIITA locus comprises a disruption of the coding region of the CIITA locus. In certain embodiments, the gene disruption of the CIITA locus comprises an insertion at the coding region of the CIITA locus. Human CIITA protein comprises 22 exons: exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, and exon 22. In certain embodiments, the gene disruption of the CIITA locus comprises a disruption at one or more of exon 1 through exon 22 of the CIITA locus. In certain embodiments, the gene disruption of the CIITA locus comprises a disruption at exon 3 of the CIITA locus. In certain embodiments, the gene disruption of the CIITA locus comprises an insertion at exon 3 of the CIITA locus.

CIITA is a transcriptional coactivator that regulates y-interferon-activated transcription of Major Histocompatibility Complex (MHC) class I and II genes (Devaiah et al., Frontiers in Immunology (2013); Vol. 4; Article 476:1-6). Thus, CIITA plays a critical role in immune responses: CIITA deficiency results in aberrant MHC gene expression and consequently in autoimmune diseases such as Type II bare lymphocyte syndrome (Devaiah 2013). Although CIITA does not bind to DNA directly, it regulates MHC transcription in two distinct ways—as a transcriptional activator and as a general transcription factor (Devaiah 2013). The CIITA is a master regulator of MHC gene expression (Devaiah 2013). CIITA induces de novo transcription of MHC class II genes and enhances constitutive MHC class I gene expression (Devaiah 2013). MHC II expression is regulated by CIITA. In certain embodiments, the gene disruption of the CIITA locus can reduce immune rejection, and improve survival of allogeneic bone marrow stem cells, thereby making the cells more suitable for an allogeneic setting.

### 5.3.4. Methods of Producing

The presently disclosed subject matter further provides a method of producing an immune cell disclosed herein, comprising providing immune cells (e.g., human immune cells, human T-cells, human NK cells) and enabling expression of a CAR and a CCR by the immune cells.

In certain embodiments, said method comprises introducing into an immune cell a nucleic acid sequence that encodes a chimeric antigen receptor and a co-stimulatory chimeric receptor. In certain embodiments, said chimeric antigen receptor comprises an antigen-binding domain coupled to an intracellular signaling domain. In certain embodiments, a CAR comprises an antigen specific binding domain binding to a first antigen; a CD28 transmembrane domain, an intracellular CD3ζ signaling domain; and a co-stimulatory CD28 intracellular domain. In certain embodiments, said co-stimulatory chimeric receptor comprises an antigen-binding domain coupled to a co-stimulatory signaling domain. In certain embodiments, a CCR comprises an antigen specific binding domain binding to a second antigen; a CD28 transmembrane domain; and an intracellular signaling domain; and a co-stimulatory CD28 intracellular domain and a co-stimulatory 4-1BB intracellulairintracellular domain; wherein the first antigen differs from the second antigen.

In certain embodiments, said immune cell is a T cell. In certain embodiments, said immune cell is a cytotoxic T cell. In certain embodiments, said immune cell is a NK cell.

### 5.4. Nucleic Acid Compositions and Vectors

The presently disclosed subject matter provides nucleic acid molecules and/or nucleic acid compositions comprising a first polynucleotide encoding a CD38 CCR (e.g., one disclosed in Section 5.2). In certain embodiments, the nucleic acid molecules and/or nucleic acid composition further comprises a second polynucleotide encoding an antigen-recognizing receptor (e.g., one disclosed in Section 5.3.1). Also provided are cells comprising the nucleic acid molecules and/or nucleic acid compositions disclosed herein.

The presently disclosed subject matter provides further provides a nucleic acid molecule that enables expression of a CAR and CCR in an immune cell (e.g., a human T cell or NK cell). Said nucleic acid molecule comprises one or more nucleic acid sequences encoding an intracellular signaling domain (e.g., a human CD3ζ domain), a co-stimulatory CD28 intracellular domain, a CD28 transmembrane domain and an antigen binding domain. In certain embodiments, said nucleic acid molecule encodes the intracellular signaling domain, the co-stimulatory CD28 intracellular domain, the CD28 transmembrane domain and an antigen binding domain of CAR; and the intracellular signaling domain, the co-stimulatory 4-1BB domain, the co-stimulatory CD28 domain, the CD28 transmembrane domain and an antigen binding domain of CCR.

In certain embodiments, the nucleic acid molecules and/or nucleic acid compositions comprise regulatory elements such as elements that provide constitutive expression of the CAR and CCR in many cell types or, elements that direct expression of the CAR and CCR in certain cell types, e.g., in T cells and/or NK cells (i.e., tissue-specific regulatory sequences). Regulatory elements include promoters, enhancers and transcription termination sequences such as a poly(A) signal.

In certain embodiments, the nucleic acid molecules and/or nucleic acid composition further comprise a first promoter that is operably linked to the first polynucleotide. In certain embodiments, the nucleic acid molecules and/or nucleic acid composition further comprise a second promoter that is operably linked to the second polynucleotide. In certain embodiments, one or both of the first and second promoters are endogenous or exogenous.

In certain embodiments, the exogenous promoter is selected from an elongation factor (EF)-1 promoter, a CMV promoter, a SV40 promoter, a PGK promoter, and a metallothionein promoter. In certain embodiments, one or both of the first and second promoters are inducible promoters. In certain embodiment, the inducible promoter is selected from a NFAT transcriptional response element (TRE) promoter, a CD69 promoter, a CD25 promoter, and an IL-2 promoter.

In certain embodiments, one or both of the first and second promoters are pol II promoters. Examples of suitable pol II promoters include, but are not limited to, retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the beta-actin promoter, the phosphoglycerol kinase (PGK) promoter, and the EF1a promoter.

In certain embodiments, the nucleic acid molecules and/or nucleic acid composition further comprise a first enhancer that is operably linked to the first polynucleotide. In certain embodiments, the nucleic acid molecules and/or nucleic acid composition further comprise a second enhancer that is operably linked to the second polynucleotide. Non-limiting examples of enhancers include CMV enhancers; the R-US' segment in LTR of HTLV-I; SV40 enhancer; and the intron sequence between exons 2 and 3 of rabbit beta-globin. It will be appreciated by those skilled in the art that the design of the expression vector can depend on such factors as the choice of the host cell to be transformed, the level of expression desired, etc. Said regulatory elements such as promoter sequences may be autologous sequences or heterologous sequences.

In certain embodiments, the CD38 CCR and/or the antigen-recognizing receptor is integrated at a locus within the genome of a T cell, e.g., a TRAC locus, a TRBC locus, a TRDC locus, a TRGC locus, a B2m locus, an AAVS1 locus, or a PD-1 locus. In certain embodiments, the locus is a TRAC locus. In certain embodiments, the expression of the CD38 CCR and/or the antigen-recognizing receptor is under the control of an endogenous promoter. Non-limiting examples of endogenous promoters include an endogenous TRAC promoter, an endogenous TRBC promoter, an endogenous TRDC promoter, and an endogenous TRGC promoter. In certain embodiments, the endogenous promoter is an endogenous TRAC promoter.

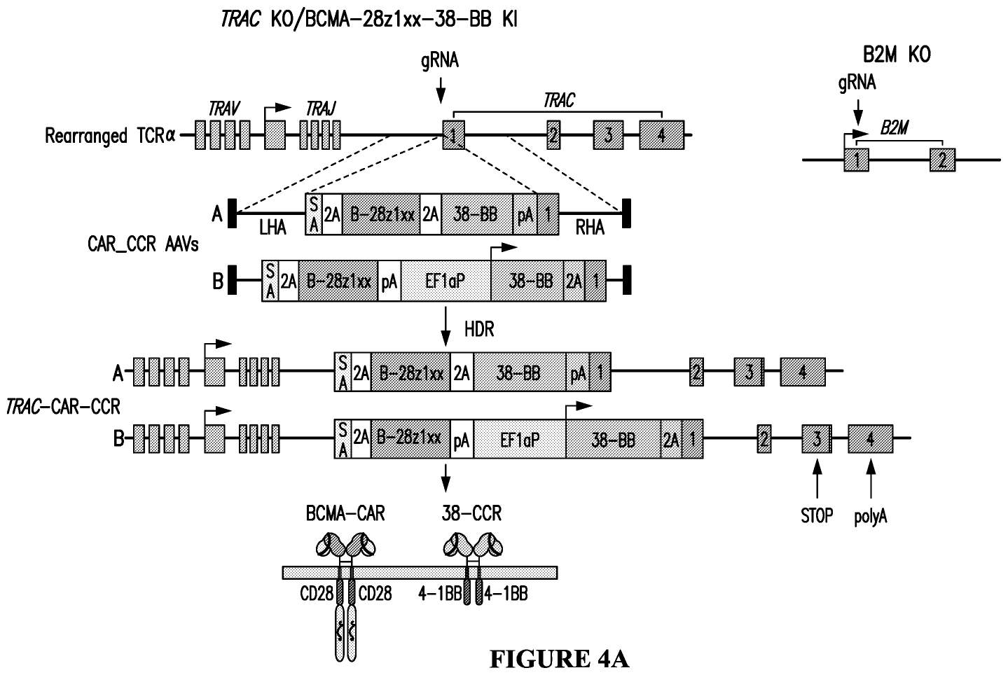

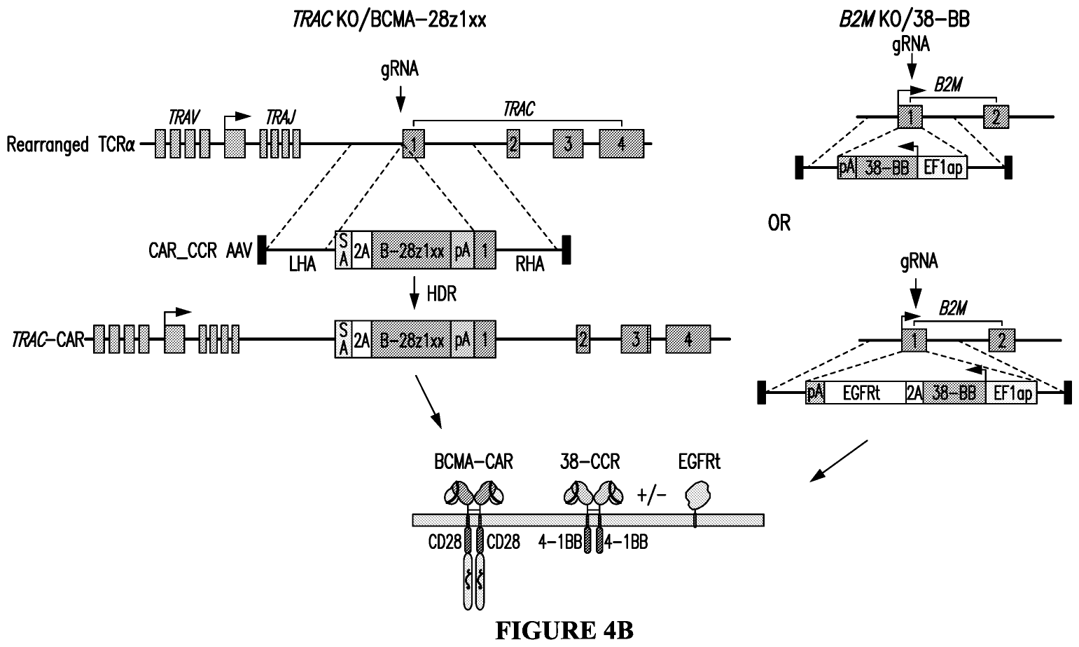

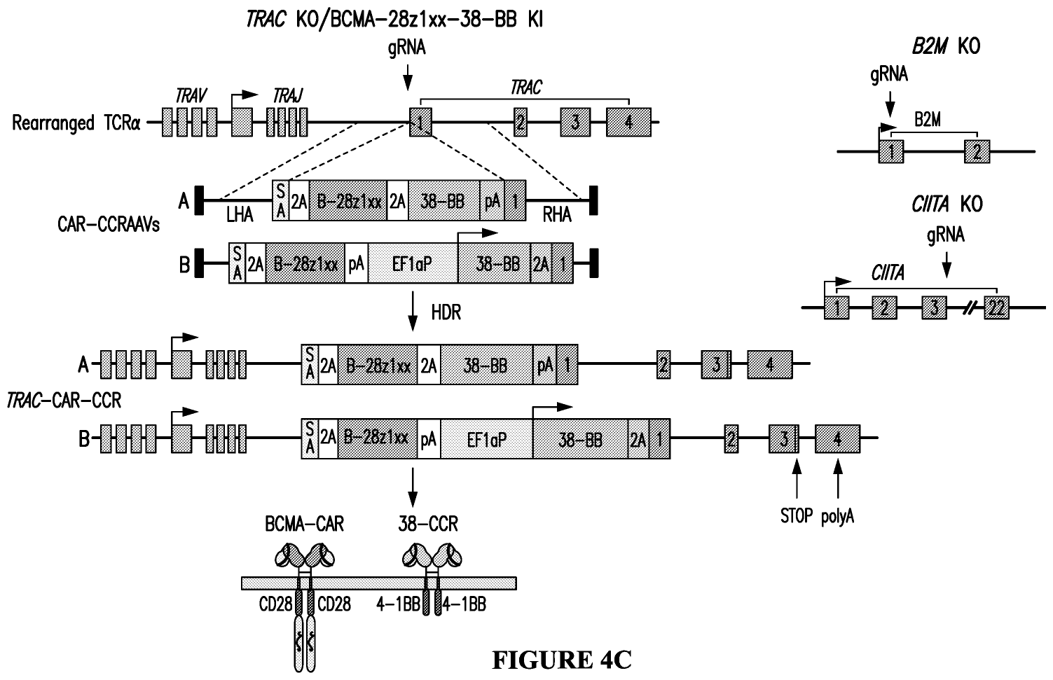

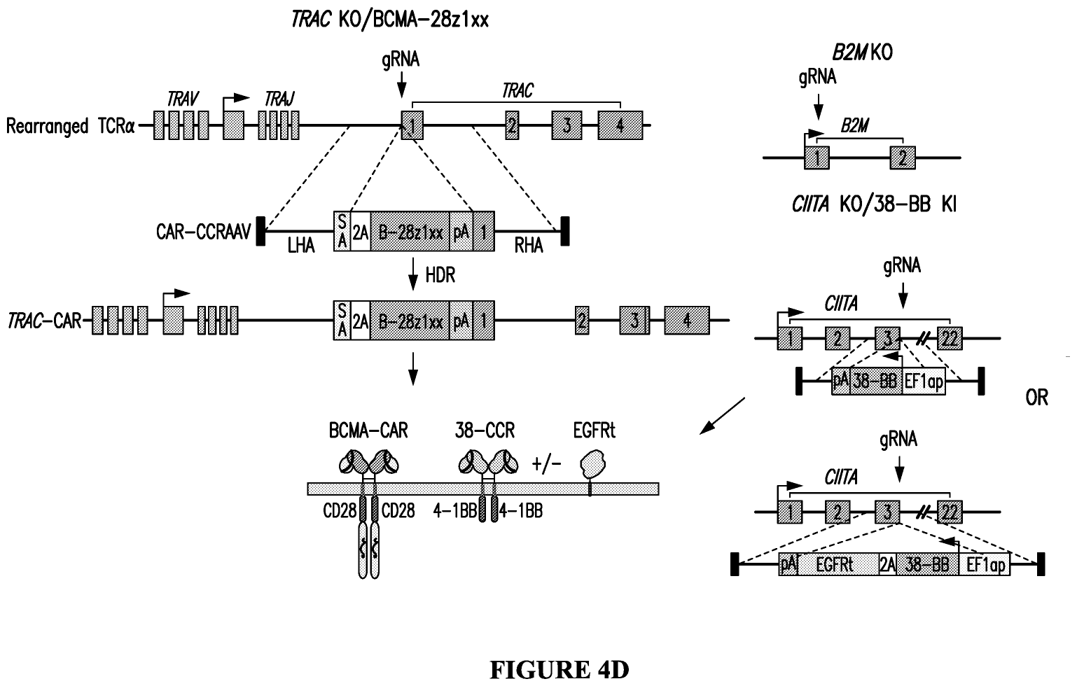

In certain embodiments, both the CD38 CCR and the antigen-recognizing receptor are integrated at a locus within the genome of a T cell. In certain embodiments, both the CD38 CCR and the antigen-recognizing receptor are integrated at the TRAC locus. See FIGS. 4A and 4C. Integration at the TRAC locus leads to knockout of the TRAC locus.

In certain embodiments, the antigen-recognizing receptor is integrated into a locus within the genome of a T cell, and the CD38 CCR is integrated at the B2M locus. In certain embodiments, the antigen-recognizing receptor is integrated at the TRAC locus. See FIG. 4B. Integration at the TRAC locus leads to knockout of the TRAC locus. And integration at the B2M locus leads to knockout of the B2M locus.

In certain embodiments, the antigen-recognizing receptor is integrated into a locus within the genome of a T cell, and the CD38 CCR is integrated at the CIITA locus. In certain embodiments, the antigen-recognizing receptor is integrated at the TRAC locus. See FIG. 4D. Integration at the TRAC locus leads to knockout of the TRAC locus. And integration at the CIITA locus leads to knockout of the CIITA locus.

The presently disclosed subject matter also provides vectors comprising the nucleic acid composition disclosed herein. In certain embodiments, the vector is a retroviral vector (e.g., a gamma-retroviral vector or a lentiviral vector). In certain embodiments, the viral vector is selected from the group consisting of adenoviral vectors, adena-associated viral vectors, vaccinia viruses, bovine papilloma viruses, and herpes viruses (e.g., such as Epstein-Barr Virus).

In certain embodiments, the nucleic acid molecule that enables the expression of a CAR and CCR in an immune cell is present in a vector. In certain embodiments, said vector additionally comprises means for high expression levels such as strong promoters, for example of viral origin (e.g., human cytomegalovirus) or promoters derived from genes that are highly expressed in a cell such as a mammalian cell (Running Deer and Allison, 2004. Biotechnol Prog 20:880-889; U.S. Pat. No. 5,888,809). In certain embodiments, the vectors comprise selection systems such as, for example, expression of glutamine synthetase or expression of dihydrofolate reductase for amplification of the vector in a suitable recipient cell, as is known to the skilled person. The nucleic acid compositions can be administered to subjects or and/delivered into cells by art-known methods or as described herein. Genetic modification of a cell (e.g., a T cell or a NK cell) can be accomplished by transducing a substantially homogeneous cell composition with a recombinant DNA construct. In certain embodiments, a retroviral vector (either gamma-retroviral or lentiviral) is employed for the introduction of the nucleic acid compositions into the cell. For example, the first polynucleotide and the second polynucleotide can be cloned into a retroviral vector and expression can be driven from its endogenous promoter, from the retroviral long terminal repeat, or from a promoter specific for a target cell type of interest. Non-viral vectors may be used as well.

In certain embodiments, the vector is a viral vector. In certain embodiments, the a viral vector is able to transduce immune cells (e.g., T cells and NK cells). In certain embodiments, said viral vector is a recombinant adeno-associated viral vector, a herpes simplex virus-based vector, or a lentivirus-based vector such as a human immunodeficiency virus-based vector. In certain embodiments, said viral vector is a retroviral-based vector such as a lentivirus-based vector such as a human immunodeficiency virus-based vector, or a gamma-retrovirus-based vector such as a vector based on Moloney Murine Leukemia Virus (MoMLV), Spleen-Focus Forming Virus (SFFV), Myeloproliferative Sarcoma Virus (MPSV) or on Murine Stem Cell Virus (MSCV). In certain embodiments, the retroviral vector is the SFG gamma retroviral vector (Rivière et al., 1995. PNAS 92:6733-6737).

Retroviruses, including a gamma-retrovirus-based vector, can be packaged in a suitable complementing cell that provides Group Antigens polyprotein (Gag)-Polymerase (Pol) and/or Envelop (Env) proteins. Suitable packaging cells are human embryonic kidney derived 293T cells, Phoenix cells (Swift et al., 2001. Curr Protoc Immunol, Chapter 10: Unit 10 17C), PG13 cells (Locw et al., 2010. Gene Therapy 17:272-280) and Flp293A cells (Schucht et al., 2006. Mol Ther 14:285-92).

In certain embodiments, said vector further comprises a nucleic acid encoding a co-stimulatory ligand, wherein said co-stimulatory ligand is expressed as a separate protein. In certain embodiments, said co-stimulatory ligand is selected from the group consisting of CD80, CD86, CD70, OX40L and/or 4-1BBL.

As an alternative, non-viral gene therapy can be used for generation of an immune cell expressing a CAR and CCR disclosed herein. Non-viral vectors include nude DNA, liposomes, polymerizers and molecular conjugates. Minicircle DNA vectors free of plasmid bacterial DNA sequences can be generated in bacteria and can express a nucleic acid acids encoding an intracellular signaling domain, a co-stimulatory CD28 intracellular domain, a CD28 transmembrane domain and an antigen binding domain at high levels in vivo. As an alternative, an immune cell expressing a CAR and CCR disclosed herein can be provided by gene editing technology, including CRISPR/Cas, zinc-finger nucleases, and transcription activator-like effector nucleases-TALEN, in order to insert the receptor transgenes into specific loci with or without an exogenous promoter (Eyquem et al., 2017. Nature 543:113-117). Exemplary genomic loci include the TRAC gene locus (constant region of the t cell receptor α-chain), the b2m gene locus, the AAVS1 locus and the PD-1 locus, as is known to a skilled person.

The nucleic acid compositions can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107: 77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of a cell. For example, a nucleic acid molecule can be delivered into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Transient expression may be obtained by RNA electroporation.

Any targeted genome editing methods can also be used to deliver the CD38 CCR and/or the antigen-recognizing receptor disclosed herein to a cell. In certain embodiments, a CRISPR system is used to deliver the CD38 CCR and/or the antigen-recognizing receptor disclosed herein to a cell. In certain embodiments, zinc-finger nucleases are used to deliver a CD38 CCR and/or the antigen-recognizing receptor disclosed herein to a cell. In certain embodiments, a TALEN system is used to deliver the CD38 CCR and/or the antigen-recognizing receptor disclosed herein to a cell.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of base pairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. cDNA expression for use in polynucleotide therapy methods can be directed from any suitable promoter (e.g., the human cytomegalovirus (CMV), simian virus 40 (SV40), metallothionein promoters, or Ubiquitin C promoter), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

### 5.5. Formulations and Administration

The presently disclosed subject matter provides compositions comprising the presently disclosed cells (e.g., those disclosed in Section 5.3). In certain embodiments, the composition is a pharmaceutical composition that further comprise a pharmaceutically acceptable excipient/carrier.

Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Compositions comprising the presently disclosed cells can be provided systemically or directly to a subject for inducing and/or enhancing an immune response to an antigen and/or treating and/or preventing a neoplasm. In certain embodiments, the presently disclosed cells or compositions comprising thereof are directly injected into an organ of interest (e.g., an organ affected by a neoplasm). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of cells in vitro or in vivo.

The quantity of cells to be administered can vary for the subject being treated. In certain embodiments, between about 10⁴ and about 10¹⁰, between about 10⁴ and about 10⁷, between about 10⁵ and about 10⁷, between about 10⁵ and about 10⁹, or between about 10⁶ and about 10⁸ of the presently disclosed cells are administered to a subject. In certain embodiments, between about 10⁵ and about 10⁷ of the presently disclosed cells are administered to a subject. More effective cells may be administered in even smaller numbers. Usually, at least about 1×10⁵ cells will be administered, eventually reaching about 1×10¹⁰ or more. In certain embodiments, at least about 1×10⁵, about 5×10⁵, about 1×10⁶, about 5×10⁶, about 1×10⁷, about 5×10⁷, about 1×10⁸, or about 5×10⁸ of the presently disclosed cells are administered to a subject. In certain embodiments, about 1×10⁵ of the presently disclosed cells are administered to a subject. In certain embodiments, about 5×10⁵ of the presently disclosed cells are administered to a subject. In certain embodiments, about 1×10⁶ of the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art.

The presently disclosed cells and compositions can be administered by any method known in the art including, but not limited to, intravenous administration, subcutaneous administration, intranodal administration, intratumoral administration, intrathecal administration, intrapleural administration, intraosseous administration, intraperitoneal administration, pleural administration, and direct administration to the subject. The presently disclosed cells can be administered in any physiologically acceptable vehicle, normally intravascularly, although they may also be introduced into bone or other convenient site where the cells may find an appropriate site for regeneration and differentiation (e.g., thymus). The cells can be introduced by injection, catheter, or the like.

Compositions comprising the presently disclosed cells can be provided systemically or directly to a subject for inducing and/or enhancing an immune response to an antigen and/or treating and/or preventing a neoplasm (e.g., cancer), pathogen infection, or infectious disease. In certain embodiments, the presently disclosed cells, compositions, or nucleic acid compositions are directly injected into an organ of interest (e.g., an organ affected by a neoplasm). Alternatively, the presently disclosed cells, compositions, or nucleic acid compositions are provided indirectly to the organ of interest, for example, by administration into the circulatory system (e.g., the tumor vasculature). Expansion and differentiation agents can be provided prior to, during or after administration of the cells, compositions, or nucleic acid compositions to increase production of the cells (e.g., T cells (e.g., CTL cells) or NK cells) in vitro or in vivo.

The presently disclosed compositions can be pharmaceutical compositions comprising the presently disclosed cells or their progenitors and a pharmaceutically acceptable carrier. Administration can be autologous or heterologous. For example, cells, or progenitors can be obtained from one subject, and administered to the same subject or a different, compatible subject. Peripheral blood derived cells or their progeny (e.g., in vivo, ex vivo or in vitro derived) can be administered via localized injection, including catheter administration, systemic injection, localized injection, intravenous injection, or parenteral administration. When administering a therapeutic composition of the presently disclosed subject matter (e.g., a pharmaceutical composition comprising a presently disclosed cell), it can be formulated in a unit dosage injectable form (solution, suspension, emulsion).

In certain embodiments, the presently disclosed subject matter further provides a pharmaceutical composition comprising an immune cell expressing a CAR and a CCR or a nucleic acid molecule disclosed herein. In certain embodiments, the pharmaceutical composition comprises a pharmaceutically acceptable carrier. A carrier, as used herein, means a non-toxic material that does not interfere with the effectiveness of the biological activity of the active ingredient. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts buffers, stabilizers, solubilizers, and other materials which are well known in the art.

### 5.6. Methods

The presently disclosed subject matter provides various methods of using a CD38 CCR, a presently disclosed cell, a presently disclosed pharmaceutical composition, or a presently disclosed nucleic acid composition.

As shown in Example 1, a CD38 CCR can abolish CD38 expression on T cells, e.g., engineered T cells, e.g., T cells expressing a CAR. Thus, a CD38 CCR can be used to knock out CD38. The presently disclosed subject matter provides methods of reducing and/or abolishing expression of CD38 in a cell. In certain embodiments, the method comprises transducing the cell to comprise a CD38 CCR (e.g., one disclosed in Section 5.2), e.g., the cell thereby expresses the CD38 CCR. In certain embodiments, the method comprises introducing into the cell a nucleic acid molecule encoding a CD38 CCR (e.g., one disclosed in Section 5.2), e.g., the cell thereby expresses the CD38 CCR. In certain embodiments, the CD38 CCR reduces and/or abolishes the cell surface expression level of CD38 (e.g., expression of CD38 on the cell surface). It was known that depletion of CD38^{high} NK cells occurs in vitro and in vivo in patients treated with anti-CD38 antibody Daratumumab (Wang et al., Clin. Cancer Res. (2018); 24:4006-4017). On the other hand, the residual CD38^{low/-} NK cells may display a high proliferative potential and a fully functional activity, and thus, such NK cells can be employed therapeutically (Wang (2018)). The presently disclosed subject matter further provides methods of reducing and/or preventing depletion of a Natural Killer (NK) cell. In certain embodiments, the method comprises transducing the NK cell to comprise a CD38 CCR (e.g., one disclosed in Section 5.2), e.g., the NK cell thereby expresses the CD38 CCR. In certain embodiments, the method comprises introducing into the NK cell a nucleic acid molecule encoding a CD38 CCR (e.g., one disclosed in Section 5.2), e.g., the NK cell thereby expresses the CD38 CCR.

As shown in Example 1, T cells expressing a CAR that does not target CD38 has high expression of CD38, which can lead to fratricide killing of the CAR-T cells, when the CAR-T cells are used in combination with a CD38 drug, e.g., an anti-CD38 antibody. Thus, CAR-T cells that further express a CD38 CCR can be used in combination with a CD38 drug, e.g., an anti-CD38 antibody (e.g., daratumumab) for treating diseases associated with and/or expressing CD38. Thus, the presently disclosed subject matter provides methods for treating and/or preventing a disease associated with CD38 and/or a disease expressing CD38 in a subject. In certain embodiments, the method comprises administering to the subject (a) a CD38 drug and (b) a cell comprising (i) an antigen-recognizing receptor that binds to an antigen expressed on a tissue or cell of the disease, and (ii) a CD38 CCR. In certain embodiments, the disease is a tumor. In certain embodiments, tumor burden in the subject is reduced, e.g., the number of tumor cells is reduced, the tumor size is reduced, and/or the tumor is eradicated in the subject. In certain embodiments, the CD38 drug is an anti-CD38 antibody. Non-limiting examples of anti-CD38 antibodies include isatuximab and daratumumab. In certain embodiments, the anti-CD38 antibody is daratumumab.

Non-limiting examples of tumors associated with CD38 and/or expressing CD38 include a hematological cancer, glioblastoma, and a solid tumor. In certain embodiments, the tumor is a hematological cancer. Non-limiting examples of hematological cancers include multiple myeloma (MM), Non-Hodgkin Lymphoma, Hodgkin Lymphoma, Chronic Lymphocytic Leukemia (CLL), Waldenstrom's Macroglobulinemia, acute lymphoblastic leukemia (ALL), lymphoma, and acute myeloid leukemia (AML). In certain embodiments, the tumor is multiple myeloma.

Non-limiting examples of antigens expressed on a tissue and/or a cell of the tumor include BCMA, GPRC5D, FcRL5, CD41, CD48, P2RY10, CD44v6, CD70, CD123, Kappa light chain, NYESO-1, BOB1, ADGRE2, CLEC12A, CCR1, LILRB2, CD5, CD7, CD10, CD19, CD20, CD22, CD30, CD33, CD34, CD38, CD56, BCMA, CS-1, CD138, Lewis antigen (LeY), WT-1, and ITGB7. In certain embodiments, the antigen is BCMA. In certain embodiments, the tumor associated with CD38 and/or expressing CD38 is a solid tumor. Solid tumors includes sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pincaloma, hemangioblastoma, acoustic neuroma, oligodenroglioma, schwannoma, meningioma, melanoma, neuroblastoma, and retinoblastoma). In certain embodiments, the solid is selected from the group consisting of breast cancer, colon cancer, non-small cell lung cancer, prostate cancer, gastric, esophageal and pancreatic cancers, hepatocellular carcinoma (HCC), and fibrolamellar carcinoma.

Furthermore, the presently subject matter provides methods of treating and/or preventing a tumor, a pathogen infection, an infectious disease, and/or an autoimmune disease in a subject. In certain embodiments, the method comprises administering to the subject the cells disclosed herein (e.g., those disclosed in Section 5.3), or compositions comprising thereof (e.g., those disclosed in Section 5.5). In certain embodiments, the tumor and/or the pathogen infection is associated with and/or expressing CD38. In certain embodiments, the method further comprises administering a CD38 drug, e.g., an anti-CD38 antibody.

For treatments, the amount administered is an amount effective in producing the desired effect. An effective amount can be provided in one or a series of administrations. An effective amount can be provided in a bolus or by continuous perfusion.

The presently disclosed subject matter further provides an immune cell expressing a CAR and a CCR disclosed herein or a nucleic acid molecule disclosed herein, for use as a medicament. In certain embodiments, the medicament is a medicament for treatment of a malignancy. In certain embodiments, the malignancy is a haematological malignancy. In certain embodiments, the immune cell expressing a CAR and a CCR disclosed herein or a nucleic acid molecule disclosed herein is used for prophylactic administration or therapeutic administration in humans that are suffering from a malignancy. In certain embodiments, the malignancy is a haematological malignancy. Thus, an immune cell expressing a CAR and a CCR disclosed herein or a nucleic acid molecule disclosed herein can be administered to an individual that is suspected of suffering from a malignancy, or can be administered to an individual already evidencing a malignancy in order to lessen signs and symptoms of said malignancy.

In certain embodiments, the administration of an immune cell expressing a CAR and a CCR disclosed herein or a nucleic acid molecule disclosed is provided in an effective amount to an individual in need thereof. In certain embodiments, an effective amount of an immune cell expressing a CAR and a CCR or a nucleic acid molecule disclosed herein is a dosage large enough to produce the desired effect in which the symptoms of the malignancy are ameliorated, or the likelihood of a malignancy is decreased. In certain embodiments, the therapeutically effective amount does not cause adverse side effects. Generally, a therapeutically effective amount can vary with the individual's age, condition, and sex, as well as the extent of the disease and can be determined by one of skill in the art. The dosage can be adjusted by the individual physician in the event of any complication. In certain embodiments, the therapeutically effective amount can vary from about 0.01 mg/kg to about 500 mg/kg, from about 0.1 mg/kg to about 200 mg/kg, from about 0.2 mg/kg to about 20 mg/kg, in one or more dose administrations daily, for one or several days. In certain embodiments, the therapeutically effective amount can vary from about 0.2 mg/kg to about 20 mg/kg, in one or more dose administrations daily, for one or several days. In certain embodiments, administration of an immune cell expressing a CAR and a CCR or a nucleic acid molecule disclosed herein occurs for 2 to 5 or more consecutive days in order to effectively treat a malignancy.

In certain embodiments, an immune cell expressing a CAR and a CCR or a nucleic acid molecule disclosed herein can be administered by injection or by gradual infusion over time. In certain embodiments, the administration of an immune cell expressing a CAR and a CCR or a nucleic acid molecule disclosed herein is parenteral (e.g., intravenous), intraperitoneal, intranasal, or intramuscular. In certain embodiments, preparations for parenteral administration include sterile aqueous or non-aqueous solutions suspensions, and emulsions. Non-limiting examples of non-aqueous solvents are propylene glycol, polyethylene glycol, vegetable oils such as olive oil, and injectable organic esters such as ethyl oleate. Aqueous carriers include, without any limitation, water, alcoholic/aqueous solutions, emulsions or suspensions, including saline and buffered media. Parenteral vehicles include, without any limitation, sodium chloride solution, Ringer's dextrose, dextrose and sodium chloride, lactated Ringer's or fixed oils. Intravenous vehicles include, without any limitation, fluid and nutrient replenishers, electrolyte replenishers (such as those based on Ringer's dextrose), and the like. Preservatives and other additives can also be present such as, for example, but without any limitation, antimicrobials, anti-oxidants, chelating agents, and inert gases and the like.

In certain embodiments, the presently disclosed subject matter also provides an immune cell expressing a CAR and a CCR or a nucleic acid molecule disclosed herein for use in a method for treatment of a malignancy and/or an infection.

In certain embodiments, the presently disclosed subject matter also provides a method of treating an individual suffering from a cancer, said method comprising providing an immune cell expressing a CAR and a CCR or a nucleic acid molecule disclosed herein to an individual in need thereof to thereby treat the individual.

In certain embodiments, the presently disclosed subject matter further provides a method of treating a malignancy, in a patient by administrating the pharmaceutical composition according to the presently disclosed subject matter. In certain embodiments, the malignancy is a haematological malignancy. In certain embodiments, said method can further comprise the administration of an immune checkpoint inhibitor.

An immune checkpoint inhibitor refers to a molecule that blocks an inhibitory interaction between immune cells and other cells or cytokines and which may thereby increase the killing of cells such as cancer cells or infected cells. Non-limiting examples of checkpoint interacting molecules are PD-1/PD-L1 and CTLA-4/B7-1/B7-2.

In certain embodiments, the immune checkpoint inhibitor is a molecule that blocks an interaction between PD-1 and PD-L1. In certain embodiments, said molecule that blocks an interaction between PD-1 and PD-L1 is an antibody against PD1 and/or an antibody against PDL1. Immune checkpoint inhibitors include, for example and without any limitation, a PD1 or PD-L1 blocker such as pembrolizumab (Merck), nivolumab (Bristol-Myers Squibb), pidilizumab (Medivation/Pfizer), MEDI0680 (AMP-514; AstraZeneca) and PDR001 (Novartis); fusion proteins such as a PD-L2 Fc fusion protein (AMP-224; GlaxoSmithKline); atezolizumab (Roche/Genentech), avelumab (Merck/Serono and Pfizer), durvalumab (AstraZeneca), cemiplimab (Regeneron/Sanofi/Genzyme); BMS-936559 (Bristol-Myers Squibb); and small molecule inhibitors such as PD-1/PD-L1 Inhibitor 1 (WO2015034820; (2S)-1-[[2,6-dimethoxy-4-[(2-methyl-3-phenylphenyl) methoxy]phenyl] methyl]piperidine-2-carboxylic acid), BMS202 (PD-1/PD-L1 Inhibitor 2; WO2015034820; N-[2-[[[2-methoxy-6-[(2-methyl [1,1′-biphenyl]-3-yl) methoxy]-3-pyridinyl]methyl]amino]ethyl]-acetamide), and PD-1/PD-L1 Inhibitor 3 (WO/2014/151634; (3S,6S,12S,15S,18S,21S,24S,27S,30R,39S,42S,47aS)-3-((1H-imidazol-5-yl)methyl)-12,18-bis((1H-indol-3-yl)methyl)-N,42-bis(2-amino-2-oxoethyl)-36-benzyl-21,24-dibutyl-27-(3-guanidinopropyl)-15-(hydroxymethyl)-6-isobutyl-8,20,23,38,39-pentamethyl-1,4,7,10,13). Further anti-PD1 molecules include ladiratuzumab vedotin (Seattle Genetics).

In certain embodiments, the immune checkpoint inhibitor that blocks CTLA4 includes ipilimumab (Bristol-Myers-Squibb).

In certain embodiments, the presently disclosed subject matter further provides use of an immune cell expressing a CAR and a CCR or a nucleic acid molecule disclosed herein in the preparation of a medicament for treating an individual suffering from a malignancy or suspected to suffer from a malignancy. In certain embodiments, the method of treating a malignancy comprises isolating immune cells (e.g., T cells or NK cells), from the patient; modifying the immune cells by enabling expression of a CAR and a CCR disclosed herein (for example by introducing a vector disclosed herein in said isolated immune cells); and administering the modified immune cells to the patient. In certain embodiments, the malignancy is a hematological malignancy.

The immune cell expressing a CAR and a CCR or the nucleic acid molecule disclosed herein will attach to cancer cells, as is indicated herein above, and couple to T-cells and/or NK cells to thereby elucidate an immune response against the cancer cells that will reduce or even eliminate said cells. The small size of an immune cell expressing a CAR and a CCR or a nucleic acid molecule disclosed herein, render said immune cell expressing a CAR and a CCR or said nucleic acid molecule disclosed herein as a treatment tool for cancer. These immune cells expressing a CAR and a CCR or nucleic acid molecules disclosed herein provide great promise for treatment of cancer.

In certain embodiments, the pathogen infection is a viral infection. Non-limiting examples of viral infections include those caused by cytomegalovirus (CMV), Epstein-Barr virus (EBV), hepatitis A, B, C, D, E, F or G, human immunodeficiency virus (HIV), adenovirus, BK polyomavirus, coronavirus, coxsackievirus, poliovirus, herpes simplex type 1, herpes simplex type 2, human cytomegalovirus, human herpesvirus type 8, varicella-zoster virus, influenza virus, measles virus, mumps virus, parainfluenza virus, respiratory syncytial virus, papillomavirus, rabies virus, and Rubella virus. Other viral targets include Paramyxoviridae (e.g., pneumovirus, morbillivirus, metapneumovirus, respirovirus or rubulavirus), Adenoviridae (e.g., adenovirus), Arenaviridae (e.g., arenavirus such as lymphocytic choriomeningitis virus), Arteriviridae (e.g., porcine respiratory and reproductive syndrome virus or equine arteritis virus), Bunyaviridae (e.g., phlebovirus or hantavirus), Caliciviridae (e.g., Norwalk virus), Coronaviridae (e.g., coronavirus or torovirus), Filoviridae (e.g., Ebola-like viruses), Flaviviridae (e.g., hepacivirus or flavivirus), Herpesviridae (e.g., simplexvirus, varicellovirus, cytomegalovirus, roscolovirus, or lymphocryptovirus), Orthomyxoviridae (e.g., influenza virus or thogotovirus), Parvoviridae (e.g., parvovirus), Picomaviridae (e.g., enterovirus or hepatovirus), Poxviridae (e.g., orthopoxvirus, avipoxvirus, or leporipoxvirus), Retroviridae (e.g., lentivirus or spumavirus), Reoviridae (e.g., rotavirus), Rhabdoviridae (e.g., lyssavirus, novirhabdovirus, or vesiculovirus), and Togaviridae (e.g., alphavirus or rubivirus). In certain embodiments, the viral infections include human respiratory coronavirus, influenza viruses A-C, hepatitis viruses A to G, and herpes simplex viruses 1-9. In certain embodiments, the subject has an immunodeficiency.

In certain embodiments, the pathogen infection is a bacterial infection. Non-limiting examples of bacterial infections include Mycobacteria, Rickettsia, Mycoplasma, Neisseria meningitides, Neisseria gonorrheoeae, Legionella, Vibrio cholerae, Streptococci, Staphylococcus aureus, Staphylococcus epidermidis, Pseudomonas aeruginosa, Corynobacteria diphtheriae, Clostridium spp., enterotoxigenic Eschericia coli, Bacillus anthracis, Rickettsia, Bartonella henselae, Bartonella quintana, Coxiella burnetii, chlamydia, Mycobacterium leprae, Salmonella, shigella, Yersinia enterocolitica, Yersinia pseudotuberculosis; Legionella pneumophila; Mycobacterium tuberculosis; Listeria monocytogenes; Mycoplasma spp., Pseudomonas fluorescens, Vibrio cholerae, Haemophilus influenzae, Bacillus anthracis, Treponema pallidum, Leptospira, Borrelia, Corynebacterium diphtheriae, Francisella, Brucella melitensis, Campylobacter jejuni, Enterobacter, Proteus mirabilis, Proteus, and Klebsiella pneumoniae.

In certain embodiments, the pathogen infection is a fungal infection. Non-limiting examples of fungal infection include Absidia corymbifera, Acremoniumfalciforme, A. kiliense, A. recifei, Ajellomyces dermatitidis, A. capsulata, Aspergillus spp., (e.g., A. flavus, A. fumigatus; A. nidulans, A. niger, A. terreus), Candida spp. (e.g., C. albicans, C. glabrata, C. guillermondii, C. krusei, C. parapsilosis, C. ke yr, C. tropicalis), C. neoformans, Cunninghamella elegans, Emmonsia parva, Epidermophyton floccosum, Exophialia dermitidis, E. werneckii, E. jeanselmei, E. spinifera, E. richardsiae, Filobasidiella neoformans, Fonsecaea compacta, F. pedrosoi, Histoplasma capsulatum, Leptoshaeria senegarlensis, Madurella mycetomatis, M. grisea, Malassezia furfur, Microsporum spp, Neotestudina rosatii, Paracoccidioides brasiliensis, Penicillium marneffei, Phialophora verrucosa, Piedraia hortae, Pneumocystis carinii, Pseudallescheria boydii, Pvrenochaeta romeroi, Rhizomucor pusillus, Sporothrix schenckii, Trichophyton spp, Trichosporon beigelii, and Xylohypha bantiana. Non-limiting examples of autoimmune diseases and inflammatory diseases or conditions thereof include arthritis, e.g., rheumatoid arthritis (RA), Type I diabetes, systemic lupus erythematosus (SLE), inflammatory bowel disease, ulcerative colitis, psoriasis, psoriatic arthritis, scleroderma, autoimmune thyroid disease, Grave's disease, Crohn's disease, multiple sclerosis, systemic sclerosis, asthma, organ transplant rejection, a disease or condition associated with transplant, Takayasu arteritis, giant-cell arteritis, Kawasaki disease, polyarteritis nodosa, Behcet's syndrome, Wegener's granulomatosis, ANCA-vasculitides, Churg-Strauss syndrome, microscopic polyangiitis, vasculitis of connective tissue diseases, Hennoch-Schonlein purpura, cryoglobulinemic vasculitis, cutaneous leukocytoclastic angiitis, Sarcoidosis, Cogan's syndrome, Wiskott-Aldrich Syndrome, primary angiitis of the CNS, thromboangiitis obliterans, parancoplastic arteritis, myelodysplastic syndrome, erythema elevatum diutinum, amyloidosis, autoimmune myositis, Guillain-Barre Syndrome, histiocytosis, atopic dermatitis, pulmonary fibrosis, glomerulonephritis, Whipple's disease, Still's disease, Sjogren's syndrome, osteomyelofibrosis, chronic inflammatory demyelinating polyneuropathy, Kimura's disease, systemic sclerosis, chronic periaortitis, chronic prostatitis, idiopathic pulmonary fibrosis, chronic granulomatous disease, idiopathic, bleomycin-induced lung inflammation, cytarabine-induced lung inflammation, autoimmune thrombocytopenia, autoimmune neutropenia, autoimmune hemolytic anemia, autoimmune lymphocytopenia, chronic autoimmune thyroiditis, autoimmune hepatitis, Hashimoto's thyroiditis, atopic thyroiditis, Graves disease, autoimmune polyglandular syndrome, autoimmune Addison syndrome, and/or myasthenia gravis. In accordance with the presently disclosed subject matter, the above-described various methods can comprise administering to the subject a checkpoint immune blockade agent.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Further modification can be introduced to the presently disclosed subject matter to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T-cell elimination by administering anti-EGFR monoclonal antibody (e.g., cetuximab). EGFRt can be covalently joined to the upstream of the CD19-targeted CAR. The suicide gene can be included within the vector comprising nucleic acids encoding, for example and without any limitation, a presently disclosed BCMA-targeted CAR. In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated cells expressing the BCMA-targeted CAR. The incorporation of a suicide gene into the a presently disclosed BCMA-targeted CAR gives an added level of safety with the ability to eliminate the majority of receptor-expressing cells within a very short time period. A presently disclosed cell incorporated with a suicide gene can be pre-emptively eliminated at a given timepoint post the cell infusion, or eradicated at the earliest signs of toxicity.

### 5.7. Kits

The presently disclosed subject matter provides kits for reducing and/or abolishing expression of CD38 in a cell, reducing and/or preventing depletion of a Natural Killer (NK) cell, treating and/or preventing a tumor or a pathogen infection (e.g., an autoimmune disease or an infectious disease) in a subject, and/or treating and/or preventing a tumor associated with CD38 in a subject. In certain embodiments, the kit comprises an effective amount of presently disclosed cells, a presently disclosed composition, or a presently disclosed nucleic acid composition. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes an isolated nucleic acid molecule encoding an antigen-recognizing receptor (e.g., a CAR or a TCR) directed toward an antigen of interest and an isolated nucleic acid molecule encoding a CD38 CCR in expressible form, which may optionally be comprised in the same or different vectors.

If desired, the cells, composition, or nucleic acid composition are provided together with instructions for administering the cells, composition, or nucleic acid composition to a subject having or at risk of developing a tumor (e.g., a cancer) or a pathogen infection (e.g., an infectious disease), or immune disorder (e.g., an autoimmune disease). The instructions generally include information about the use of the cell, composition or nucleic acid composition for reducing and/or abolishing expression of CD38 in a cell, reducing and/or preventing depletion of a Natural Killer (NK) cell, treating and/or preventing a tumor or a pathogen infection (e.g., an autoimmune disease or an infectious disease) in a subject, and/or treating and/or preventing a tumor associated with CD38 in a subject. In certain embodiments, the instructions include at least one of the following: description of the therapeutic agent; dosage schedule and administration for reducing and/or abolishing expression of CD38 in a cell, reducing and/or preventing depletion of a Natural Killer (NK) cell, treating and/or preventing a tumor or a pathogen infection (e.g., an autoimmune disease or an infectious disease) in a subject, and/or treating and/or preventing a tumor associated with CD38 in a subject; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### 5.8. Exemplary Embodiments

A1. In certain non-limiting embodiments, the presently disclosed subject matter provides for a method of reducing and/or abolishing expression of CD38 in a cell, comprising transducing the cell to comprise a chimeric co-stimulating receptor (CCR) that binds to CD38.

A2. In certain non-limiting embodiments, the presently disclosed subject matter provides for a method of reducing and/or abolishing expression of CD38 in a cell, comprising introducing into the cell a nucleic acid molecule that encodes a chimeric co-stimulating receptor (CCR) that binds to CD38.

A3. The foregoing method of A1 or A2, wherein the expression of CD38 is an expression level of CD38 on the cell surface of the cell.

A4. The foregoing method of any one of A1-A3, wherein the cell comprises an antigen-recognizing receptor that binds to an antigen.

A5. In certain non-limiting embodiments, the presently disclosed subject matter provides for a method of reducing and/or preventing depletion of a Natural Killer (NK) cell, comprising transducing the cell to comprise a chimeric co-stimulating receptor (CCR) that binds to CD38.

A6. In certain non-limiting embodiments, the presently disclosed subject matter provides for a method of reducing and/or preventing depletion of a Natural Killer (NK) cell, comprising introducing into the NK cell a nucleic acid molecule that encodes a chimeric co-stimulating receptor (CCR) that binds to CD38.

A7. The foregoing method of A5 or A6, wherein the NK cell comprises an antigen-recognizing receptor that binds to an antigen.

A8. In certain non-limiting embodiments, the presently disclosed subject matter provides for a method of treating and/or preventing a disease associated with and/or expressing CD38 in a subject, comprising administering to the subject (a) a CD38 drug and (b) a cell comprising (i) an antigen-recognizing receptor that binds to an antigen expressed on a tissue or cell of the disease, and (ii) a chimeric co-stimulating receptor (CCR) that binds to CD38.

A9. The foregoing method of A8, wherein the disease is a tumor.

A10. The foregoing method of A9, wherein the tumor is selected from the group consisting of a hematological cancer, glioblastoma, and a solid tumor.

A11. The foregoing method of A10, wherein the tumor is a hematological cancer.

A12. The foregoing method of A10 or A11, wherein the hematological cancer is selected from the group consisting of multiple myeloma (MM), Non-Hodgkin Lymphoma, Hodgkin Lymphoma, Chronic Lymphocytic Leukemia (CLL), Waldenstrom's Macroglobulinemia, acute lymphoblastic leukemia (ALL), lymphoma, and acute myeloid leukemia (AML).

A13. The foregoing method of A12, wherein the tumor is multiple myeloma.

A14. The foregoing method of any one of A8-A13, wherein the antigen is selected from the group consisting of BCMA, GPRC5D, FcRL5, CD41, CD48, P2RY10, CD44v6, CD70, CD123, Kappa light chain, NYESO-1, BOB1, ADGRE2, CLEC12A, CCR1, LILRB2, CD5, CD7, CD10, CD19, CD20, CD22, CD30, CD33, CD34, CD38, CD56, BCMA, CS-1, CD138, Lewis antigen (LeY), WT-1, and ITGB7.

A15. The foregoing method of A10, wherein the tumor is a solid tumor.

A16. The foregoing method of A10 or A15, wherein the solid tumor is selected from breast cancer, colon cancer, non-small cell lung cancer, prostate cancer, gastric, esophageal and pancreatic cancers, hepatocellular carcinoma (HCC), and fibrolamellar carcinoma.

A17. The foregoing method of any one of A8-A16, wherein the CD38 drug is an anti-CD38 antibody.

A18. The foregoing method of A17, wherein the anti-CD38 antibody is isatuximab or daratumumab.

A19. The foregoing method of any one of A1-A4 and A8-A18, wherein the cell is an immunoresponsive cell.

A20. The foregoing method of any one of A1-A4 and A8-A19, wherein the cell is a cell of the lymphoid lineage or a cell of the myeloid lineage.

A21. The foregoing method of any one of A1-A4 and A8-A20, wherein the cell is selected from the group consisting of T cells, B cells, Natural Killer (NK) cells, and dendritic cells.

A22. The foregoing method of any one of A1-A4 and A8-A21, wherein the cell is a T cell.

A23. The foregoing method of A22, wherein the T cell is selected from the group consisting of a cytotoxic T lymphocyte (CTL), a γδ T cell, a tumor-infiltrating lymphocyte (TIL), a regulatory T cell, and a Natural Killer T (NKT) cell.

A24. The foregoing method of any one of A1-A4 and A8-A21, wherein the cell is a Natural Killer (NK) cell.

A25. The foregoing method of any one of A4 and A7-A24, wherein the antigen-recognizing receptor is selected from the group consisting of a T cell receptor (TCR), a TCR like fusion molecule, or a chimeric antigen receptor (CAR).

A26. The foregoing method of A25, wherein the TCR like fusion molecule is a recombinant T cell receptor (TCR) comprising an antigen binding chain that comprises: (a) an antigen-binding fragment of an immunoglobulin variable region that binds to an antigen in an HLA-independent manner; and (b) a constant domain that is capable of associating with a CD3ζ polypeptide.

A27. The foregoing method of A26, wherein the constant domain comprises a native or modified TRAC polypeptide or a native or modified TRBC polypeptide.

A28. The foregoing method of A26 or A27, wherein the CD3ζ polypeptide is fused to an intracellular domain of a co-stimulatory molecule or a portion thereof.

A29. The foregoing method of A25, wherein the antigen-recognizing receptor is a CAR.

A30. The foregoing method of A29, wherein the CAR comprises an intracellular domain and an extracellular antigen-binding domain that binds to an antigen.

A31. The foregoing method of A30, wherein the intracellular domain of the CAR comprises a CD3ζ polypeptide.

A32. The foregoing method of any one of A26-A28 and A31, wherein the CD3ζ polypeptide is a native or modified CD3ζ polypeptide.

A33. The foregoing method of A32, wherein the modified CD3ζ polypeptide comprises a native ITAM1, an ITAM2 variant consisting of two loss-of-function mutations, and an ITAM3 variant consisting of two loss-of-function mutations.

A34. The foregoing method of any one of A30-A33, wherein the intracellular domain of the CAR further comprises an intracellular domain of a co-stimulatory molecule or a portion thereof.

A35. The foregoing method of A4 or A7, wherein the antigen is a tumor antigen or a pathogen antigen.

A36. The foregoing method of A35, wherein the antigen is a tumor antigen.

A37. The foregoing method of A35, wherein the antigen is a phosphoantigen.

A38. The foregoing method of A35 or A36, wherein the tumor antigen is selected from the group consisting of CD19, carbonic anhydrase IX (CAIX), carcinoembryonic antigen (CEA), CD8, CD7, CD10, CD20, CD22, CD30, CD33, CLL1, CD34, CD41, CD44, CD49f, CD56, CD74, CD133, CD138, CD123, CD44V6, an antigen of a cytomegalovirus (CMV) infected cell, ENPP3, epithelial glycoprotein-2 (EGP-2), epithelial glycoprotein-40 (EGP-40), epithelial cell adhesion molecule (EpCAM), receptor tyrosine-protein kinase Erb-B2, Erb-B3, Erb-B4, folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, GPRC5D, FcRL5, ITGB7, NKCS1, EGF1R, EGFR-VIII, CD99, CD70, ADGRE2, CCR1, LILRB2, PRAME, CD48, P2RY10, CD70, BOB1, CLEC12A, CCR1, LILRB2, CD5, CD38, CS-1, Lewis antigen (LeY), and ERBB.

A39. The foregoing method of any one of A1-A38, wherein the CCR comprises an extracellular antigen-binding domain that binds to CD38, and an intracellular domain that is capable of delivering a costimulatory signal to the cell but does not alone deliver an activation signal to the cell.

A40. The foregoing method of A39, wherein the intracellular domain of the CCR comprises an intracellular domain of a co-stimulatory molecule or a portion thereof.

A41. The foregoing method of any one of A28, A34, and A40, wherein the co-stimulatory molecule is selected from the group consisting of CD28, 4-1BB, OX40, CD17, CD40, CD154, CD97, CD11a/CD18, ICOS, DAP-10, CD2, CD244, CD27, and NKG2D.

A42. The foregoing method of A41, wherein the co-stimulatory molecule is 4-1BB.

A43. The foregoing method of A41, wherein the co-stimulatory molecule is CD28.

A44. The foregoing method of A40, wherein the intracellular domain of the CCR comprises an intracellular domain of a CD28 co-stimulatory molecule and an intracellular domain of a 41-BB co-stimulatory molecule.

B1. In certain non-limiting embodiments, the presently disclosed subject matter provides for a cell comprising:
(a) an antigen-recognizing receptor that binds to an antigen; and
(b) a chimeric co-stimulating receptor (CCR) that binds to CD38,
wherein the cell exhibits substantial cytolytic activity against cells that are single positive for the antigen.

B2. The foregoing cell of B1, wherein the antigen-recognizing receptor binds to an antigen with a dissociation constant (Kd) of 1×10-8 M or less.

B3. The foregoing cell of B1 or B2, wherein the antigen is a tumor antigen or a pathogen antigen.

B4. The foregoing cell of B3, wherein the antigen is a tumor antigen.

B5. The foregoing cell of B3, wherein the antigen is a phosphoantigen.

B6. The foregoing cell of B4 or B5, wherein the tumor antigen is selected from the group consisting of CD19, carbonic anhydrase IX (CAIX), carcinoembryonic antigen (CEA), CD8, CD7, CD10, CD20, CD22, CD30, CD33, CLL1, CD34, CD41, CD44, CD49f, CD56, CD74, CD133, CD138, CD123, CD44V6, an antigen of a cytomegalovirus (CMV) infected cell, ENPP3, epithelial glycoprotein-2 (EGP-2), epithelial glycoprotein-40 (EGP-40), epithelial cell adhesion molecule (EpCAM), receptor tyrosine-protein kinase Erb-B2, Erb-B3, Erb-B4, folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, GPRC5D, FcRL5, ITGB7, NKCS1, EGF1R, EGFR-VIII, CD99, CD70, ADGRE2, CCR1, LILRB2, PRAME, CD48, P2RY10, CD70, BOB1, CLEC12A, CCR1, LILRB2, CD5, CD38, CS-1, Lewis antigen (LeY), and ERBB.

B7. The foregoing cell of any one of B1-B6, wherein the antigen-recognizing receptor is selected from the group consisting of a T cell receptor (TCR), a TCR like fusion molecule, or a chimeric antigen receptor (CAR).

B8. The foregoing cell of B7, wherein the TCR like fusion molecule is a recombinant T cell receptor (TCR) comprising an antigen binding chain that comprises: (a) an antigen-binding fragment of an immunoglobulin variable region that binds to an antigen in an HLA-independent manner; and (b) a constant domain that is capable of associating with a CD3ζ polypeptide.

B9. The foregoing cell of B8, wherein the constant domain comprises a native or modified TRAC polypeptide or a native or modified TRBC polypeptide.

B10. The foregoing cell of B8 or B9, wherein the CD3ζ polypeptide is fused to an intracellular domain of a co-stimulatory molecule or a portion thereof.

B11. The foregoing cell of B8, wherein the antigen-recognizing receptor is a CAR.

B12. The foregoing cell of B11, wherein the CAR comprises an intracellular domain and an extracellular antigen-binding domain that binds to an antigen.

B13. The foregoing cell of B11, wherein the intracellular domain of the CAR comprises a CD3ζ polypeptide.

B14. The foregoing cell of any one of B8-B10 and B13, wherein the CD3ζ polypeptide is a native or modified CD3ζ polypeptide.

B15. The foregoing cell of B14, wherein the modified CD3ζ polypeptide comprises a native ITAM1, an ITAM2 variant consisting of two loss-of-function mutations, and an ITAM3 variant consisting of two loss-of-function mutations.

B16. The foregoing cell of any one of B10-B15, wherein the intracellular domain of the CAR further comprises an intracellular domain of a co-stimulatory molecule or a portion thereof.

B17. The foregoing cell of any one of B1-B16, wherein the CCR comprises an extracellular antigen-binding domain that binds to CD38, and an intracellular domain that is capable of delivering a costimulatory signal to the cell but does not alone deliver an activation signal to the cell.

B18. The foregoing cell of B17, wherein the intracellular domain of the CCR comprises an intracellular domain of a co-stimulatory molecule or a portion thereof.

B19. The foregoing cell of any one of B16-B18, wherein the co-stimulatory molecule is selected from the group consisting of CD28, 4-1BB, OX40, CD17, CD40, CD154, CD97, CD11a/CD18, ICOS, DAP-10, CD2, CD244, CD27, and NKG2D.

B20. The foregoing cell of B19, wherein the co-stimulatory molecule is 4-1BB.

B21. The foregoing cell of B19, wherein the co-stimulatory molecule is CD28.

B22. The foregoing cell of B18, wherein the intracellular domain of the CCR comprises an intracellular domain of a CD28 co-stimulatory molecule and an intracellular domain of a 41-BB co-stimulatory molecule

B23. The foregoing cell of any one of B1-B22, wherein the cell is an immunoresponsive cell.

B24. The foregoing cell of any one of B1-B23, wherein the cell is selected from the group consisting of T cells, B cells, Natural Killer (NK) cells, and dendritic cells.

B25. The foregoing cell of any one of B1-B24, wherein the cell is a T cell.

B26. The foregoing cell of B25, wherein the T cell is selected from the group consisting of a cytotoxic T lymphocyte (CTL), a γδ T cell, a tumor-infiltrating lymphocyte (TIL), a regulatory T cell, and a Natural Killer T (NKT) cell.

B27. The foregoing cell of any one of B1-B24, wherein the cell is a Natural Killer (NK) cell.

C1. In certain non-limiting embodiments, the presently disclosed subject matter provides for a composition comprising the cell of any one of B1-B27.

C2. The foregoing composition of C1, which is a pharmaceutical composition that comprises a pharmaceutically acceptable carrier.

D1. In certain non-limiting embodiments, the presently disclosed subject matter provides for a nucleic acid composition comprising (a) a first polynucleotide encoding an antigen-recognizing receptor that binds to an antigen; and (b) a second polynucleotide encoding a chimeric co-stimulating receptor (CCR) that binds to CD38, wherein a cell comprising the composition exhibits substantial cytolytic activity against cells that are single positive for the antigen.

E1. In certain non-limiting embodiments, the presently disclosed subject matter provides for a vector comprising the nucleic acid composition of D1.

F1. In certain non-limiting embodiments, the presently disclosed subject matter provides for a cell comprising the nucleic acid composition of D1.

F2. The foregoing cell of F1, wherein the cell is an immunoresponsive cell.

F3. The foregoing cell of F1 or F2, wherein the cell is selected from the group consisting of T cells, B cells, Natural Killer (NK) cells, and dendritic cells.

F4. The foregoing cell of any one of F1-F3, wherein the cell is a T cell.

F5. The foregoing cell of F4, wherein the T cell is selected from the group consisting of a cytotoxic T lymphocyte (CTL), a γδ T cell, a tumor-infiltrating lymphocyte (TIL), a regulatory T cell, and a Natural Killer T (NKT) cell.

F6. The foregoing cell of any one of F1-F3, wherein the cell is a Natural Killer (NK) cell.

G1. In certain non-limiting embodiments, the presently disclosed subject matter provides for a method of treating and/or preventing a tumor and/or a pathogen infection in a subject, comprising administering to the subject the cell of any one of B1-B27 and F1-F6 or the composition of C1 or C2.

G2. The foregoing method of G1, wherein the tumor or pathogen infection is associated with CD38, and/or expressing CD38.

G3. The foregoing method of G1 or G2, further comprising to the subject a CD38 drug.

G4. The foregoing method of G3, wherein the CD38 drug is an anti-CD38 antibody.

H1. In certain non-limiting embodiments, the presently disclosed subject matter provides for an immune cell, such as a T cell or NK cell, expressing a chimeric antigen receptor (CAR) and a chimeric co-stimulatory receptor (CCR),
a) wherein the CAR comprises:
   i. an intracellular CD3ζ signaling domain;
   ii. a co-stimulatory CD28 intracellular domain;
   iii. a CD28 transmembrane domain; and
   iv. an antigen specific binding domain binding to a first antigen;
b) wherein the CCR comprises:
   i. a co-stimulatory CD28 intracellular domain and a co-stimulatory 4-1BB intracellular domain;
   ii. a CD28 transmembrane domain; and
   iii. an antigen specific binding domain binding to a second antigen;
wherein the antigen specific binding domains are present on the surface of the immune cell, and wherein the first antigen differs from the second antigen.

H2. The foregoing immune cell according to H1, wherein the first and second antigen are selected from CD41, CD48, P2RY10, CD44v6, CD70, CD123, Kappa light chain, NYESO-1, BOB1, ADGRE2, CLEC12A, CCR1, LILRB2, CD5, CD7, CD10, CD19, CD20, CD22, CD30, CD33, CD34, CD38, CD56, BCMA, CS-1, CD138, Lewis antigen (LeY), and WT-1.

H3. The foregoing immune cell according to H1 or H2, wherein the first antigen is CD19.

H4. The foregoing immune cell according to any one of H1-H3, wherein the second antigen is CD38.

H5. The foregoing immune cell according to any one of H1-H4, wherein the intracellular signaling domain of the CAR comprises the intracellular domain of human CD3ζ or a variant thereof.

H6. The foregoing immune cell according to any one of H1-H5, wherein the intracellular domain of the CCR comprises the human co-stimulatory CD28 intracellular domain and human co-stimulatory 4-1BB intracellular domain, or a variant thereof.

H7. The foregoing immune cell according to any one of H1-H6, wherein:
a) the antigen specific binding domain of the CAR is a single chain binding domain, such as a single chain Fv fragment,
b) the antigen specific binding domain of the CCR is a single chain binding domain, such as a single chain Fv fragment, or
c) both the antigen specific binding domain of the CAR is a single chain binding domain, such as a single chain Fv fragment and the antigen specific binding domain of the CCR is a single chain binding domain, such as a single chain Fv fragment.

H8. The foregoing immune cell according to any one of H1-H7, wherein the co-stimulatory CD28 intracellular domains of the CAR and CCR are substantially identical.

J1. In certain non-limiting embodiments, the presently disclosed subject matter provides for a nucleic acid molecule that enables expression of a chimeric antigen receptor (CAR) and a chimeric co-stimulatory receptor (CCR) as defined in any one of H1-H8 in a suitable immune cell, such as a human T-cell, or human NK cell.

J2. The foregoing nucleic acid molecule of J1, which is present in a vector, such as in a viral vector.

K1. In certain non-limiting embodiments, the presently disclosed subject matter provides for a pharmaceutical composition, comprising the immune cell according to any one of H1-H8, or the nucleic acid molecule of J1 or J2.

I1. In certain non-limiting embodiments, the presently disclosed subject matter provides for a method of producing an immune cell according to any one of H1-H8, comprising
a) providing immune cells, such as human immune cells, e.g., human T-cells, or human NK cells, and
b) modifying the immune cells by enabling expression of a CAR and a CCR as defined in any one of H1-H8 in the immune cells.

L1. In certain non-limiting embodiments, the presently disclosed subject matter provides for a method of treating a malignancy, such as a haematological malignancy, in a patient, the method comprising
a) isolating immune cells, such as T cells or NK cells, whereby said immune cells are isolated from the patient;
b) modifying the immune cells by enabling expression of a CAR and a CCR as defined in any one of H1-H8 in the immune cells; and
c) administering the modified immune cells to the patient.

M1. In certain non-limiting embodiments, the presently disclosed subject matter provides for a method of treating a malignancy, such as a haematological malignancy, in a patient, the method comprising the administration of the pharmaceutical composition of K1.

M2. The foregoing method of M1, further comprising the administration of a checkpoint inhibitor.

### 6. EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the presently disclosed cells and compositions, and are not intended to limit the scope of what the inventors regard as their invention.

### Example 1—Knockout CD38 by CD38 CCR

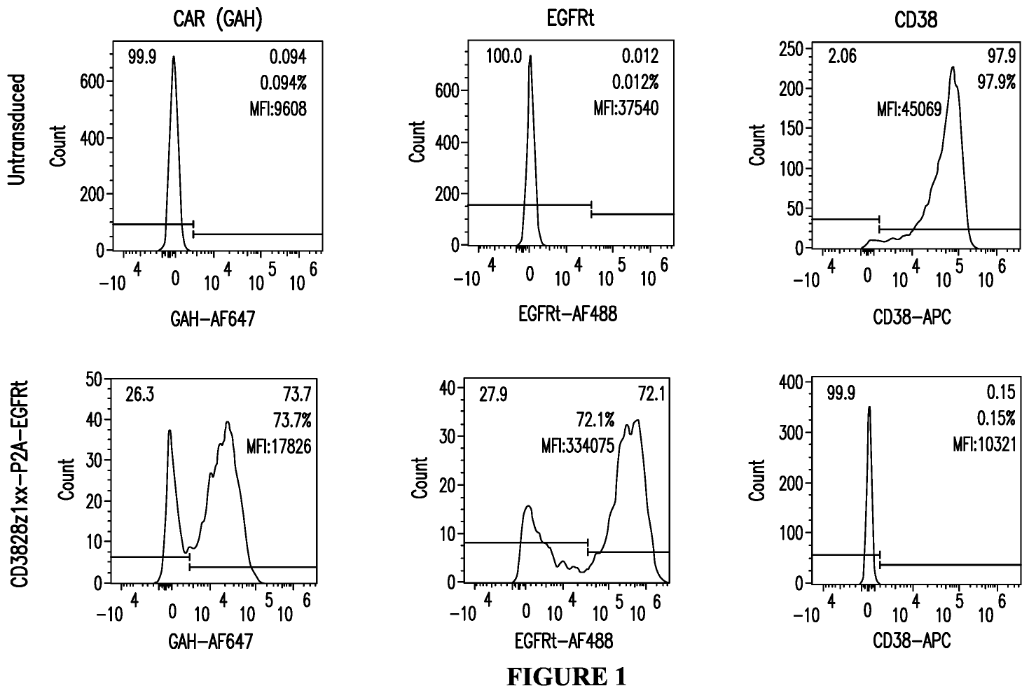

First, the expression levels of CD38 on T cells expressing a CD38-targeted CAR and untransduced T cells were measured. As shown in FIG. 1, untransduced T cells expressed high levels of CD38, while T cells expressing a CD38-targeted CAR lost CD38 expression, which suggests fratricide killing and/or internalization of CD38 with the CD38-targeted CAR.

Next, a CD38 CCR was expressed on the cell surface of T cells expressing a CD19-targeted CAR. As shown in FIG. 2, CD38 expression was abolished on the cell surface of the T cells expressing the CD19-targeted CAR and the CD38 CCR, which suggests that CD38 was internalized with the CD38-CCR. Thus, T cells expressing a CAR and a CD38 CCR were CD38 negative without any genetic manipulation of the CD38 gene locus.

In summary, CD38 is internalized with a CD38 CCR, which leads to abolished expression of CD38 on CAR-T cells. Thus, a CD38 CCR can be used to reduce and/or abolish CD38 expression (e.g., knock out) on T cells, e.g., engineered T cells (e.g., T cells expressing a CAR). With a CD38 CCR, it is not necessary to knockout CD38 on T cells for production of CAR-T cells. A CD38 CCR can also avoid fratricide killing of CAR-T cells by a CD38 drug, e.g., an anti-CD38 antibody. Thus, CAR-T cells that further express a CD38 CCR can be used in combination with a CD38 drug (e.g., an anti-CD38 antibody (e.g., daratumumab) for treating diseases associated with and/or expressing CD38.

### Example 2—TRAC Targeting Construct Designs for BCMA-CAR+CD38-CCR

CRISPR/Cas9 is used to induce a double stranded break, and a BCMA-targeted CAR and a CD38 CCR are be inserted in the TRAC locus using homology-directed repair. See FIG. 3A. Both the BCMA-targeted CAR and the CD38 CCR are expressed under the TRAC promoter. See FIG. 3A. Using the same approach, a BCMA-targeted CAR is expressed under the TRAC promoter, while a CD38-CCR is expressed under another promoter of choice (e.g. the EF1a promoter) in the TRAC locus. See FIG. 3B.

### Example 3—Delivery of CAR and CD38 CCR

FIGS. 4A-4D represent various delivery methods of a CAR and a CD38 CCR to cells. As shown in FIG. 4A, a BCMA-targeted CAR and a CD38 CCR are integrated at the TRAC locus (knock-in (KI)), thereby knocking out the TRAC locus. In addition, a B2M knockout is introduced using a CRISPR approach comprising a gRNA molecule.

As shown in FIG. 4B, a BCMA-targeted CAR is integrated at the TRAC locus (knock-in (KI)), thereby knocking out the TRAC locus. A CD38 CCR is integrated at exon 1 of the B2M locus, thereby introducing a B2M knockout.

As shown in FIG. 4C, a BCMA-targeted CAR and a CD38 CCR are integrated at the TRAC locus (knock-in (KI)), thereby knocking out the TRAC locus. In addition, a B2M knockout is introduced using a CRISPR approach comprising a gRNA molecule. Furthermore, a CIITA knockout is introduced using a CRISPR approach comprising a gRNA molecule.

As shown in FIG. 4D, a BCMA-targeted CAR is integrated at the TRAC locus (knock-in (KI)), thereby knocking out the TRAC locus. A CD38 CCR is integrated at exon 3 of the CIITA locus, thereby introducing a B2M CIITA knockout. In addition, a B2M knockout is introduced using a CRISPR approach comprising a gRNA molecule.

### Example 4—Combining a CAR and CCR Simultaneously Enhances T Cell Sensitivity to Low Antigen Density and Persistence

### Study Design

The purpose of this example was to investigate the hypothesis that dual targeting of tumor with a CAR and a CCR would improve the therapeutic outcomes of engineered T cells. To test this strategy in hematological tumor models of MM and ALL, CD38 was selected as a CCR target, a molecule expressed in various hematologic tumors. At first, it was investigated the cytotoxic potential of CAR+CCR T cells compared to conventional single targeted BCMA- or CD19-CAR T cells. The mechanism of the observed increase of cytolytic capacity and the contribution of the extracellular and intracellular part of the CCR were investigated. Next, MM and ALL tumor cell lines with low BCMA and CD19 density were used to investigate the sensitivity of recognition from CAR+CCR T cells compared to CAR T cells. A second objective was the assessment of the cytokine production, expansion potential and differentiation of CAR+CCR T cells and the contribution of the extracellular and intracellular part of the CCR.

For the in vivo models, mice (with a minimum of n=4 per group) were randomly assigned to treatment groups. Further, the anti-tumor efficacy and persistence of CAR+CCR T cells and their efficacy against tumor variants with low target-antigen density were analyzed. The animal experiments were performed under the approval of the central authority for scientific procedures on animals (CCD). All primary samples (healthy donor and patient material) were obtained after informed consent and approval by the institutional medical ethical committee. Each experiment was performed multiple times using T cells from at least four different donors and we present pooled data. There were no outliers in the experimental data.

### Materials and Methods

Cell lines. Human cell lines MM1.S, Um9, K562, U266 and NALM6 (unmodified or expressing luciferase) were cultured in RPMI1640 (Thermo Fisher Scientific) with 10% HyClone Fetal Clone I (Fisher Scientific) and antibiotics (penicillin; 100 U/mL, streptomycin; 100 mg/mL). K562 cells were lentivirally transduced to express human CD38 and human BCMA and sorted for gradually increasing levels of BCMA expression. The mouse fibroblast NIH/3T3 cell line (ATCC) and Phoenix Ampho cells were maintained in DMEM GlutaMAX+10% fetal bovine serum (FBS) (Invitrogen)+antibiotics (penicillin 10.000 U/ml, streptomycin 10.000 μg/ml). NIH/3T3 cells were transduced to express human CD38 (3T3-CD38). Banks of all cell lines were checked with Short Tandem Repeat (STR) analysis and were regularly tested for mycoplasma.

Primary cells from MM patients and healthy individuals. Healthy donor peripheral blood mononuclear cells (PBMCs) from buffy coats (Sanquin blood-bank) or bone marrow mononuclear cells (BM-MNCs) from MM patient's bone marrow aspirates [˜10-40% malignant cells, determined by flow cytometry (CD138+/CD38+)], were isolated by Ficoll-Paque (GE Healthcare Life Sciences) density centrifugation. Isolated cells were directly used in cytotoxicity assays or cryopreserved in liquid nitrogen until use. All primary samples were obtained after informed consent and approval by the institutional medical ethical committee.

CAR and CCR vector constructs. CAR and CCR constructs were cloned into SFG Y-retroviral vectors (Rivière et al., 1995. PNAS 19:6733) using standard molecular biology techniques. BCMA-CAR constructs used an extracellular single chain variable fragment (scFv) published and described in WO2016/094304 A2 (BCMA02, drug product name bb2121). The scFv was followed by a CD8a transmembrane domain and CD3ζ signaling domain (BCMA-ζ) or by a CD8a transmembrane domain and the 4-1BB and CD3ζ signaling domains (BCMA-BBζ) or a CD28 transmembrane and intracellular sequence fused to CD3ζ intracellular domain (BCMA-28ζ). The CAR sequences were linked by a P2A sequence to a dsRed fluorescent expression marker or to a truncated LNGFR sequence. The CD19 CAR constructs used the SJ25C1 scFv and have been previously described (van der Stegen et al., 2015. Nat Rev Drug Discov 14:499-509). For the CCR construct the CD38-specific scFvs, previously described from our group (Drent et al., 2016. Haematologica 101:616), were followed by a CD28 transmembrane and intracellular sequence and the 4-1BB intracellular domain or by a CD8a transmembrane domain and the 4-1BB signaling domain. The CCR sequences were linked by a P2A element to a truncated LNGFR sequence.

Generation of Retroviral Particles and Transduction of T Cells. Phoenix-Ampho packaging cells were calcium phosphate transfected with 10 μg CAR constructs. 16 hours post-transfection complete medium (DMEM+10% FBS) was refreshed. Two and three days after transfection, cell free supernatants containing retroviral particles were collected and directly used for transduction. Peripheral blood mononuclear cells (PBMCs) from healthy donors (3×10⁶/well) were stimulated with lectin-like phytohemagglutinin (PHA-L) in a 6 well plate (Greiner Bio-One) in culture medium (RPMI-1640, 10% FBS, penicillin; 100 U/ml, streptomycin; 100 μg/ml). After 48 hours, 3×10⁶/ml of cells were transferred to retronectin coated (15 μg/ml) (Takara) 6-well plates (Falcon). Retroviral transduction was performed by addition of 2 ml virus per well followed by spinoculation (1500 g, 1 hour at room temperature) in the presence of 4 μg/ml hexadimethrine bromide (Polybrene). A second transduction was conducted after 16 hours, replacing ⅔rd of the cell supernatant with freshly obtained virus (2 ml). 6-8 hours after the second transduction, half of the cell supernatant was replaced by fresh culture RPMI-1640+10% fetal bovine serum (FBS) and 50 IU/ml rhIL-2 (Proleukin®, Novartis). Transduction efficiencies were determined 72h later by flow cytometric detection of LNGFR (CD271) or dsRed expression. In order to isolate double transduced CAR+CCR T cells the EasySep™ APC Positive Selection Kit II (Stemcell Technologies) was used to isolate T cells labeled with an CD271 (NGFR)-APC antibody (Biolegend), by positive selection. Sorted T cells bearing the CD38CCR (a mix of double CAR+CCR transduced and CCR-transduced) were further used in assays, assuming that CD38CCR-transduced T cells would remain non-functional upon antigen engagement.

Bioluminescent Imaging based cytotoxicity assay. Seven to ten days after transduction serial dilutions (effector:target 2:1, 1:1, 1:2) of CAR T cells were incubated with luciferase expressing cell lines. The luciferase signal produced by surviving cells was determined after 16-24 hours with a GloMax® 96 Microplate Luminometer (Promega) within 15 minutes after the addition of 125 μg/mL beetle luciferin (Promega). % lysis cells=1−(BLI signal in treated wells/BLI signal in untreated wells)×100%.

Calcein-AM release assay. Target cells (3T3-CD38) were suspended in complete medium at a final concentration of 106/ml and labeled with Calcein-AM, purchased from Molecular Probes (Eugene, Oreg.). CAR T cells were incubated together with labeled target cells at E:T ratios ranging from 2:1 to 0.5:1 for 4 hours. Controls included wells for spontaneous (mock transduced T cells from the same donor) and maximum release (only target cells in medium plus 2% Triton X-100). After incubation, clarified supernatant was sampled for Calcein-AM detection in a SpectraMax Gemini dual-scanning microplate spectrofluorimeter (Molecular Devices, Sunnyvale, Calif.) (excitation filter: 485±9 nm; band-pass filter: 530±9 nm). Data are expressed as arbitrary fluorescent units (AFU). Percent lysis was calculated by Specific Lysis (%)=(AFU in treated wells-AFU in spontaneous release)/(AFU in maximum release-AFU in spontaneous release)×100%.

Flow cytometry-based cytotoxicity assay. CAR T cells were incubated in serial dilutions (effector:target 2:1, 1:1, or 0.5:1) with Violet tracer (Thermo Fisher Scientific) labeled BM-MNC for 16h. Different cell subsets were identified after staining with different CD markers (CD3, CD14, CD19, CD38, BCMA, CD56, and CD138 (BD Biosciences). Flow-Count Fluorospheres (Beckman 7547053) were added and cells were quantitatively analyzed through Flow-Count-equalized measurements. Percentage cell lysis was calculated by % lysis cells=1−((# viable target cells in treated wells/# of beads)/(# viable target cells in untreated wells/# of beads))×100%.

Avidity measurement. Cellular avidity of single or double targeting CAR T cells to MM1.S tumor cells was investigated as described previously (Kamsma et al., 2018. Cell Rep 24:3008-3016; Sitters et al., 2015. Nat Methods 12:47-50). In brief, MM1.S cells were allowed to adhere to the surface of a temperature-controlled microfluidic chip (LUMICKS CA B.V.) for a total of 2.5 hours. Chips were placed into the z-Movi Cell Avidity Analyzer (LUMICKS) where experiments were performed at 37° C. Indicated CAR T cells were subsequently flowed into the microfluidics chip and allowed to interact with the MM1.S monolayer for 5 minutes, after which an acoustic force ramp was applied (1000 pN relative force over 150 seconds). All experiments were performed in triplicate.

Proliferation assay. BCMA-CAR T cells were counted and stimulated weekly with irradiated (60 Gy) BCMA+CD38+MM1.S cells. Starting 7 days after transduction, 0.5×10⁶ CAR T cells were seeded in a 24-well plate containing 0.25×10⁶ MM1.S, to final volume of 1 mL. No additional cytokines were added.

Intracellular phosho-AKT1 assessment. Stimulation and staining of cells were performed in 5 mL Round Bottom Polystyrene FACS Tubes. CAR T cells (0.5×10⁶) were co-cultured together with UM9 cells (0.5×10⁶) for 15 minutes at room temperature in complete RPMI-1640 medium with the recommended amount of APC-conjugated anti-CD271 antibody (CD271; clone ME20.4, BioLegend). At the end of the incubation time, tubes were transferred on ice and cells were washed twice with FACs buffer (phosphate-buffered saline with 0.5 to 1% bovine serum albumin or 5 to 10% FBS and 0.1% sodium azide). The eBioscience Intracellular Fixation & Permeabilization Buffer Set was used according to manufacturer's protocol. After permeabilization and without washing, the recommended amount of the Phospho-AKT11 (Ser473) Monoclonal Antibody (clone SDRNR, eBioscience) was added and the cells were incubated for 30 minutes at room temperature, protected from light. Cells were washed twice with Permeabilization Buffer and flow cytometry was performed on BD LSR Fortessa.

Monocyte activation. CD14+ monocytes were isolated from PBMCs by positive selection. Isolated monocytes (10,000 cells) were plated in a 96-wells flat-bottom plate. 24 hours later, 10,000 CAR T cells and 10,000 tumor cells (MM1.S) were added in each well and were incubated for 24 hours. After incubation, the wells were washed with phosphate-buffered saline twice and detached from the wells with Trypsin/EDTA solution (Lonza) for 20 minutes. Monocytes were stained with CD3 (clone SK7, BD Biosciences), CD14 (clone HCD14, BioLegend), HLA-DR (clone G46-6, BD Biosciences), CD86 (clone HA5.2B7, Beckman Coulter), and CD80 (clone 16-10A1, BioLegend) (staining with 1 μg per mL of sample, incubation for 30 minutes at 4° C. in PBS with 1% BSA, 10% FBS and 0.1% NaN3 sodium azide). Flow cytometry was performed on BD LSR Fortessa.

Flow cytometry. Flow cytometry was performed on BD LSR Fortessa. Transduction efficiency was measured with an APC-conjugated antibody toward NGFR (CD271; clone ME20.4, BioLegend) and Protein L for CCR, while for CARs it was measured in the PE channel to detect dsRed and AffiniPure F (ab′)₂ Fragment Goat Anti-Mouse IgG (Jackson ImmunoResearch). The following antibodies were used for FACS staining: Anti-Human CD3 (clone UCHT1, BD bioscience), Anti-Human CD4 (clone L200, BD bioscience), Anti-Mouse CD45 (clone 30-F11, BD bioscience), Anti-Human CD8 (clone SK1, BD bioscience), Anti-Human CD45 (clone HI30, BD bioscience), Anti-human CD366 (TIM-3) (clone F38-2E2, Biolegend or clone F38-2E2, ThermoFisher), Anti-human CD279 (PD-1) (clone EH12.2H7, Biolegend or clone cBioJ105, ThermoFisher), Anti-human CD62L (clone DREG-56, Biolegend), Anti-human CD269 (BCMA) (clone 19F2, Biolegend), CD45RA (clone HI100, ThermoFisher), LAG-3 (CD223; clone 3DS223H, ThermoFisher). Flow cytometry data analysis was performed with FCS Express 6 flow software.

Cytokine and Granzyme B measurements. To determine cytokine production, cell supernatants were harvested 16 hours after co-culture with target cells. The Cytokine Bead Array (CBA) Human Th1/Th2/Th17 cytokine Kit (BD Biosciences) was used according to the manufacturer's protocol. Human Granzyme B was quantitatively determined by the Human Granzyme B ELISA development kit (Mabtech, 3485-1H-6) according to the manufacturer's guidelines.

In vivo xenograft studies. For the MM in vivo model, RAG-2^{-/-}γc^{-/-} female mice were bred and maintained at the Amsterdam Animal Research Center (Universitair proefdiercentrum, AARC-UPC). The animal experiments were performed under the approval of the central authority for scientific procedures on animals (protocol number AVD114002015345). Animal welfare was monitored, and euthanasia was induced in strict accordance with the Dutch Animal Experimentation Act. The scaffold-based xenograft murine model was prepared as previously described (Groen et al., 2012. Blood 120: e9-e16). Mice were subcutaneously implanted with hybrid scaffolds, each consisting of three 2- to 3-mm³ biphasic calcium phosphate particles, coated in vitro with human bone marrow mesenchymal stromal cells (BM-MSC; 2×10⁵ cells/scaffold). Eight to twelve weeks after implantation, mice were injected with bisulvex intraperitoneally (18 mg/kg in 500 μl). Next day, they were intravenously injected with luciferase-transduced multiple myeloma cells (10×10⁶ UM9 or 2.5×10⁶ MM1S). Seven days after injection of tumor cells, mice received transduced T cells (2.5×10⁶ cells/mice) intravenously. Tumor growth was monitored by weekly bioluminescence (BLI) measurements using a PhotonIMAGER (Biospace Lab) (intraperitoneal injection of 100 μl D-luciferine). Postmortem BM and scaffolds were harvested from each mouse, dissociated (scaffolds) and filtered through a 70 μm filter. Single-cell suspensions were counted, stained, and measured by flow cytometry. For the ALL in vivo model, female 8-12-week-old NOD.Cg-PrkdcscidI12rgtmWjl/SzJ (NSG) mice (Jackson Laboratory) were used. A total of 0.5×10⁶ FFLuc-GFP NALM6 cells were administered intravenously. Six days later 0.2×10⁶ CAR or CAR+CCR T cells were administered. Tumor burden was measured by Bioluminescence imaging using the Xenogen IVIS Imaging System (Xenogen).

Statistical analysis. Data analysis and visualization was performed using GraphPad Prism 8.2.1 (GraphPad Prism, RRID: SCR_002798) software. No pre-specified effect size was used to determine sample sizes. Graphs represent individual values±SEM, for in vitro and in vivo experiments. The statistical tests, that were used to calculate the p values, are described in the relevant figure legend. Differences were considered significant at p<0.05 and p values are denoted with asterisks as follows: p>0.5, not significant (ns), *p<0.05, **p<0.01, ***p<0.001, ****p<0.0001.

### Results

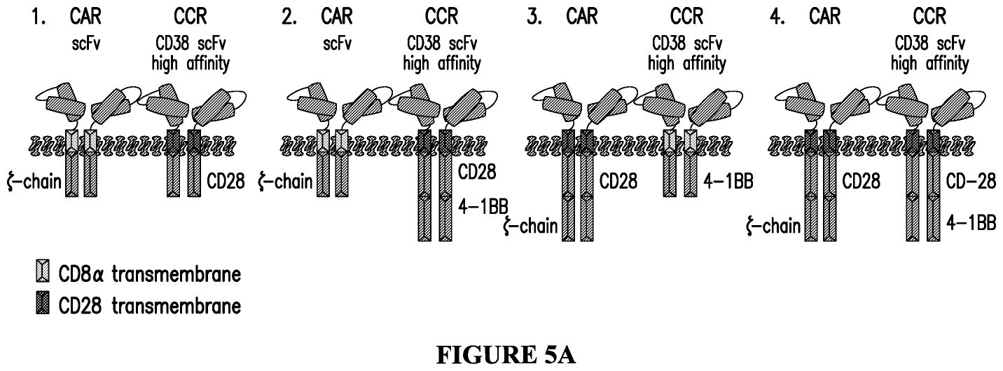

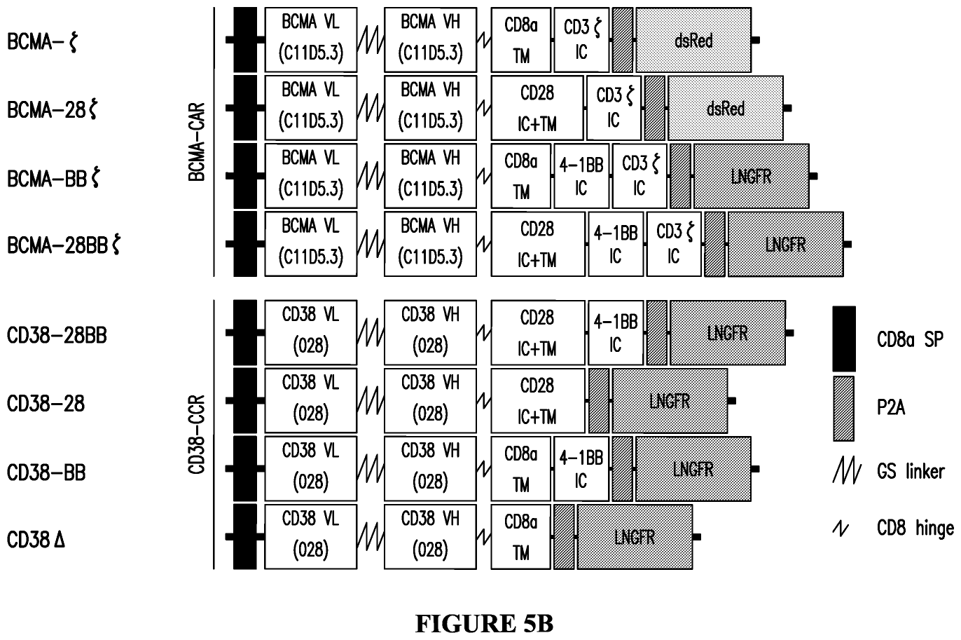

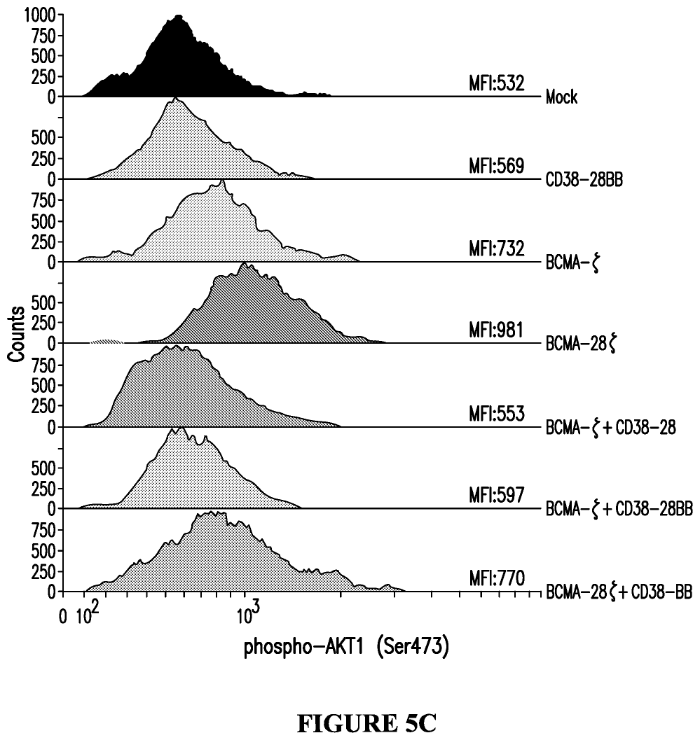

Co-expression of a CCR improves cytotoxicity of CAR-engineered T cells. In order to test the concept of combinatorial tumor targeting with a CAR and a CCR (FIG. 5A), the present example was focused on hematological malignancies, such as multiple myeloma (MM) and B cell leukemia and lymphoma. First- and second-generation CARs targeting BCMA

(FIG. 5B) were generated. CD38 was chosen to serve as a second antigen targeted by the CCR, as CD38 is robustly expressed in almost all hematologic malignancies (Naik et al., 2019. Haematologica 104: e100-e103; Krejcik et al., 2016. Blood 128:384-394) and therefore a CD38-CCR could be applicable to a variety of diseases. The CD38-CCR constructs were designed either to contain only one of the two most popular costimulatory domains, CD28 or 4-1BB (CD38-28 or CD38-BB), or to combine them (CD38-28BB) (FIGS. 5A and 5B). The CAR and the CCR constructs were linked to functionally irrelevant markers (dsRed and low-affinity nerve growth factor receptor (LNGFR), respectively) (FIG. 5B).

Peripheral blood T cells transduced with CAR+CD38-CCR combinations expressed equal abundance of the CAR on their surface to single CAR-transduced T cells (data not shown). CAR+CD38-CCR T cells showed reduced expression of CD38 on their surface indicating binding (in-cis or in-trans) of the CCR. However, it was not observed any effect on the differentiation of the produced CAR-, CCR- and CAR+CCR-expressing T cells (data not shown), nor fratricide during the manufacturing of T cells transduced with the CD38-CCR (data not shown). Moreover, compared to mock transduced T cells, CD38-28BB CCR, BCMA-ζ+CD38-28 and BCMA-ζ+CD38-28BB-expressing T cells showed no tonic signaling mediated through the CCR, as evidenced by similar phosphorylation of AKT1 Serine/Threonine Kinase 1 (or AKT1), which reflects CD3ζ and CD28 activation (FIG. 5C).

Interestingly, the addition of CD38-28 and CD38-28BB CCR in first-generation (BCMA-ζ) and CD38-BB CCR in second-generation BCMA-CAR T cells (BCMA-28ζ) (FIG. 5A), resulted in remarkable increase of their cytotoxic capacity against MM cell lines and primary CD138+CD38+ patient-derived MM cells (data not shown). The presence of a CD28 transmembrane (TM) and intracellular (IC) domain have been linked to improved cytotoxicity of a CAR and to better response to lower antigen expression (Majzner et al., 2020. Cancer Discov 10:702-723; Hamich et al., 2019. Nature 568:112-116). The effect of the positioning of the CD28 TM/IC in the BCMA-CAR instead of in the CD38-CCR was investigated. No difference was observed in the cytotoxic capacity between BCMA-28ζ+38-BB and BCMA-ζ+38-28BB T cells (data not shown). Overall, lysis of MM tumor cells by BCMA-CAR+CD38-CCR T cells was significantly higher, in low effector:target (E:T) ratios as compared to lysis by single-targeting second generation BCMA-CAR T cells of both costimulatory designs (BCMA-28ζ or BCMA-BBζ) (P<0.0001), or compared to lysis by third generation BCMA-CAR T cells (BCMA-28BBζ). These last findings indicate that the increased cytotoxic potential of BCMA-CAR+CD38-CCR T cells is not solely a result of the CD28/4-1BB costimulatory combination. In addition, T cells bearing the CD38-CCR alone did not induce cell lysis of CD38 expressing targets (FIG. 5D) and BCMA-CAR+CD38-CCR T cells were unable to induce lysis of NIH-3T3 cells that were transduced to express CD38, but do not express BCMA (3T3-38+ cells) (FIG. 5E). Thus, CD38-CCR expression was not sufficient to induce lysis of tumor by itself or off-tumor lysis in combination with the BCMA-CAR. Notably, the increase in cytotoxicity was completely diminished in the absence of CD38 expression on the target cells when U266 cells (BCMA+CD38−) were used as targets (data not shown). Together, these results demonstrate that the engagement of the co-expressed CD38-CCR with CD38 results in enhanced cytotoxicity of CAR T cells against tumor targets.

High affinity CCR binding increases CAR T cell functional avidity. The present example further aimed to elucidate whether the increase of cytotoxicity observed with CCR co-expression is a result of the CCR binding to the target or of the initiation of downstream costimulatory signals. To investigate this, we constructed a CD38 binding receptor, which lacks the intracellular costimulatory tail (CD38Δ) and therefore is able to bind to the CD38 antigen, with the same affinity as the CD38-CCR, but it is not able to induce activation, nor co-stimulation in the T cells (FIG. 5B). Surprisingly, the double-targeting BCMA-ζ+CD38Δ T cells lysed MM tumor cells as effective as the BCMA-CAR+CD38-28BB T cells (FIG. 6A). Therefore, it seems that the engagement of the second target-antigen by the CCR, CD38 in this case, can by itself increase the lytic capacity of double-targeting CAR+CCR T cells independent of the addition of costimulatory signaling. Indeed, when the phosphorylation of AKT1 in T cells after target cell encounter was measured, it was found that double binding by BCMA-ζ+CD38Δ resulted in increase of pAKT1 mean fluorescence intensity (MFI), compared to single binding by BCMA-ζ (FIG. 6B), indicating that the multivalent binding enhances the downstream signaling of CAR T cell activation. Phosphorylation of AKT1 was further increased in BCMA-ζ+CD38-28BB T cells (FIG. 6C), although that did not translate into better tumor cell lysis in this system. Additionally, it was found significantly increased secretion of granzyme B by BCMA-ζ+CD38Δ compared to BCMA-ζ as well as BCMA-28ζ CAR T cells (P=0.0004 and P<0.0001 respectively). Moreover, granzyme B secretion by BCMA-ζ+CD38-28 T cells was higher compared to BCMA-28ζ CAR T cells (data not shown), demonstrating once more the effect of multivalent tumor engagement beyond the addition of CD28 costimulation. Further, double targeting and combinatorial costimulation with BCMA-ζ+CD38-28BB and BCMA-28ζ+CD38-BB T cells also showed higher concentrations of secreted granzyme B (data not shown).

It was hypothesized that the improved cytotoxic efficacy was a result of an increased functional avidity and the formation of stronger T cell binding due to the multivalent engagement through the BCMA-CAR and the high affinity CD38-CCR. We tested this hypothesis by determining the actual binding strength between tumor cells and CAR T cells with or without CCR co-expression. MM1.S cells were seeded in z-Movi Cell Avidity Analyzer microfluidic chips, following which, mono- and double-targeting CAR T cells were sequentially flushed in and allowed to bind to the target cells. The binding avidity between tumor cells and T cells was quantified by applying acoustic forces (ultrasound waves) of increasing magnitude (pN). It was found that the interaction of BCMA-ζ+CD38Δ T cells with tumor cells could withstand the application of higher acoustic forces compared to the interaction of BCMA-ζ with tumor cells, as there were significantly more BCMA-ζ+CD38Δ T cells attached compared to BCMA-cells (P=0.0082, FIG. 6C). Addition of costimulatory domains resulted in further slight increase of functional avidity of BCMA-ζ+CD38-28BB T cells, which overall showed significantly higher binding strength compared to both BCMA-ζ and BCMA-28ζ CAR T cells (P=0.0002 and P=0.0274, FIG. 6C).

The specific affinity requirements of CD38 engagement, which resulted in an increase of cytotoxicity, were further interrogated. To this end, four single-chain variable fragments (scFvs) binding to the same CD38 epitope with gradually lower affinity were used (Drent et al., 2017. Mol Ther 25:1946-1958) (FIG. 6D). Only the high affinity (scFv 028, KD=1.8 nM) binding of the CD38-CCR resulted in superior cytotoxicity (FIG. 6E) of BCMA-CAR+CD38-28BB T cells compared to single targeted BCMA-CAR T cells. Affinity reduction of approximately 10-fold (scFv 38A1, KD=16.5 nM) reduced this effect (FIG. 6E) and led to decreased granzyme B secretion and functional avidity (data not shown). Lowering the expression of CD38 on the tumor cells also reduced the cytotoxic benefit of BCMA-CAR+CD38-CCR T cells (data not shown), indicating the requirement for a high expression of the CCR-targeting secondary antigen. Overall, these data demonstrate that a high affinity engagement of a highly expressed secondary antigen, such as CD38, is necessary in order to increase the functional avidity and enhance the cytotoxic capacity of BCMA-CAR T cells.

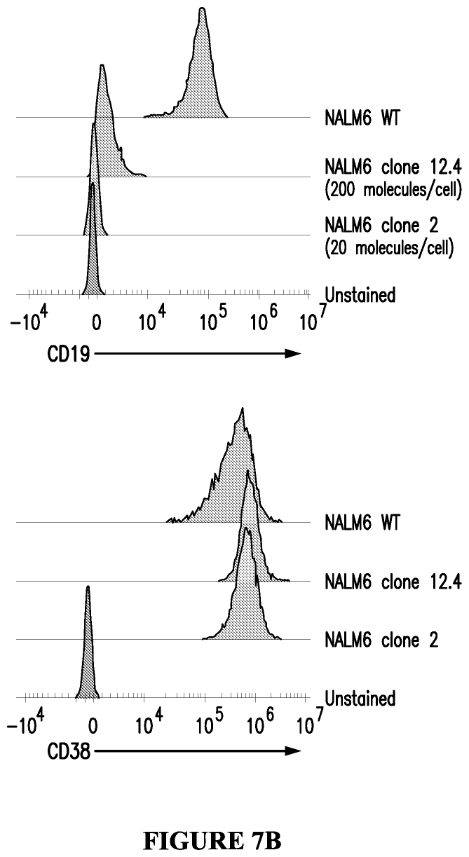

CAR+CCR combination enables the lysis of low-antigen expressing tumor variants. A decreased target-antigen density is one of the major mechanisms of tumor escape and relapse after adoptive therapy with CAR T cells. Given the superior cytotoxic capacity of double-targeted CAR+CCR T cells, their efficacy was examined against tumor variants with low antigen density. K562 cell lines (CD38+) were transduced and sorted for the expression of variable concentrations of BCMA. The sorted lines were further characterized as negative (K562-BCMAneg), low (K562-BCMAlow), medium (K562-BCMAmed), and high (K562-BCMAhigh), after quantitative comparison of BCMA expression with MM cell lines and primary MM cells (Frerichs et al., 2020. Clin Cancer Res 26:2203-2215). BCMA-CAR+CD38-CCR T cells (BCMA-ζ+CD38-28, BCMA-ζ+CD38-28BB or BCMA-28ζ+CD38-BB) exhibited increased lysis of all K562-BCMA lines compared to BCMA-ζ, BCMA-28ζ and BCMA-28BBζ CAR T cells (FIG. 7A). Notably, K562-BCMAlow cells, which are representative of the median BCMA expression on primary MM, were resistant to lysis by single targeting BCMA-CAR T cells but remained sensitive to BCMA-CAR+CD38-CCR T cells (FIG. 7A). More importantly the lysis of K562-BCMAlow and K562-BCMAmed K562 cells by BCMA-CAR+CD38-CCR T cells was comparable to that of K562-BCMAhigh by BCMA-28ζ CAR T cells (FIG. 7A).

This observation was additionally confirmed in a model of acute lymphoblastic leukemia NALM6 cells engineered and subcloned to express low CD19 antigen density (Hamich et al., 2019. Nature 568:112-116). The NALM6 clone 12.4 was generated after biallelic disruption of the Cd19 gene and consecutive lentiviral transduction to express CD19 at low concentrations. NALM6 clone 12.4 carries approximately 200 molecules per cell as determined by flow cytometry-based quantification (FIG. 7B). The NALM6 clone 2 was generated only by monoallelic disruption of the Cd19 gene (Hamich et al., 2019. Nature 568:112-116) and displays an ultra-low CD19 surface expression (approximately 20 molecules per cell), which is negative by conventional flow cytometry analysis and only detectable by means of flow cytometry after signal amplification (FIG. 7B). Also, in this system CD19-CAR+CCR T cells specifically recognized CD19 expressing NALM6 targets and not CD19 knockout NALM6 cells (data not shown). Interestingly, the addition of CD38Δ did not result in increase of cytotoxicity in this model for the wild type (WT) nor for the CD19-low NALM6 (data not shown). This could be attributed to the fact that the expression of CD38 on NALM6 was 10 times lower than in the MM cell lines. Clone 12.4 was still sensitive to killing by CD19- and CD19-28ζ CAR T cells (data not shown). The addition of CD38-28 or CD38-28BB CCR equally enhanced the in vitro cytotoxicity of CD19-whereas no difference in lytic capacity was observed between CD19-28° C. and CD19-28ζ+CD38-BB (data not shown). Thus, the addition of a CD28 domain was necessary to achieve maximum lysis of clone 12.4. The ultra-low density of CD19 renders NALM6 clone 2 unsusceptible to lysis by both CD19- and second generation CD19-28ζ CAR T cells. In this case, combination of CD19-CAR and CD38-CCR providing double CD28 and 4-1BB costimulation was necessary to restore lysis (FIG. 7C). CD19-ζ+CD38-28 combination failed to increase lysis, whereas both CD19-2+CD38-28BB and CD19-28ζ+CD38-BB T cells elicited significantly higher lysis against the NALM6 clone 2 cells (P=0.0125, FIG. 7C), indicating that in this system the costimulatory design benefits the cytotoxic outcomes, rather than the CD38 engagement. Since the presence of the CD28 endodomain was important for the optimal lysis of clone 12.4, it was interrogated whether the addition of a second CD28 domain would have an additive effect. Interestingly, although CD19-28ζ+CD38-28BB T cells did not elicit a higher lysis of clone 12.4, these cells significantly increased lysis of clone 2 (P<0.0001), which was almost similar to the lysis activity observed when targeting the CD19high wild type NALM6 cell line by conventional CD19-28ζ CAR T cells (FIG. 7D). Altogether, our results suggest that the addition of the CD38-CCR can improve the sensitivity for antigen recognition in different hematological tumor models.

Co-expression of a CCR increases the proliferative capacity of CAR T cells. Previous studies have elegantly demonstrated that CD28 and 4-1BB intracellular co-stimulatory signaling domains equip CAR T cells with distinct kinetics and functions (Kawalekar et al., 2016. Immunity 44:712; Zhao et al., 2015. Cancer Cell 28:415-428), which can be integrated to improve T cell potency. It was hypothesized that the combination of CD28 and 4-1BB costimulatory signals delivered by the co-expression of a CCR could enhance not only the cytolytic potency but also the cytokine secretion and the proliferative potential of CAR T cells. Indeed, when challenged with MM cells, BCMA-CAR+CD38-CCR T cells produced significantly higher concentrations of interferon (IFN)-γ (P=0.0216) and interleukin (IL)-2 (P=0.0433) compared to BCMA-ζ, BCMA-28ζ and BCMA-BBζ CAR T cells, and higher tumor necrosis factor (TNF)-α compared to BCMA-S and BCMA-28ζ CAR T cells (FIG. 8). This was a consequence of the CCR-mediated costimulatory signaling in combination to CAR-mediated activation and not solely of the CCR engagement, since no cytokine secretion increase was observed by BCMA-ζ+CD38Δ compared to BCMA-ζ((FIG. 8). Secretion of cytokines was reduced accordingly, when lowering the affinity of CD38-CCR (data not shown). Furthermore, in long-term proliferation assays, double-targeting BCMA-CAR+CD38-CCR T cells (BCMA-ζ+CD38-28, BCMA-ζ+CD38-28BB and BCMA-28ζ+CD38-BB) showed a high expansion potential, outperforming their respective single targeting CARs (BCMA-ζ and BCMA-28ζ) (data not shown). Despite the fact that BCMA-CAR+CD38-CCR T cells showed no difference in cytokine secretion compared to 3rd generation BCMA-28BBζ cells, they clearly expanded better in vitro. Notably, the enhanced proliferative capacity of the double-targeting BCMA-CAR+CD38-CCR T cells was similar to BCMA-BBζ T cells and was corroborated with lower surface expression of programmed cell death protein 1 (PD-1), compared to BCMA-ζ, BCMA-28ζ and BCMA-28BBζ CAR T cells (data not shown). T cells bearing only CD38-BB or CD38-28BB CCR showed no secretion of IFN-γ and TNF-α in response to antigen engagement, and we observed a similar secretion of IL-2 (FIG. 8) and expansion (data not shown) to mock T cells. However, comparable IL-2 secretion was secreted by the CD38Δ T cells, indicating that it is not mediated through the costimulatory domains of the CCR; rather it is induced by the engagement of CD38 and attributed to enhanced alloreactivity. Interestingly, the highly proliferating BCMA-CAR+CD38-CCR T cells were not characterized by the retention of a central memory phenotype, as observed with BCMA-BBζ T cells, as the majority of BCMA-CAR+CD38-CCR T cells differentiated to a CD45RA-CD62L-effector memory phenotype (data not shown).

Since CD38-CCR engagement is a successful strategy to provide co-stimulation to CAR T cells, it was verified whether it is mandatory that the CCR target is expressed on the tumor cells or whether it is possible that the CCR-mediated costimulatory signaling is initiated after binding in-trans to an antigen expressed on accessory cells. To investigate this, we set up a co-culture cytotoxicity assay where luciferase-positive BCMA+CD38-U266 cells were co-cultured with calcein-loaded 3T3-38+ cells. When BCMA-CAR+CD38-CCR T cells were added in the co-culture they preserved their cytotoxicity against the U266 cells while leaving 3T3-38+ cells intact (FIG. 9A). Although, the binding of the CD38-CCR to CD38 expressed on the 3T3 cells did not induce off-tumor toxicity, it was sufficient to induce the costimulatory signaling leading to improved cytokine secretion and expansion of the BCMA-CAR+CD38-CCR T cells compared to respective single-targeting BCMA-CAR T cells (FIGS. 9B and 9C). The BCMA-CAR+CD38-CCR strategies combining both CD28 and 4-1BB costimulation showed higher cytokine secretion and expansion with BCMA-28ζ+CD38-BB T cells outperforming BCMA-ζ+CD38-28BB in IFN-γ and TNF-α secretion (FIGS. 9B and 9C). Thus, the co-expression of a CD38-CCR can enhance the expansion potential of CAR T cells, not only through engagement of the tumor-expressed target-antigen, but also through binding in-trans of the antigen expressed on accessory cells without off-tumor toxicity. When wild type (CD38 negative) 3T3 cells were used in the co-culture, lysis of U266 was preserved and 3T3 cells remained intact (data not shown) but no increase of cytokines and proliferation was observed (data not shown), indicating that in-cis or in-trans binding of CD38 expressed on the CAR T cells did not contribute to the improved expansion. Since monocytes express CD38 and their activation has been linked to toxicity, primary CD14+ monocytes were used as accessory cells in the same co-culture system and indicators of their activation were measured. Binding of the CD38-CCR still induced the secretion of cytokines by the CAR+CCR T cells but did not induce excessive activation of the monocytes measured by surface expression of CD80, CD86, Human Leukocyte Antigen-DR isotype (HLA-DR) and secretion of IL-6 (data not shown).

CCR co-expression increases in vivo anti-tumor efficacy of CAR T cells and improves elimination of antigen-low variants. The in vitro studies revealed that the combination of BCMA-28ζ+CD38-BB modestly outperformed BCMA-ζ+CD38-28BB in cytokine secretion (through in-cis and in-trans binding) and proliferative potential. Thus, the cumulative benefit of tumor co-targeting with BCMA-28ζ+CD38-BB was evaluated in vivo using a previously described xenograft MM murine model under challenging conditions with low T cell dosing (Drent et al., 2019. Clin Cancer Res 25:4014-4025; Groen et al., 2012. Blood 120: e9-e16). Mice bearing humanized bone marrow (BM)-like scaffold niches were engrafted with luciferase-expressing MM cells and treated 7 days post tumor injection with CD38-28BB, BCMA-28ζ, BCMA-BBζ, BCMA-28ζ+CD38-BB or mock T cells. Post-infusion monitoring with bioluminescence imaging (BLI) revealed that treatment with CD38-28BB alone had no effect on tumor progression, confirming in vitro findings that CD38-CCR does not function without an activation signal (data not shown). Treatment with BCMA-BBζ and BCMA-28ζ CAR T cells also failed to control tumor growth in this system (FIG. 10A). However, mice treated with double-targeting BCMA-CAR/CD38-CCR T cells (BCMA-28ζ+CD38-BB) showed an impressive delay of tumor growth (FIG. 10A). Post-mortem analysis of the scaffold at 7 weeks after T cell injection revealed lower number of UM9 [Green Fluorescent Protein (GFP)+/CD38+] tumor cells in the BCMA-28ζ+CD38-BB group compared to all other treatment groups (FIG. 10B). No change in the expression of BCMA on the remaining UM9 tumor cells was observed between the evaluable treatment groups (data not shown).

BCMA-28ζ+CD38-BB and BCMA-BBζ T cells showed significantly better persistence than BCMA-28ζ T cells (P=0.0016, FIG. 10C). Interestingly, there was no difference in the remaining absolute T cell numbers in the scaffolds for the BCMA-28ζ+CD38-BB group and the BCMA-BBζ group, despite the difference in the tumor burden (FIG. 10C), indicating the improved anti-tumor cytotoxic potency of BCMA-28ζ+CD38-BB cells. In addition, an almost 1:1 balanced CD4:CD8 ratio was maintained in the remaining CAR T cells of BCMA-282+CD38-BB group, whereas in the BCMA-28ζ and BCMA-BBζ groups the persisting cells were, to various extents, enriched for CD4+ T cells (data not shown). Further evaluation of the expression of surface markers related to exhaustion on the CAR T cells revealed that BCMA-28ζ+CD38-BB cells expressed significantly lower concentrations of PD-1 than BCMA-28ζ CAR T cells (P=0.0142) and significantly lower concentrations of lymphocyte-activation gene 3 (LAG-3) compared to BCMA-BBζ (P=0.0015). There was no difference in T cell immunoglobulin domain (TIM-3) abundance between the groups (data not shown). Collectively, the BCMA-28ζ+CD38-BB group had lower percentage of triple positive (PD-1+LAG-3+TIM-3+) cells than BCMA-28ζ and BCMA-BBζ (data not shown). These data demonstrate that the combination of a BCMA-CAR and a CD38-CCR not only enhanced cytotoxic anti-tumor function, but also increased persistence and prevented exhaustion of therapeutic T cells, compared to second generation BCMA-targeting CARs. Interestingly, remaining BCMA-BBζ CAR T cells displayed a higher MFI of BCMA expression as compared to persisting BCMA-28ζ+CD38-BB cells (FIG. 10D), which was correlated with the co-existence with remaining tumor cells (FIG. 10E).

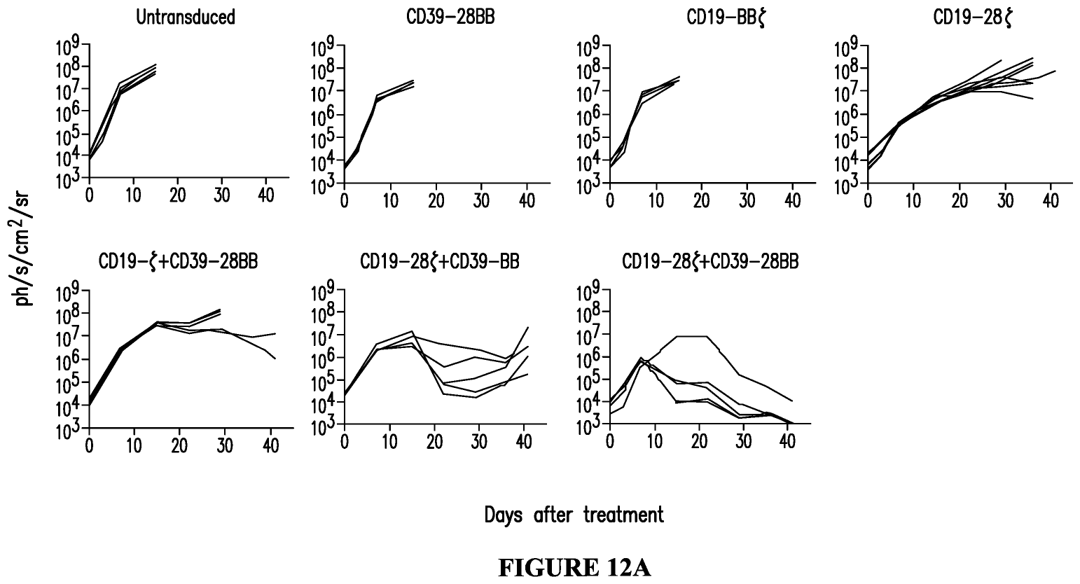

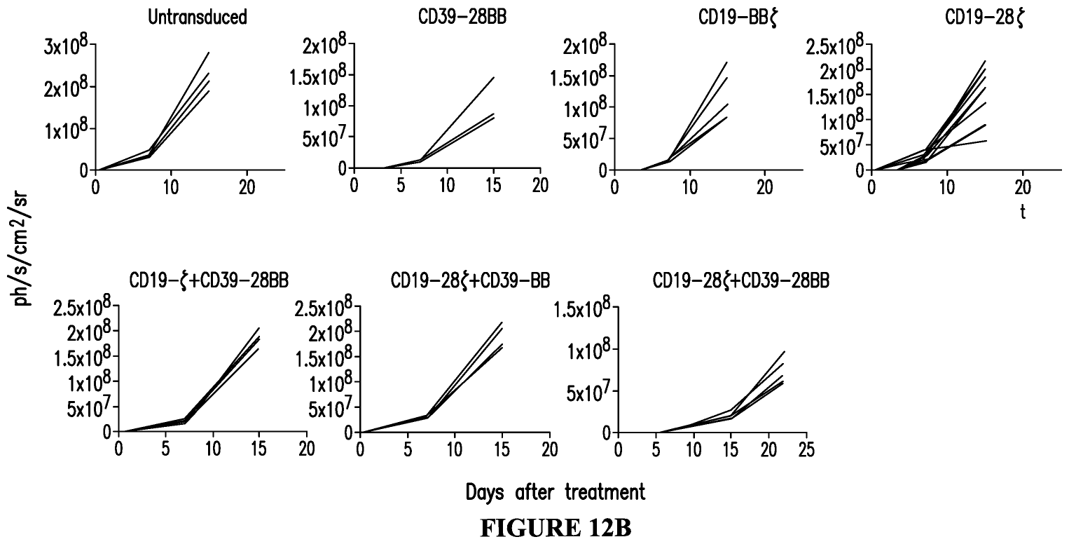

Next, it was interrogated whether the functional boost provided by the CCR co-expression to CAR T cells would also lead to eradication of tumor clones with low antigen expression. In order to investigate that, an in vivo “stress test” was employed, where mice engrafted with the NALM6 clone 12.4 (200 mol of CD19 per cell) or the NALM6 clone 2 (approx. 20 mol per cell) were treated. CD19-CAR+CD38-CCR combination delayed tumor growth of both CD19low NALM6 clones and improved the survival of mice compared to both CD19-28ζ and CD19-BBζ CAR T cells. More specifically, although differences in the in vitro lysis of NALM6 clone 12.4 between CD19-28ζ and the different CAR+CCR combinations were observed, these in vivo studies revealed that mice treated with CD19-28ζ+CD38-BB and CD19-282+CD38-28BB T cells had significantly lower tumor burden and survived longer than mice treated with CD19-28ζ, CD19-BBζ or CD19-2+CD38-28BB T cells (P=0.0283, FIGS. 11A and 11B). Most notably, CD19-28ζ+CD38-28BB T cells outperformed all other treatments by inducing long term tumor remissions (FIGS. 11B-11D and 12A).

Importantly, the ultra-low CD19 antigen density NALM6 clone 2 was resistant to killing by both CD19-28ζ and CD19-BBζ CAR T cells as well as CD19-2+CD38-28BB and CD19-28ζ+CD38-BB (FIGS. 11B-D and 12B).

Nevertheless, the addition of a second CD28 domain in CD19-28ζ+CD38-28BB cells effectively delayed tumor progression and significantly prolonged the survival of mice (P<0.0001, FIGS. 11B, 11E, 11F and 12B).

### Example 5—Off-Tumor Toxicity of CAR28ζ+CCR28BB T Cells

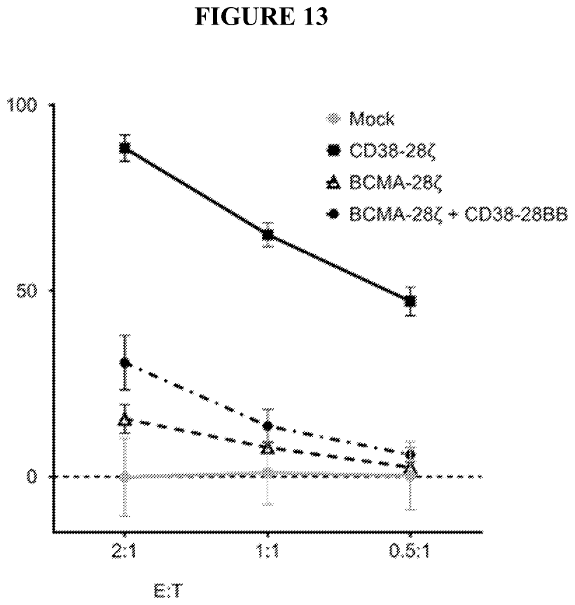

Previous studies using CAR282+CCR28BB T cells comprising CD8a transmembrane domains have shown that the engagement of CCR only mediates activation of the T cell and subsequent lysis of target cells after dimerization of the CD8a transmembrane domains (Hirabayashi et al., 2021. Nature Cancer 2:904-918). CD28 is expressed as a dimer in the cell membrane. However, as shown in FIG. 13, when using BCMA-28ζ (BCMA-CAR construct comprising an extracellular single chain variable fragment (see BCMA02, WO2016/094304A), followed by a CD28 transmembrane and intracellular sequence fused to CD3ζ intracellular domain), and CD38-28BB (CCR construct comprising a CD38-specific scFv ((Drent et al., 2016. Haematologica 101:616), a CD28 transmembrane and intracellular sequence and the 4-1BB intracellular domain), no significantly higher lysis of CD38 expressing target cells was observed compared to negative controls. Therefore, no off-tumor toxicity is expected by CAR28ζ+CCR28BB T cells.

### EMBODIMENTS OF THE PRESENTLY DISCLOSED SUBJECT MATTER

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1-100. (canceled)

101.-125. (canceled)

126. A cell comprising:
(a) an antigen-recognizing receptor that binds to an antigen; and
(b) a chimeric co-stimulating receptor (CCR) that binds to CD38,
wherein the cell exhibits substantial cytolytic activity against cells that are single positive for the antigen.

127. The cell of claim 126, wherein the cell is a cytotoxic T lymphocyte (CTL), a γδ T cell, a tumor-infiltrating lymphocyte (TIL), a regulatory T cell, a Natural Killer T (NKT) cell or a NK cell.

128. The cell of claim 126, wherein the antigen-recognizing receptor comprises a T cell receptor (TCR), a TCR like fusion molecule, or a chimeric antigen receptor (CAR).

129. The cell of claim 126, wherein the antigen is a tumor antigen or a pathogen antigen.

130. The cell of claim 126, wherein the tumor antigen comprises CD19, carbonic anhydrase IX (CAIX), carcinoembryonic antigen (CEA), CD8, CD7, CD10, CD20, CD22, CD30, CD33, CLL1, CD34, CD41, CD44, CD49f, CD56, CD74, CD133, CD138, CD123, CD44V6, an antigen of a cytomegalovirus (CMV) infected cell, epithelial glycoprotein-2 (EGP-2), epithelial glycoprotein-40 (EGP-40), epithelial cell adhesion molecule (EpCAM), receptor tyrosine-protein kinase Erb-B2, Erb-B3, Erb-B4, folate-binding protein (FBP), fetal acetylcholine receptor (AChR), folate receptor-«, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, GPRC5D, FcRL5, NKCS1, EGF1R, EGFR-VIII, CD99, CD70, ADGRE2, CCR1, LILRB2, PRAME, CD48, P2RY10, CD70, BOB1, CLEC12A, CCR1, LILRB2, CD5, CD38, CS-1, Lewis antigen (LeY), or ERBB.

131. The cell of claim 128, wherein the TCR like fusion molecule is a recombinant T cell receptor (TCR) comprising an antigen binding chain that comprises: (a) an antigen-binding fragment of an immunoglobulin variable region that binds to an antigen in an HLA-independent manner; and (b) a constant domain that is capable of associating with a CD3ζ polypeptide.

132. The cell of claim 131, wherein the constant domain comprises a native or modified TRAC polypeptide or a native or modified TRBC polypeptide.

133. The cell of claim 131, wherein the CD3ζ polypeptide is fused to an intracellular domain of a co-stimulatory molecule or a portion thereof.

134. The cell of claim 128, wherein the antigen-recognizing receptor is a CAR, wherein the CAR comprises:
an intracellular CD3ζ signaling domain;
a CD28 co-stimulatory intracellular domain;
a transmembrane domain; and
an extracellular antigen specific binding domain.

135. The cell of claim 134, wherein the CD3ζ polypeptide is a native or modified CD3ζ polypeptide.

136. The cell of claim 135, wherein the modified CD3ζ polypeptide comprises a native ITAM1, an ITAM2 variant consisting of two loss-of-function mutations, and an ITAM3 variant consisting of two loss-of-function mutations.

137. The cell of claim 136, wherein the CCR comprises an extracellular antigen-binding domain that binds to CD38, and an intracellular domain that is capable of delivering a costimulatory signal to the cell but does not alone deliver an activation signal to the cell.

138. The cell of claim 126, wherein the intracellular domain of the CCR comprises two or more intracellular domains of a co-stimulatory molecule or a portion thereof.

139. The cell of claim 133, wherein the co-stimulatory molecule comprises CD28, 4-1BB, OX40, CD17, CD40, CD154, CD97, CD11a/CD18, ICOS, DAP-10, CD2, CD244, CD27, or NKG2D.

140. The cell of claim 138, wherein the co-stimulatory domains are a CD28 co-stimulatory domain and a co-stimulatory 4-1BB intracellular domain.

141. A method of reducing and/or abolishing expression of CD38 in a cell, comprising introducing into the cell a nucleic acid molecule that encodes a chimeric co-stimulating receptor (CCR) that binds to CD38.

142. The method of claim 141, wherein the cell is a T cell, a B cell, a Natural Killer (NK) cell, or a dendritic cell.

143. A method of treating and/or preventing a disease associated with and/or expressing CD38 in a subject, comprising administering to the subject (a) a CD38 drug and (b) a cell according to claim 126.

144. A pharmaceutical composition, comprising the cell according to claim 126, and a pharmaceutically acceptable carrier.

145. A method of treating a tumor, comprising the administration of the pharmaceutical composition of claim 144 to a subject.


SEQUENCE LISTING (103 sequences)

SEQ ID NO: 1 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 2 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 3 - DNA, 123 bp, Artificial Sequence
  Synthetic
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 4 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 5 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 6 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 7 - protein, 112 aa, Artificial Sequence
  Synthetic
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 22 aa, Artificial Sequence
  Synthetic
QNQLYNELNLGRREEYDVLDKR

SEQ ID NO: 9 - DNA, 66 bp, Artificial Sequence
  Synthetic
cagaaccagctctataacgagctcaatctaggacgaagagaggagtacgatgttttggacaagaga

SEQ ID NO: 10 - protein, 22 aa, Artificial Sequence
  Synthetic
QNQLFNELNLGRREEFDVLDKR

SEQ ID NO: 11 - DNA, 66 bp, Artificial Sequence
  Synthetic
cagaaccagctctttaacgagctcaatctaggacgaagagaggagttcgatgttttggacaagaga

SEQ ID NO: 12 - protein, 23 aa, Artificial Sequence
  Synthetic
QEGLYNELQKDKMAEAYSEIGMK

SEQ ID NO: 13 - DNA, 69 bp, Artificial Sequence
  Synthetic
caggaaggcctgtacaatgaactgcagaaagataagatggcggaggcctacagtgagattgggatgaaa

SEQ ID NO: 14 - protein, 23 aa, Artificial Sequence
  Synthetic
QEGLFNELQKDKMAEAFSEIGMK

SEQ ID NO: 15 - DNA, 69 bp, Artificial Sequence
  Synthetic
caggaaggcctgttcaatgaactgcagaaagataagatggcggaggccttcagtgagattgggatgaaa

SEQ ID NO: 16 - protein, 22 aa, Artificial Sequence
  Synthetic
HDGLYQGLSTATKDTYDALHMQ

SEQ ID NO: 17 - DNA, 66 bp, Artificial Sequence
  Synthetic
cacgatggcctttaccagggtctcagtacagccaccaaggacacctacgacgcccttcacatgcag

SEQ ID NO: 18 - protein, 22 aa, Artificial Sequence
  Synthetic
HDGLFQGLSTATKDTFDALHMQ

SEQ ID NO: 19 - DNA, 66 bp, Artificial Sequence
  Synthetic
cacgatggccttttccaggggctcagtacagccaccaaggacaccttcgacgcccttcacatgcag

SEQ ID NO: 20 - protein, 112 aa, Artificial Sequence
  Synthetic
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 21 - DNA, 336 bp, Artificial Sequence
  Synthetic
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccaggggctcagtacagccaccaaggacaccttcgacgcccttcacatgcaggccctgccccctcgc

SEQ ID NO: 22 - protein, 20 aa, Artificial Sequence
  Synthetic
MYRMQLLSCIALSLALVTNS

SEQ ID NO: 23 - protein, 20 aa, Artificial Sequence
  Synthetic
MYSMQLASCVTLTLVLLVNS

SEQ ID NO: 24 - protein, 20 aa, Artificial Sequence
  Synthetic
METPAQLLFLLLLWLPDTTG

SEQ ID NO: 25 - protein, 20 aa, Artificial Sequence
  Synthetic
METDTLLLWVLLLWVPGSTG

SEQ ID NO: 26 - protein, 21 aa, Artificial Sequence
  Synthetic
MALPVTALLLPLALLLHAARP

SEQ ID NO: 27 - protein, 18 aa, Artificial Sequence
  Synthetic
MALPVTALLLPLALLLHA

SEQ ID NO: 28 - protein, 16 aa, Artificial Sequence
  Synthetic
MKWVTFISLLFSSAYS

SEQ ID NO: 29 - protein, 30 aa, Artificial Sequence
  Synthetic
MDSKGSSQKGSRLLLLLVVSNLLLCQGVVS

SEQ ID NO: 30 - protein, 140 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 31 - DNA, 4627 bp, Homo sapiens
atatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaatttgcttttcttttttttttttttttttttttttttttttgagatggagttttgctcttgttgcccaggctggagtgcaatggtgcatcttggctcactgcaacctccgcctcccaggttcaagtgattctcctgcctcagcctcccgagtagctgagattacaggcacccgccaccatgcctggctaattttttgtatttttagtagagacagggtttcactatgttggccaggctggtctcgaactcctgacctcaggtgatccacccgcttcagcctcccaaagtgctgggattacaggcgtgagccaccacacccggcctgcttttcttaaagatcaatctgagtgctgtacggagagtgggttgtaagccaagagtagaagcagaaagggagcagttgcagcagagagatgatggaggcctgggcagggtggtggcagggaggtaaccaacaccattcaggtttcaaaggtagaaccatgcagggatgagaaagcaaagaggggatcaaggaaggcagctggattttggcctgagcagctgagtcaatgatagtgccgtttactaagaagaaaccaaggaaaaaatttggggtgcagggatcaaaactttttggaacatatgaaagtacgtgtttatactctttatggcccttgtcactatgtatgcctcgctgcctccattggactctagaatgaagccaggcaagagcagggtctatgtgtgatggcacatgtggccagggtcatgcaacatgtactttgtacaaacagtgtatattgagtaaatagaaatggtgtccaggagccgaggtatcggtcctgccagggccaggggctctccctagcaggtgctcatatgctgtaagttccctccagatctctccacaaggaggcatggaaaggctgtagttgttcacctgcccaagaactaggaggtctggggtgggagagtcagcctgctctggatgctgaaagaatgtctgtttttccttttagaaagttcctgtgatgtcaagctggtcgagaaaagctttgaaacaggtaagacaggggtctagcctgggtttgcacaggattgcggaagtgatgaacccgcaataaccctgcctggatgagggagtgggaagaaattagtagatgtgggaatgaatgatgaggaatggaaacagcggttcaagacctgcccagagctgggtggggtctctcctgaatccctctcaccatctctgactttccattctaagcactttgaggatgagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccgggtttaatctgctcatgacgctgcggctgtggtccagctgaggtgaggggccttgaagctgggagtggggtttagggacgcgggtctctgggtgcatcctaagctctgagagcaaacctccctgcagggtcttgcttttaagtccaaagcctgagcccaccaaactctcctacttcttcctgttacaaattcctcttgtgcaataataatggcctgaaacgctgtaaaatatcctcatttcagccgcctcagttgcacttctcccctatgaggtaggaagaacagttgtttagaaacgaagaaactgaggccccacagctaatgagtggaggaagagagacacttgtgtacaccacatgccttgtgttgtacttctctcaccgtgtaacctcctcatgtcctctctccccagtacggctctcttagctcagtagaaagaagacattacactcatattacaccccaatcctggctagagtctccgcaccctcctcccccagggtccccagtcgtcttgctgacaactgcatcctgttccatcaccatcaaaaaaaaactccaggctgggtgcgggggctcacacctgtaatcccagcactttgggaggcagaggcaggaggagcacaggagctggagaccagcctgggcaacacagggagaccccgcctctacaaaaagtgaaaaaattaaccaggtgtggtgctgcacacctgtagtcccagctacttaagaggctgagatgggaggatcgcttgagccctggaatgttgaggctacaatgagctgtgattgcgtcactgcactccagcctggaagacaaagcaagatcctgtctcaaataataaaaaaaataagaactccagggtacatttgctcctagaactctaccacatagccccaaacagagccatcaccatcacatccctaacagtcctgggtcttcctcagtgtccagcctgacttctgttcttcctcattccagatctgcaagattgtaagacagcctgtgctccctcgctccttcctctgcattgcccctcttctccctctccaaacagagggaactctcctacccccaaggaggtgaaagctgctaccacctctgtgcccccccggcaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctgcagaatgttgtgaggggtttatttttttttaatagtgttcataaagaaatacatagtattcttcttctcaagacgtggggggaaattatctcattatcgaggccctgctatgctgtgtatctgggcgtgttgtatgtcctgctgccgatgccttc

SEQ ID NO: 32 - protein, 178 aa, Homo sapiens
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 33 - protein, 175 aa, Homo sapiens
LNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 34 - DNA, 1448 bp, Homo sapiens
aggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcagacaagactagatccaaaaagaaaggaaccagcgcacaccatgaaggagaattgggcacctgtggttcattcttctcccagattctcagcccaacagagccaagcagctgggtcccctttctatgtggcctgtgtaactctcatctgggtggtgccccccatccccctcagtgctgccacatgccatggattgcaaggacaatgtggctgacatctgcatggcagaagaaaggaggtgctgggctgtcagaggaagctggtctgggcctgggagtctgtgccaactgcaaatctgactttacttttaattgcctatgaaaataaggtctctcatttattttcctctccctgctttctttcagactgtggctttacctcgggtaagtaagcccttccttttcctctccctctctcatggttcttgacctagaaccaaggcatgaagaactcacagacactggagggtggagggtgggagagaccagagctacctgtgcacaggtacccacctgtccttcctccgtgccaacagtgtcctaccagcaaggggtcctgtctgccaccatcctctatgagatcctgctagggaaggccaccctgtatgctgtgctggtcagcgcccttgtgttgatggccatggtaagcaggagggcaggatggggccagcaggctggaggtgacacactgacaccaagcacccagaagtatagagtccctgccaggattggagctgggcagtagggagggaagagatttcattcaggtgcctcagaagataacttgcacctctgtaggatcacagtggaagggtcatgctgggaaggagaagctggagtcaccagaaaacccaatggatgttgtgatgagccttactatttgtgtggtcaatgggccctactactttctctcaatcctcacaactcctggctcttaataacccccaaaactttctcttctgcaggtcaagagaaaggatttctga

SEQ ID NO: 35 - DNA, 1489 bp, Homo sapiens
aggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcggacaagactagatccagaagaaagccagagtggacaaggtgggatgatcaaggttcacagggtcagcaaagcacggtgtgcacttcccccaccaagaagcatagaggctgaatggagcacctcaagctcattcttccttcagatcctgacaccttagagctaagctttcaagtctccctgaggaccagccatacagctcagcatctgagtggtgtgcatcccattctcttctggggtcctggtttcctaagatcatagtgaccacttcgctggcactggagcagcatgagggagacagaaccagggctatcaaaggaggctgactttgtactatctgatatgcatgtgtttgtggcctgtgagtctgtgatgtaaggctcaatgtccttacaaagcagcattctctcatccatttttcttcccctgttttctttcagactgtggcttcacctccggtaagtgagtctctcctttttctctctatctttcgccgtctctgctctcgaaccagggcatggagaatccacggacacaggggcgtgagggaggccagagccacctgtgcacaggtgcctacatgctctgttcttgtcaacagagtcttaccagcaaggggtcctgtctgccaccatcctctatgagatcttgctagggaaggccaccttgtatgccgtgctggtcagtgccctcgtgctgatggccatggtaaggaggagggtgggatagggcagatgatgggggcaggggatggaacatcacacatgggcataaaggaatctcagagccagagcacagcctaatatatcctatcacctcaatgaaaccataatgaagccagactggggagaaaatgcagggaatatcacagaatgcatcatgggaggatggagacaaccagcgagccctactcaaattaggcctcagagcccgcctcccctgccctactcctgctgtgccatagcccctgaaaccctgaaaatgttctctcttccacaggtcaagagaaaggattccagaggctag

SEQ ID NO: 36 - protein, 173 aa, Homo sapiens
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 37 - protein, 189 aa, Homo sapiens
DKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSAYYTYLLLLLKSVVYFAIITCCLLRRTAFCCNGEKS

SEQ ID NO: 38 - DNA, 5591 bp, Homo sapiens
ataaacaacttgatgcagatgtttcccccaagcccactatttttcttccttcaattgctgaaacaaagctccagaaggctggaacatacctttgtcttcttgagaaatttttccctgatgttattaagatacattggcaagaaaagaagagcaacacgattctgggatcccaggaggggaacaccatgaagactaacgacacatacatgaaatttagctggttaacggtgccagaaaagtcactggacaaagaacacagatgtatcgtcagacatgagaataataaaaacggagttgatcaagaaattatctttcctccaataaagacaggtatgtgtttacgcatatcatctgtcagaacacttctttgaaagtgaatgctgcattttttcctttcagtattaatgaaaaacaaacataaatctttcttaaatattgttacatttaatggtagcataaatgccctgctacttttctatagaattaaaatggtataggttttggagaaaacaaaattgaaaaagttactgaaggtttgtcagcctcagctccattatccaaaataagaaagtcacgtgctggtttttagggttgttagatggattaaagaaacaacatacacagaagcatctagcaacgtgacacgtggtaaacgctcaaaaagtgttctcccttcttttgatgactttacttgatcaggaaataacatatatatgtctttcaggaatgttctgcccaagcaggagagtcactcacctcaatcttgctacccacaaagtttaacctaaaaacaacgggttcattgttgacaaaatgatgtttatctgttgttgacagaatgatgtttatctaaaaacagttccaattttctatttcctttgctgagacacaaaggggaggcaaatgtgcaaagcttgagggtagtcttaccactgtgcttaagtgttctgatttttctagtgatcagggcaaaataaaaagtatagtaagttccaaggcagtgaatattatacaggagagaagttacagttttataatgtgttttcctttacactaaattctaaaagtaaaaagtctttttttttttttgacagagtttcactcttgttgcccaagcaggtgtgctatggtatgatctcagctcactgcaacctccacctcccgggttcaagtgattctcttacttcagcctcccgacaggctgggattgcaggcgcctgccaccacacctggctaatttttgtgtttttagtagagatggggtttcaccatgttggccaggctggtctcaaattcctgacctcaagtgatccatccacctcggcctccaagtgctgggattatgggcgtcagccactgtgcccagcctaaaagtaaaatgtctttcatgagcttcccaaggcagctacgttaaggaggacacttctcttaatgtcattctacagtagatttctaatgctctttcttggaagtttgtttttctgagaaaagctaaaaatataacatggaagtgatcatattatataatcaatgaagtgcttttcaaggagataaaactaatctggtccacacttgcaaccaaccttgattgagagagagagagaactcaggatacacttgaagattttattatggggaacagttactttattctttttacctcaatcaatgcatggaaataagtgatagtcattttcatttatcttttaataaatgaagtcaccatgaggaaaataaaaagacattgaaaacccattaaagtcagcccttaaagatatttggacatgcagacttgataactaacgtttgcattcttgagacttacccaaaacccatacctcaagtccaagtttttagaattcatgaaataaagatctcagtgagtgcataaaattgcgcaccagaatcatatccgtatagacaagaacacatctactagaaaaataataaaccaacacaccaatgcaactgtgttttcttctgttttaaagtatgttgtctttgtatgcatgtttgcttcttcctttttttttttaacatcacagataaattcaactctcacctcaggttttattgagagaactgtcaatgtgacttggcctctgtctttctagtcccagaaagaattgcactgaaatctgagctcctgtaataaaaacaaccatttgctgagagtaattaacatactgaaagagattttcttagagtacacaatggtgacattatattgcctctttataaataactttctatctatttctgtggattattcctacaaagtacttttcatatgtccaatttcttttcttcccctacaactactgtctgaatactggctctgctatttgctgatatgattctcggcaagttgcctgcactttttaaactttatttcctcattcagaacatggggccatacataatacaactcacttcagtgttattggggaattaaacaaaaaatgcatgggaagcatttaacatagtgcctgacacaataatgagtactcagtagatgttagcttttattaatattgttgttgttatgtccagaaacactatacctccagaaaatcatgggtacttgctggggacattggggatatgcatgatttggaaaagaatgactgctttttttgcttagatgagaaatttttctaagccagactccttcaaatatgtaagattctgttgtggattcaaggactgaaagaattcttggccgagtgtggtggcttatccctgtaatcccagcattttgtgaggacaaggcaggaagattgcttgagtccaggagtttgaaaccagcctgcgcaacatggcgaaaccctgtctctacaaaaaatacaaacattagctcggagtgagtgctgacatgtgcctgtactcccagctactcagaaggctgagatgggaggatctcatgagcctggggagtttgaggcttcagtgagccgtgatgacaccgtactatactccactccagcctgggtgacagtgagaccctgcctcaaaaaacaaacaaacaaacaaacaaaacaaaattaatctttttgctgatgtcatgtcagcagtgtgtgttgaaggctgtaaagcagccatttgttcagtttatttttccattgaacaagtatttatcaaaaacatactttgtggcagtcactatgctaggagctatgaatacagaaggaaaagtaaatgctcttggatactacactccagttgtgataaaaaagaaaaaatgtattcttcaccaacttcaacatcttgatgtgcaaaaacataatacatgaattagatctacctaattacacagaattagaccaattgtttctggaattgtgggctcatatttttaataactgtcctcctgcctctctgtcgacaggttttataaatattcatttaattacacacacacacacgaacaattgactagtacttgctctcattcttctagatgtcatcacaatggatcccaaagacaattgttcaaaagatgcaaatggtaagcttttgtgtttttcccttcctcctgatcattttgttttgaacttctctggcttgaaaaatcagggaatggattttgctaggttggatgctgcagaatggacctagtgatattttaaattagtccctcattttctaggagttgtattaacaaacctaactactgctttggggtatgagatgactgtaaattagagagggtacagtggtatagtgatatgcttttaattatttcaaaaaaaagattttattcattcatgtgtcttttttctttttcttttcttttttttttttttttggacagagtcttgctctgtcacccaggctggagtgcggtggcagtatctcagctcaccacaacctccgcctcccggcttcaagtgattctcctgcctcagcttctcgagtagctgggactacaggcgcgtgccaccatgcccggctaatttttgtatttttagtagagttggggtttcaccatgttggccaggatggcctcgaatttgtgacctcgtgatctgccccctcgccctcccgaactgttgggattacaggcgtgagtcactgtgcccggcctcctgtcctgtcttttgtttaatgactgggaaaaacatgataccatgttgcttctcgagttgttttgttttagtctttggtctttgctagtagctaataacacgaactagtgtttatcaagtgctttttacacagaagggcttgggctgtgttctgcattttcttgtttaaccctcttaaaactcctataaaatggtacatatttttctcccaatttacagtccctttaaagcaaataattataaaaatccctatacatgtcacacagctagatctgggatttcaaatcaggccatcaaacaaagagtttatgtacttagtaagttttctgttctttttctacaatagagtcagatagcaagaaattaccaagccaggaacctgaaacaaaacggacatcatgtggggctgggtgggtgcatgggctttgcagactggactttcactccagctcttttaatgattaggtgtaagtgacctacattttgtgagcaacagttttctcatcagccaacaaagaataattacaccagattcacagttattgaagagataaaggcatgaatgtgagatgtctggcatagggcatctcatttagcagacacagaatgagtacttgtttctggctttttctctctacatatgcacaaagaatgcgactagaagcatgggctctagccctgctcaactttcctctatttccaataccaaggggctctgacttaggctgccacaccaggcaaggagggcagtaccacctcacttgaccaagggcagggagtcacggacacatcacttcttgagatccttttccacaccaaggactgatgtttctggaattctcactttatgaagacaaaacatataaatggaaattttctcaggtagagactcactcttgtagctcattgagtaggcactagtggtccacccccactgtctttacttattccttgacatcacatatctcttgcaaaacctcaaataatattaaatgcaatcacccaataatagcatagccataattagaggcatttaggaaagacaggtgagtgtgccacaactacctaacacatcagcaaatctggattaaccactttctttgattttccacaatgcaaccttactttttaatagttgggaatgttctaagtgaatttagcagaggttgttaatcaacttgaaagctgaattctgacttgtctgactcttggtggtgctggtagcagtagatgtttacttttaggttttggtggtggtggaatatcacttcaacgtaaatcatcagaaataagtatttgtgaacccctctcgcattaatgtatcttattctgtaaaaagaacatgtgcaatttctcttagatacactactgctgcagctcacaaacacctctgcatattacatgtacctcctcctgctcctcaagagtgtggtctattttgccatcatcacctgctgtctgcttagaagaacggctttctgctgcaatggagagaaatcataa

SEQ ID NO: 39 - DNA, 9549 bp, Homo sapiens
ataaacaacttgatgcagatgtttcccccaagcccactatttttcttccttcgattgctgaaacaaaactccagaaggctggaacatacctttgtcttcttgagaaatttttcccagatattattaagatacattggcaagaaaagaagagcaacacgattctgggatcccaggaggggaacaccatgaagactaacgacacatacatgaaatttagctggttaacggtgccagaagagtcactggacaaagaacacagatgtatcgtcagacatgagaataataaaaacggaattgatcaagaaattatctttcctccaataaagacaggtatgtgtttacacatatcatctgtcagaacacttctttgaaagtgaatgctgcattttttcctttcagtattaatgaaaaacataaatctttcttaaaaattgttacatttaatggtagcgtaaatgccctgctacttttctatagaattaaaatggtataggttttggagaaaacaaaattgaaaaagttgctgaaggtttgtcagcctcagctccattatccaaaataagaaagtcacgtgctggtttttagggttgttagatggattaaagaaacaacatacacagaagcatctagcaacgtgacacgtggtaaacgctcaaaaagtgttctcccttcttttgatgactttacttgatcaggaaataacatatatatgtctttcaggaatgttctgcccaagcaggagagtcactcacctcaatcttgctacccacaaagtttaacctaaaaacaacgggttcattgttgacaaaataatgtttatctgaagataactgtagatcatatttatctgtagataatgtttatctgtggagtgtggctctacaaaacatagaatagtcttggtcactgcagttttatagaggccttgggtttttcagagtttcattttatatatcaccataaagtaacatttcataattacaggttggtaaggcttacatgtacaaacattcttccattttccataataaatgcatttcctgccattggtgaatgcagctcaataaacatttattgtacaattatgacacgccaggcttagtggaaatgtggatgaacagacaaggatgagttactgtcctaaggatgatgcatgacagtgcagagaatatactctcttcctgatcactcagggtcactcatgattcatgcgcgaggtcccaaaacagtgcctttgatgcagattctgtacatctctagacgattggtccaagggctgaatgtgctctggcccagtggtccagtctgtcactatatgtcaacatcctgaatatgaacataacagtccaacatctcaagagtgggcatgaaaaggactcattttgtgctttttcctgtggttaacaagtcctttttagcctgggggaacaagcattaacaaaatgtttgaagatctttgccacgtaccattccaaatttctagggtaagtctttagcttttcagatcctgagtttctgcaatgatcaaatgtgatttggacagttgcgttgactttctcctggggctataatggagtgcaaaggaaacaatggcagggaaaatgcttgctttcaaaatggtagcatggatgtgttcattcgtgtagttactgtattaggtatagcctttcctgaaactaactgaagtggggttataaaaacagtcccaattttctatttcctttgctgagacacaaagaggagacaaaagagcaaagcttgagggtagttttaccactgtgcttaagtgttctgatttttccagtgatcagggtgaaataaaaagcatagtaagttccagggcagtgaataccatacaggagacaagttacagttttataatgtgttttactttacactaaattctaaaagtaaaatgtctttttttttttccgagacagagtttcactcttgtagcccaggcaggagtgctatggtgtgatctcggctcacagcaacctccacctcccagtttcaagcgattcttctgcctcagcctcccgagaagttgaaattacaggtgcctggcaccatatctcgctaattattctatttttagtagagatcgggttttaccatgttggccaggctggtctcgaactcctgacttcaagtgatccacccgcctcagcctcccaaagtgctgggattacaggtgtgagtcactgtgccggacctaacagtaaaatgtctttcatgtgcttctcaaggcaactacattaaggaggacacatctcttaatgtcattctacagtagatttctaatgctctttcttggaagtttgtttttctgagaagagctaaaaatataataacatggaagtgatcatattatataatcaatgaagtgctttcaaaggagataaaactaacctggtctgcatttgcaaccagccttgattgagagagagagaactcaggatacacttagagattttattatggggaatagttactttattcattttacctcaatcaatgcatggaaataagtgacagtcattttcatttatcttttaataaataaagtcaccatgaggaaaatgaaaacccattaaagtcagtccttaaagatatttggacatgcagacatgataactaacatttccattcgtgagacttacccaaaacctatacctcaagtccatttcttagaatacatgaaataaagatctcagtgagtgtataaaactgcacaccagaatcatatccgtatagacaagaatacatctactagaaaaatataaaccaaaacaccaaggtgactctgtttttttctgttttaaaatatgttgtctttgtatgcatgtttgcttcttcctttttttttttaaacatcgcagataaattcaactctcacctcagttgagagagaactgtcaatgtgacttggcctctctctttctagtcccagaaagaattgcactgaaatgctgagctcctgtaataaaaatgaccatttgctgagagtaattaacatactgaaagagattttcttagaatagtgcacaatggcccaatggtgacattatattgtctctttataaattattttctatctatttctgtggattatttctacaaagcacttttcatatgtccaattccttttattcccctacaagtactgactgactactggctctgctgttcactgatatgactttcggcaagttgcctgcactttttaaacgttatttcctcattcagaacatggggccatacaaaatacaactcacttcagtgttattggggaattaaacaaataaatgcatgggaagcatttaacatagtgcctgacacaataatgagcactcagtagatgttagcttttattaatattgttgttgctatgtccagaaacactatacctccagaaaatcatgggtacttgctggggacgttggggatatgcatgattttgaaaggagtgactgctctttactgctcagatgagaaatttttctaagccagactccttcaaacatgtaagattctgttgtggattctaggactgaaagaattcttggccgagtgtggtggcttatcctggtaatctcatcatttgggaggacaaggcaggaagattgcttgagcccaggagttggaaacaagcctggacaacatggcgaaaccctgtctctacaaaaaatacaaacattagctggtcatgggagtgagtgcctgtactcccagctactcaggaggctaagataggaggatcacctgagcctgggcagtttgaggtttcagtgagccgtgatgacaccatactatactccactccagcctgggtgacagtgacatcctgcctcaaaaaaacccccaaaattattctttttgctgatttcatgtcagcagtgtgtgctgaaggctgtaaagtagccacttgttctgtttatttttccattgaacaagtatttatcaaaaacgtactttgtggaaggcactgtgctaggaactatgcatacagaaggaaaaccaaatgttcttggatactacactccagttgtgataaaaaagaaaaaagtattcttcacaaacttcaacattttgatgtgcaaaaacataatatatgaattagatctacctaactacacagaattagaccaattatttctgggattatgggctcatatttttaataactgtcctcctacctctctgttgacaggttttataaatattcatttaattacacacagtcacagacacactcagacacacacacatacacacacacacacaccttgacaaataatgggcatgaacaattgactggtacttgctctcattcttctagatgtcaccacagtggatcccaaatacaattattcaaaggatgcaaatggtaagtttttgtgttttttatttcctcctgatcattttaagttttgaacttctctggcttgaaaaatcagggaatggattttgctaggttggatgctgcagaatggacctaatcatattttaaattagtccctctttttctaggagttgtattaacaaacctaactactgcttcatgtaagagatgactgtaaattgaagggtacagtgatatgctttcagttatttcaaaaaacagactttactcatccatgtgtcttttttcttttcttttttttcttttttgagacggagtctcgctctgttgaacaggctggattgcagtgacgcgatctcacctcactacaacctccgcctctggagttcaagcgattctccagcctcagcttctcaagtagctgggactacaggcacatgccaccatgtccgggtcatctttgtatttttagcagagaccgggtttcactatgttggccaggctggtctagaattcctgacttcgtgatctgccccctcagccctccgaagtgctgggattacagacgtgagtcactgtgcccggcctaacagtaaaatgtctttcatgcgcttctcaaggcaactacgttaaggaggacacttctcttaatgtcattctacagtagatttctaatgctctttcttggaagtttgtttttctgagaaaagctaaaaatataacatggaagtgatcatattgtataatcaatgaagtgcttttcaaggagataaaactaatctggtccacgtttgcaaccaaccttgattgagagagagagagaactcaggatacacttggagattttattatggggaatagttactttattcttttttcctcaatcaattcatggaaataagtgatagtcatattcatttatcttttaataaatgaagtcaccatgaggaaaataaaaagacattgaaaacccattaaagttagcccttaaagatatttggacatgcagacttgataactaacgtttgcattcttgagacttacccaaaacccatacctcaagtccatgtttttagaattcatgaaataaagatctcagtgagtgcataaaattgcgcaccagaatcatatccgtatagacaagaacacatctactagaaaaataataaaccaacacaccaatgcaactgtgttttcttctgttttaaaatatgttgtctttgtatgcatgtttgcttcttccttttttttttttaacatcacagataaattcaactctcacctcaggttttattgagagaactgtcaatgtgacttggcctctgtctttctagtcccagaaagaatcgcactgaaatgctgagctcctgtaataaaaatgaccatttgctgagagtaattaacatactgaaagagattttcttagagtacacaatggtgacattatattgtctctttataaataactttctatctatttctgtggattattcctacaaagtacttttcatatgtccagtttcttttcttcccctacaactaccgtctgaatactggctctgctatttgctgatatgattctcggcaagttgcctgcactttttaaactttatttcctcattcagaacatggggccatgtaatactcatgtacgtgagtattacgtaataatgctcacttaagtgttactggggaattaaacaaaaaaatgcatggcaagcatttaacatagtgcctgacacaataatgagcactcagtagatgttagattttattaatattgttgttgttatgtccggaaacactatacctccagaaaatcatgggtacttgcttgggatgttggggatatgcatgatttggaaaggtatgactgcttttttctgcttagatgagaaatttttctaagccagactccttcaaatatgtaagattctgttgtggattctaggacggaaagaattcttggtcaggtgtggtttcttatccctgtaatcccagaattttgggaggacaaggcaggaagattgcttgagcccaggagtttgaaaccagcctgggcaacaagacgaaaccctgtctctacaaaagtacataaattagcttggcttggtggtgtgtgcctgtattaccagctattcgggagactgagatgggaggatctcctgaacctgtgaagtttgaggcttcagtgagccgtgatgacaccatactatactcgactccagcctgtgcgacagtgagactctgcgtcaaaaaaaaaaccccaaaattattgtttttgctgatttcaggtcagcagtgtgtgctgaagggtgtaaagtagccacttgatcagtttatttttccactgaacaagtatttatcaaaaacatactttgtggtctgtttttgataaataaaaaggcactgtgctaggagccatgaatacagaaggaaaaccaaatgttcttggatactacactccagttgtgataaaaaagaaaaatgtattcttcacgaacttcaacattttgatatgcaaaaacatagtatataaattagatctacctgattacgtagaatcagaccaattatttctggaattgagggctcatatttttaataactgtcctcctgcctctctgttgacaggttttataaatattcatttaattacacacacacacacacacaccttgacaaataatggacatgaacaattgactagtacttgctctcattcttctagatgtcatcacaatggatcccaaagacaattggtcaaaagatgcaaatggtaagcttttgtgtttttcctttcctcctgatcattttaagttttgaacttctctggcttgaaaaatcagggaatgggccgggtgcggtggctcacgcctgtaatcccagcactttgggaggccgaggcgggcggatcacgaggtcaggagatcgagaccatcccggctaaaacggtgaaaccccgtctctactaaaaatacaaaaaattagccgggcttagtggcgggcgcctgtagtcccagctacttgggaggctgaggcaggagaatggcgtgaacccgggaggcggagcttgcagtgagccgagattgcgccactgcactccactccagcctgggcgacagagcgagactccgtctcaaaaaaaaaaaaaaaaaaaaaaaaagaaaaatcagggaatggattttgctaggttggatgctgcagaatggacctagtgatattttaaattagtccctctttttctaggagttgtattaacaaacctaactactgcttcgggtatgagatgactgtaaattagagggtacagtgatatgctttcagttatttcaaaaaacagactttattcatccgtctgtcttttttttttttttttttttttttttttgagacggaggagtctcactctatcacccaggctggagtgcagtggcgcgatctcggctcaccataacctccgccttactggttcaagcgattctccagcctcagcttctcaagtagctgggactacaggtgcacaccaccatacctggctaatttttgtatttttaatagagatggggtttcaccacgctggccaggatggtcttgaattcttgacctcgtgatctgccccctcgggctcccaaacttctgggattataggcgtgagccactgtgcccggccttctgtcttttgttataatgactggggaaaacatgataccatgttgcttcttgagttgttttgttttagtctttggtctttgctagtagctaataacacgaactagtgtttatcaagtgctttttacacagaagggcttgttctgcattttctagtttaatcatcttaatactcctataaagtagtacaatatattttctcccattttacagtccctttaaagtaaataactataaaaatcccttatacatgtcacacagctaggtctggcatttcaaatcaggacatcaaacaaagaattcgtgcagttactaagtcctctattttttctacaatagaaaaaatagcaagaattacagatagcaagacattacaaggcaggaatctgaaacgaaagggacataatgtggggctgggtgggtgcatgagctttgcagactagactttcattccagctcttttaatgattaggtgtaagtgacctacattttgtgagtaacagttttctcatcagccaactaagaataattacaccagattcacagttattgaagagataagggcatgaatgtgagatgtctggcgtagggtatctcatttagcagacacagaatgaatacttgtttctggctttttctctctacatatgcacaaagaatgtgactagaagcattggctctagccctgctcaactttcctctatttccaataccaaggggctctgacttaggctgccacaccaggcaaggaggggcagtaccacctcacttgaccaagggcagggagtcacggacacatcacttcctgagatccttttccacaccaaggactgatgtttctggaattctcactttatgaagacaaaacatataaatggaaatttctgcaggaagagactcactcttgtagctcattgagtaggcactagtggtccacccccactgtctttacttattccttgacatcacatatctcttgtaaaacctcaaataatgttaaatgcaatcacccaataatagcatagccataattagaggcatttaggaaagacaggtgagtgtgccacaactacctaacacatcagcaaatctggattaaccactttctttgattttccacaatgcaaccttactttttaatagttgggaatgttctaagtgaatttagcagaggttgttaatcaacttgaaagctgaattctgacttgtctgactcttggtggtgctggtagcagtagatgtttacttttaggttttggtggtggtggaatatcacttcaacgtaaatcatcagaaataagtatttgtgaacccctctcgcattaatatatcttattctgtaaaaagaacatgtgcaatttctcttagatacactactgctgcagctcacaaacacctctgcatattacacgtacctcctcctgctcctcaagagtgtggtctattttgccatcatcacctgctgtctgcttagaagaacggctttctgctgcaatggagagaaatcataa

SEQ ID NO: 40 - protein, 153 aa, Homo sapiens
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 41 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 42 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 43 - protein, 127 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRTADTQALLRNDQVYQPLRDRDDAQYSHLGGNWARNK

SEQ ID NO: 44 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 45 - protein, 48 aa, Artificial Sequence
  Synthetic
QRRKYRSNKGESPVEPAEPCHYSCPREEEGSTIPIQEDYRKPEPACSP

SEQ ID NO: 46 - protein, 67 aa, Artificial Sequence
  Synthetic
VLVFIKKVAKKPTNKAPHPKQEPQEINFPDDLPGSNTAAPVQETLHGCQPVTQEDGKESRISVQERQ

SEQ ID NO: 47 - protein, 51 aa, Artificial Sequence
  Synthetic
MGWIRGRRSRHSWEMSEFHNYNLDLKKSDFSTRWQKQRCPVVKSKCRENAS

SEQ ID NO: 48 - protein, 256 aa, Mus musculus
MGNNCYNVVVIVLLLVGCEKVGAVQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGPGGHSLQVLTLFLALTSALLLALIFITLLFSVLKWIRKKFPHIFKQPFKKTTGAAQEEDACSCRCPQEEEGGGGGYEL

SEQ ID NO: 49 - protein, 5 aa, Artificial Sequence
  Synthetic
SYAIS

SEQ ID NO: 50 - protein, 17 aa, Artificial Sequence
  Synthetic
RIIRFLGIANYAQKFQG

SEQ ID NO: 51 - protein, 12 aa, Artificial Sequence
  Synthetic
EPGERDPDAVDI

SEQ ID NO: 52 - protein, 11 aa, Artificial Sequence
  Synthetic
RASQGIRSWIA

SEQ ID NO: 53 - protein, 7 aa, Artificial Sequence
  Synthetic
AASSLQS

SEQ ID NO: 54 - protein, 9 aa, Artificial Sequence
  Synthetic
QQYNSYPLT

SEQ ID NO: 55 - protein, 121 aa, Artificial Sequence
  Synthetic
QVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSS

SEQ ID NO: 56 - protein, 107 aa, Artificial Sequence
  Synthetic
DIQMTQSPSSISASVGDRVTITCRASQGIRSWIAWYQQKPEKAPKSLIYAASSLQSGVPSRFSGSGSGTDFTLTISSLQPEDFATYYCQQYNSYPLTFGGGTKVEIK

SEQ ID NO: 57 - protein, 243 aa, Artificial Sequence
  Synthetic
QVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSGGGGSGGGGSGGGGSDIQMTQSPSSISASVGDRVTITCRASQGIRSWIAWYQQKPEKAPKSLIYAASSLQSGVPSRFSGSGSGTDFTLTISSLQPEDFATYYCQQYNSYPLTFGGGTKVEIK

SEQ ID NO: 58 - protein, 5 aa, Artificial Sequence
  Synthetic
SYAIS

SEQ ID NO: 59 - protein, 15 aa, Artificial Sequence
  Synthetic
IIVFLGKVNYAQRFQ

SEQ ID NO: 60 - protein, 12 aa, Artificial Sequence
  Synthetic
EPGARDPDAFDI

SEQ ID NO: 61 - protein, 11 aa, Artificial Sequence
  Synthetic
RASQGIRSWLA

SEQ ID NO: 62 - protein, 7 aa, Artificial Sequence
  Synthetic
AASSLQS

SEQ ID NO: 63 - protein, 9 aa, Artificial Sequence
  Synthetic
QQYNNYPLT

SEQ ID NO: 64 - protein, 121 aa, Artificial Sequence
  Synthetic
QVQLVQSGAEVKKPGSSVKVSCKPSGGTFRSYAISWVRQAPGQGLEWMRIIVFLGKVNYAQRFQGVTLTADKSTTTAYMELSSLRSEDTAVYYCTGEPGARDPDAFDIWGQGTMVTVSSVL

SEQ ID NO: 65 - protein, 107 aa, Artificial Sequence
  Synthetic
DIQMTQSPSSLSASVGDRVTITCRASQGIRSWLAWYQQKPEKAPKSLIYAASSLQSGVPSRFSGSGSGTDFTLTISSLQPEDFATYYCQQYNNYPLTFGGGTKVEIK

SEQ ID NO: 66 - protein, 243 aa, Artificial Sequence
  Synthetic
QVQLVQSGAEVKKPGSSVKVSCKPSGGTFRSYAISWVRQAPGQGLEWMRIIVFLGKVNYAQRFQGVTLTADKSTTTAYMELSSLRSEDTAVYYCTGEPGARDPDAFDIWGQGTMVTVSSVLGGGGSGGGGSGGGGSDIQMTQSPSSLSASVGDRVTITCRASQGIRSWLAWYQQKPEKAPKSLIYAASSLQSGVPSRFSGSGSGTDFTLTISSLQPEDFATYYCQQYNNYPLTFGGGTKVEIK

SEQ ID NO: 67 - protein, 5 aa, Artificial Sequence
  Synthetic
SYAIS

SEQ ID NO: 68 - protein, 17 aa, Artificial Sequence
  Synthetic
RIIRFLGKTNHAQKFQG

SEQ ID NO: 69 - protein, 12 aa, Artificial Sequence
  Synthetic
EPGDRDPDAVDI

SEQ ID NO: 70 - protein, 11 aa, Artificial Sequence
  Synthetic
RASQGIRSWLA

SEQ ID NO: 71 - protein, 7 aa, Artificial Sequence
  Synthetic
AASSLQS

SEQ ID NO: 72 - protein, 9 aa, Artificial Sequence
  Synthetic
QQYNSYPLT

SEQ ID NO: 73 - protein, 121 aa, Artificial Sequence
  Synthetic
QVQLVQSGAEVKKPGSSVKVSCKAFGGTFSSYAISWVRQAPGQGLEWMGRIIRFLGKTNHAQKFQGRVTLTADKSTNTAYMELSSLRSEDTAVYYCAGEPGDRDPDAVDIWGQGTMVTVSS

SEQ ID NO: 74 - protein, 107 aa, Artificial Sequence
  Synthetic
DIQMTQSPSSLSASVGDRVTITCRASQGIRSWLAWYQQKPEKAPKSLIYAASSLQSGVPSRFSGSGSGTDFTLTISSLQPEDFATYYCQQYNSYPLTFGGGTKVEIK

SEQ ID NO: 75 - protein, 233 aa, Artificial Sequence
  Synthetic
QVQLVQSGAEVKKPGSSVKVSCKAFGGTFSSYAISWVRQAPGQGLEWMGRIIRFLGKTNHAQKFQGRVTLTADKSTNTAYMELSSLRSEDTAVYYCAGEPGDRDPDAVDIWGQGTMVTVSSGGSGGDIQMTQSPSSLSASVGDRVTITCRASQGIRSWLAWYQQKPEKAPKSLIYAASSLQSGVPSRFSGSGSGTDFTLTISSLQPEDFATYYCQQYNSYPLTFGGGTKVEIK

SEQ ID NO: 76 - protein, 5 aa, Artificial Sequence
  Synthetic
SFAMS

SEQ ID NO: 77 - protein, 17 aa, Artificial Sequence
  Synthetic
AISGSGGGTYYADSVKG

SEQ ID NO: 78 - protein, 13 aa, Artificial Sequence
  Synthetic
DKILWFGEPVFDY

SEQ ID NO: 79 - protein, 11 aa, Artificial Sequence
  Synthetic
RASQSVSSYLA

SEQ ID NO: 80 - protein, 7 aa, Artificial Sequence
  Synthetic
DASNRAT

SEQ ID NO: 81 - protein, 9 aa, Artificial Sequence
  Synthetic
QQRSNWPPT

SEQ ID NO: 82 - protein, 122 aa, Artificial Sequence [sequence omitted]

SEQ ID NO: 83 - protein, 107 aa, Artificial Sequence [sequence omitted]

SEQ ID NO: 84 - protein, 234 aa, Artificial Sequence
  Synthetic
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSGGSGGEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIK

SEQ ID NO: 85 - protein, 19 aa, Artificial Sequence
  Synthetic
GGGGSGGGGSGGGSGGGGS

SEQ ID NO: 86 - protein, 15 aa, Artificial Sequence
  Synthetic
GGGGSGGGGSGGGGS

SEQ ID NO: 87 - protein, 24 aa, Artificial Sequence
  Synthetic
GGGGSGGGGSGGGGSGGGSGGGGS

SEQ ID NO: 88 - protein, 29 aa, Artificial Sequence
  Synthetic
GGGGSGGGGSGGGGSGGGGSGGGSGGGGS

SEQ ID NO: 89 - protein, 5 aa, Artificial Sequence
  Synthetic
GGGGS

SEQ ID NO: 90 - protein, 10 aa, Artificial Sequence
  Synthetic
GGGGSGGGGS

SEQ ID NO: 91 - protein, 5 aa, Artificial Sequence
  Synthetic
GGSGG

SEQ ID NO: 92 - protein, 41 aa, Artificial Sequence
  Synthetic
RSKRSRLLHSDYMNMTPRRPGPTRKHYQPYAPPRDFAAYRS

SEQ ID NO: 93 - protein, 41 aa, Artificial Sequence
  Synthetic
RGRKKLLYIFKQPFMRPVQTTQEEDGCSCRFPEEEEGGCEL

SEQ ID NO: 94 - DNA, 123 bp, Artificial Sequence
  Synthetic
cggggcagaaagaaactcctgtatatattcaaacaaccatttatgagaccagtacaaactactcaagaggaagatggctgtagctgccgatttccagaagaagaagaaggaggatgtgaactg

SEQ ID NO: 95 - protein, 27 aa, Artificial Sequence
  Synthetic
FWVLVVVGGVLACYSLLVTVAFIIFWV

SEQ ID NO: 96 - DNA, 81 bp, Artificial Sequence
  Synthetic
ttttgggtgctggtggtggttggtggagtcctggcttgctatagcttgctagtaacagtggcctttattattttctgggtg

SEQ ID NO: 97 - protein, 267 aa, Artificial Sequence
  Synthetic
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 98 - protein, 250 aa, Artificial Sequence
  Synthetic
QVQLLESGAELVRPGSSVKISCKASGYAFSSYWMNWVKQRPGQGLEWIGQIWPGDGDTNYNGKFKGKATLTADESSSTAYMQLSSLRSEDSAVYSCARRETTTVGRYYYAMDYWGQGTTVTGGGGSGGGGSGGGGSKLVLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKRRS

SEQ ID NO: 99 - protein, 114 aa, Artificial Sequence
  Synthetic
LRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 100 - protein, 47 aa, Artificial Sequence
  Synthetic
LAILLALYLLRRDQRLPPDAHKPPGGGSFRTPIQEEQADAHSTLAKI

SEQ ID NO: 101 - protein, 38 aa, Artificial Sequence
  Synthetic
CWLTKKKYSSSVHDPNGEYMFMRAVNTAKKSRLTDVTL

SEQ ID NO: 102 - protein, 121 aa, Artificial Sequence
  Synthetic
VWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 103 - protein, 243 aa, Artificial Sequence
  Synthetic
DIQMTQSPSSLSASVGDRVTITCRASQGIRSWLAWYQQKPEKAPKSLIYAASSLQSGVPSRFSGSGSGTDFTLTISSLQPEDFATYYCQQYNSYPLTFGGGTKVEIKGGGGSGGGGSGGGGSQVQLVQSGAEVKKPGSSVKVSCKAFGGTFSSYAISWVRQAPGQGLEWMGRIIRFLGKTNHAQKFQGRVTLTADKSTNTAYMELSSLRSEDTAVYYCAGEPGDRDPDAVDIWGQGTMVTVSS